(19) United States
(12) Patent Application Publication
(10) Pub. No.: US 2010/0119479 A1
(43) Pub. Date: May 13, 2010

(54) THERAPEUTIC ANTIVIRAL PEPTIDES

(75) Inventors: BRAD BUCKMAN, OAKLAND, CA (US); VLADIMIR SEREBRYANY, BURLINGAME, CA (US); SCOTT SEIWERT, PACIFICA, CA (US); LEONID BEIGELMAN, SAN MATEO, CA (US); ANTITSA STOYCHEVA, HALF MOON BAY, CA (US)

Correspondence Address: KNOBBE MARTENS OLSON & BEAR LLP, 2040 MAIN STREET, FOURTEENTH FLOOR, IRVINE, CA, 92614, US

(73) Assignee: INTERMUNE, INC., BRISBANE, CA (US)

(21) Appl. No.: 12/579,275

(22) Filed: Oct. 14, 2009

Related U.S. Application Data
(60) Provisional application No. 61/105,746, filed on Oct. 15, 2008.
(60) Provisional application No. 61/236,741, filed on Aug. 25, 2009.

(51) Int. Cl. A61K 38/21; C07D 209/12; C07D 401/14; C07D 215/16; C07D 413/14; A61K 31/427; A61K 31/708; A61K 31/52; A61K 31/7076; A61K 31/4035; A61K 31/4439; A61K 31/5355; A61K 31/4709; A61P 31/12

(52) U.S. Cl. 424/85.5; 548/467; 546/277.1; 546/155; 544/143; 424/85.7; 514/365; 514/43; 514/263.4; 514/48; 514/46; 514/414; 514/339; 514/235.2; 514/312

(57) ABSTRACT

Disclosed herein are compounds represented by a formula:

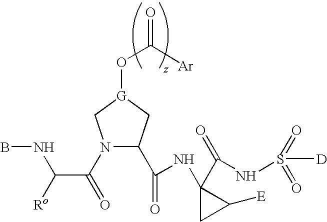

Therapeutic methods, compositions, medicaments, and dosage forms related thereto are also disclosed.

### CROSS-REFERENCE TO RELATED APPLICATIONS

This patent application claims the benefit of U.S. Provisional Patent Application No. 61/105,746, filed Oct. 15, 2008. This application also claims the benefit of U.S. Provisional Patent Application No. 61/236,741, filed Aug. 25, 2009. The disclosures of both of these documents are incorporated by reference in their entireties herein.

### BACKGROUND OF THE INVENTION

1. Field of the Invention

The present invention relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for NS3-mediated processing events and enhances proteolytic efficiency at all sites recognized by NS3. The NS3 protease also exhibits nucleoside triphosphatase and RNA helicase activities. NS5B is an RNA-dependent RNA polymerase involved in the replication of HCV RNA.

### SUMMARY OF THE INVENTION

Some embodiments provide a compound represented by Formula 1:

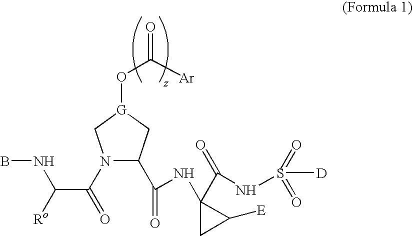

or a pharmaceutically acceptable salt thereof, wherein Ar is optionally substituted fused bicyclic heteroaryl, optionally substituted C₆₋₁₀ aryl, or optionally substituted isoindolinyl; z is 0 or 1; G is

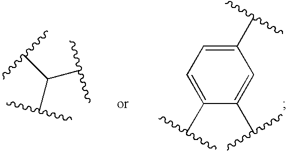

B is optionally substituted C₆₋₁₀ aryl or optionally substituted heteroaryl; R^{o} is H or C₁₋₁₂ hydrocarbyl; D is C₁₋₁₀ alkyl or NR¹¹R¹², wherein R¹¹ and R¹² are independently H or C₁₋₅ alkyl and wherein R¹¹ and R¹² may be connected to form one or more rings; and E is C₁₋₆ hydrocarbyl.

These definitions of Ar, z, G, B, D, and E are understood to apply to structures depicted herein for which any one of those variables are not expressly defined.

One embodiment is a method of inhibiting NS3/NS4 protease activity comprising contacting a NS3/NS4 protease with a compound disclosed herein.

Another embodiment is a method of treating hepatitis by modulating NS3/NS4 protease comprising contacting a NS3/NS4 protease with a compound disclosed herein.

Another embodiment is a pharmaceutical composition comprising: a) a compound disclosed herein; and b) a pharmaceutically acceptable carrier.

Another embodiment is a method of treating a hepatitis C virus infection in an individual, the method comprising administering to the individual an effective amount of a composition comprising a compound disclosed herein.

Another embodiment is a method of treating liver fibrosis in an individual, the method comprising administering to the individual an effective amount of a composition comprising a compound disclosed herein.

Another embodiment is a method of increasing liver function in an individual having a hepatitis C virus infection, the method comprising administering to the individual an effective amount of a composition comprising a compound disclosed herein.

These and other embodiments are described in greater detail below.

### DETAILED DESCRIPTION OF THE PREFERRED EMBODIMENT

### Definitions

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, murines, primates, including simians and humans, mammalian farm animals, mammalian sport animals, and mammalian pets.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

“Treat,” “treating,” “treatment,” or another form thereof refers to the use of a compound, composition, therapeutically active agent, or drug in the diagnosis, cure, mitigation, treatment, or prevention of disease or other undesirable condition in a mammal.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “Type II interferon receptor agonist” refers to any naturally occurring or non-naturally occurring ligand of human Type II interferon receptor that binds to and causes signal transduction via the receptor. Type II interferon receptor agonists include native human interferon-γ, recombinant IFN-γ species, glycosylated IFN-γ species, pegylated IFN-γ species, modified or variant IFN-γ species, IFN-γ fusion proteins, antibody agonists specific for the receptor, non-peptide agonists, and the like.

As used herein, the term “a Type III interferon receptor agonist” refers to any naturally occurring or non-naturally occurring ligand of human IL-28 receptor α (“IL-28R”), the amino acid sequence of which is described by Sheppard, et al., infra., that binds to and causes signal transduction via the receptor.

As used herein, the term “interferon receptor agonist” refers to any Type I interferon receptor agonist, Type II interferon receptor agonist, or Type III interferon receptor agonist.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

The term “aryl” refers to an aromatic ring or aromatic ring system such as phenyl, naphthyl, biphenyl, and the like. The term “C₆₋₁₀ aryl” refers to an aromatic ring or ring system having from 6 to 10 carbon atoms.

The term “heteroaryl” refers to an aromatic ring or aromatic ring system having one or more oxygen atoms, nitrogen atoms, sulfur atoms, or a combination thereof, which are part the ring or ring system. Examples include thienyl, furyl, pyridinyl, quinolinyl, thiazolyl, benzooxazolyl, benzothiazolyl, benzoimidazolyl, benzothiazolyl, benzothienyl, benzofuryl, isoindolinyl, pyridinyl, imidazolyl, thiazolyl, oxazolyl, and the like. The term “fused bicyclic heteroaryl” refers to heteroaryl having a ring system of two rings, wherein two adjacent ring atoms are shared by both rings of the system. Examples include, but are not limited to, quinolinyl, benzooxazolyl, benzothiazolyl, benzoimidazolyl, benzothiazolyl, benzothienyl, benzofuryl, isoindolinyl, and the like

The term “optionally substituted” is intended to mean that the feature which is “optionally substituted” may be unsubstituted, or have one or more substituents. Thus, for example, “optionally substituted phenyl” may be unsubstituted phenyl, or may be phenyl with one or more substituents. A “substituent” refers to a moiety that replaces one or more hydrogen atoms of the parent group for which it is a substituent. In some embodiments, a substituent consists of from 0-10 carbon atoms, from 0-26 hydrogen atoms, from 0-5 oxygen atoms, from 0-5 nitrogen atoms, from 0-5 sulfur atoms, from 0-7 fluorine atoms, from 0-3 chlorine atoms, from 0-3 bromine atoms, and/or from 0-3 iodine atoms. Examples include C₁-C₆ alkyl (such as methyl; ethyl; propyl isomers including n-propyl, isopropyl, etc.; butyl isomers such as n-butyl, t-butyl, etc.; pentyl isomers; hexyl isomers; etc.), C₁-C₆ alkenyl, C₁-C₆ alkynyl, C₃-C₆ cycloalkyl (such as cyclopropyl; cyclobutyl isomers including cyclobutyl, methylcyclopropyl, etc.; cyclpentyl isomers; cyclohexyl isomer; etc.), C₃-C₆ heterocycloalkyl (e.g., tetrahydrofuryl), halo (e.g., chloro, bromo, iodo and fluoro), C₁-C₆ haloalkyl (such as C₁-C₆ fluoroalkyl, including C₁-C₆ perfluoroalkyl, e.g. CF₃, C₂F₅, C₃F₇, etc), cyano, hydroxy, C₁-C₆ alkoxy (such as methoxy, ethoxy, propoxy isomers, butoxy isomers, pentoxy isomers, hexoxy isomers, etc.), other C₁-C₆ ethers (such as alkylethylene oxide, alkyldiethylene oxide,

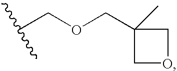

etc.), C₁-C₆ haloalkoxy (such as C₁-C₆ fluoroalkoxy, including C₁-C₆ perfluoroalkoxy such as OCF₃), C₁-C₆ carboxylate esters, C₁-C₁₀ amides (such as CONCH₂CH₂N(CH₃)₂,

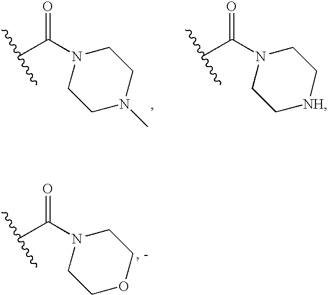

NCOCH₂OCH₂CH₂OCH₂CH₂OCH₃, —NCOCH₂OCH₃, (such as —CO₂CH₃, —CO₂CH₂CH₃, etc.), C₁-C₁₀ sulfonamides (such as

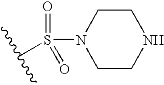

), C₁-C₆ aryloxy, sulfhydryl (mercapto), C₁-C₆ alkylthio, arylthio, mono- and di-(C₁-C₆)alkylamino, quaternary ammonium salts, amino(C₁-C₆)alkoxy, hydroxy(C₁-C₆)alkylamino, amino(C₁-C₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, thiocarboxy,

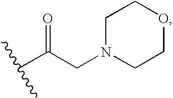

optionally substituted aryl (e.g. any aryl, such as C₆-C₁₂ aryl, optionally substituted with any of the above substituents), optionally substituted heteroaryl (e.g. any heteroaryl, such as optionally substituted C₃-C₁₀ heteroaryl, including optionally substituted thiazolyl, optionally substituted with any of the above substituents such as alkyl, including isopropyl) and combinations thereof. The protecting groups that can form the protective derivatives of the above substituents are known to those of skill in the art and can be found in references such as Greene and Wuts Protective Groups in Organic Synthesis; John Wiley and Sons: New York, 1999.

The term “hydrocarbyl” refers to a moiety containing only hydrogen and carbon atoms including alkyl, alkenyl, and alkynyl moieties. The term “C₁₋₁₀ hydrocarbyl” refers to hydrocarbyl having 1, 2, 3, 4, 5, 6, 7, 8, 9, or 10 carbon atoms. The term “C₁₋₆ hydrocarbyl” refers to hydrocarbyl having 1, 2, 3, 4, 5, or 6 carbon atoms. The term “C₄₋₆ hydrocarbyl” refers to hydrocarbyl having 4, 5, or 6 carbon atoms.

The term “alkyl” refers to a hydrocarbon moiety which has no double or triple bonds. “C₁₋₁₀ alkyl” refers to alkyl having 1, 2, 3, 4, 5, 6, 7, 8, 9, or 10 carbon atoms. “C₁₋₆ alkyl” refers to alkyl having 1, 2, 3, 4, 5, or 6 carbon atoms. “C₁₋₄ alkyl” refers to alkyl having 1, 2, 3, or 4 carbon atoms. Examples include methyl, ethyl, propyl isomer, cyclopropyl, butyl isomers, cyclobutyl, etc. “C₁₋₃ alkyl” refers to alkyl having 1, 2, or 3 carbon atoms such as methyl, ethyl, propyl, isopropyl, cyclopropyl, etc.

The term “alkyl ether” refers to a moiety composed of carbon, hydrogen, and at least one —O— group. In some embodiments, if the alkyl ether comprises more than one —O— group, there may be at least 2 carbon atoms for every —O— group in alkyl ether. C₁₋₁₀alkyl ether is composed of 1, 2, 3, 4, 5, 6, 7, 8, 9, or 10 carbon atoms, hydrogen, and 1, 2, 3, 4, or 5-O— groups. Examples include —OCH₃, —CH₂OCH₃, —OCH₂CH₂, —OCH₂CH₂OCH₂CH₂OCH₃, etc. Also included are cyclic ether structures such as oxetanyl, tetrahydropyranyl, tetrahydrofuranyl, etc.

The term “alkoxy” refers to a moiety of the formula —O-alkyl. The term “C₁₋₆ alkoxy” refers to alkoxy wherein the alkyl group has 1, 2, 3, 4, 5, or 6 carbon atoms.

The term “alkyl amine” refers a moiety composed of carbon, hydrogen, and at least one nitrogen atom. “C₁₋₁₀ alkyl amine” refers to an amine composed of 1, 2, 3, 4, 5, 6, 7, 8, 9, or 10 carbon atoms, hydrogen, and from 1 to 3 nitrogen atoms. Examples include —NHCH₃, —N(CH₃)₂, —NHCH₂CH₂NH₂, etc. Also included are cyclic amine structures such as piperidinyl, piperazinyl, etc.

A combination C₁₋₁₀ alkyl, C₁₋₁₀ alkyl ether, and C₁₋₁₀ alkyl amine is a moiety composed of any combination of alkyl, alkyl ether, and alkyl amine, which has from 1 to 10 carbon atoms, provided that there at least 2 carbon atoms for every nitrogen atom or —O-group. For example, moieties such as —CH₂OCH₂CH₂NHCH₃, —CH₂NCH₂CH₂OCH₂CH₃, etc., are contemplated. Also included are cyclic ether-amine structures such as morpholino.

The term “perfluoroalkyl” refers to a moiety composed of carbon and fluorine which has no double or triple bonds. “C₁₋₆ perfluoroalkyl” refers to perfluoroalkyl having, 1, 2, 3, 4, 5, or 6 carbon atoms. Examples include CF₃, C₂F₅, C₃F₇, C₄F₉, C₅F₁₁, etc.

The term “perfluoroalkoxy” refers to a moiety of the formula —O-perfluoroalkyl. The term “C₁₋₆ perfluoroalkoxyl” refers to perfluoroalkoxy wherein the perfluoroalkyl group has 1, 2, 3, 4, 5, or 6 carbonatoms.

Use of the term “having,” such as in “having from 0 to 3 substituents” is intended to indicate that the number of substituents is 0, 1, 2, or 3. Similarly, “having from 1 to 3” carbon atoms is intended to indicate that the number of carbon atoms is 1, 2, or 3. Similar use of the word “having” where it refers to a number of atoms, moieties, or substituents are intended to have the same meaning.

“4-Fluoroisoindolin-2-yl” refers to:

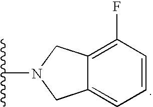

“4-chloroisoindolin-2-yl” refers to:

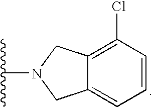

“4-Fluorophenyl” refers to:

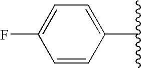

“3-Trifluoromethylphenyl” refers to:

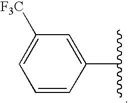

“3-Chlorophenyl” refers to:

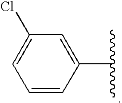

“Thiazolyl” refers to the basic ring structure below. Attachment to the rest of the molecule may occur at any possible position. When optionally substituted, the addition of a substituent may occur at any possible position.

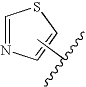

“Quinolinyl” refers to the basic ring structure below. Attachment to the rest of the molecule may occur at any possible position. When optionally substituted, the addition of a substituent may occur at any possible position.

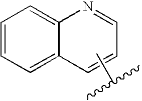

“Quinolin-4-yl” refers to the basic ring structure below. When optionally substituted, the addition of a substituent may occur at any possible position.

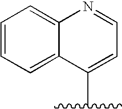

“Isoquinolinyl” refers to the basic ring structure below. Attachment to the rest of the molecule may occur at any possible position. When optionally substituted, the addition of a substituent may occur at any possible position.

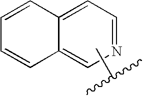

“3-(Thiazol-2-yl)isoquinolinyl” refers to the basic ring structure below. Attachment to the rest of the molecule may occur at any possible position on the isoquinolinyl ring system. When optionally substituted, the addition of a substituent may occur at any possible position.

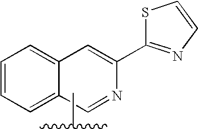

“3-(Thiazol-2-yl)isoquinolin-1-yl” refers to the basic ring structure below. When optionally substituted, the addition of a substituent may occur at any possible position.

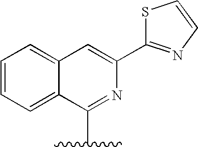

“Isoindolinyl” refers to the basic ring structure below. Attachment to the rest of the molecule may occur at any possible position. When optionally substituted, the addition of a substituent may occur at any possible position.

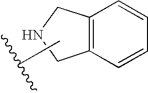

“Benzooxazolyl” refers to the basic ring structure below. Attachment to the rest of the molecule may occur at any possible position. When optionally substituted, the addition of a substituent may occur at any possible position.

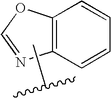

“Benzooxazol-2-yl” refers to the basic ring structure below. When optionally substituted, the addition of a substituent may occur at any possible position.

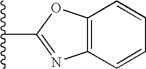

“Benzothiazolyl” refers to the basic ring structure below. Attachment to the rest of the molecule may occur at any possible position. When optionally substituted, the addition of a substituent may occur at any possible position.

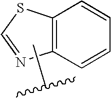

“Benzothiazol-2-yl” refers to the basic ring structure below. When optionally substituted, the addition of a substituent may occur at any possible position.

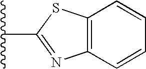

“Benzoimidazol-2-yl” refers to the basic ring structure below. When optionally substituted, the addition of a substituent may occur at any possible position.

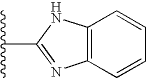

“Isoindolin-2-yl” refers to the basic ring structure below. When optionally substituted, the addition of a substituent may occur at any possible position.

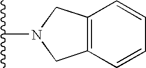

The term “five or six-membered heteroaryl” refers to a monocyclic heteroaryl ring having 5 or 6 atoms in the ring. Examples include, but are not limited to, pyridinyl, thienyl, pyridinyl, imidazolyl, thiazolyl, oxazolyl, furyl, pyrazinyl, pyrimidinyl, and the like. With respect to Formula 1, in embodiments where D is NR¹¹R¹², wherein R¹¹ and R¹² are independently H or C₁₋₅ alkyl, R¹¹ and R¹² may be connected to form one or more rings, this refers to the possibility that NR¹¹R¹² may be a group such as:

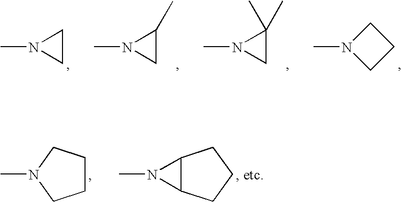

as well as the possibility that NR¹¹R¹² may not have any bond connecting them, such as:

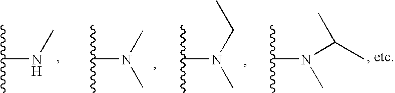

Asymmetric carbon atoms may be present in the compounds described. All such stereoisomers, both in a pure form or as a mixture of isomers, are intended to be included in the scope of the recited compound. In certain cases, compounds can exist in tautomeric forms. All tautomeric forms are intended to be included in the scope. Likewise, when compounds contain a double bond, there exists the possibility of cis- and trans-type isomeric forms of the compounds. Both cis- and trans-isomers, both in pure form as well as mixtures of cis- and trans-isomers, are contemplated. Thus, reference herein to a compound includes all of the aforementioned isomeric forms unless the context clearly dictates otherwise.

Alternate forms, including alternate solid forms, are included in the embodiments. Alternate solid forms such as polymorphs, solvates, hydrates, and the like, are alternate forms of a chemical entity that involve at least one of: differences in solid packing arrangements, non-covalent interactions with at least one solvent, and non-covalent interactions with water. Salts involve at least one ionic interaction between an ionic form of a chemical entity of interest and a counter-ion bearing an opposite charge. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) associated with administration of the compound to an animal before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

### Compounds

Unless otherwise indicated, if a term is used to describe more than one structural feature of the compounds disclosed herein, it should be assumed that the term has the same meaning for all of those features. Similarly, a subgroup of that term applies to every structural feature described by that term.

In some embodiments, the compound for the uses described herein is not:

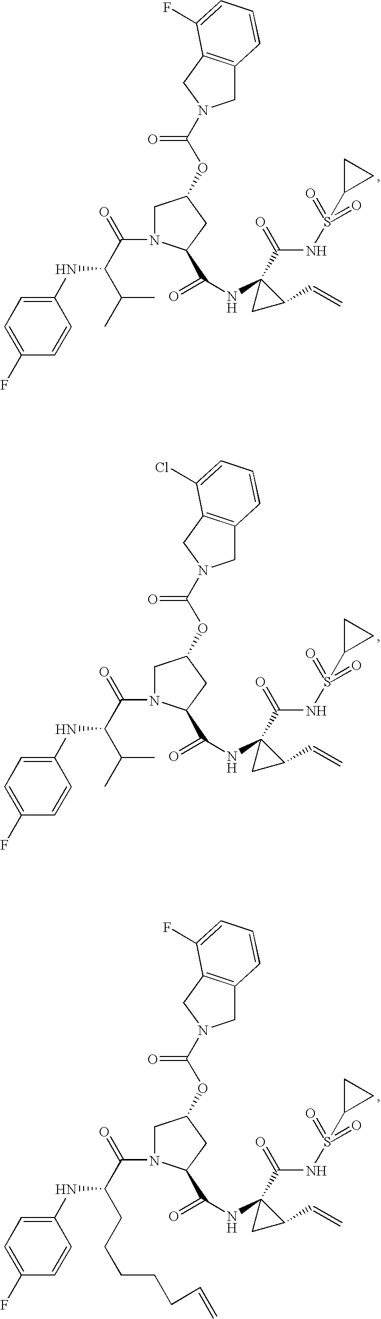

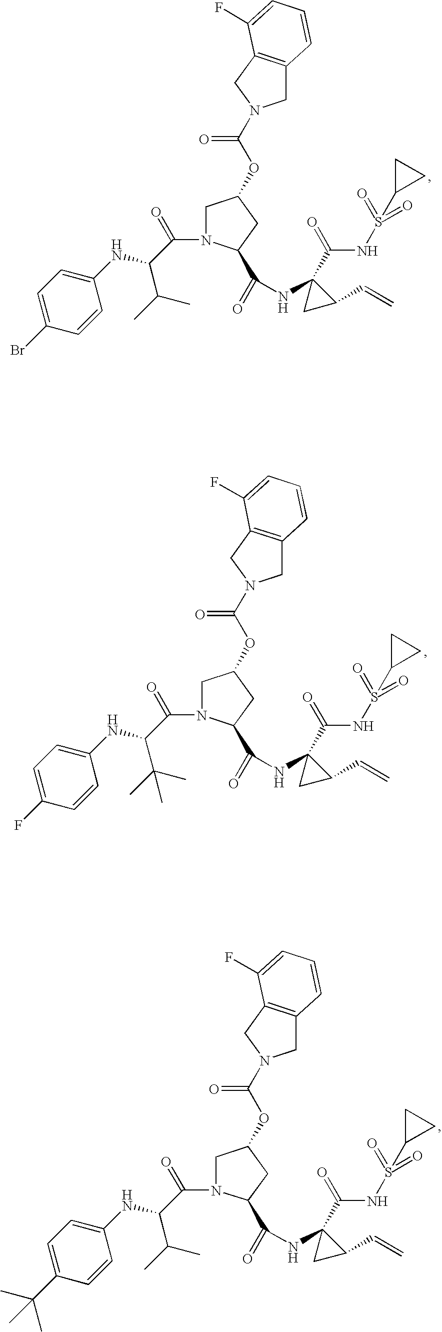

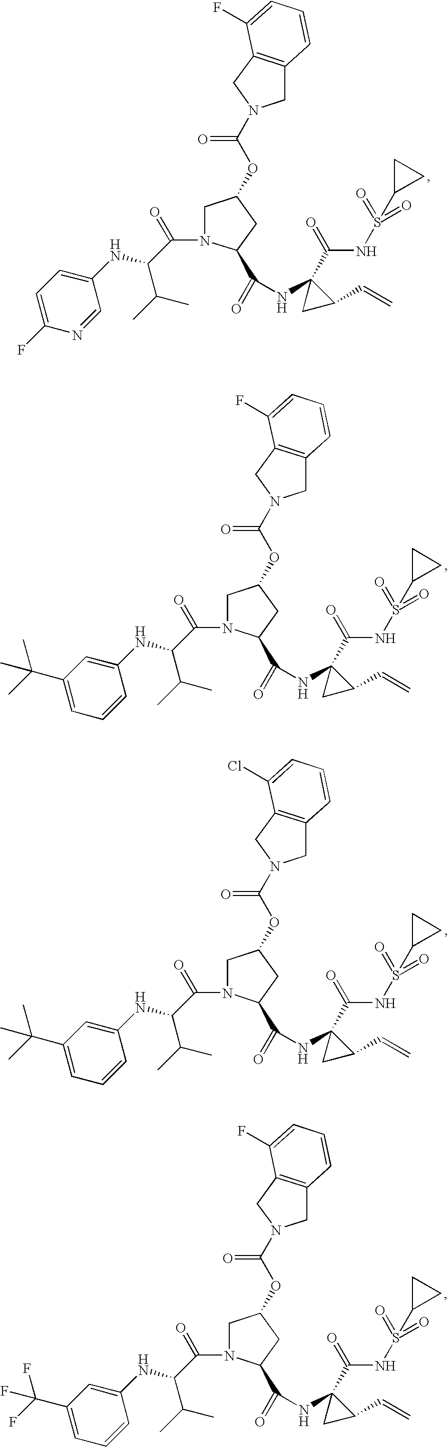

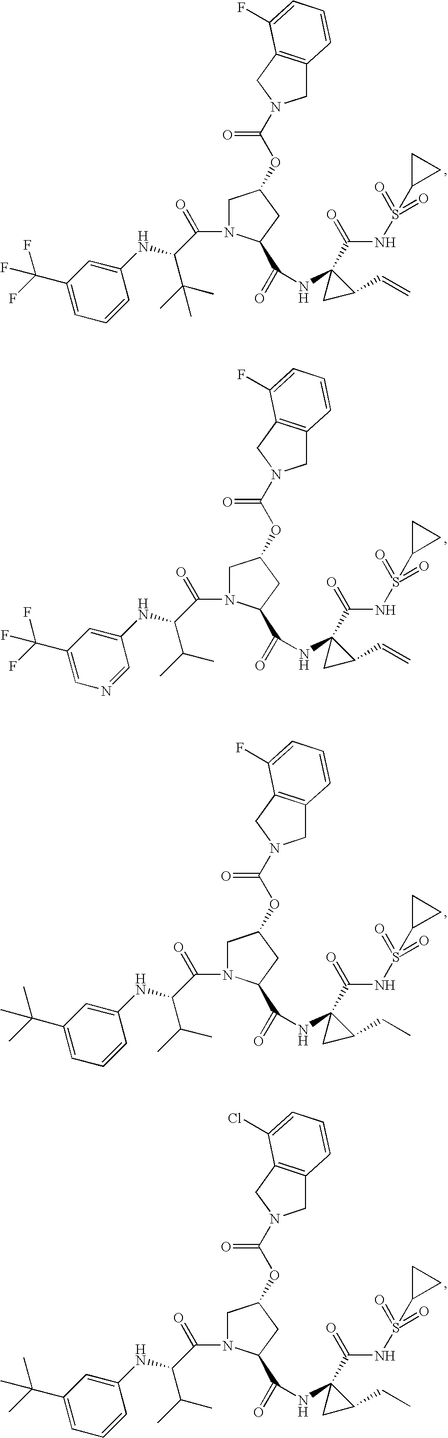

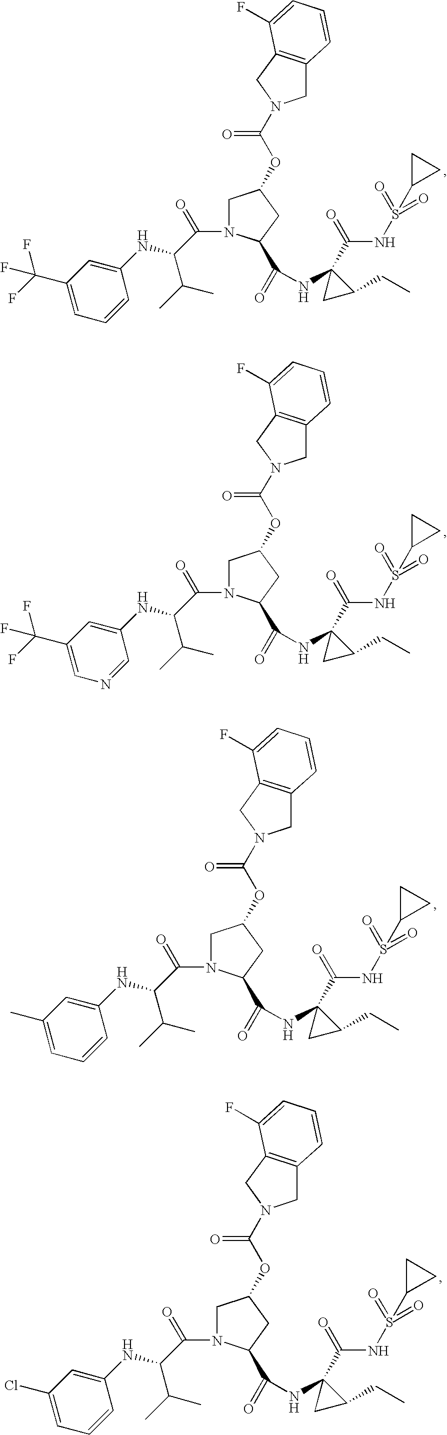

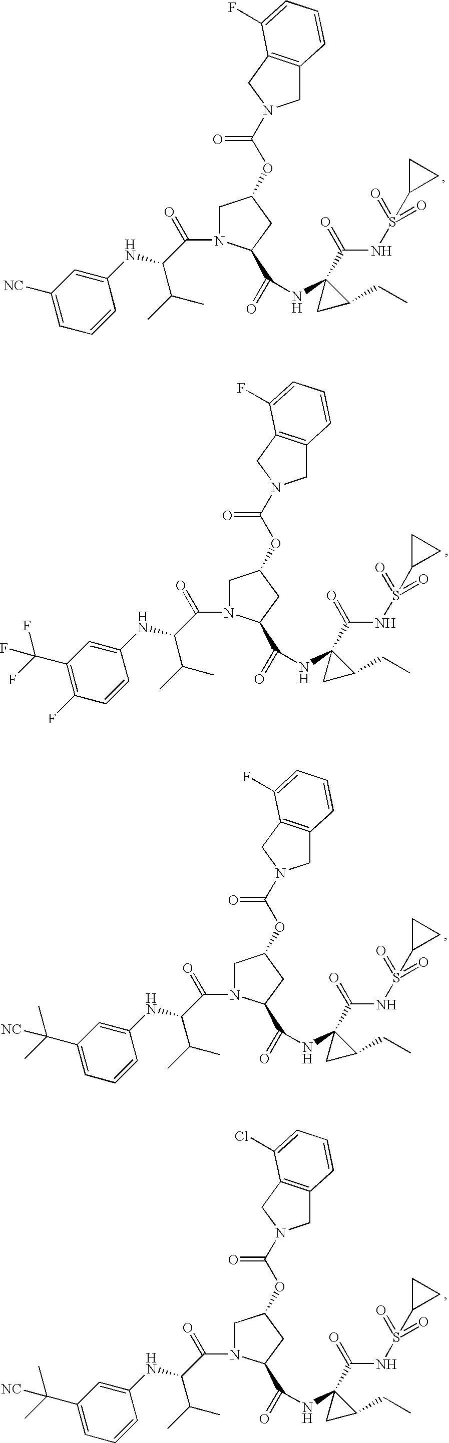

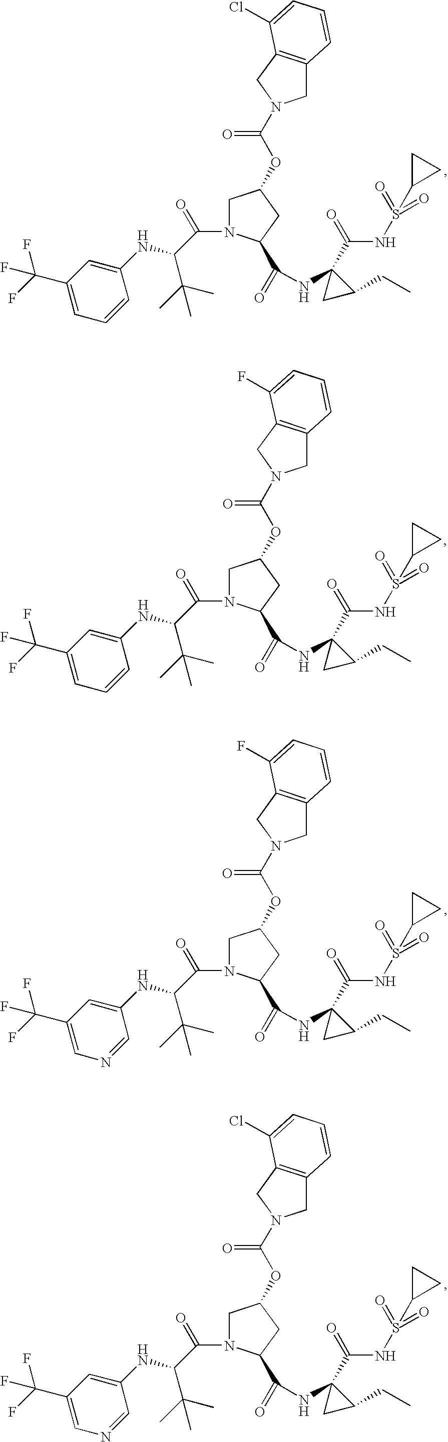

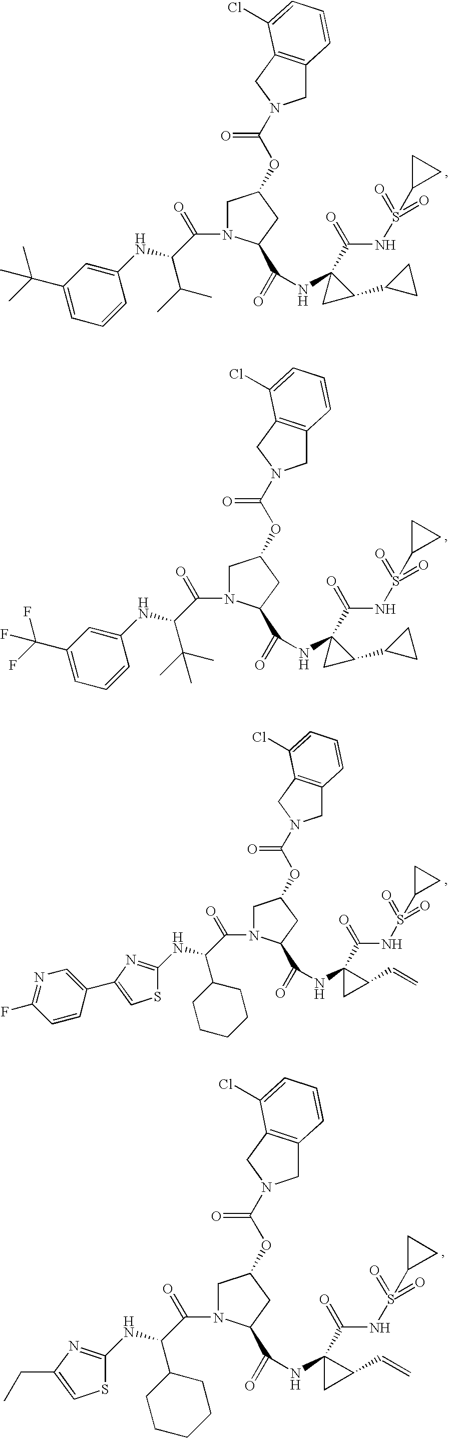

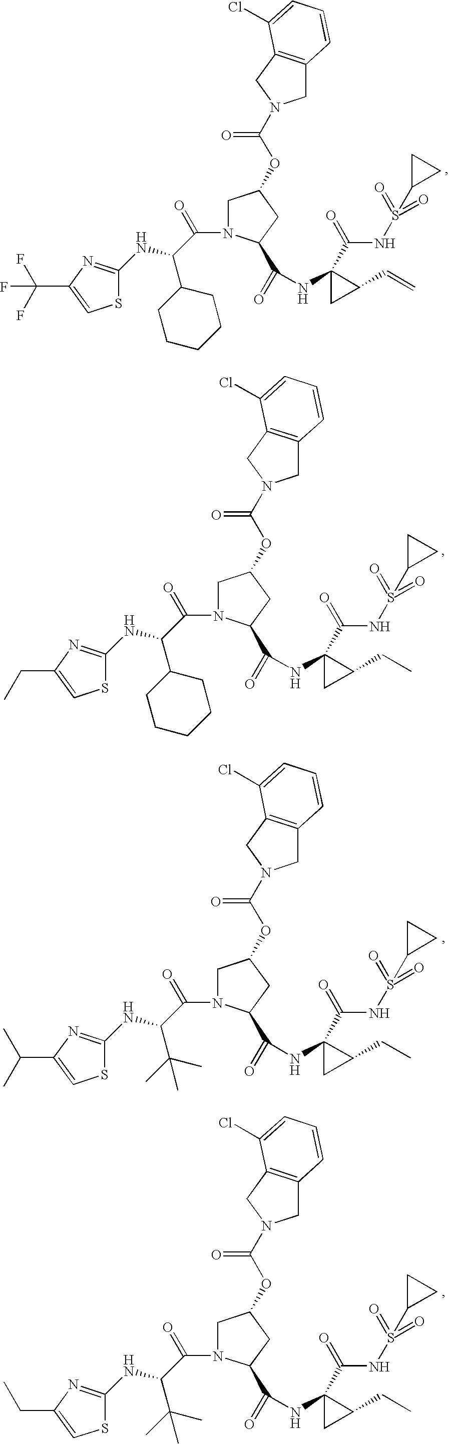

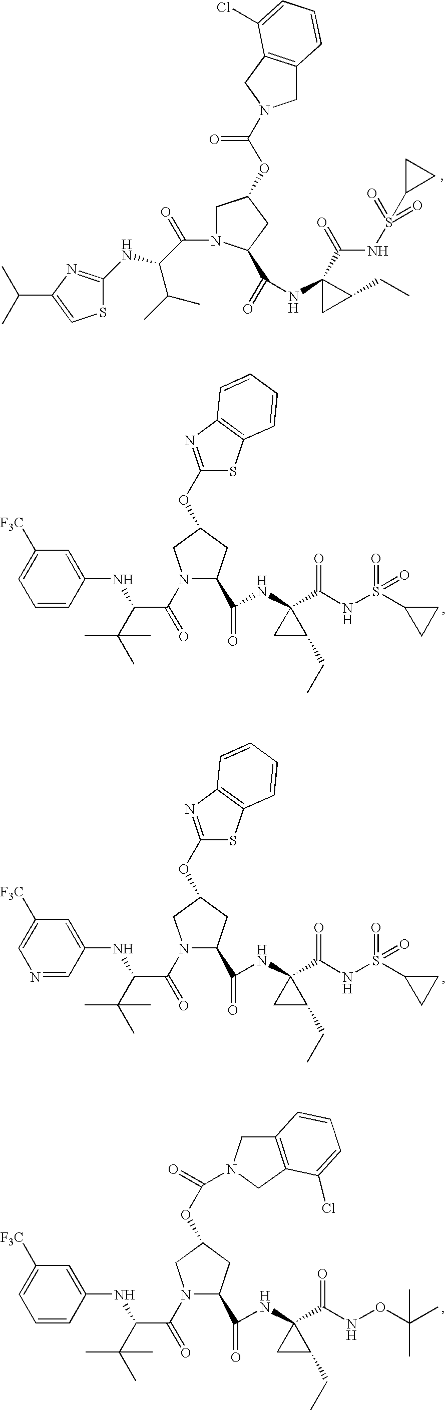

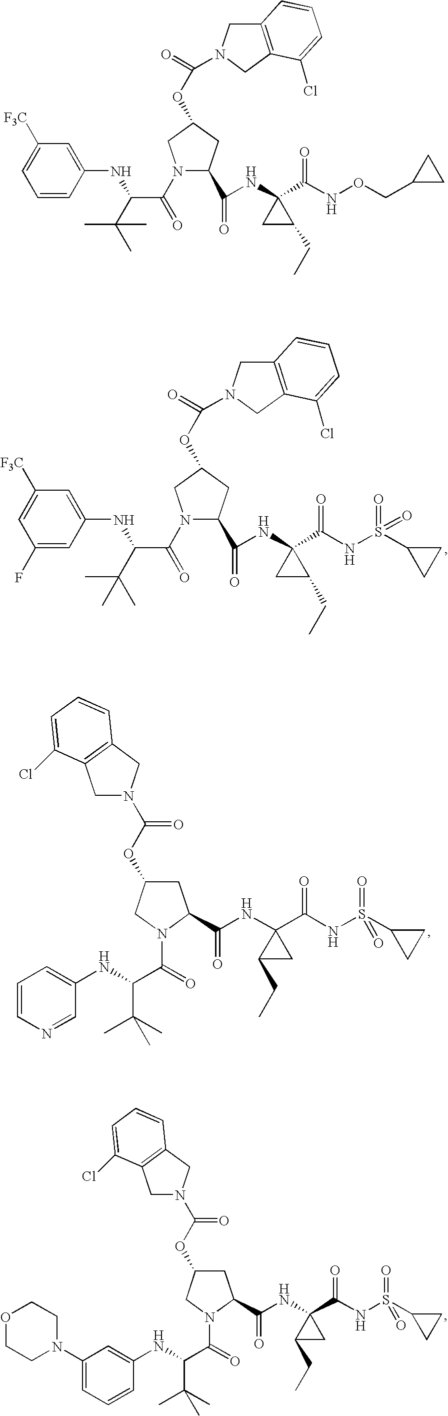

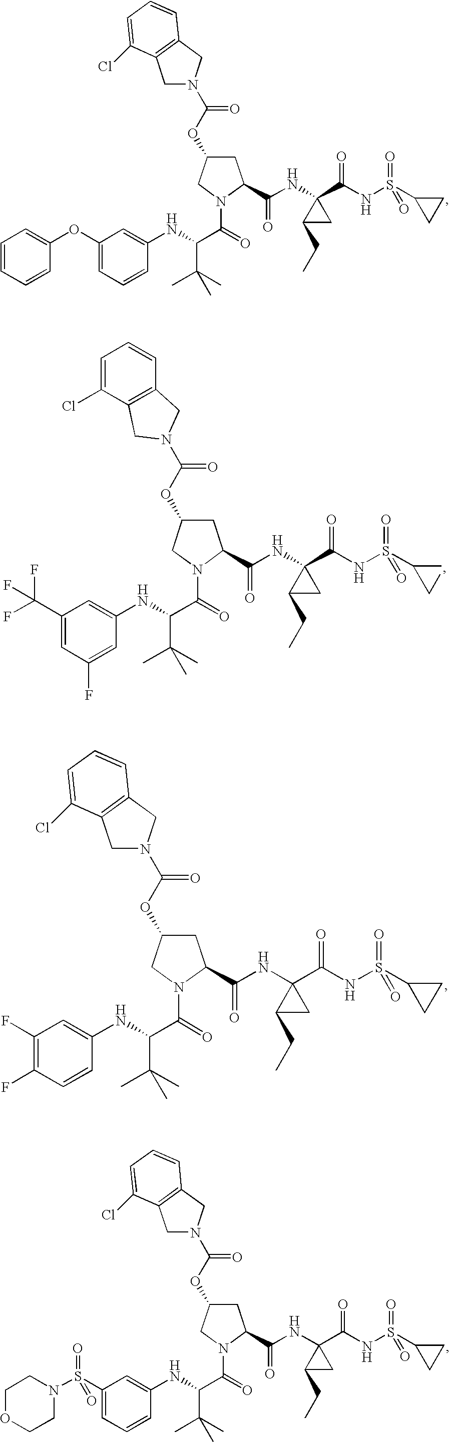

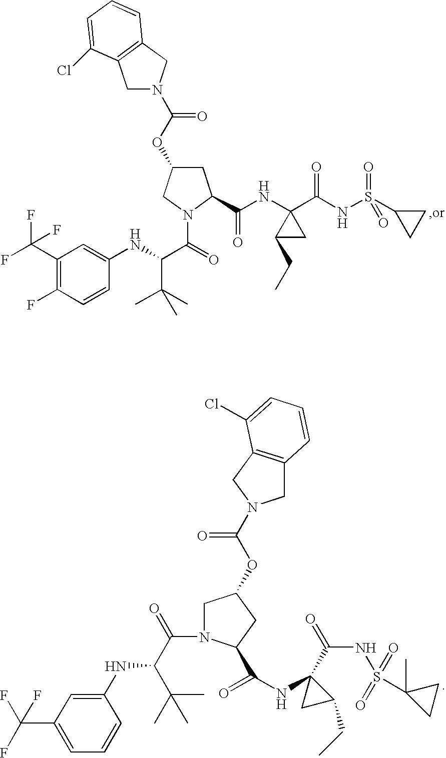

Some embodiments are represented by Formula 2:

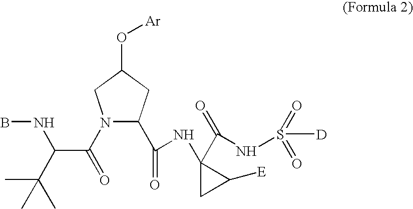

In some embodiments represented by Formula 1 or Formula 2, certain specific moieties are contemplated for Ar, B, D, and E:

In some embodiments, including those represented by Formula 1 or Formula 2, Ar may be optionally substituted quinolinyl, including optionally substituted quinolin-4-yl, optionally substituted

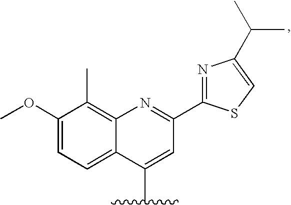

optionally substituted

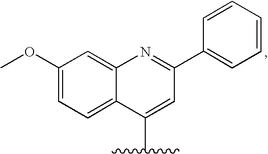

and the like; optionally substituted 3-(thiazol-2-yl)isoquinolinyl; or unsubstituted isoquinolinyl. In some embodiments, Ar may have one or more substituents independently selected from: optionally substituted phenyl, optionally substituted thiazolyl, C₁₋₆ alkoxy, C₁₋₆ alkyl, CF₃, F, Cl, Br, I, OCF₃, and

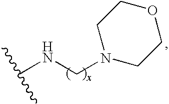

wherein x is 1, 2, or 3. In some of these embodiments, Ar may have from 0 to 3 substituents

independently selected from: CF₃, F, Cl, Br, I, CH₃, CH₂CH₃, CH₂CH₂CH₃, CH(CH₃)₂, OCH₃, OCF₃, and

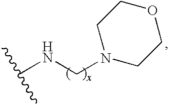

wherein x is 1, 2, or 3.

In some embodiments, including those represented by Formula 1 or Formula 2, B may be: optionally substituted phenyl; optionally substituted benzooxazol-2-yl; optionally substituted benzothiazol-2-yl; optionally substituted benzoimidazol-2-yl; optionally substituted benzothiazol-2-yl; optionally substituted isoindolin-2-yl; or an optionally substituted 5- or 6-membered heteroaryl, including but not limited to: pyridinyl, imidazolyl, thiazolyl, oxazolyl, thienyl, or furyl. In some embodiments, including those where B is one of the specific rings or ring systems above, B may have one or more substituents independently selected from: OH, C₁₋₆ alkyl, C₁₋₆ alkoxy, C₁₋₆ perfluoroalkyl, CF₃, halo, C₁₋₆ perfluoroalkoxy. In some embodiments, including those where B is one of the specific rings or ring systems above, B may have from 1 to 3 substituents independently selected from: CF₃, F, Cl, Br, I, C₁₋₃ alkyl, OCH₃, and OCF₃. In some embodiments, including those represented by Formula 1 or Formula 2, B may be one of:

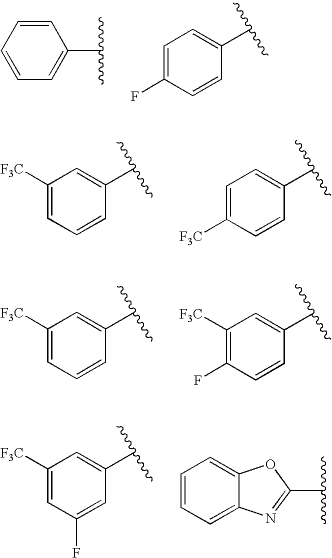

In some embodiments, including those represented by Formula 1 or Formula 2, D may be 1-methylcyclopropyl, cyclopropyl, or N(CH₃)₂.

In some embodiments, including those represented by Formula 1 or Formula 2, E may be ethyl, vinyl, or cyclopropyl. In some embodiments, including those represented by Formula 1 or Formula 2, E may be C₁₋₆ alkyl.

Some embodiments, including those represented by Formula 1 or Formula 2, contemplate specific combinations of one or more of Ar, B, D, and E as listed above.

In some embodiments, including those represented by Formula 1 or Formula 2, Ar is optionally substituted benzoimidazol-2-yl and B is optionally substituted phenyl. In some of these embodiments, Ar is benzoimidazol-2-yl having from 0 to 3 substituents independently selected from: CF₃, F, Cl, Br, I, CH₃, CH₂CH₃, CH₂CH₂CH₃, CH(CH₃)₂, OCH₃, OCF₃, and

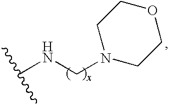

wherein x is 1, 2, or 3. Some of these embodiments further contemplate specific combinations of one or more of D (i.e. 1-methylcyclopropyl, cyclopropyl, or N(CH₃)₂) and E (i.e. ethyl, vinyl, or cyclopropyl) as listed above.

In some embodiments, including those represented by Formula 1 or Formula 2, Ar is optionally substituted benzothiazol-2-yl, B is optionally substituted phenyl, and D is C₄₋₆ hydrocarbyl. In some of these embodiments, Ar may be benzothiazol-2-yl having from 0 to 3 substituents independently selected from: CF₃, F, Cl, Br, I, CH₃, CH₂CH₃, CH₂CH₂CH₃, CH(CH₃)₂, OCH₃, OCF₃, and

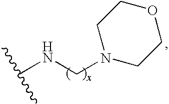

wherein x is 1, 2, or 3. In some of these embodiments, E may be ethyl, vinyl, or cyclopropyl.

In some embodiments, including those represented by Formula 1 or Formula 2, Ar is unsubstituted isoquinolinyl and E is C₁₋₆ alkyl. Some of these embodiments further contemplate specific combinations of one or more of B and D as listed above.

In some embodiments, Ar is optionally substituted isoindolin-2-yl; z is 1; and B is optionally substituted phenyl. In some of these embodiments, Ar is isoindolin-2-yl having from 0 to 3 substituents independently selected from: CF₃, F, Cl, Br, I, CH₃, CH₂CH₃, CH₂CH₂CH₃, CH(CH₃)₂, OCH₃, OCF₃, and

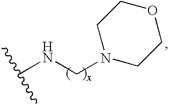

wherein x is 1, 2, or 3. Some of these embodiments further contemplate specific combinations of one or more of D (i.e. 1-methylcyclopropyl, cyclopropyl, or N(CH₃)₂) and E (i.e. ethyl, vinyl, or cyclopropyl) as listed above. Some of these embodiments include a proviso that if D is cyclopropyl, then: B is fluorotrifluoro-methylphenyl and E is cyclopropyl.

Some embodiments provide compounds of Formula 1, wherein the compound is not one of the compounds depicted below.

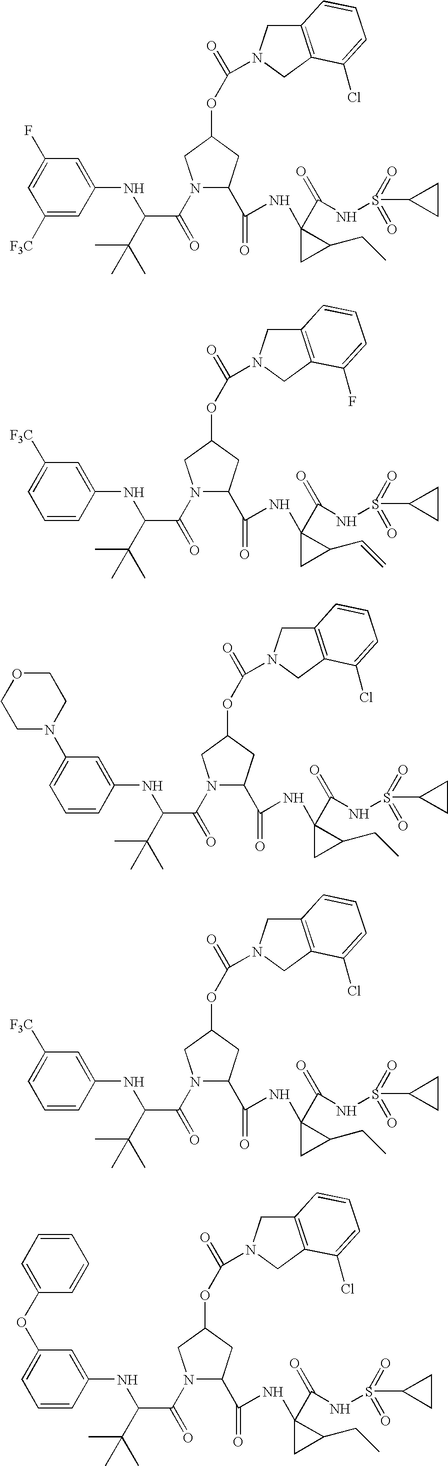

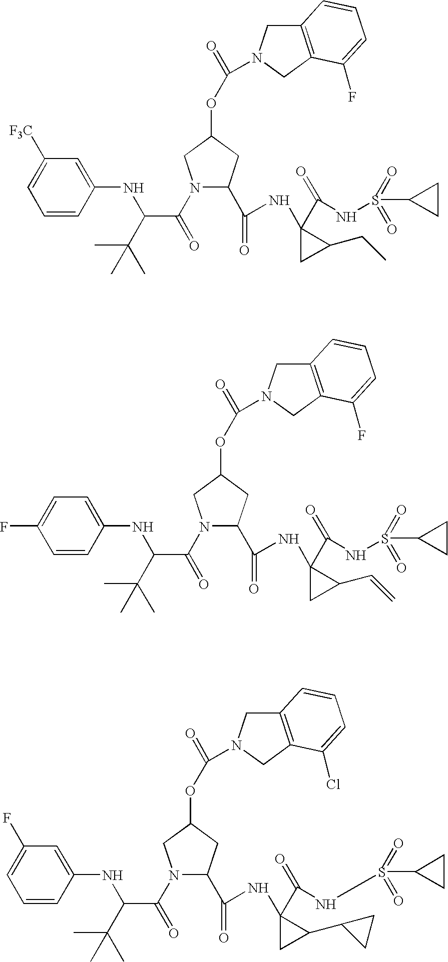

Some embodiments provide a compound represented by Formula 3:

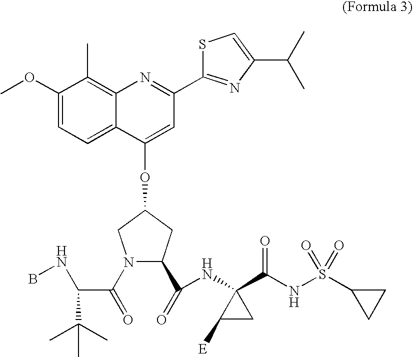

wherein B and E are the same as those of any embodiments above related to Formula 1 or Formula 2.

Some embodiments provide a compound represented by Formula 4:

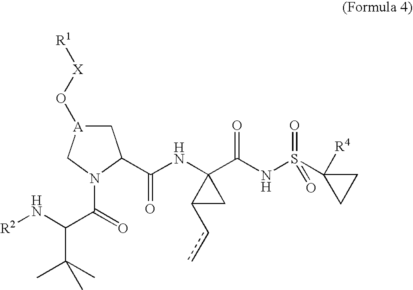

wherein a dashed line represents the presence or absence of a bond; X is —CO— or a single bond; R² is aryl or heteroaryl having from 0 to 3 substituents independently selected from: —CO₂H, —OO₂—C₁₋₄-alkyl, halo, —CF₃, —OCF₃, —CN, —CO(CH₂)₂NMe₂,

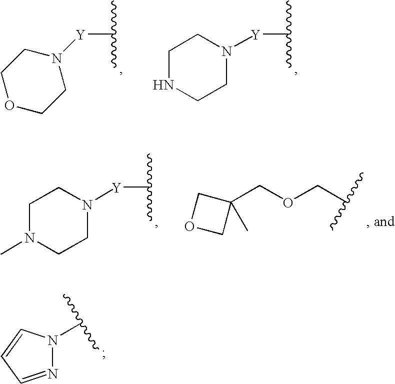

Y is —CO— or SO₂—; R⁴ is hydrogen or C₁₋₄ alkyl; and

1) A is

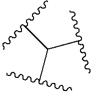

and R¹ is isoquinolinyl having from 0 to 6 substituents; or isoindolinyl having from 1 to 3 substituents independently selected from —F and —NHCOR³; and R³ is C₁₋₁₀ alkyl, C₁₋₁₀ alkyl ether, C₁₋₁₀ alkyl amine, or a combination thereof, provided that if R³ is 4-fluoroisoindolin-2-yl, R² is not 4-fluorophenyl, 3-trifluoromethylphenyl, or 5-trifluoromethylpyridin-3-yl; or

2) A is

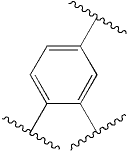

and R¹ is 3-chlorophenyl, provided that if R⁴ is hydrogen, R² is not 4-fluorophenyl.

A dashed line represents the presence or absence of a bond. Thus, the structural formulas below represent individual embodiments that are contemplated.

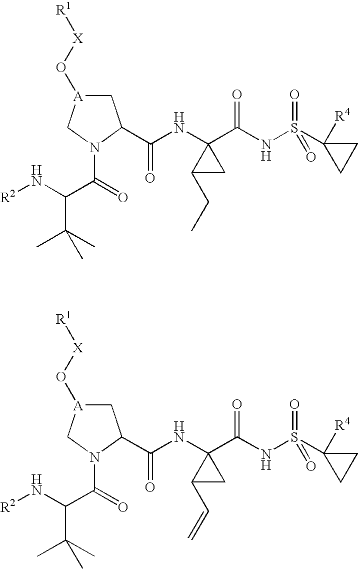

X is CO or a single bond. Thus, the structural formulas below represent individual embodiments that are contemplated.

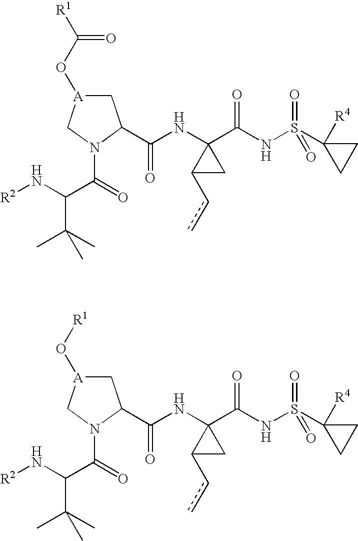

R² is phenyl having from 0 to 3 substituents independently selected from: CO₂H, CO₂CH₃, —CO₂CH₂CH₃, F, CF₃, OCF₃, CN, CO(CH₂)₂NMe₂,

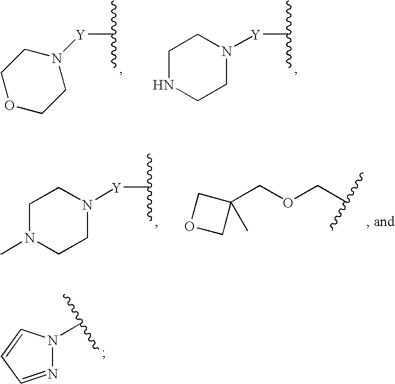

wherein Y is CO or SO₂.

The textually depicted structural features: CO₂H, CO₂CH₃, CO₂CH₂CH₃, CF₃, OCF₃, —CN, and CO(CH₂)₂NMe₂, are also represented by the pictorial structural formulas below.

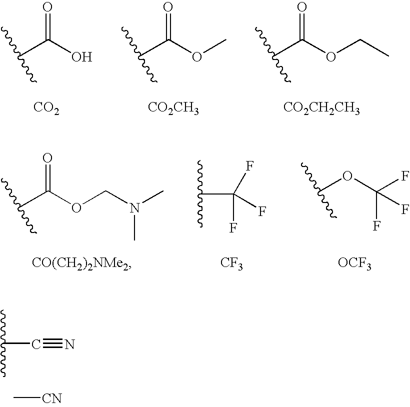

Unless otherwise indicated, similar textually depicted structural features have analogous structures.

Since Y is CO or SO₂, R² may also be phenyl with one of the substituents depicted below.

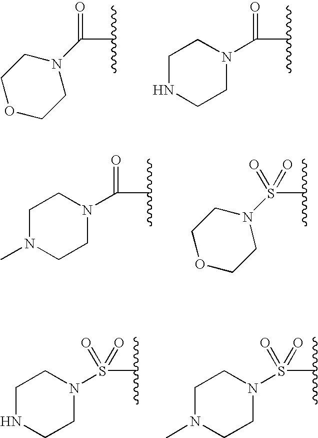

In some embodiments, R² or B is:

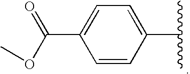

In some embodiments, R² or B is:

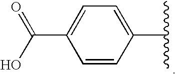

In some embodiments, R² or B is:

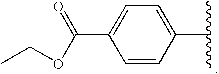

In some embodiments, R² or B is:

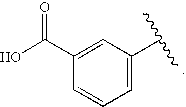

In some embodiments, R² or B is:

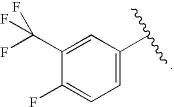

In some embodiments, R² or B is:

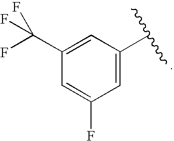

In some embodiments, R² or B is:

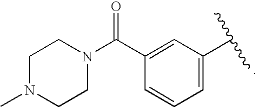

In some embodiments, R² or B is:

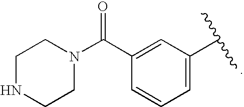

In some embodiments, R² or B is:

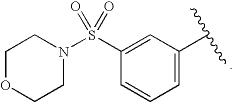

In some embodiments, R² or B is:

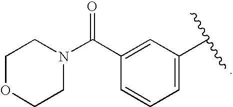

In some embodiments, R² or B is:

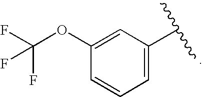

In some embodiments, R² or B is:

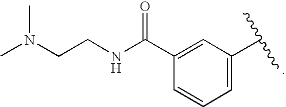

In some embodiments, R² or B is:

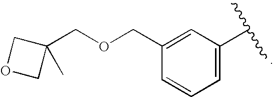

In some embodiments, R² or B is:

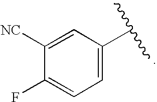

In some embodiments, R² or B is:

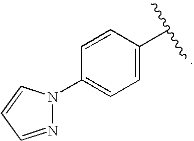

In some embodiments, R² or B is:

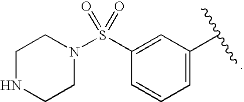

R⁴ is hydrogen or C₁₋₄ alkyl. Thus, each structural formula below represents some embodiments that are contemplated.

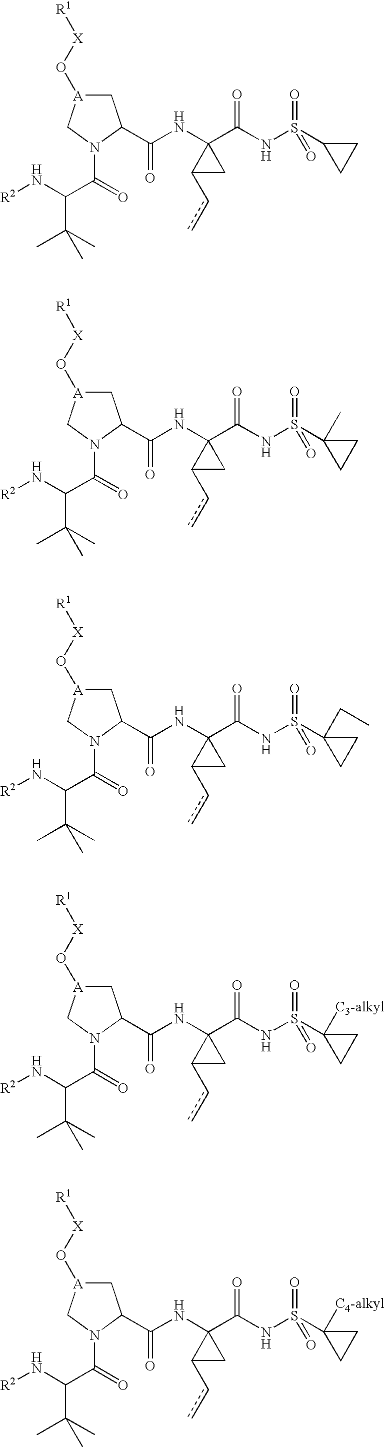

C₃-alkyl is cyclopropane, propane, or an isomer thereof.

C₄-alkyl is cyclobutane or an isomer thereof, or butane or an isomer thereof.

Some embodiments provide a compound represented by a formula:

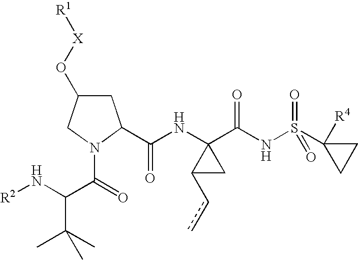

wherein R¹ is isoquinolinyl having from 0 to 6 substituents; or isoindolinyl having from 1 to 3 substituents independently selected from —F and —NHCOR³; and R³ is C₁₋₁₀ alkyl, C₁₋₁₀ alkyl ether, C₁₋₁₀ alkyl amine, or a combination thereof; provided that if R¹ is 4-fluoroisoindolin-2-yl, R² is not 4-fluorophenyl or 3-trifluoromethylphenyl.

Some embodiments provide a compound represented by a formula:

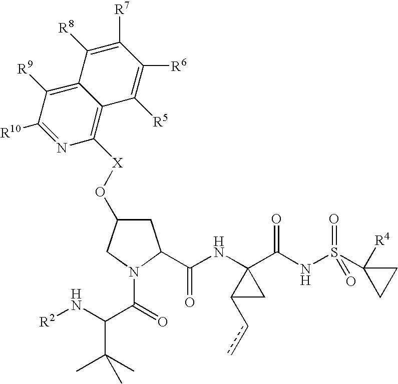

wherein R⁵, R⁶, R⁷, R⁸, R⁹, and R¹⁰ are independently substituents.

In some embodiments, R⁵, R⁶, R⁷, R⁸, R⁹, and R¹⁰ are independently selected from —F, —Cl, —Br, —CF₃, C₁₋₄ alkyl, and —NHCOR³, wherein R³ is C₁₋₁₀ alkyl, C₁₋₁₀ alkyl ether, C₁₋₁₀ alkyl amine, or a combination thereof.

Some embodiments provide a compound represented by a formula:

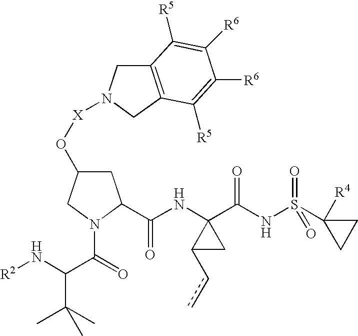

wherein each R⁵ and R⁶ is independently selected from hydrogen, —F, and —NHCOR³; wherein R³ is C₁₋₁₀ alkyl, C₁₋₁₀ alkyl ether, C₁₋₁₀ alkyl amine, or a combination thereof, provided that at least 1 of R⁵ or R⁶ is hydrogen.

Some embodiments provide a compound represented by a formula:

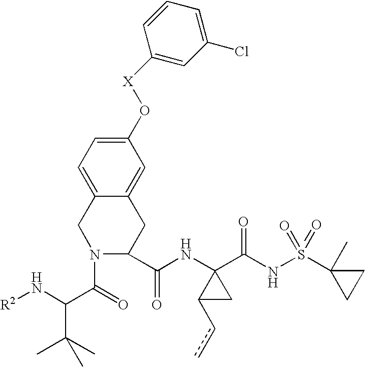

provided that if leis hydrogen, R² is not 4-fluorophenyl.

Some embodiments provide a compound represented by a formula:

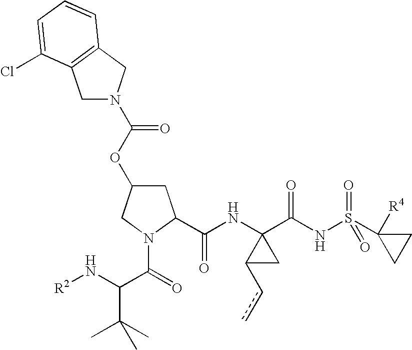

wherein R² is phenyl having from 0 to 3 substituents independently selected from: —CO₂H, —CO₂CH₃, —CO₂CH₂CH₃, —OCF₃, —CN, —CO(CH₂)₂NMe₂,

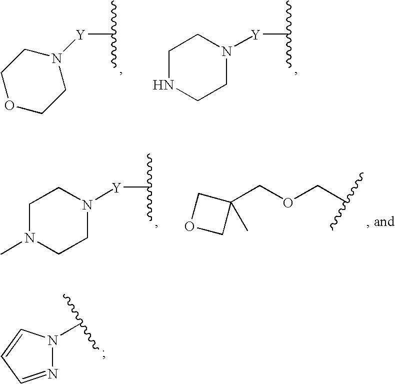

Y is —CO— or —SO₂—.

In some embodiments R⁴ is hydrogen.

Some embodiments provide a compound represented by a formula:

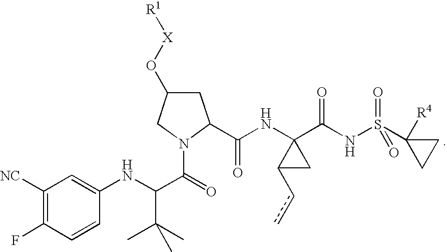

Some embodiments provide a compound represented by a formula:

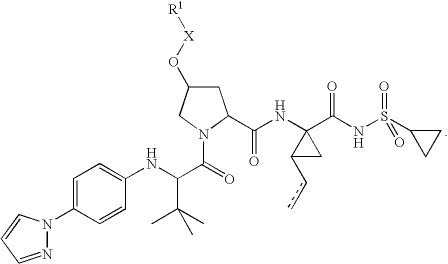

Some embodiments provide a compound represented by a formula:

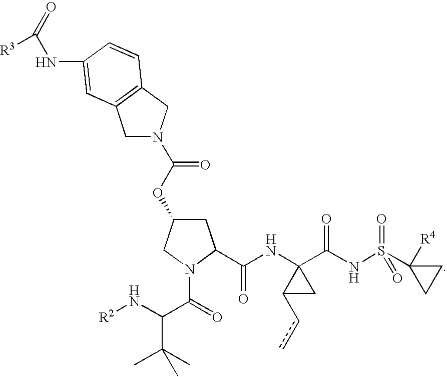

Some embodiments provide a compound represented by a formula:

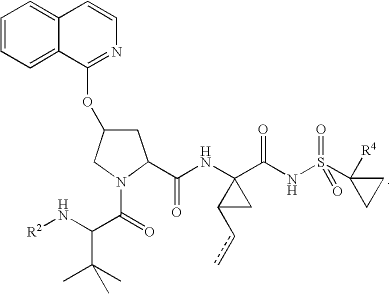

Some embodiments provide a compound represented by a formula:

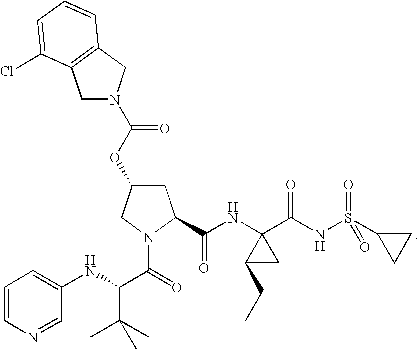

Some embodiments provide a compound represented by a formula:

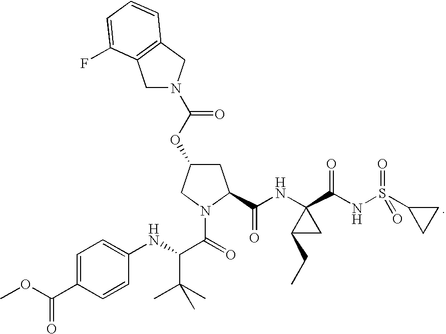

Some embodiments provide a compound represented by a formula:

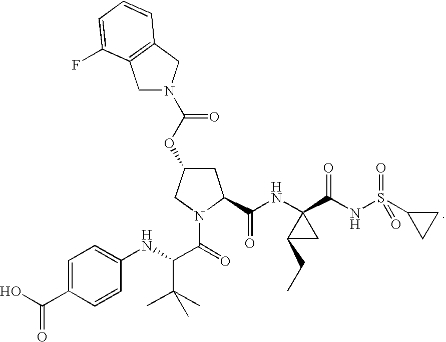

Some embodiments provide a compound represented by a formula:

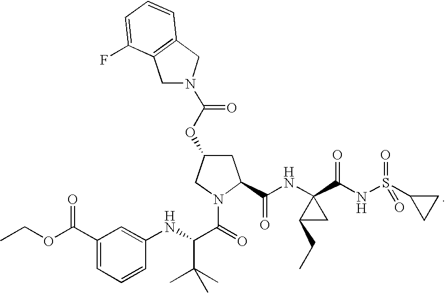

Some embodiments provide a compound represented by a formula:

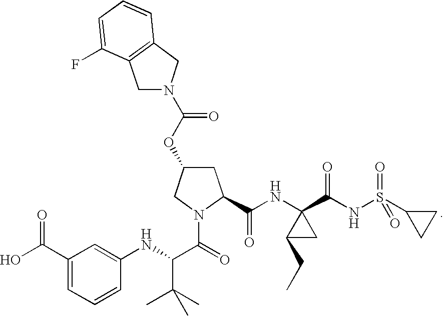

Some embodiments provide a compound represented by a formula:

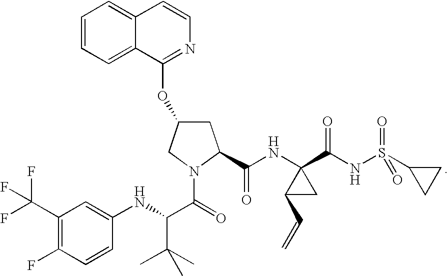

Some embodiments provide a compound represented by a formula:

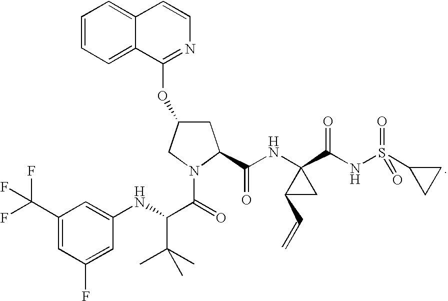

Some embodiments provide a compound represented by a formula:

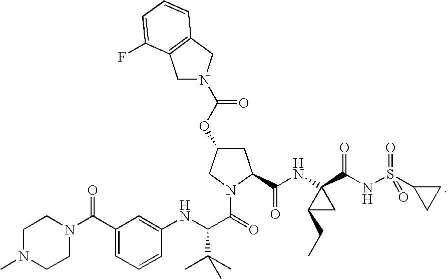

Some embodiments provide a compound represented by a formula:

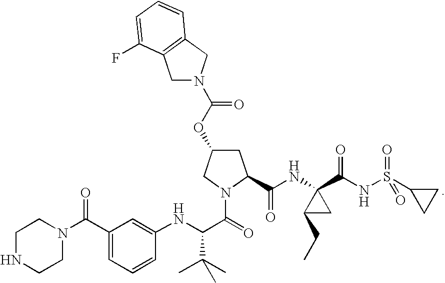

Some embodiments provide a compound represented by a formula:

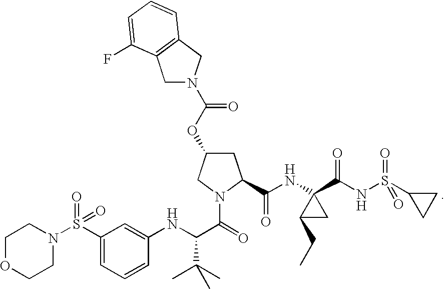

Some embodiments provide a compound represented by a formula:

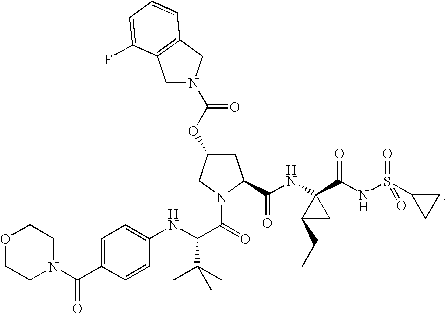

Some embodiments provide a compound represented by a formula:

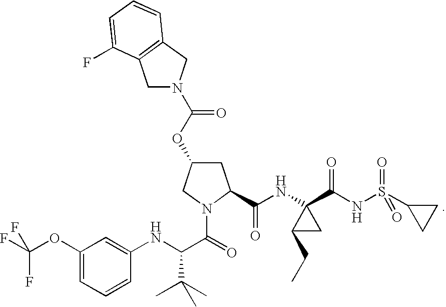

Some embodiments provide a compound represented by a formula:

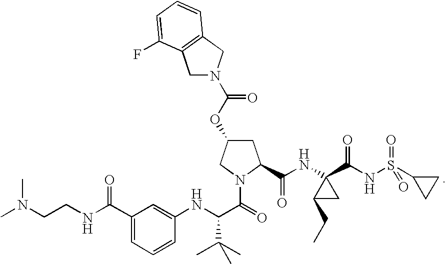

Some embodiments provide a compound represented by a formula:

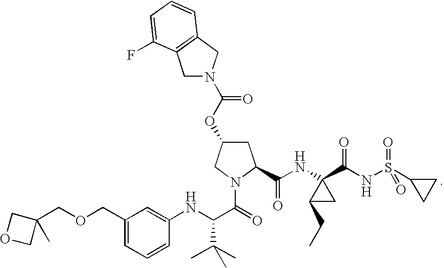

Some embodiments provide a compound represented by a formula:

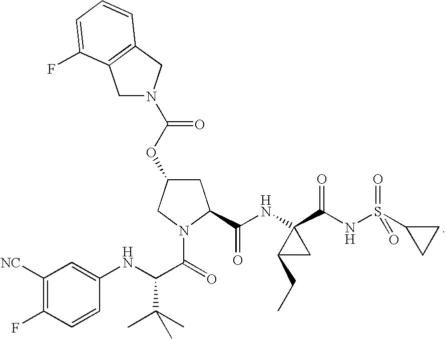

Some embodiments provide a compound represented by a formula:

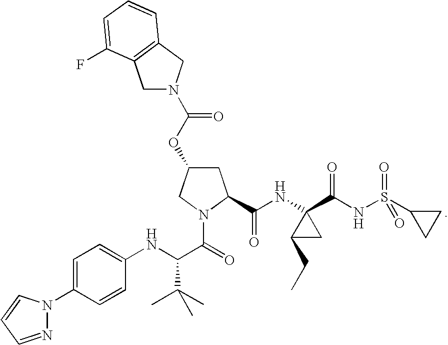

Some embodiments provide a compound represented by a formula:

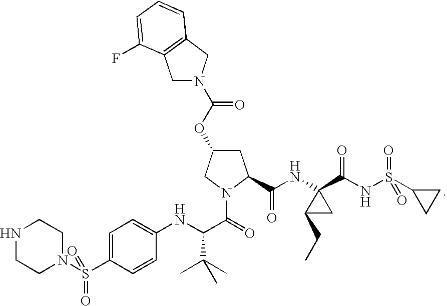

Some embodiments provide a compound represented by a formula:

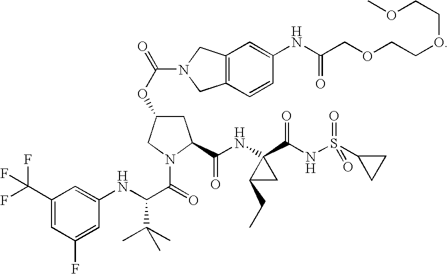

Some embodiments provide a compound represented by a formula:

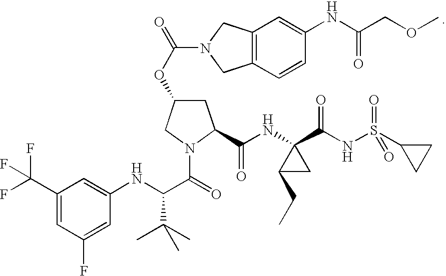

Some embodiments provide a compound represented by a formula:

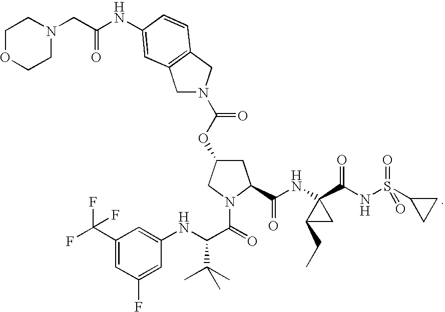

Some embodiments provide a compound represented by a formula:

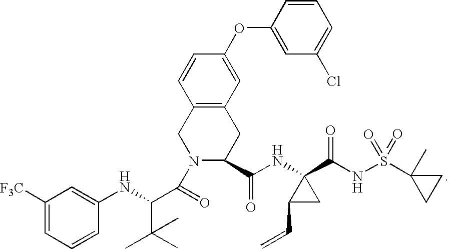

Some embodiments provide a compound represented by a formula:

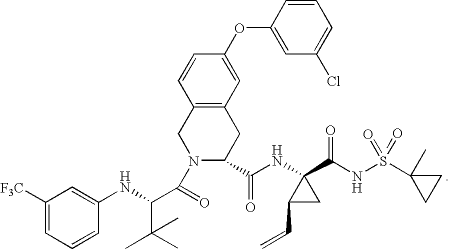

Some embodiments provide a compound represented by a formula:

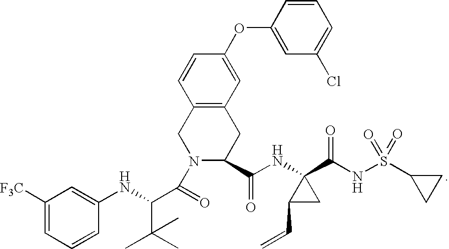

Some embodiments provide a compound represented by the formula:

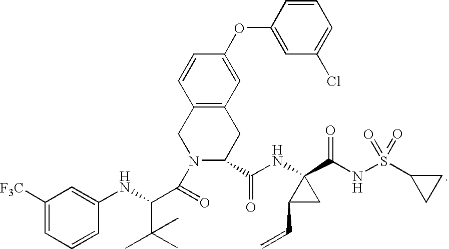

Some embodiments provide a compound represented by the formula:

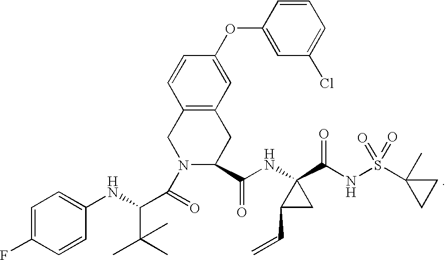

Some embodiments provide a compound represented by the formula:

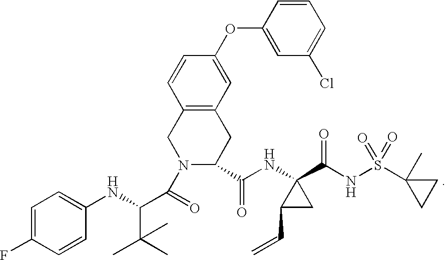

Some embodiments provide a compound represented by the formula:

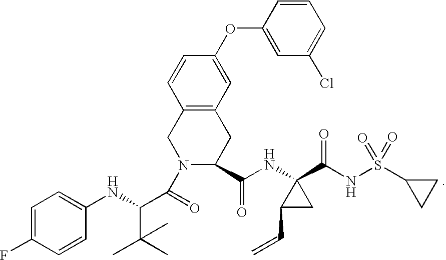

Some embodiments provide a compound represented by the formula:

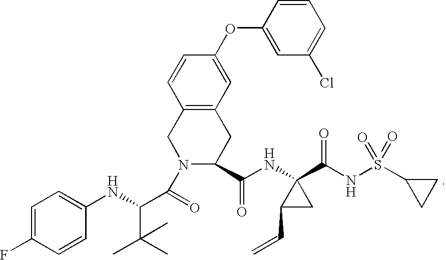

Some embodiments provide a compound selected from:

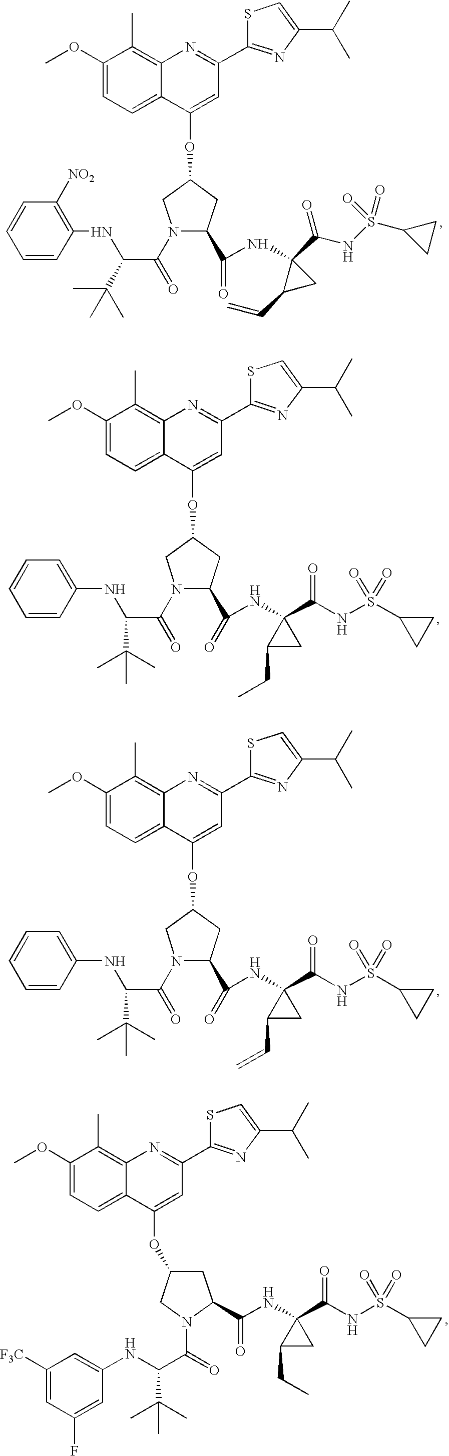

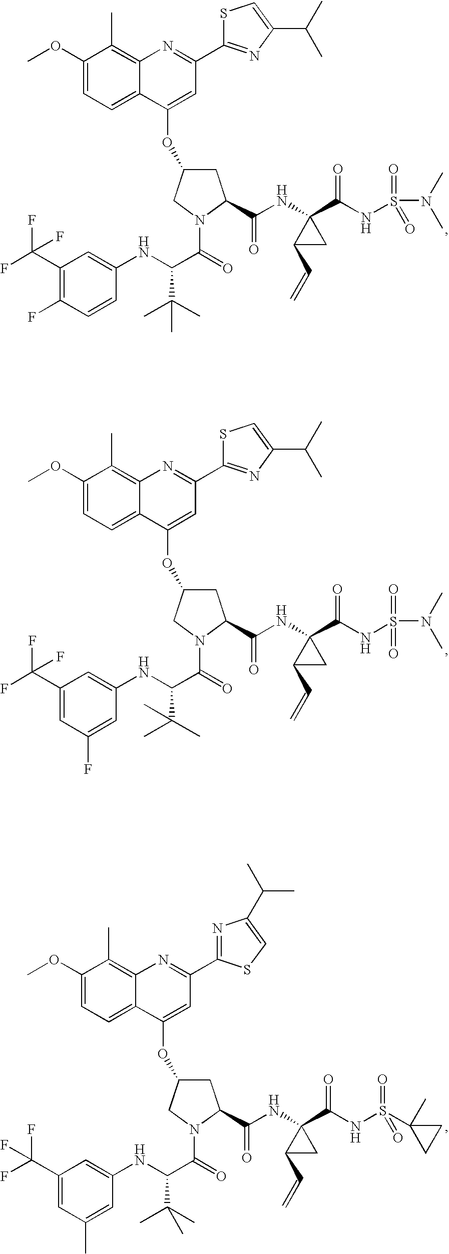

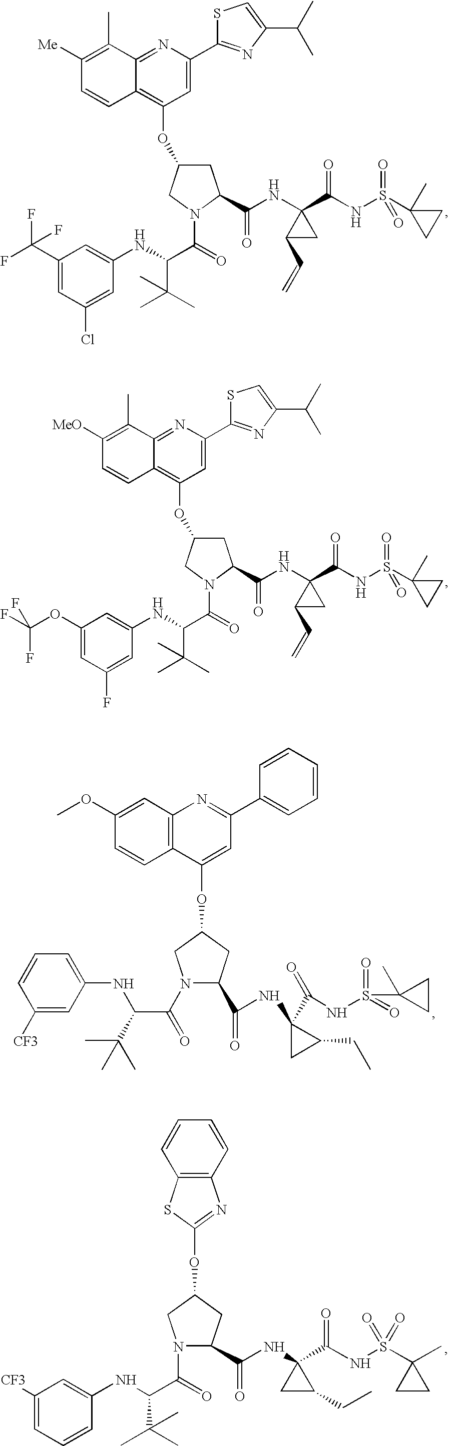

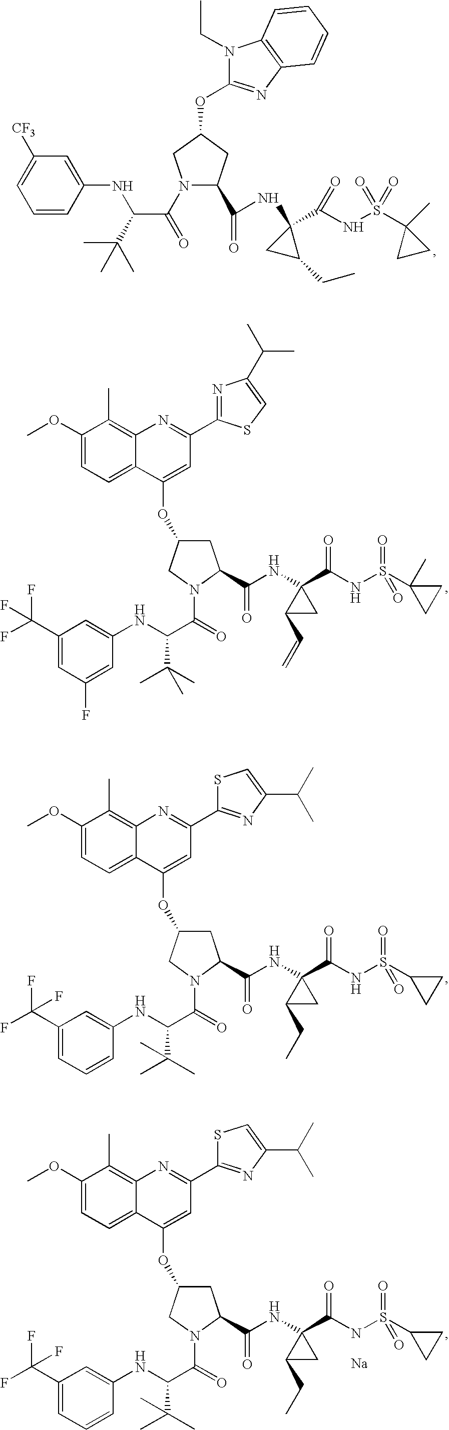

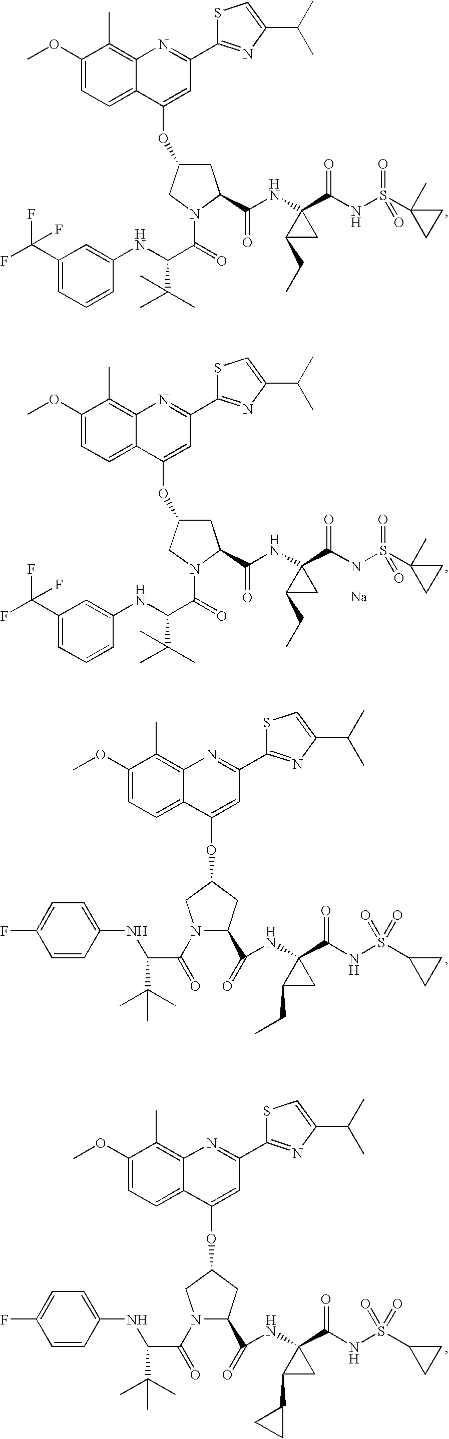

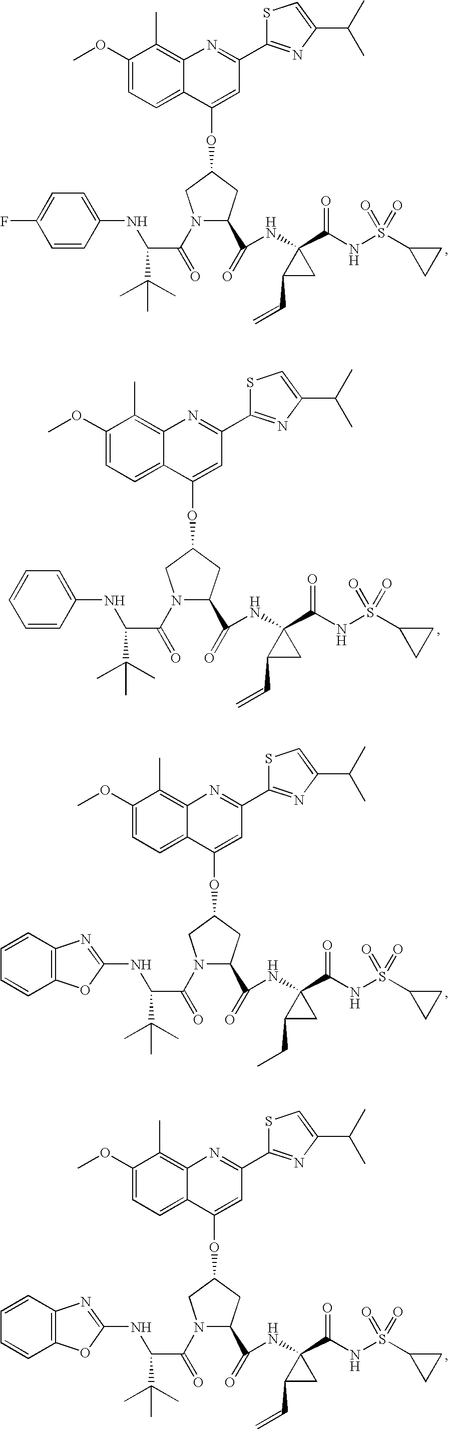

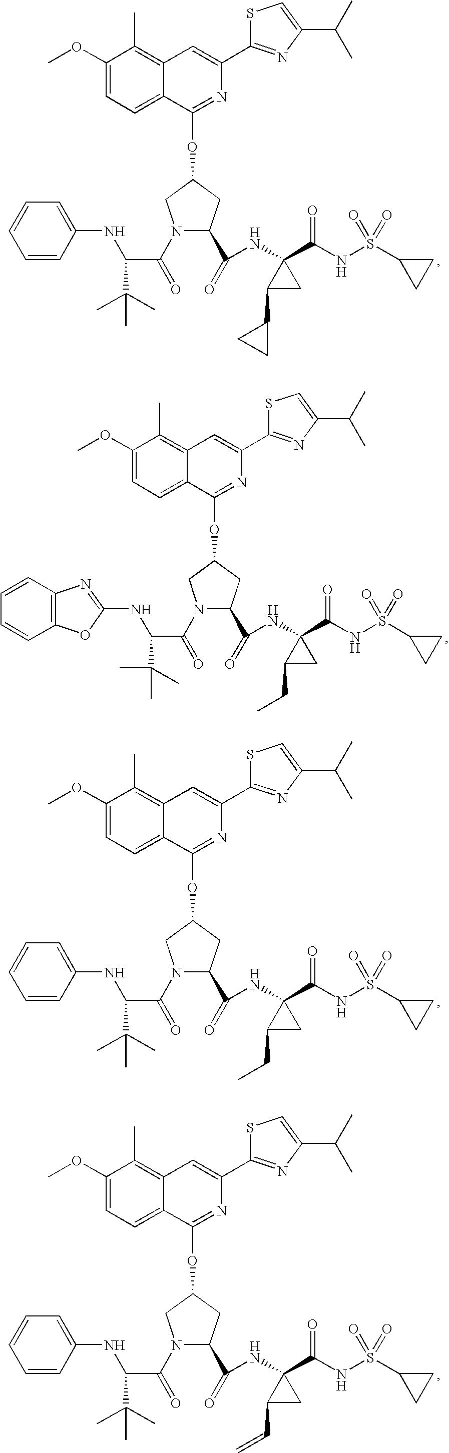

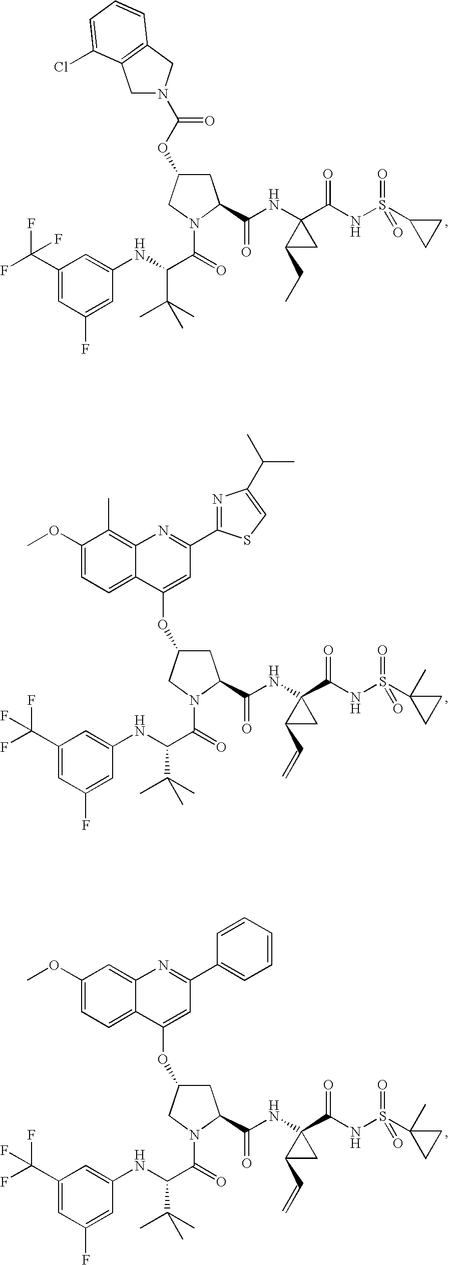

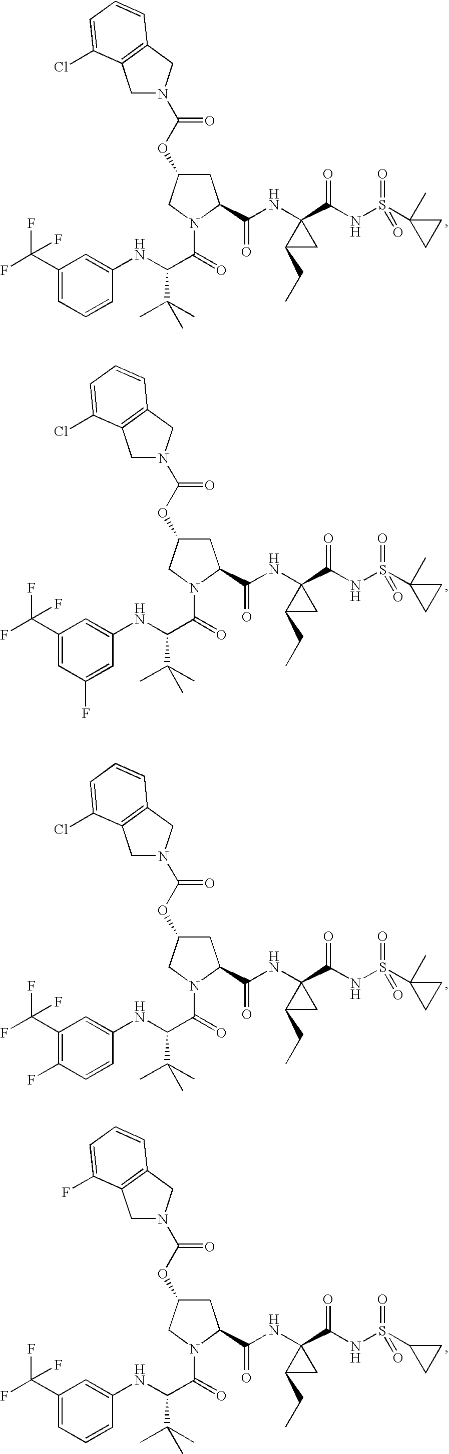

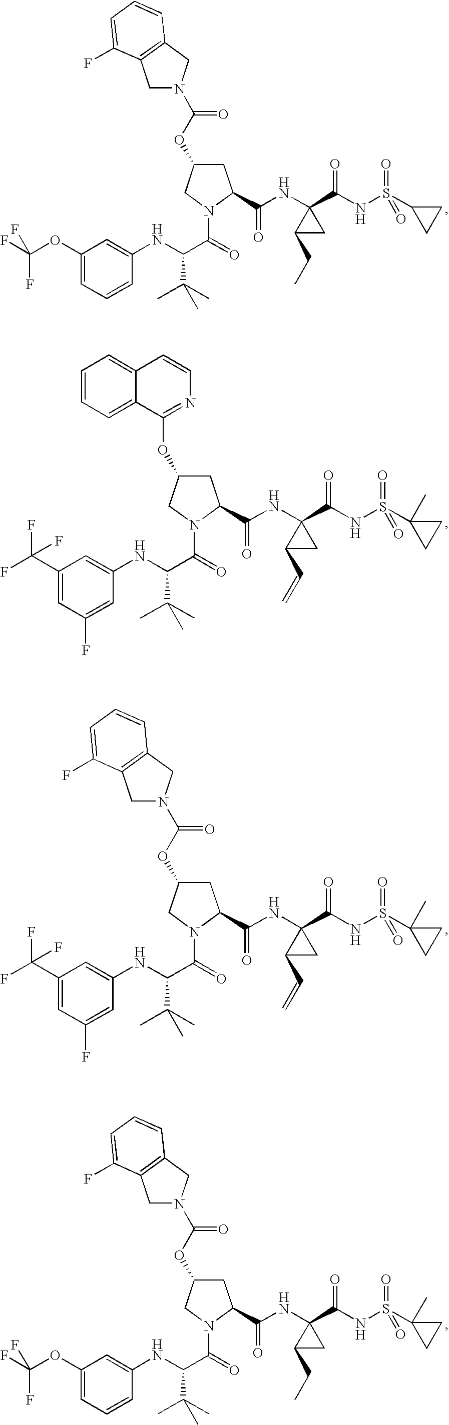

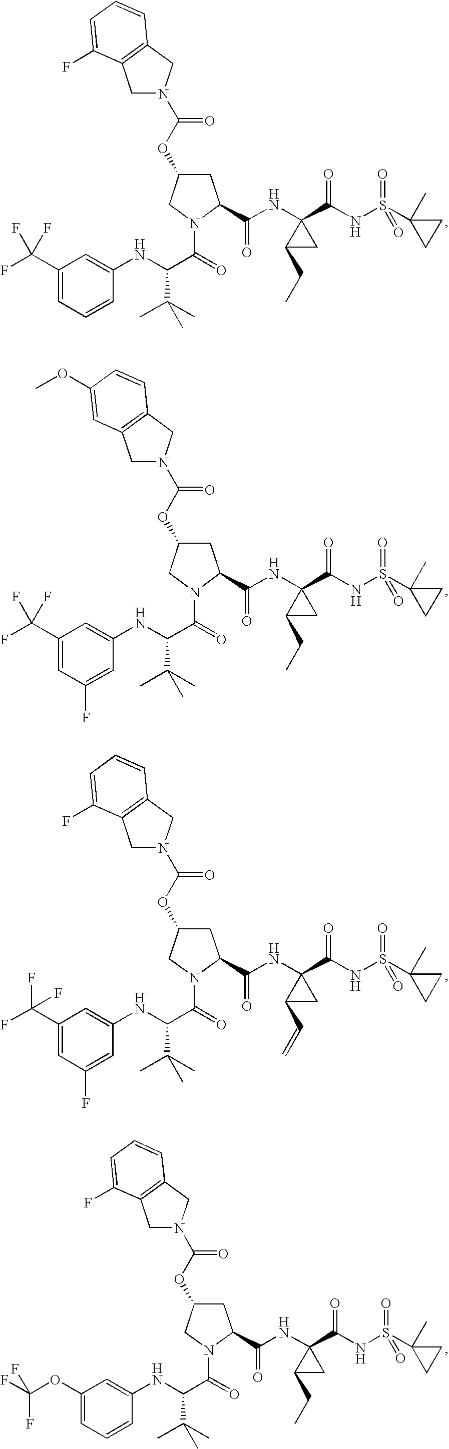

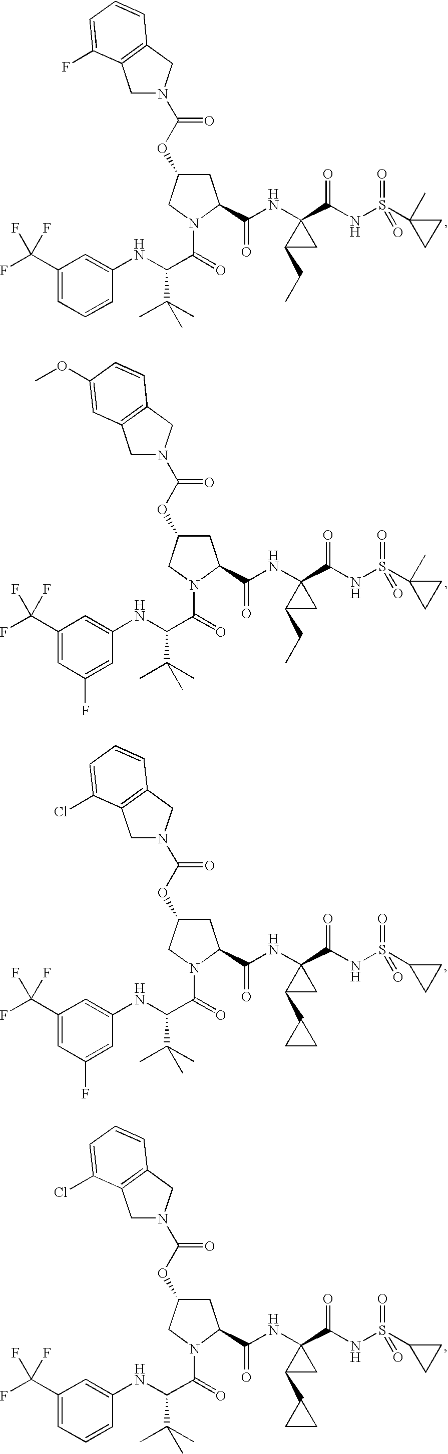

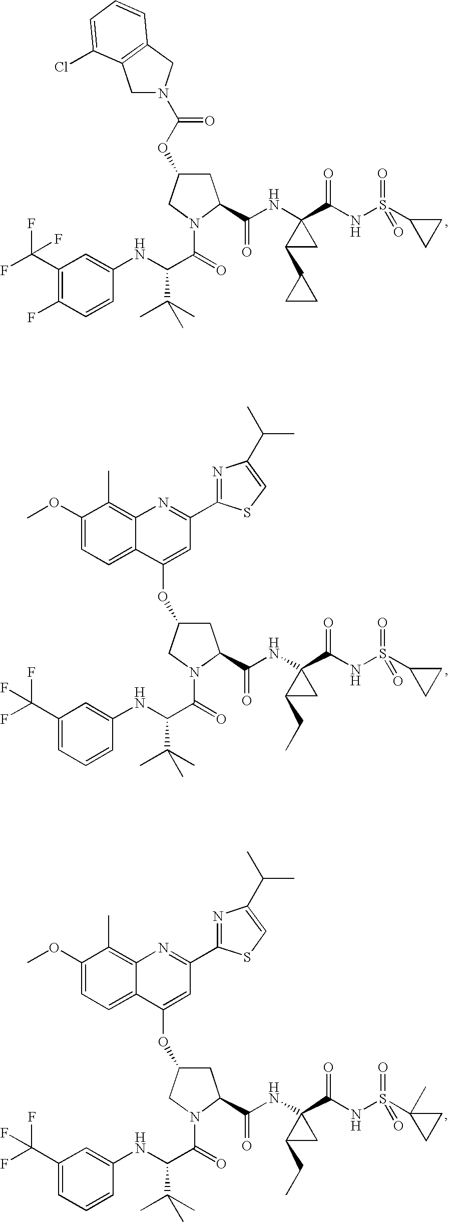

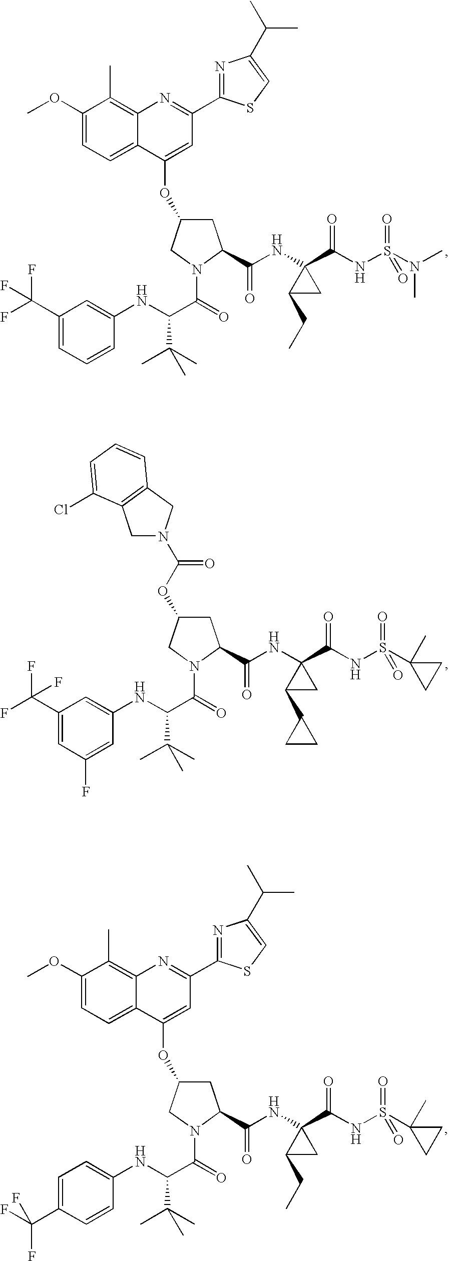

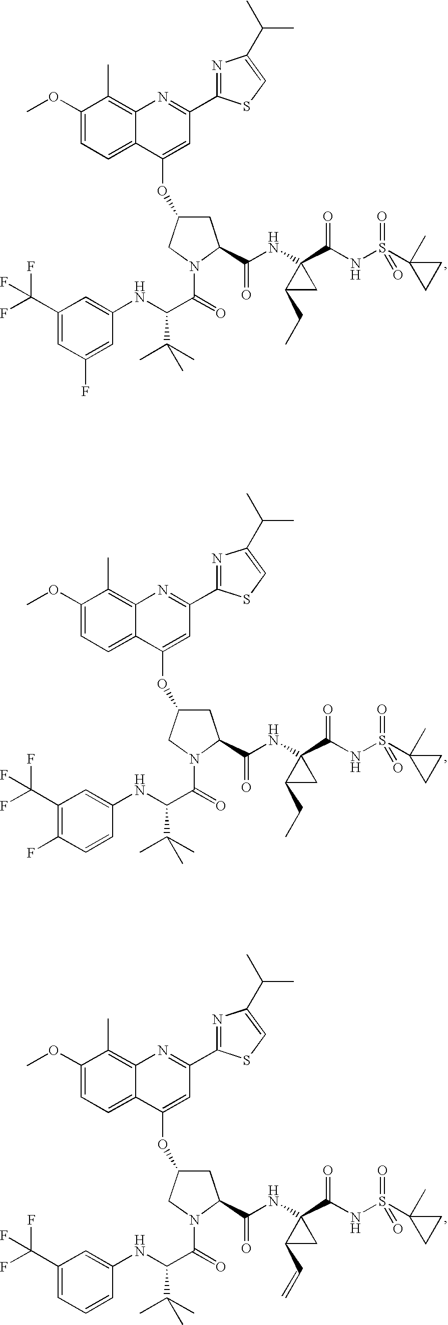

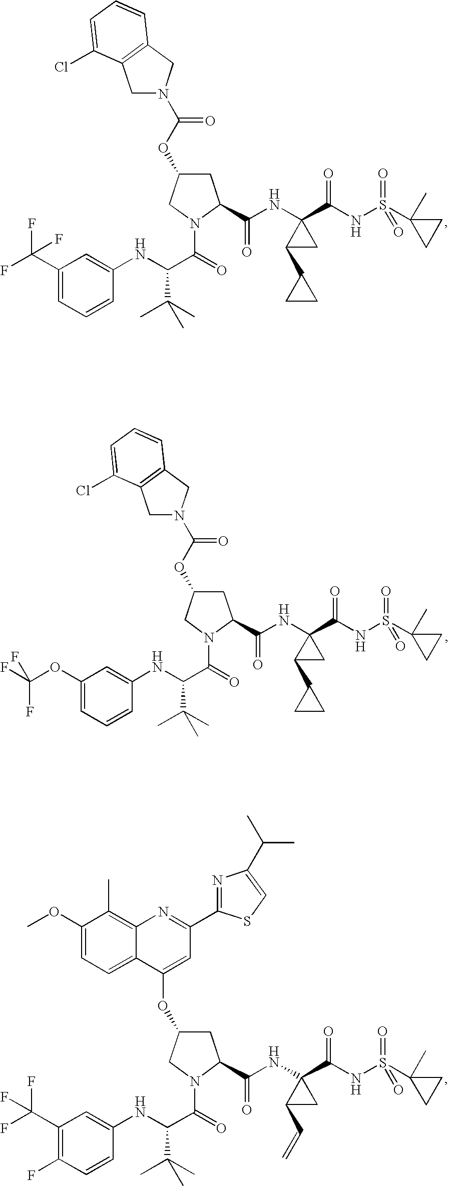

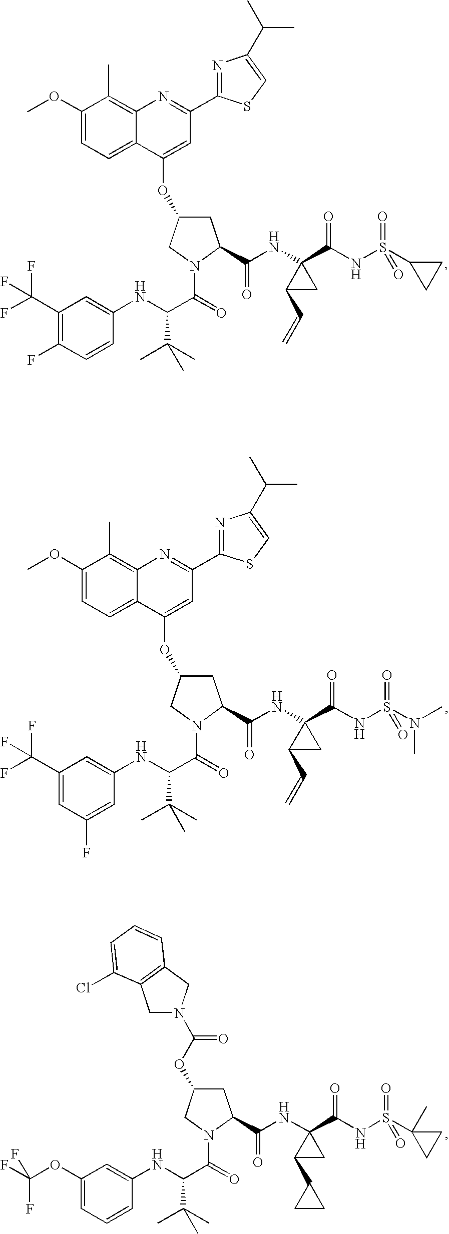

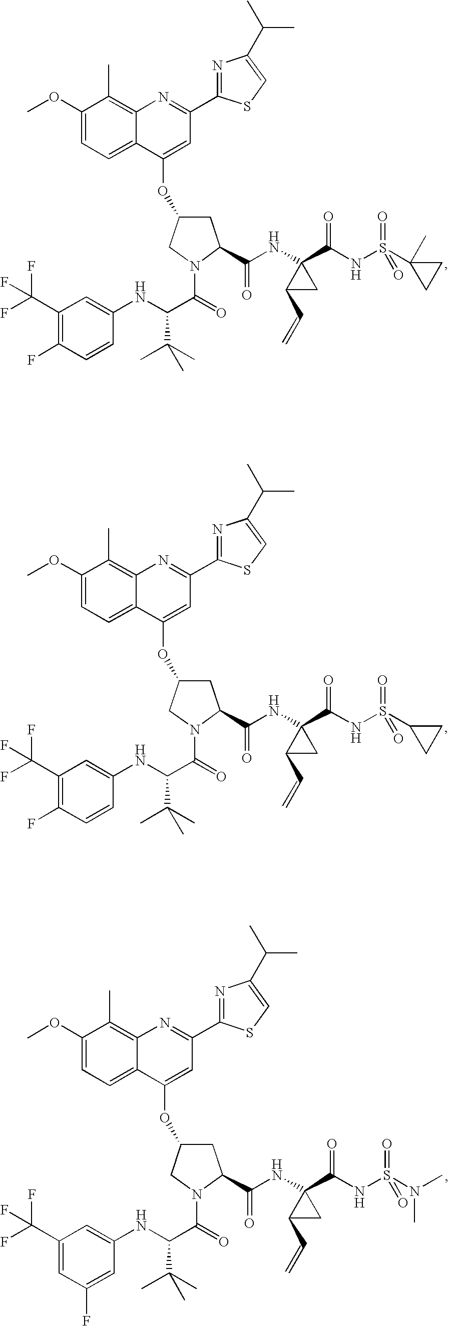

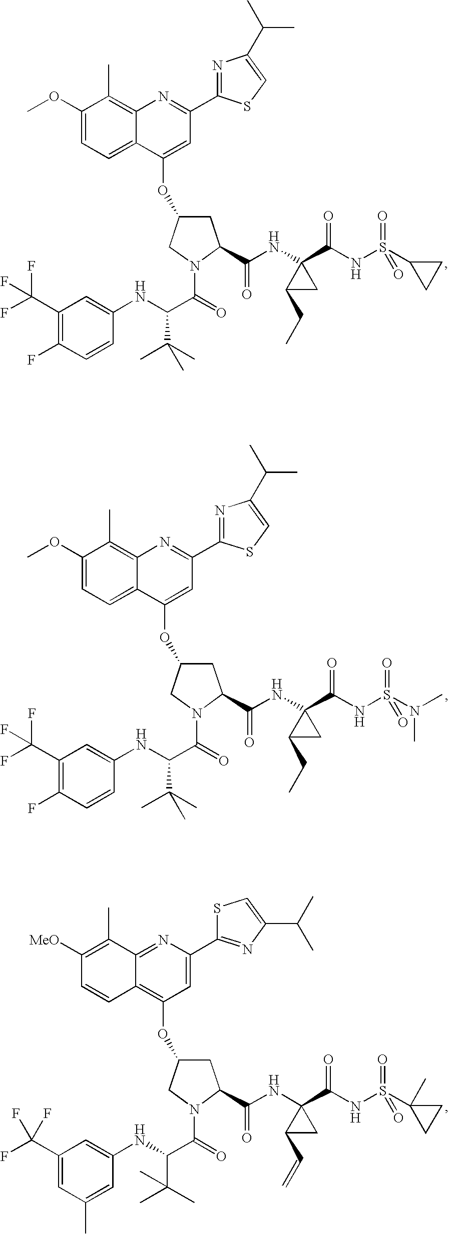

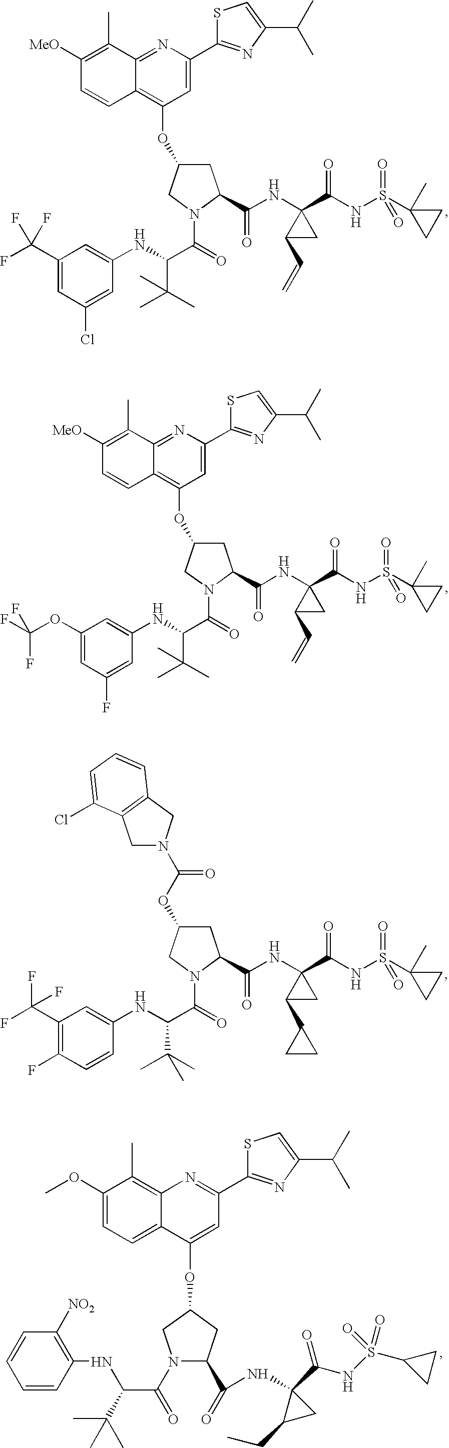

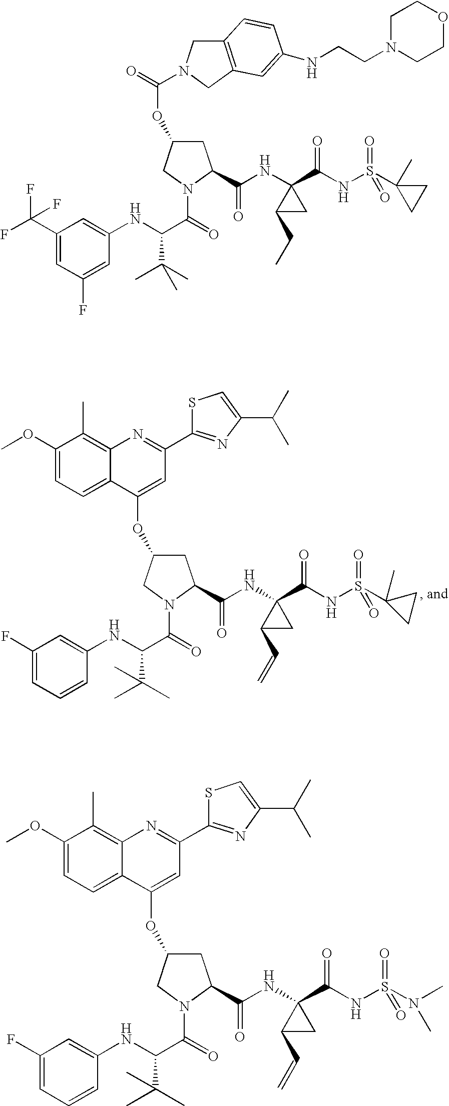

The present embodiments provide for a method of inhibiting NS3/NS4 protease activity comprising contacting a NS3/NS4 protease with a compound disclosed herein.

The present embodiments provide for a method of treating hepatitis by modulating NS3/NS4 protease comprising contacting a NS3/NS4 protease with a compound disclosed herein.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In many embodiments, a subject compound inhibits the enzymatic activity of a hepatitis virus C(HCV) NS3 protease. Whether a subject compound inhibits HCV NS3 protease can be readily determined using any known method. Typical methods involve a determination of whether an HCV polyprotein or other polypeptide comprising an NS3 recognition site is cleaved by NS3 in the presence of the agent. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits enzymatic activity of an HCV NS3 protease with an IC₅₀ of less than about 50 μM, e.g., a subject compound inhibits an HCV NS3 protease with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, or less than about 1 nM, or less.

In many embodiments, a subject compound inhibits the enzymatic activity of a hepatitis virus C(HCV) NS3 helicase. Whether a subject compound inhibits HCV NS3 helicase can be readily determined using any known method. In many embodiments, a subject compound inhibits NS3 enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS3 in the absence of the compound.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Treating a Hepatitis Virus Infection

The methods and compositions described herein are generally useful in treatment of an of HCV infection.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound disclosed herein, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound disclosed herein optionally in combination with an effective amount of one or more additional antiviral agents. In some embodiments, an effective amount of a compound disclosed herein, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound disclosed herein, and optionally one or more additional antiviral agents, is an amount that is effective to reduce viral load to lower than 100 genome copies/mL serum.

In some embodiments, an effective amount of a compound disclosed herein, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound disclosed herein, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound disclosed herein, and optionally one or more additional antiviral agents, is an amount effective to reduce ALT levels to less than about 45 IU/mL serum.

A therapeutically effective amount of a compound disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound disclosed herein and an additional antiviral agent is a synergistic amount. As used herein, a “synergistic combination” or a “synergistic amount” of a compound disclosed herein and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of a compound disclosed herein when administered at that same dosage as a monotherapy and (ii) the therapeutic or prophylactic benefit of the additional antiviral agent when administered at the same dosage as a monotherapy.

In some embodiments, a selected amount of a compound disclosed herein and a selected amount of an additional antiviral agent are effective when used in combination therapy for a disease, but the selected amount of a compound disclosed herein and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of a compound disclosed herein when used in combination therapy for a disease, where the selected amount of the additional antiviral agent provides no therapeutic benefit when used in monotherapy for the disease (2) regimens in which a selected amount of a compound disclosed herein enhances the therapeutic benefit of a selected amount of the additional antiviral agent when used in combination therapy for a disease, where the selected amount of a compound disclosed herein provides no therapeutic benefit when used in monotherapy for the disease and (3) regimens in which a selected amount of a compound disclosed herein and a selected amount of the additional antiviral agent provide a therapeutic benefit when used in combination therapy for a disease, where each of the selected amounts of a compound disclosed herein and the additional antiviral agent, respectively, provides no therapeutic benefit when used in monotherapy for the disease. As used herein, a “synergistically effective amount” of a compound disclosed herein and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound disclosed herein, and optionally one or more additional antiviral agents. Effective amounts of compounds disclosed herein, with and without one or more additional antiviral agents, as well as dosing regimens, are as discussed below.

Whether treatment with a compound disclosed herein, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound disclosed herein, and optionally one or more additional antiviral agents, is an amount that effects a change of one unit or more in the fibrosis stage based on pre- and post-therapy liver biopsies. In particular embodiments, a therapeutically effective amount of a compound disclosed herein, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound disclosed herein. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound disclosed herein, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound disclosed herein, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound disclosed herein, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound disclosed herein, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound disclosed herein, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound disclosed herein, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound disclosed herein, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compounds as described herein, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) can be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} eds., a Lippincott, Williams, & Wilkins; and Handbook of Pharmaceutical Excipients (2000) A. H. Kibbe et al., eds., 3^{rd}. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Other Antiviral or Antifibrotic Agents

As discussed above, a subject method will in some embodiments be carried out by administering a compound disclosed herein, and optionally one or more additional antiviral agent(s).

In some embodiments, the method further includes administration of one or more interferon receptor agonist(s). Interferon receptor agonists are described herein.

In other embodiments, the method further includes administration of pirfenidone or a pirfenidone analog. Pirfenidone and pirfenidone analogs are described herein.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester [5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man. Because of its strong inhibitory effect on cytochrome P450 3A and the inhibitory effect on cytochrome P450 2D6, ritonavir at doses below the normal therapeutic dosage may be combined with other protease inhibitors to achieve therapeutic levels of the second protease inhibitor while reducing the number of dosage units required, the dosing frequency, or both.

Coadministration of low-dose ritonavir may also be used to compensate for drug interactions that tend to decrease levels of a protease inhibitor metabolized by CYP3A. Its structure, synthesis, manufacture and formulation are described in U.S. Pat. No. 5,541,206 U.S. Pat. No. 5,635,523 U.S. Pat. No. 5,648,497 U.S. Pat. No. 5,846,987 and U.S. Pat. No. 6,232,333. The ritonavir may be administered orally in capsule or tablet or oral solution form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, an additional antiviral agent is administered during the entire course of NS3 inhibitor compound treatment. In other embodiments, an additional antiviral agent is administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The compounds described herein may be used in acute or chronic therapy for HCV disease. In many embodiments, the compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound is administered as a continuous infusion.

In many embodiments, a compound described herein is administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound are readily determinable by those of skill in the art by a variety of means. A preferred means is to measure the physiological potency of a given interferon receptor agonist.

In many embodiments, multiple doses of NS3 inhibitor compound are administered. For example, an NS3 inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Ribavirin

In some embodiments, the methods provide for combination therapy comprising administering an NS3 inhibitor compound as described above, and an effective amount of ribavirin. Ribavirin can be administered in dosages of about 400 mg, about 800 mg, about 1000 mg, or about 1200 mg per day.

One embodiment provides any of the above-described methods modified to include co-administering to the patient a therapeutically effective amount of ribavirin for the duration of the desired course of NS3 inhibitor compound treatment.

Another embodiment provides any of the above-described methods modified to include co-administering to the patient about 800 mg to about 1200 mg ribavirin orally per day for the duration of the desired course of NS3 inhibitor compound treatment. In another embodiment, any of the above-described methods may be modified to include co-administering to the patient (a) 1000 mg ribavirin orally per day if the patient has a body weight less than 75 kg or (b) 1200 mg ribavirin orally per day if the patient has a body weight greater than or equal to 75 kg, where the daily dosage of ribavirin is optionally divided into to 2 doses for the duration of the desired course of NS3 inhibitor compound treatment.

Combination Therapies with Levovirin

In some embodiments, the methods provide for combination therapy comprising administering an NS3 inhibitor compound as described above, and an effective amount of levovirin. Levovirin is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day. In some embodiments, levovirin is administered orally in dosages of about 400, about 800, about 1000, or about 1200 mg per day for the desired course of NS3 inhibitor compound treatment.

Combination Therapies with Viramidine

In some embodiments, the methods provide for combination therapy comprising administering an NS3 inhibitor compound as described above, and an effective amount of viramidine. Viramidine is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day. In some embodiments, viramidine is administered orally in dosages of about 800, or about 1600 mg per day for the desired course of NS3 inhibitor compound treatment.

Combination Therapies with Ritonavir

In some embodiments, the methods provide for combination therapy comprising administering an NS3 inhibitor compound as described above, and an effective amount of ritonavir. Ritonavir is generally administered in an amount ranging from about 50 mg to about 100 mg, from about 100 mg to about 200 mg, from about 200 mg to about 300 mg, from about 300 mg to about 400 mg, from about 400 mg to about 500 mg, or from about 500 mg to about 600 mg, twice per day. In some embodiments, ritonavir is administered orally in dosages of about 300 mg, or about 400 mg, or about 600 mg twice per day for the desired course of NS3 inhibitor compound treatment.

Combination Therapies with Alpha-Glucosidase Inhibitors

Suitable α-glucosidase inhibitors include any of the above-described imino-sugars, including long-alkyl chain derivatives of imino sugars as disclosed in U.S. Patent Publication No. 2004/0110795; inhibitors of endoplasmic reticulum-associated α-glucosidases; inhibitors of membrane bound α-glucosidase; miglitol (Glyset®), and active derivatives, and analogs thereof; and acarbose (Precose®), and active derivatives, and analogs thereof.

In many embodiments, the methods provide for combination therapy comprising administering an NS3 inhibitor compound as described above, and an effective amount of an α-glucosidase inhibitor administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

An α-glucosidase inhibitor can be administered 5 times per day, 4 times per day, tid (three times daily), bid, qd, qod, biw, tiw, qw, qow, three times per month, or once monthly. In other embodiments, an α-glucosidase inhibitor is administered as a continuous infusion.

In many embodiments, an α-glucosidase inhibitor is administered orally.

In connection with the above-described methods for the treatment of a flavivirus infection, treatment of HCV infection, and treatment of liver fibrosis that occurs as a result of an HCV infection, the methods provide for combination therapy comprising administering an NS3 inhibitor compound as described above, and an effective amount of α-glucosidase inhibitor administered to the patient at a dosage of from about 10 mg per day to about 600 mg per day in divided doses, e.g., from about 10 mg per day to about 30 mg per day, from about 30 mg per day to about 60 mg per day, from about 60 mg per day to about 75 mg per day, from about 75 mg per day to about 90 mg per day, from about 90 mg per day to about 120 mg per day, from about 120 mg per day to about 150 mg per day, from about 150 mg per day to about 180 mg per day, from about 180 mg per day to about 210 mg per day, from about 210 mg per day to about 240 mg per day, from about 240 mg per day to about 270 mg per day, from about 270 mg per day to about 300 mg per day, from about 300 mg per day to about 360 mg per day, from about 360 mg per day to about 420 mg per day, from about 420 mg per day to about 480 mg per day, or from about 480 mg to about 600 mg per day.

In some embodiments, the methods provide for combination therapy comprising administering an NS3 inhibitor compound as described above, and an effective amount of α-glucosidase inhibitor administered in a dosage of about 10 mg three times daily. In some embodiments, an α-glucosidase inhibitor is administered in a dosage of about 15 mg three times daily. In some embodiments, an α-glucosidase inhibitor is administered in a dosage of about 20 mg three times daily. In some embodiments, an α-glucosidase inhibitor is administered in a dosage of about 25 mg three times daily. In some embodiments, an α-glucosidase inhibitor is administered in a dosage of about 30 mg three times daily. In some embodiments, an α-glucosidase inhibitor is administered in a dosage of about 40 mg three times daily. In some embodiments, an α-glucosidase inhibitor is administered in a dosage of about 50 mg three times daily. In some embodiments, an α-glucosidase inhibitor is administered in a dosage of about 100 mg three times daily. In some embodiments, an α-glucosidase inhibitor is administered in a dosage of about 75 mg per day to about 150 mg per day in two or three divided doses, where the individual weighs 60 kg or less. In some embodiments, an α-glucosidase inhibitor is administered in a dosage of about 75 mg per day to about 300 mg per day in two or three divided doses, where the individual weighs 60 kg or more.

The amount of active ingredient (e.g., α-glucosidase inhibitor) that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific α-glucosidase inhibitor, the severity of the symptoms and the susceptibility of the subject to side effects. Preferred dosages for a given α-glucosidase inhibitor are readily determinable by those of skill in the art by a variety of means. A typical means is to measure the physiological potency of a given active agent.

In many embodiments, multiple doses of an α-glucosidase inhibitor are administered. For example, the methods provide for combination therapy comprising administering an NS3 inhibitor compound as described above, and an effective amount of α-glucosidase inhibitor administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Thymosin-α

In some embodiments, the methods provide for combination therapy comprising administering an NS3 inhibitor compound as described above, and an effective amount of thymosin-α. Thymosin-α (Zadaxin™) is generally administered by subcutaneous injection. Thymosin-α can be administered tid, bid, qd, qod, biw, tiw, qw, qow, three times per month, once monthly, substantially continuously, or continuously for the desired course of NS3 inhibitor compound treatment. In many embodiments, thymosin-α is administered twice per week for the desired course of NS3 inhibitor compound treatment. Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

Thymosin-α can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In one embodiment, thymosin-α is administered for the desired course of NS3 inhibitor compound treatment.

Combination Therapies with Interferon(s)

In many embodiments, the methods provide for combination therapy comprising administering an NS3 inhibitor compound as described above, and an effective amount of an interferon receptor agonist. In some embodiments, a compound disclosed herein and a Type I or III interferon receptor agonist are co-administered in the treatment methods described herein. Type I interferon receptor agonists suitable for use herein include any interferon-α (IFN-α). In certain embodiments, the interferon-α is a PEGylated interferon-α. In certain other embodiments, the interferon-α is a consensus interferon, such as INFERGEN® interferon alfacon-1. In still other embodiments, the interferon-α is a monoPEG (30 kD, linear)-ylated consensus interferon.

Effective dosages of an IFN-α range from about 3 μg to about 27 μg, from about 3 MU to about 10 MU, from about 90 μg to about 180 μg, or from about 18 μg to about 90 μg. Effective dosages of Infergen® consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS®PEGylated IFN-α2a contain an amount of about 90 μg to 270 μg, or about 180 μg, of drug per dose. Effective dosages of PEG-INTRON®PEGylated IFN-α2b contain an amount of about 0.5 μg to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 μg, or about 60 μg to about 180 μg, or about 90 μg to about 120 μg, of drug per dose. IFN-α can be administered daily, every other day, once a week, three times a week, every other week, three times per month, once monthly, substantially continuously or continuously.

In many embodiments, the Type I or Type III interferon receptor agonist and/or the Type II interferon receptor agonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations. Some embodiments provide any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration. In other embodiments, any of the above-described methods may be practiced in which the desired dosage of PEGylated IFN-α (PEG-IFN-α) is administered subcutaneously to the patient by bolus delivery qw, qow, three times per month, or monthly for the desired treatment duration.

In other embodiments, an NS3 inhibitor compound and a Type II interferon receptor agonist are co-administered in the treatment methods of the embodiments. Type II interferon receptor agonists suitable for use herein include any interferon-γ (IFN-γ).

Effective dosages of IFN-γ can range from about 0.5 μg/m² to about 500 μg/m², usually from about 1.5 μg/m² to 200 μg/m², depending on the size of the patient. This activity is based on 10⁶ international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m² to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In some embodiments, a Type I or a Type III interferon receptor agonist is administered in a first dosing regimen, followed by a second dosing regimen. The first dosing regimen of Type I or a Type III interferon receptor agonist (also referred to as “the induction regimen”) generally involves administration of a higher dosage of the Type I or Type III interferon receptor agonist. For example, in the case of Infergen® consensus IFN-α (CIFN), the first dosing regimen comprises administering CIFN at about 9 μg, about 15 μg, about 18 μg, or about 27 μg. The first dosing regimen can encompass a single dosing event, or at least two or more dosing events. The first dosing regimen of the Type I or Type III interferon receptor agonist can be administered daily, every other day, three times a week, every other week, three times per month, once monthly, substantially continuously or continuously.

The first dosing regimen of the Type I or Type III interferon receptor agonist is administered for a first period of time, which time period can be at least about 4 weeks, at least about 8 weeks, or at least about 12 weeks.

The second dosing regimen of the Type I or Type III interferon receptor agonist (also referred to as “the maintenance dose”) generally involves administration of a lower amount of the Type I or Type III interferon receptor agonist. For example, in the case of CIFN, the second dosing regimen comprises administering CIFN at a dose of at least about 3 μg, at least about 9 μg, at least about 15 μg, or at least about 18 μg. The second dosing regimen can encompass a single dosing event, or at least two or more dosing events.

The second dosing regimen of the Type I or Type III interferon receptor agonist can be administered daily, every other day, three times a week, every other week, three times per month, once monthly, substantially continuously or continuously.

In some embodiments, where an “induction”/“maintenance” dosing regimen of a Type I or a Type III interferon receptor agonist is administered, a “priming” dose of a Type II interferon receptor agonist (e.g., IFN-γ) is included. In these embodiments, IFN-γis administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase.

In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, the Type II interferon receptor agonist treatment is discontinued once Type I or a Type III interferon receptor agonist treatment begins.

In other embodiments, the Type I or Type III interferon receptor agonist is administered in single dosing regimen. For example, in the case of CIFN, the dose of CIFN is generally in a range of from about 3 μg to about 15 μg, or from about 9 μg to about 15 μg. The dose of Type I or a Type III interferon receptor agonist is generally administered daily, every other day, three times a week, every other week, three times per month, once monthly, or substantially continuously. The dose of the Type I or Type III interferon receptor agonist is administered for a period of time, which period can be, for example, from at least about 24 weeks to at least about 48 weeks, or longer.

In some embodiments, where a single dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase. In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, Type II interferon receptor agonist treatment is discontinued once Type I or a Type III interferon receptor agonist treatment begins.

In additional embodiments, an NS3 inhibitor compound, a Type I or III interferon receptor agonist, and a Type II interferon receptor agonist are co-administered for the desired duration of treatment in the methods described herein. In some embodiments, an NS3 inhibitor compound, an interferon-α, and an interferon-γ are co-administered for the desired duration of treatment in the methods described herein.

In some embodiments, the invention provides methods using an amount of a Type I or Type III interferon receptor agonist, a Type II interferon receptor agonist, and an NS3 inhibitor compound, effective for the treatment of HCV infection in a patient. Some embodiments provide methods using an effective amount of an IFN-α, IFN-γ, and an NS3 inhibitor compound in the treatment of HCV infection in a patient. One embodiment provides a method using an effective amount of a consensus IFN-α, IFN-γ and an NS3 inhibitor compound in the treatment of HCV infection in a patient.

In general, an effective amount of a consensus interferon (CIFN) and IFN-γ suitable for use in the methods of the embodiments is provided by a dosage ratio of 1 μg CIFN:10 μg IFN-γ, where both CIFN and IFN-γ are unPEGylated and unglycosylated species.

In one embodiment, the invention provides any of the above-described methods modified to use an effective amount of INFERGEN®consensus IFN-α and IFN-γ in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN® consensus IFN-α and IFN-γ in the treatment of virus infection in a patient comprising administering to the patient a dosage of INFERGEN® containing an amount of about 1 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 50 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN® consensus IFN-α and IFN-γ in the treatment of a virus infection in a patient comprising administering to the patient a dosage of INFERGEN® containing an amount of about 9 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 90 μg to about 100 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 30 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 200 μg to about 300 μg of drug per dose of IFN-γ, subcutaneously qd, qod, tiw, biw, qw, qow, three times per month, once monthly, or per day substantially continuously or continuously, for the desired duration of treatment with an NS3 inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a total weekly dosage of IFN-γ containing an amount of about 30 μg to about 1,000 μg of drug per week in divided doses administered subcutaneously qd, qod, tiw, biw, or administered substantially continuously or continuously, for the desired duration of treatment with an NS3 inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a total weekly dosage of IFN-γ containing an amount of about 100 μg to about 300 μg of drug per week in divided doses administered subcutaneously qd, qod, tiw, biw, or substantially continuously or continuously, for the desired duration of treatment with an NS3 inhibitor compound.

In general, an effective amount of IFN-α 2a or 2b or 2c and IFN-γ suitable for use in the methods of the embodiments is provided by a dosage ratio of 1 million Units (MU) IFN-α 2a or 2b or 2c: 30 μg IFN-γ, where both IFN-α 2a or 2b or 2c and IFN-γ are unPEGylated and unglycosylated species.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 1 MU to about 20 MU of drug per dose of IFN-α 2a, 2b or 2c subcutaneously qd, qod, tiw, biw, or per day substantially continuously or continuously, in combination with a dosage of IFN-γ containing an amount of about 30 μg to about 600 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of IFN-γ containing an amount of about 100 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of IFN-α 2a or 2b or 2c and IFN-γ in the treatment of a virus infection in a patient comprising administering to the patient a dosage of IFN-α 2a, 2b or 2c containing an amount of about 10 MU of drug per dose of IFN-α 2a, 2b or 2c subcutaneously qd, qod, tiw, biw, or per day substantially continuously or continuously, in combination with a dosage of IFN-γ containing an amount of about 300 μg of drug per dose of IFN-γ, subcutaneously qd, qod, tiw, biw, or per day substantially continuously or continuously, for the desired duration of treatment with an NS3 inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a total weekly dosage of IFN-γ containing an amount of about 30 μg to about 1,000 μg, of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 180 μg of drug per dose of PEGASYS®, subcutaneously qw, qow, three times per month, or monthly, in combination with a total weekly dosage of IFN-γ containing an amount of about 100 μg to about 300 μg, of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a total weekly dosage of IFN-γ containing an amount of about 30 μg to about 1,000 μg of drug per week administered in divided doses subcutaneously qd, qod, tiw, or biw, or administered substantially continuously or continuously, for the desired duration of treatment with an NS3 inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a total weekly dosage of IFN-γ containing an amount of about 100 μg to about 300 μg of drug per week administered in divided doses subcutaneously qd, qod, tiw, or biw, or administered substantially continuously or continuously, for the desired duration of treatment with an NS3 inhibitor compound.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 25 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; 200 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 25 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 9 μg INFERGEN® consensus IFN-α administered subcutaneously qd or tiw; and 200 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 100 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 150 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS3 inhibitor; and a regimen of 200 μg monoPEG(30 kD, linear)-ylated consensus IFN-α administered subcutaneously every 10 days or qw; and 100 μg Actimmune® human IFN-γ1b administered subcutaneously tiw, where the duration of therapy is 48 weeks.

Any of the above-described methods involving administering an NS3 inhibitor, a Type I interferon receptor agonist (e.g., an IFN-α), and a Type II interferon receptor agonist (e.g., an IFN-γ), can be augmented by administration of an effective amount of a TNF-α antagonist (e.g., a TNF-α antagonist other than pirfenidone or a pirfenidone analog). Exemplary, non-limiting TNF-α antagonists that are suitable for use in such combination therapies include ENBREL®, REMICADE®, and HUMIRA™.

One embodiment provides a method using an effective amount of ENBREL®; an effective amount of IFN-α; an effective amount of IFN-γ; and an effective amount of an NS3 inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment.

One embodiment provides a method using an effective amount of REMICADE®, an effective amount of IFN-α; an effective amount of IFN-γ; and an effective amount of an NS3 inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment.

One embodiment provides a method using an effective amount of HUMIRA™, an effective amount of IFN-α; an effective amount of IFN-γ; and an effective amount of an NS3 inhibitor in the treatment of an HCV infection in a patient, comprising administering to the patient a dosage of HUMIRA™ containing an amount of from about 0.1 to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a H′HUMIRA™, subcutaneously qd, qod, tiw, biw, qw, qow, three times per month, once monthly, or once every other month, or per day substantially continuously or continuously, for the desired duration of treatment.

Combination Therapies with Pirfenidone

In many embodiments, the methods provide for combination therapy comprising administering an NS3 inhibitor compound as described above, and an effective amount of pirfenidone or a pirfenidone analog. In some embodiments, an NS3 inhibitor compound, one or more interferon receptor agonist(s), and pirfenidone or pirfenidone analog are co-administered in the treatment methods of the embodiments. In certain embodiments, an NS3 inhibitor compound, a Type I interferon receptor agonist, and pirfenidone (or a pirfenidone analog) are co-administered. In other embodiments, an NS3 inhibitor compound, a Type I interferon receptor agonist, a Type II interferon receptor agonist, and pirfenidone (or a pirfenidone analog) are co-administered. Type I interferon receptor agonists suitable for use herein include any IFN-α, such as interferon alfa-2a, interferon alfa-2b, interferon alfacon-1, and PEGylated IFN-α's, such as peginterferon alfa-2a, peginterferon alfa-2b, and PEGylated consensus interferons, such as monoPEG (30 kD, linear)-ylated consensus interferon. Type II interferon receptor agonists suitable for use herein include any interferon-γ.

Pirfenidone or a pirfenidone analog can be administered once per month, twice per month, three times per month, once per week, twice per week, three times per week, four times per week, five times per week, six times per week, daily, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or a specific pirfenidone analog include a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally in one to five divided doses per day. Other doses and formulations of pirfenidone and specific pirfenidone analogs suitable for use in the treatment of fibrotic diseases are described in U.S. Pat. Nos. 5,310,562; 5,518,729; 5,716,632; and 6,090,822.

One embodiment provides any of the above-described methods modified to include co-administering to the patient a therapeutically effective amount of pirfenidone or a pirfenidone analog for the duration of the desired course of NS3 inhibitor compound treatment.

Combination Therapies with TNF-α Antagonists

In many embodiments, the methods provide for combination therapy comprising administering an effective amount of an NS3 inhibitor compound as described above, and an effective amount of TNF-α antagonist, in combination therapy for treatment of an HCV infection.

Effective dosages of a TNF-α antagonist range from 0.1 μg to 40 mg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, effective dosages of a TNF-α antagonist are expressed as mg/kg body weight. In these embodiments, effective dosages of a TNF-α antagonist are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, a TNF-α antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The TNF-α antagonist can be administered tid, bid, qd, qod, biw, tiw, qw, qow, three times per month, once monthly, substantially continuously, or continuously.

In many embodiments, multiple doses of a TNF-α antagonist are administered. For example, a TNF-α antagonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A TNF-α antagonist and an NS3 inhibitor are generally administered in separate formulations. A TNF-α antagonist and an NS3 inhibitor may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

One embodiment provides a method using an effective amount of a TNF-α antagonist and an effective amount of an NS3 inhibitor in the treatment of an HCV infection in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides a method using an effective amount of ENBREL® and an effective amount of an NS3 inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment with an NS3 inhibitor compound.

One embodiment provides a method using an effective amount of REMICADE® and an effective amount of an NS3 inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment with an NS3 inhibitor compound.

One embodiment provides a method using an effective amount of HUMIRA™ and an effective amount of an NS3 inhibitor in the treatment of an HCV infection in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment with an NS3 inhibitor compound.

Combination Therapies with Thymosin-α

In many embodiments, the methods provide for combination therapy comprising administering an effective amount of an NS3 inhibitor compound as described above, and an effective amount of thymosin-α, in combination therapy for treatment of an HCV infection.

Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

One embodiment provides a method using an effective amount of ZADAXIN™ thymosin-α and an effective amount of an NS3 inhibitor in the treatment of an HCV infection in a patient, comprising administering to the patient a dosage of ZADAXIN™ containing an amount of from about 1.0 mg to about 1.6 mg per dose, subcutaneously twice per week for the desired duration of treatment with the NS3 inhibitor compound.

Combination Therapies with a TNF-α Antagonist and an Interferon

Some embodiments provide a method of treating an HCV infection in an individual having an HCV infection, the method comprising administering an effective amount of an NS3 inhibitor, and effective amount of a TNF-α antagonist, and an effective amount of one or more interferons.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 30 μg to about 1,000 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 100 μg to about 300 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 1 MU to about 20 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 10 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 180 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS3 inhibitor compound.

Combination Therapies with Other Antiviral Agents

Other agents such as inhibitors of HCV NS3 helicase are also attractive drugs for combinational therapy, and are contemplated for use in combination therapies described herein. Ribozymes such as Heptazyme™ and phosphorothioate oligonucleotides which are complementary to HCV protein sequences and which inhibit the expression of viral core proteins are also suitable for use in combination therapies described herein.

In some embodiments, the additional antiviral agent(s) is administered during the entire course of treatment with the NS3 inhibitor compound described herein, and the beginning and end of the treatment periods coincide. In other embodiments, the additional antiviral agent(s) is administered for a period of time that is overlapping with that of the NS3 inhibitor compound treatment, e.g., treatment with the additional antiviral agent(s) begins before the NS3 inhibitor compound treatment begins and ends before the NS3 inhibitor compound treatment ends; treatment with the additional antiviral agent(s) begins after the NS3 inhibitor compound treatment begins and ends after the NS3 inhibitor compound treatment ends; treatment with the additional antiviral agent(s) begins after the NS3 inhibitor compound treatment begins and ends before the NS3 inhibitor compound treatment ends; or treatment with the additional antiviral agent(s) begins before the NS3 inhibitor compound treatment begins and ends after the NS3 inhibitor compound treatment ends.

The NS3 inhibitor compound can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional antiviral agents.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 100 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 150 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 200 μg of drug per dose, subcutaneously once weekly, once every 8 days, or once every 10 days for the desired treatment duration with an NS3 inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 9 μg of drug per dose, subcutaneously once daily or three times per week for the desired treatment duration with an NS3 inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 15 μg of drug per dose, subcutaneously once daily or three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 25 μg of drug per dose, subcutaneously three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 50 μg of drug per dose, subcutaneously three times per week for the desired treatment duration with an NS3 inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 100 μg of drug per dose, subcutaneously three times per week for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring a TNF antagonist regimen can be modified to replace the subject TNF antagonist regimen with a TNF antagonist regimen comprising administering a dosage of a TNF antagonist selected from the group of: (a) etanercept in an amount of 25 mg of drug per dose subcutaneously twice per week, (b) infliximab in an amount of 3 mg of drug per kilogram of body weight per dose intravenously at weeks 0, 2 and 6, and every 8 weeks thereafter, or (c) adalimumab in an amount of 40 mg of drug per dose subcutaneously once weekly or once every 2 weeks; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γcombination regimen with an IFN-α and IFN-γ combination regimen comprising: (a) administering a dosage of monoPEG (30 kD, linear)-ylated consensus IFN-αcontaining an amount of 200 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 25 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 50 μg of drug per dose, subcutaneously three times per week; for the desired treatment duration with an NS3 inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 100 μg of drug per dose, subcutaneously three times per week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 9 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 15 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 25 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 50 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 100 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS3 inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2a comprising administering a dosage of peginterferon alfa-2a containing an amount of 180 μg of drug per dose, subcutaneously once weekly for the desired treatment duration with an NS3 inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2b comprising administering a dosage of peginterferon alfa-2b containing an amount of 1.0 μg to 1.5 μg of drug per kilogram of body weight per dose, subcutaneously once or twice weekly for the desired treatment duration with an NS3 inhibitor compound.

As non-limiting examples, any of the above-described methods can be modified to include administering a dosage of ribavirin containing an amount of 400 mg, 800 mg, 1000 mg or 1200 mg of drug orally per day, optionally in two or more divided doses per day, for the desired treatment duration with an NS3 inhibitor compound.

As non-limiting examples, any of the above-described methods can be modified to include administering a dosage of ribavirin containing (i) an amount of 1000 mg of drug orally per day for patients having a body weight of less than 75 kg or (ii) an amount of 1200 mg of drug orally per day for patients having a body weight of greater than or equal to 75 kg, optionally in two or more divided doses per day, for the desired treatment duration with an NS3 inhibitor compound.

As non-limiting examples, any of the above-described methods can be modified to replace the subject NS3 inhibitor regimen with an NS3 inhibitor regimen comprising administering a dosage of 0.01 mg to 0.1 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 0.1 mg to 1 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 1 mg to 10 mg of drug per kilogram of body weight orally daily, optionally in two or more divided doses per day, for the desired treatment duration with the NS3 inhibitor compound.

As non-limiting examples, any of the above-described methods can be modified to replace the subject NS3 inhibitor regimen with an NS3 inhibitor regimen comprising administering a dosage of 10 mg to 100 mg of drug per kilogram of body weight orally daily, optionally in two or more divided doses per day, for the desired treatment duration with the NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 0.01 mg to 0.1 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 0.1 mg to 1 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 1 mg to 10 mg of drug per kilogram of body weight orally daily, optionally in two or more divided doses per day, for the desired treatment duration with an NS3 inhibitor compound.

As non-limiting examples, any of the above-described methods featuring an NS5B inhibitor regimen can be modified to replace the subject NS5B inhibitor regimen with an NS5B inhibitor regimen comprising administering a dosage of 10 mg to 100 mg of drug per kilogram of body weight orally daily, optionally in two or more divided doses per day, for the desired treatment duration with an NS3 inhibitor compound.

### Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, some embodiments provide any of the above-described methods for the treatment of HCV infection in which the subject method is modified to treat a treatment failure patient for a duration of 48 weeks.

Other embodiments provide any of the above-described methods for HCV in which the subject method is modified to treat a non-responder patient, where the patient receives a 48 week course of therapy.

Other embodiments provide any of the above-described methods for the treatment of HCV infection in which the subject method is modified to treat a relapser patient, where the patient receives a 48 week course of therapy.

Other embodiments provide any of the above-described methods for the treatment of HCV infection in which the subject method is modified to treat a naïve patient infected with HCV genotype 1, where the patient receives a 48 week course of therapy.

Other embodiments provide any of the above-described methods for the treatment of HCV infection in which the subject method is modified to treat a naïve patient infected with HCV genotype 4, where the patient receives a 48 week course of therapy.

Other embodiments provide any of the above-described methods for the treatment of HCV infection in which the subject method is modified to treat a naïve patient infected with HCV genotype 1, where the patient has a high viral load (HVL), where “HVL” refers to an HCV viral load of greater than 2×10⁶ HCV genome copies per mL serum, and where the patient receives a 48 week course of therapy.

One embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per ml of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per ml of patient serum and then (2) administering to the patient the drug therapy of the subject method for a time period of about 24 weeks to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

Another embodiment provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

### Subjects Suitable for Treatment

Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (MBA). Such individuals may also, but need not, have elevated serum ALT levels.

Individuals who are clinically diagnosed as infected with HCV include nave individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system).

### Preparation of NS3 Inhibitors

### Methodology

The HCV protease inhibitors in the following sections can be prepared according to the procedures and schemes shown in each section. The numberings in each of the following Preparation of NS3 Inhibitor sections are meant for that specific section only, and should not be construed or confused with the same numberings in other sections.

### Preparation of N-Aryl Tert-Leucine Amino Acids

### General Procedure:

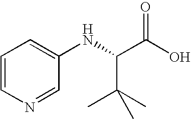

A suspension of 3-iodopyridine (156 mg, 0.76 mmol, 1.0 eq), L-tert-leucine (200 mg, 1.53 mmol, 2.0 eq), potassium carbonate (316 mg, 2.29 mmol, 3.0 eq) and copper(I) iodide (29 mg, 0.15 mmol, 0.2 eq) in tert-butanol (5 mL) was degassed by purging with nitrogen for 5 minutes at 40° C. in a pressurised reaction tube. The pressure tube was sealed and the reaction mixture stirred at 120° C. overnight. The reaction mixture was then evaporated in vacuo. The residue was absorbed onto silica gel (1 mL), placed onto a silica gel column and purified, eluting with methanol:dichloromethane (1:9) to give 120 mg (75%) of the desired product.

1H NMR (250 MHz, MeOD) δ 8.21 (d, J=8.07 Hz, 1H), 7.14-8.31 (m, 3H), 3.81 (s, 1H), 1.13 (s, 9H)

LC-MS: purity 74% (UV), tR 0.90 min m/z [M+H]+ 209.05

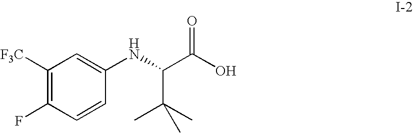

Procedure as described for I-1. Yield: 745 mg (67%). 1H NMR (500 MHz, CHLOROFORM-d) δ 6.97-7.06 (m, 1H), 6.84 (qq, 1H), 6.73-6.81 (m, 1H), 3.73 (s, 1H), 1.10 (d, J=0.92 Hz, 9H). LC-MS: purity 99% (UV), tR 2.15 min m/z [M+H]+ 294.00

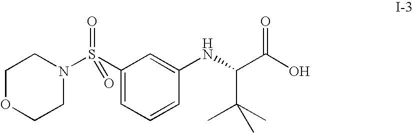

Procedure as described for I-1. Yield: 137 mg (50%)

1H NMR (250 MHz, CHLOROFORM-d) 07.27-7.36 (m, 1H), 7.08 (dd, J=0.84, 7.69 Hz, 1H), 6.94-7.02 (m, 1H), 6.86 (dt, J=1.16, 8.19 Hz, 1H), 3.85 (s, 1H), 3.58-3.79 (m, 4H), 2.84-3.13 (m, 4H), 1.11 (s, 9H)

LC-MS: purity 91% (UV), tR 1.81 min m/z [M+H]+ 357.05

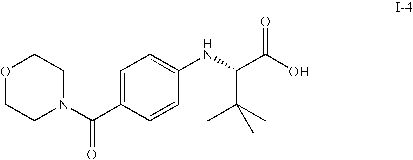

Procedure as described for I-1. Yield: 414 mg (97%)

1H NMR (250 MHz, CHLOROFORM-d) δ ppm 7.23 (d, J=8.68 Hz, 2H) 6.57 (d, J=8.68 Hz, 2H) 3.76 (s, 1H) 3.67 (br. s., 8H) 1.08 (s, 9H)

LC-MS: purity 92% (UV), tR 1.60 min m/z [M+H]+ 321.10

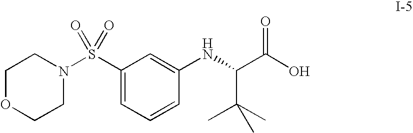

Procedure as described for I-1. Yield: 276 mg (58%)

1H NMR (250 MHz, CHLOROFORM-d) δ 7.29-7.39 (m, 1H), 7.06-7.15 (m, 1H), 6.97-7.06 (m, 1H), 6.82-6.93 (m, 1H), 3.86 (s, 1H), 3.68-3.79 (m, 4H), 2.96-3.05 (m, 4H), 1.12 (s, 9H).

LC-MS: purity 94% (UV), tR 1.94 min m/z [M+H]+ 357.05

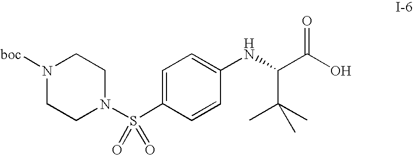

Procedure as described for I-1. Yield: 61 mg (10%)

1H NMR (250 MHz, CHLOROFORM-d) δ ppm 7.54 (d, J=8.68 Hz, 2H) 6.69 (d, J=8.38 Hz, 2H) 4.58-4.76 (m, 1H) 3.89 (br. s., 1H) 3.43-3.57 (m, 4H) 2.86-3.00 (m, 4H) 1.42 (s, 9H) 1.12 (s, 9H).

LC-MS: purity 89% (UV), tR 2.06 min m/z [M+Na]+478.15.

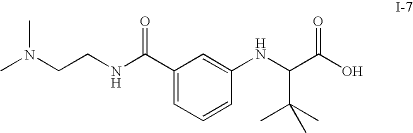

Procedure as described for I-1. Yield: 234 mg (55%)

1H NMR (250 MHz, MeOD)-δ ppm 7.10-7.20 (m, 1H) 6.99-7.10 (m, 2H) 6.77-6.91 (m, 1H) 3.64 (br. s., 1H) 3.51 (t, J=6.93 Hz, 2H) 2.60 (t, J=6.85 Hz, 2H) 2.34 (s, 6H) 1.05-1.13 (m, 9H)

LC-MS: purity 97% (UV), tR 1.16 min m/z [M+H]+ 322.10

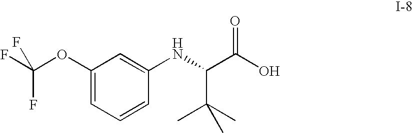

Procedure as described for I-1. Yield: 64.7 mg (40%)

1H NMR (250 MHz, CHLOROFORM-d) ⊖ 7.17 (t, J=8.15 Hz, 1H), 6.53-6.67 (m, 2H), 6.50 (s, 1H), 3.78 (s, 1H), 1.05-1.15 (m, 9H)

LC-MS: purity 85% (UV), tR 2.18 min m/z [M+H]+ 292.00

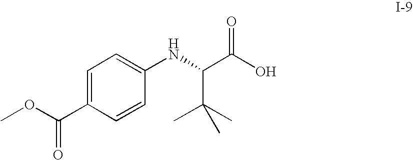

Procedure as described for I-1. Yield: 1.22 g (86%)

1H NMR (250 MHz, CHLOROFORM-d) δ 7.56-7.86 (m, 2H), 6.29-6.70 (m, 2H), 3.63-3.86 (m, 3H), 3.03-3.39 (m, 1H), 0.95 (s, 9H)

LC-MS: purity 90% (UV), tR 1.26 min m/z [M+H]+ 266.10

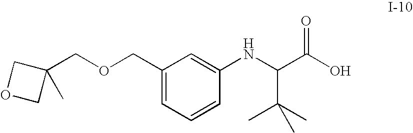

Procedure as described for I-1. Yield: 250 mg (58%)

1H NMR (250 MHz, CHLOROFORM-d) δ ppm 7.16 (t, J=7.92 Hz, 1H) 6.68-6.78 (m, 2H) 6.63 (d, J=8.22 Hz, 1H) 4.44-4.60 (m, 4H) 4.33-4.41 (m, 2H) 3.78 (s, 1H) 3.47-3.54 (m, 2H) 1.29-1.35 (m, 3H) 1.12 (s, 9H)

LC-MS: purity 93% (UV), tR 1.87 min m/z [M+H]+ 322.20

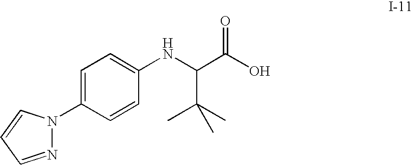

Procedure as described for I-1. Yield: 287 mg (79%)

1H NMR (250 MHz, CHLOROFORM-d) δ 7.67-7.81 (m, 2H), 7.41 (d, J=8.83 Hz, 2H), 6.71 (d, J=8.83 Hz, 2H), 6.40-6.46 (m, 1H), 3.79 (s, 1H), 2.08 (d, J=10.20 Hz, 1H), 1.05-1.18 (m, 9H)

LC-MS: purity 93% (UV), tR 1.77 min m/z [M+H]+ 274.15

### Syntheses of Isoindoline Intermediates

Preparation of 1-N-Boc-5-amino-isoindoline

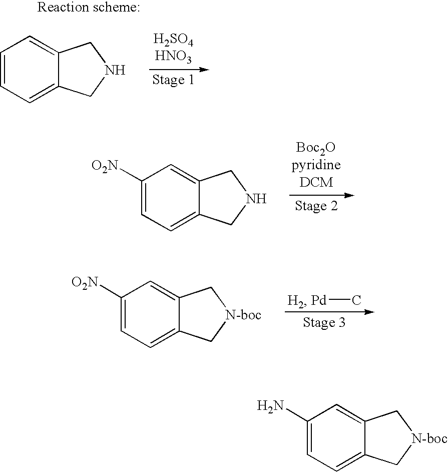

### Stage 1: 5-nitro-isoindoline (I-12)

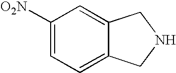

A solution of isoindoline (3.9 g, 32.8 mmol, 1.0 eq.) in dichloromethane (20 mL) was stirred below −20° C. with exclusion of moisture while adding dropwise sulphuric acid (98%, 16.0 mL). The 2-layer mixture was allowed to reach 20° C. and then dichloromethane was removed under vacuum.

The resulting pale brown solution was stirred and kept below 20° C. while adding nitric acid (70%, 3.9 mL) dropwise. The resulting pale orange-red solution was added with stirring to ice/water (300 mL) and tert-butyl methyl ether (100 mL). Sodium hydrogen carbonate (59 g) was added in portions and finally 4M aqueous sodium hydroxide (10 mL).

The layers were separated and the aqueous phase extracted with tert-butyl methyl ether (4×150 mL). The combined organic phases were dried (sodium sulphate) and evaporated giving a red-brown gum (4.6 g, 85%) which was used in the next stage without purification.

1H NMR (250 MHz, CHLOROFORM-d) δ ppm 8.05-8.19 (m, 2H) 7.38 (d, J=8.83 Hz, 1H) 4.32 (s, 4H) 2.14 (br. s., 1H)

### Stage 2: 2-N-boc-5-nitro-isoindoline (I-13)

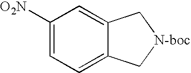

5-Nitroisoindoline (4.6 g, 28.0 mmol, 1.0 eq.) was dissolved in dry pyridine (10 mL) and dichloromethane (25 mL) was added (some precipitation noticed). Di-tert-butyl dicarbonate (6.8 g, 31.2 mmol, 1.1 eq.) was added causing gentle boiling of the solution. The solution was allowed to stand for 3 h then evaporated under vacuum. The resulting gum was triturated with methanol (25 mL) to give the title product as a pale beige solid (5.1 g; 69%).

1H NMR (500 MHz, CHLOROFORM-d) δ ppm 8.03-8.25 (m, 2H) 7.34-7.51 (m, 1H) 4.76 (d, J=15.59 Hz, 4H) 1.53 (s, 9H)

### Stage 3: 2-N-boc-5-amino-isoindoline (I-14)

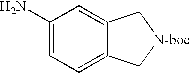

2-N-Boc-5-nitro-isoindoline (4.45 g, 16.86 mmol, 1.0 eq.) was dissolved in ethanol (300 mL). The solution was added to a 1 L round bottom flask containing 10% Palladium on charcoal (1.0 g, 50% wet paste). The reaction flask was purged three times with nitrogen gas and another three times with hydrogen gas. The flask was connected to a hydrogenator so that the volume of consumed hydrogen could be monitored. After 20 min, the uptake of hydrogen stopped. Reaction was stopped and t.l.c analysis (neat dichloromethane) revealed the reaction to be complete. The catalyst was separated by filtration and the solvent removed under vacuum to give 4.4 g (99% corrected for residual ethanol) of the title compound as an olive oil which contained residual ethanol. The product was used in the next step without further purification.

1H NMR (500 MHz, CHLOROFORM-d) δ ppm 6.90-7.10 (m, 1H) 6.45-6.65 (m, 2H) 4.50-4.63 (m, 4H) 3.52 (br. s., 2H) 1.51 (s, 9H)

LC-MS: purity 96% (UV), tR 0.98 min, m/z [M+H]+ 235.10

Synthesis of 5-substituted-isoindolines

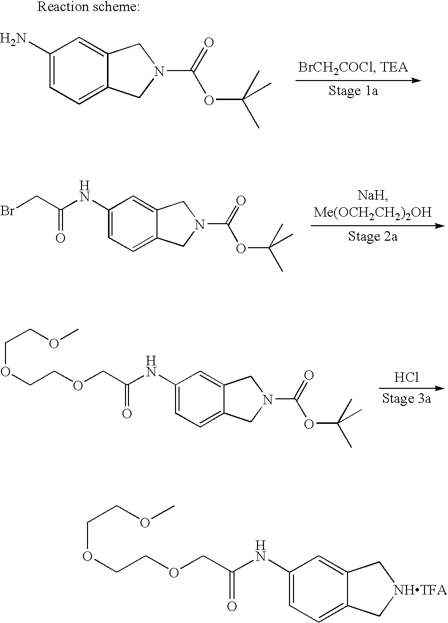

### Stage 1a: 5-(Bromoacetylamino)-isoindoline (I-15)

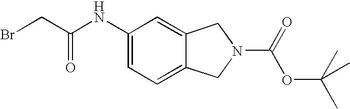

5-Amino-isoindoline (3.68 g, 15.7 mmol, 1.0 eq.) was dissolved in tetrahydrofuran (40 mL). Pyridine (2.5 mL, 31.4 mmol, 2.0 eq.) was added as a single portion and the reaction mixture cooled to 0° C. Bromoacetyl chloride (2.6 mL, 31.4 mmol, 2.0 eq.) was added dropwise and the reaction mixture left to warm to ambient temperature. Stirring was continued at ambient temperature for a further 15 hours, by when LCMS analysis showed all the starting material to be consumed. The solvent was removed in vacuo and the residue purified by flash column chromatography using a ethyl acetate:heptanes gradient (2:8 to 4:6). After combining the relevant fractions and removing the solvent under vacuum, 3.5 g (65%) of the title compound was isolated.

1H NMR (500 MHz, CHLOROFORM-d) δ ppm 8.12-8.42 (m, 1H) 7.45-7.73 (m, 1H) 7.14-7.43 (m, 2H) 4.55-4.78 (m, 4H) 4.01-4.25 (m, 2H) 1.52 (s, 9H)

LC-MS: purity 97% (UV), tR 1.28 min m/z [M+H]+ 255.00

### Stage 2a: I-16

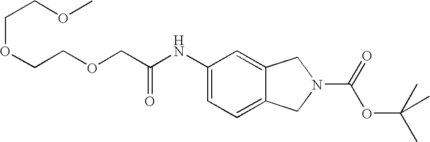

Sodium hydride (60% dispersion in oil, 40 mg, 1 mmol, 1.3 eq.) and acetonitrile (2 mL) were changed into a 10 mL round bottom flask. Methoxyethoxyethanol (120 mg, 1 mmol, 1.3 eq.) was added dropwise and the reaction mixture stirred at ambient temperature for 30 minutes. 5-(Bromoacetylamino)-isoindoline (272 mg, 0.77 mmol, 1.0 eq.) was added as single portion followed by acetonitrile (1 mL) and the reaction mixture was stirred at ambient temperature for 15 hours. The formation of a pale beige suspension was noticed to start after c.a. 1 hour. The solvent was removed under vacuum and the residue was partitioned between water (10 mL) and tert-butylmethyl ether (10 mL). The organic phase was collected and the aqueous phase was further extracted with tert-butylmethyl ether (10 mL). The organic phases were combined, dried over sodium sulphate, filtered and the solvent removed under vacuum to give 309 mg (99%) of the title compound as a yellow oil which was used in the next step without further purification.

LC-MS: purity 85% (UV), tR 1.85 min m/z [M+Na]+417.20

### Stage 2a: I-17

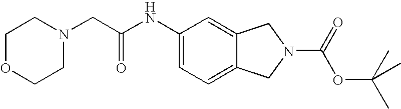

5-(Bromoacetylamino)-isoindoline (400 mg, 1.12 mmol, 1.0 eq.), morpholine (0.108 mL, 1.24 mmol, 1.1 eq.), potassium carbonate (156 mg, 1.12 mmol, 1.0 eq.) and acetonitrile (59 mL) were charged into a 100 mL round bottom flask. The reaction mixture was heated at 80° C. for 15 hours. The solvent was evaporated and the residue partitioned between water (25 mL) and dichloromethane (25 mL). The organic phase was collected and the aqueous phase back extracted with dichloromethane (25 mL). The organic phases were combined, dried over sodium sulphate, filtered and the solvent removed under vacuum to give 380 mg (95%) of the title compound as a greyish sticky solid.

1H NMR (250 MHz, MeOD) [symbol] ppm 7.61 (s, 1H) 7.40-7.51 (m, 1H) 7.21-7.29 (m, 1H) 4.57-4.67 (m, 4H) 3.74-3.81 (m, 4H) 3.18 (s, 2H) 2.53-2.66 (m, 4H) 1.53 (s, 9H)

LC-MS: purity 97% (UV), tR 1.33 min m/z [M+H]+ 362.55

### Stage 3a: I-18

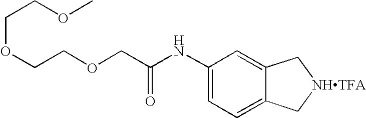

Stage 2a intermediate (300 mg, 0.761 mmol, 1.0 eq.) was dissolved in dichloromethane (15 mL) and cooled to 0° C. Trifluoroacetic acid (1 mL) was added dropwise and the reaction mixtured stirred at 0° C. for 30 min. The reaction mixture was left to warm to ambient temperature and stirred at this temperature for a further 2 hours. LCMS after 2 hours showed full conversion to the desired product. The solvent was removed under vacuum to give 305 mg (99%) of the title compound which was used in the next step without further purification.

LC-MS: purity 100% (UV), tR 0.80 min m/z [M+H]+ 295.15

### Stage 3a: I-19

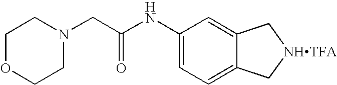

Stage 2a intermediate (380 mg, 1.05 mmol, 1.0 eq.) was dissolved in a trifluoroacetic acid:dichloromethane solution (2:8, 5 mL) and the reaction mixture was stirred at ambient temperature for a further 0.5 hours. LCMS showed full conversion to the desired product. The solvent was removed under vacuum to give 390 mg (99%) of the title compound which was used in the next step without further purification.

LC-MS: purity 100% (ELS), tR 0.24 min m/z [M+H]+ 262.10

Synthesis of N-(2,3-dihydro-1H-isoindol-5-yl)-2-methoxy-acetamide,trifluoro-acetamide

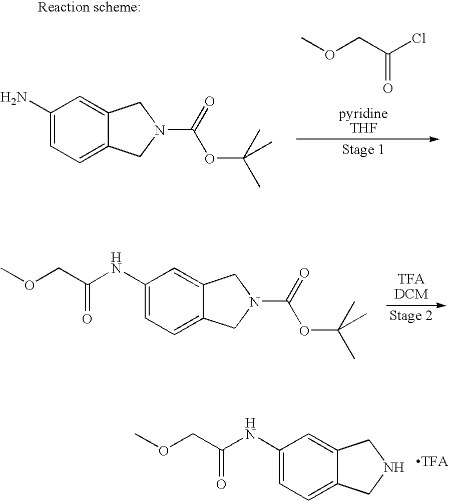

### Stage 1:

Methoxyacetyl chloride (562 μL, 6.14 mmol, 2.0 eq.) was added dropwise to a solution of the isoindoline (720 mg, 3.07 mmol, 1.0 eq.) and pyridine (497 μL, 6.14 mmol, 2.0 eq.) in tetrahydrofuran (10 mL) at 0° C. and stirred overnight whilst allowing to warm to ambient temperature. The reaction mixture was evaporated under vacuum, purification by silica gel column chromatography, eluting with ethyl acetate:heptanes (2:8 to 6:4) gave 850 mg (90%) of the desired product.

1H NMR (250 MHz, CHLOROFORM-d) δ 8.27 (br. s., 1H), 7.06-7.89 (m, 3H), 4.45-4.85 (m, 4H), 3.94-4.20 (m, 2H), 3.36-3.65 (m, 3H), 1.52 (s, 9H).

LC-MS: purity 100% (UV), tR 1.26 min m/z 206.00

### Stage 2:

A solution of TFA in dichloromethane was added to the BOC isoindoline at 0° C. and stirred for 4 hours. The reaction mixture was then evaporated in vacuo and used without further purification.

### Syntheses of Tripeptide Final Products:

### General Procedure for the Preparation of Tripeptide Analogues: Synthesis of 1

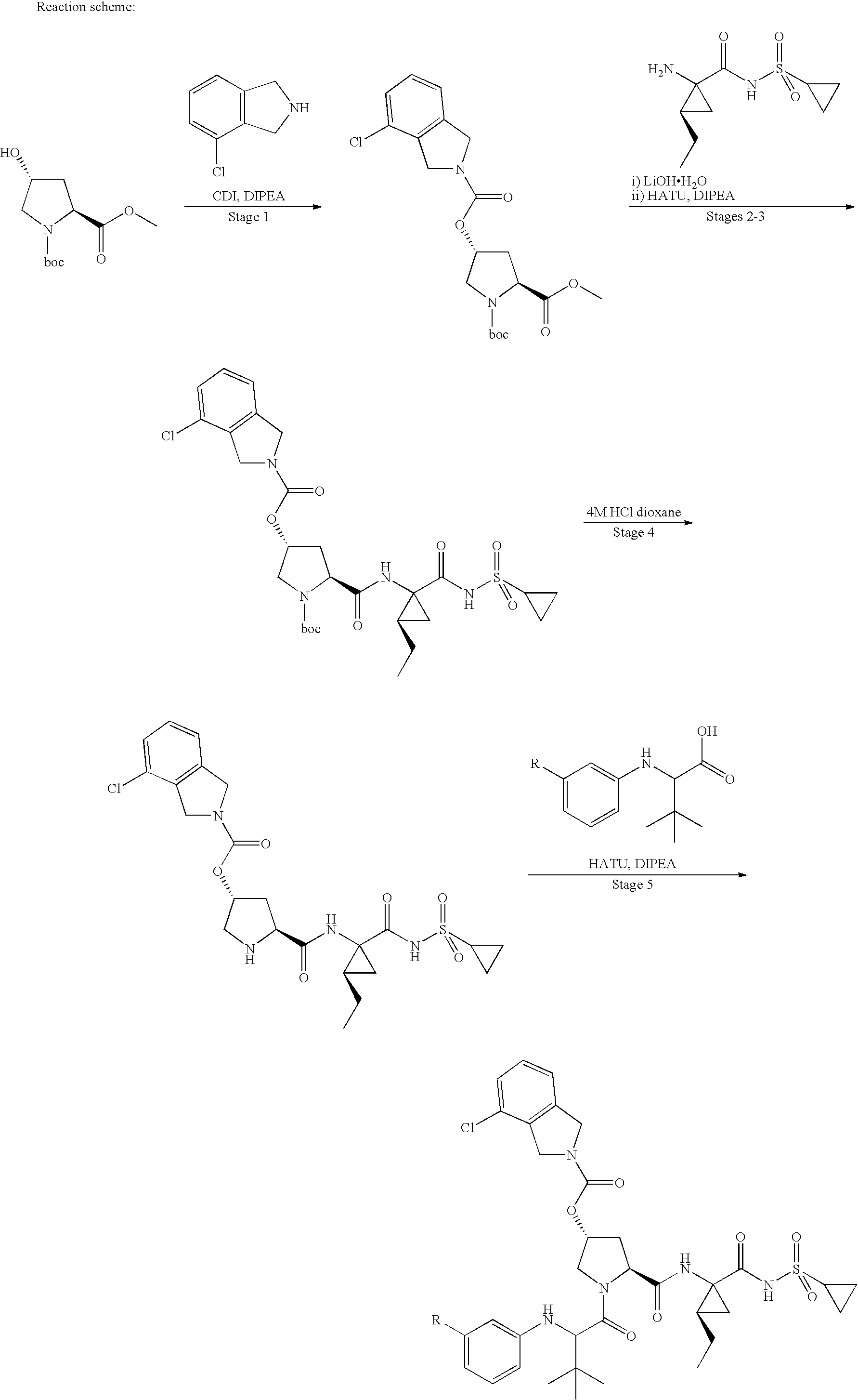

### Stage 1: I-20

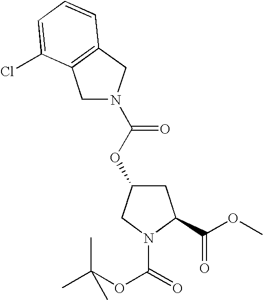

CDI (0.99 g, 6.12 mmol, 1.5 eq) was added to a solution of N—BOC-trans-4-hydroxy-L-proline methyl ester (1.00 g, 4.08 mmol, 1.0 eq) in tetrahydrofuran (26 mL) at 0° C. and stirred overnight whilst allowing to warm to ambient temperature. 4-Chloroisoindoline hydrochloride (0.74 g, 3.87 mmol, 0.95 eq) was then added to the reaction mixture followed by triethylamine (1.14 mL, 8.15 mmol, 0.95 eq) and stirred overnight at ambient temperature. The reaction mixture was diluted with ethyl acetate (100 mL) and washed with 0.5 M hydrochloric acid (100 mL), then with sat. aqueous sodium hydrogen carbonate (100 mL), dried over sodium sulphate, filtered, and the solvent removed in vacuo. Purification by flash column chromatography, eluting with ethyl acetate:heptanes (2:3) gave 1.1 g (66%) of the desired product.

1H NMR (250 MHz, CHLOROFORM-d) δ 6.88-7.38 (m, 3H), 5.29-5.38 (m, 1H), 4.66-4.81 (m, 4H), 4.31-4.56 (m, 1H), 3.60-3.86 (m, 5H), 2.36-2.60 (m, 1H), 2.17-2.33 (m, 1H), 1.32-1.59 (m, 9H)

LC-MS: purity 95% (UV), tR 1.49 min m/z [M+1-100]+325.00

### Stage 2: I-21

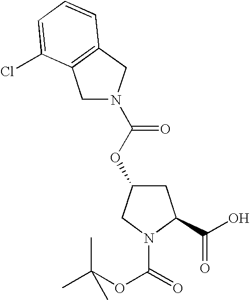

A solution of lithium hydroxide monohydrate (148 mg, 3.53 mmol, 1.5 eq) in water (5 mL) was added to a solution of the methyl ester (1.00 g, 2.35 mmol, 1.0 eq) in tetrahydrofuran:methanol (2:1, 15 mL) at 0° C. and stirred for 15 minutes before continuing at ambient temperature for a further 2 hours. The reaction mixture was then concentrated in vacuo. Ethyl acetate (25 mL) and brine (25 mL) were added and the mixture was acidified to pH 3 with 1M hydrochloric acid. The organic layer was separated and the aqueous layer was further extracted with ethyl acetate (25 mL). The combined organic layers were dried over sodium sulphate, filtered and evaporated in vacuo, to give 0.85 g (88%) of the desired product.

1H NMR (250 MHz, CHLOROFORM-d) δ ppm 7.10-7.27 (m, 3H) 5.34 (br. s., 1H) 4.62-4.86 (m, 4H) 4.49-4.62 (m, 1H) 4.32-4.50 (m, 1H) 3.65-3.83 (m, 2H) 2.43-2.63 (m, 1H) 2.21-2.43 (m, 0H) 1.38-1.53 (m, 9H)

LC-MS: purity 94% (UV), tR 1.34 min m/z [M+Na]+433.10

### Stage 3: I-22

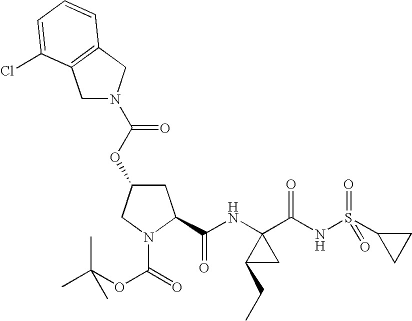

Diisopropylethylamine (1.08 mL, 6.20 mmol, 3.0 eq) was added to a stirred suspension of the above proline (0.85 g, 2.07 mmol, 1.0 eq) and HATU (1.18 g, 3.10 mmol, 1.5 eq) in dichloromethane at 0° C. After 1 hour cyclopropanesulfonic acid ((1R,2R)-1-amino-2-ethyl-cyclopropanecarbonyl)-amide (0.55 g, 2.07 mmol, 1.0 eq) was added and this was stirred overnight whilst allowing to warm to ambient temperature. The reaction mixture was washed with brine (50 mL) then the aqueous phase was extracted with dichloromethane (50 mL). The combined organic layers were dried over sodium sulphate, filtered and evaporated in vacuo. Purification by flash column chromatography, eluting with a methanol:dichloromethane:gradient (1:99 to 2:98) and then again with ethyl acetate:heptanes gradient (7:3) gave 0.61 g (47%) of the desired product.

1H NMR (500 MHz, CHLOROFORM-d) δ 10.01 (br. s., 1H), 6.87-7.27 (m, 3H), 5.31-5.40 (m, 1H), 4.62-4.87 (m, 4H), 4.27 (d, J=7.89 Hz, 1H), 3.56-3.84 (m, 2H), 2.88-3.09 (m, 1H), 2.26-2.56 (m, 2H), 1.70 (d, J=5.96 Hz, 1H), 1.62 (br. s., 2H), 1.50 (d, J=3.30 Hz, 9H), 1.29-1.47 (m, 3H), 1.15-1.26 (m, 1H), 0.95-1.10 (m, 5H)

LC-MS: purity 100% (UV), tR 2.25 min m/z [M+Na]+647.25

### Stage 4: I-23

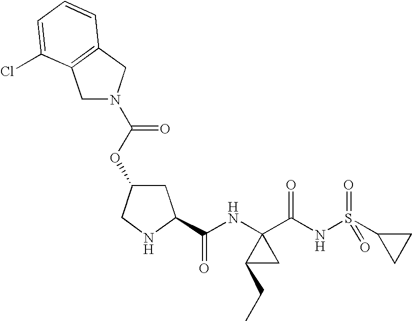

4M HCl in Dioxane (16 mL) was added to a solution of the BOC derivative (668 mg, 1.06 mmol, 1.0 eq) at 0° C. and stirred for 15 minutes then for a further 2 hours at ambient temperature. The reaction mixture was allowed to stand overnight then evaporated to dryness. The residue was then evaporated from dichloromethane (2×25 mL) and used in the next stage without any further purification.

LC-MS: purity 99% (UV), tR 1.40 min m/z [M+H]+ 525.00

### Stage 5-1 (Compound 1)

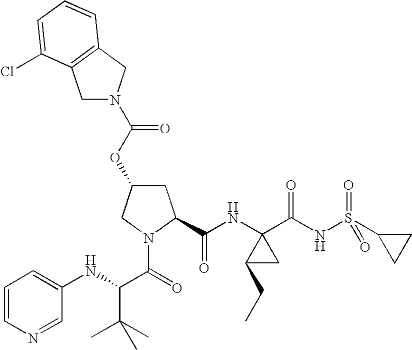

Diisopropylethylamine (111 μL, 0.64 mmol, 3.0 eq) was added to a solution of (S)-3,3-dimethyl-2-(pyridin-3-ylamino)-butyric acid (43 mg, 0.21 mmol, 1.0 eq), HATU (106 mg, 0.28 mmol, 1.3 eq) and stage 4 intermediate (1.06 mmol) in dimethylformamide (2 mL) at 0° C. and stirred overnight whilst warming to ambient temperature. The reaction mixture was diluted with ethyl acetate (30 mL) and washed with water (2×25 mL). The organic layer was dried over sodium sulphate and evaporated. Purification by flash column chromatography eluting with methanol:dichloromethane (5:95) gave 27.7 mg (18%) of the desired product as a beige solid.

1H NMR (250 MHz, MeOD) δ 8.06 (br. s., 1H), 7.06-7.62 (m, 5H), 6.87-7.01 (m, 1H), 5.26-5.46 (m, 1H), 4.61-4.84 (m, 3H), 4.32-4.56 (m, 2H), 4.13-4.30 (m, 3H), 3.91 (m, 1H), 2.98 (m, 1H), 2.25-2.46 (m, 1H), 2.04-2.24 (m, 1H), 1.44-1.71 (m, 4H), 1.27-1.36 (m, 1H), 1.16-1.21 (m, 1H), 1.05-1.15 (m, 11H), 0.98 (m, 3H).

LC-MS: purity 100% (UV), tR 3.35 min m/z [M+H]+ 715.45.

Syntheses of Final Products with 4-Cl and 4-F-Isoindoline P2 Groups

### Stage 5: Compound 2

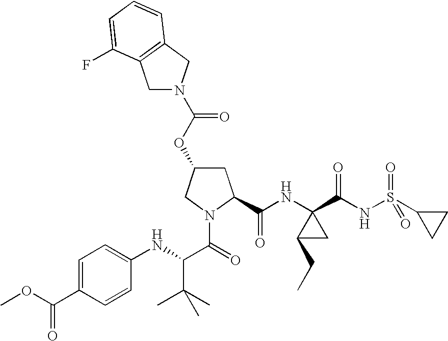

Procedure as described for 1. Yield: 370 mg (74%)

1H NMR (250 MHz, MeOD) δ 7.54-7.73 (m, 2H), 7.21-7.43 (m, 1H), 6.86-7.17 (m, 2H), 6.73 (dd, J=2.66, 8.91 Hz, 2H), 5.28-5.49 (m, 1H), 4.65 (s, 2H), 4.33-4.56 (m, 2H), 4.27 (d, J=3.81 Hz, 2H), 3.97-4.17 (m, 1H), 3.90 (dd, J=2.97, 12.26 Hz, 1H), 3.43-3.77 (m, 3H), 2.99 (m, 1H), 2.36 (ddd, J=6.74, 7.01, 13.21 Hz, 1H), 2.00-2.21 (m, 1H), 1.42-1.76 (m, 4H), 1.22-1.41 (m, 2H), 1.03-1.23 (m, 12H), 0.99 (t, J=6.85 Hz, 3H)

LC-MS: purity 100% (UV), tR 4.46 min m/z [M+H]+ 756.05

### Stage 5: Compound 3

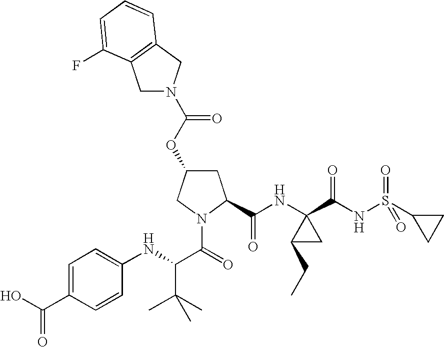

Procedure as described for 1.

A solution of lithium hydroxide monohydrate (35.5 mg, 0.84 mmol) in water (1 mL) was added to a solution of the methyl ester (320 mg, 0.42 mmol) in tetrahydrofuran:methanol (2:1, 3 mL) and stirred overnight at ambient temperature after which a further aliquot of lithium hydroxide monohydrate (17.8 mg, 0.42 mmol) in tetrahydrofuran:methanol:H2O (2:1:1, 4 mL) and stirred for 1 hour. A further aliquot of lithium hydroxide monohydrate (35.5 mg, 0.84 mmol) was added and the reaction was stirred overnight at 50° C. The solvent was evaporated in vacuo. Ethyl acetate (10 mL) was added followed by water (5 mL) then acidified to pH 3 with 1M hydrochloric acid (approx. 3 mL). The organic layer was collected, dried over sodium sulphate, filtered and evaporated. Purification by flash column chromatography, eluting with a ethyl acetate:heptanes gradient (8:2 to 1) and then further purification by ‘Prep HPLC’ gave the desired product, 30 mg (10%).

1H NMR (250 MHz, MeOD) δ 7.67 (dd, J=8.83, 18.88 Hz, 2H), 7.20-7.51 (m, 1H), 6.86-7.22 (m, 2H), 6.75 (t, J=8.60 Hz, 1H), 5.20-5.57 (m, 1H), 4.67 (d, J=6.70 Hz, 2H), 4.37-4.58 (m, 2H), 4.06-4.37 (m, 3H), 3.76-4.06 (m, 1H), 2.96-3.09 (m, 1H), 2.27-2.59 (m, 1H), 2.05-2.30 (m, 1H), 1.43-1.76 (m, 4H), 1.26-1.38 (m, 3H), 1.08-1.25 (m, 11H), 0.94-1.07 (m, 3H)

LC-MS: purity 100% (UV), tR 4.24 min m/z [M+H]+ 742.40

### Stage 5: Compound 4

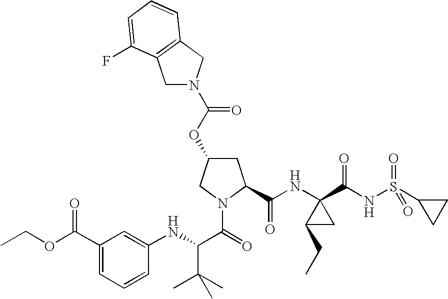

Procedure as described for 1. Yield: 410 mg (65%)

1H NMR (250 MHz, MeOD) δ 7.30-7.55 (m, 2H), 6.83-7.25 (m, 5H), 5.39 (d, J=3.05 Hz, 1H), 4.67 (s, 2H), 4.29-4.53 (m, 2H), 3.81-4.28 (m, 6H), 2.84-3.18 (m, 1H), 2.24-2.52 (m, 1H), 1.99-2.25 (m, 1H), 1.40-1.76 (m, 4H), 1.25-1.41 (m, 5H), 1.04-1.28 (m, 12H), 0.92-1.10 (m, 3H)

LC-MS: purity 97% (UV), tR 4.85 min m/z [M+H]+ 770.05

### Stage 5: Compound 5

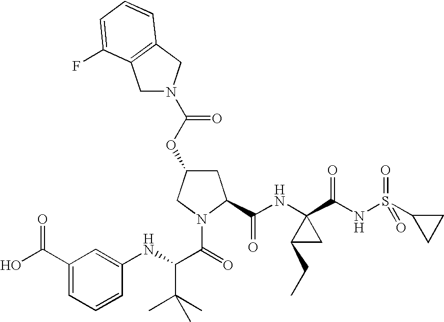

Procedure as described for 1. Yield: 200 mg (59%).

The ethyl ester was hydrolysed as for Compound 3.

1H NMR (250 MHz, MeOD) δ 7.67 (dd, J=8.83, 18.88 Hz, 2H), 7.20-7.51 (m, 1H), 6.86-7.22 (m, 2H), 6.75 (t, J=8.60 Hz, 2H), 5.20-5.57 (m, 1H), 4.67 (d, J=6.70 Hz, 2H), 4.37-4.58 (m, 2H), 4.06-4.37 (m, 3H), 3.76-4.06 (m, 1H), 2.96-3.09 (m, 1H), 2.27-2.59 (m, 1H), 2.05-2.30 (m, 1H), 1.43-1.76 (m, 4H), 1.26-1.38 (m, 3H), 1.08-1.25 (m, 11H), 0.94-1.07 (m, 3H)

LC-MS: purity 100% (UV), tR 4.37 min m/z [M+H]+ 742.30

### Stage 5: Compound 8

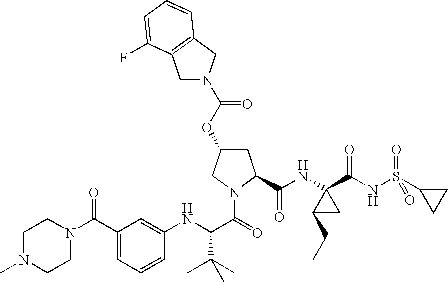

Procedure as described for 1. Yield: 26 mg (29%)

1H NMR (250 MHz, MeOD) δ 7.28-7.48 (m, 1H), 6.97-7.26 (m, 3H), 6.77-6.93 (m, 2H), 6.52 (d, J=7.16 Hz, 1H), 5.36 (br. s., 1H), 4.29-4.80 (m, 6H), 3.81-4.29 (m, 4H), 3.39-3.67 (m, 2H), 2.99-3.32 (m, 4H), 2.97 (s, 3H), 2.26-2.49 (m, 1H), 2.01-2.22 (m, 1H), 1.43-1.80 (m, 4H), 1.23-1.45 (m, 3H), 1.06-1.24 (m, 12H), 0.94-1.07 (m, 3H)

LC-MS: purity 100% (UV), tR 3.29 min m/z [M+H]+ 824.50

### Stage 5: Compound 9

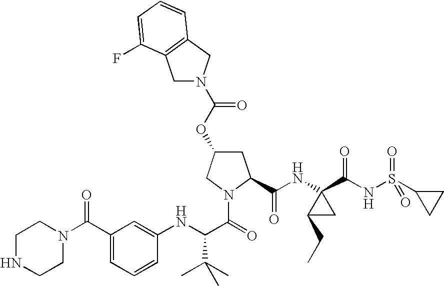

Procedure as described for 1 and then as follows.

4M HCl in dioxane (2 mL) was added to the BOC derivative at 0° C. and stirred for 15 minutes. The ice bath was removed and the reaction was allowed to continue whilst warming to ambient temperature. After 1 hour the reaction mixture was evaporated in vacuo. Purification by flash column chromatography, eluting with a methanol:dichloromethane:gradient (6:94 to 8:92) gave 46 mg (58% over two steps) of the desired product.

1H NMR (500 MHz, MeOD) δ 7.28-7.42 (m, 1H), 6.95-7.26 (m, 3H), 6.60-6.94 (m, 2H), 6.36-6.58 (m, 1H), 5.33 (d, J=2.93 Hz, 1H), 4.61-4.78 (m, 2H), 4.26-4.64 (m, 3H), 3.98-4.23 (m, 2H), 3.90 (d, J=11.92 Hz, 1H), 3.46-3.87 (m, 4H), 2.97-3.28 (m, 4H), 2.89-2.99 (m, 1H), 2.36 (dd, J=7.34, 13.75 Hz, 1H), 2.01-2.22 (m, 1H), 1.39-1.73 (m, 4H), 1.17-1.33 (m, 2H), 1.06-1.18 (m, 10H), 0.92-1.08 (m, 5H)

LC-MS: purity 97% (UV), tR 3.19 min m/z [M+H]+ 810.45

### Stage 5: Compound 10

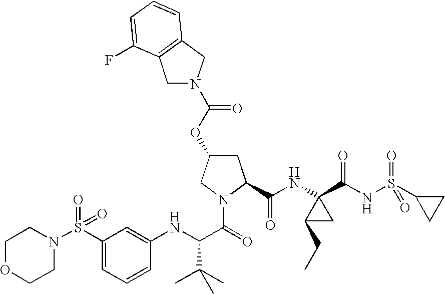

Procedure as described for 1. Yield: 115 mg (66%)

1H NMR (500 MHz, CHLOROFORM-d) δ 10.16 (br. s., 1H), 7.30 (dd, J=5.41, 7.43 Hz, 1H), 6.92-7.17 (m, 5H), 6.77-6.89 (m, 2H), 5.41 (br. s., 1H), 4.62-4.84 (m, 3H), 4.50 (s, 1H), 4.27-4.38 (m, 2H), 3.88-4.04 (m, 2H), 3.62-3.80 (m, 4H), 2.91-3.04 (m, 5H), 2.24-2.46 (m, 2H), 1.71 (dd, J=5.59, 8.16 Hz, 1H), 1.62-1.67 (m, 1H), 1.33-1.47 (m, 3H), 1.27-1.34 (m, 1H), 1.12 (s, 9H), 1.02-1.10 (m, 2H), 0.97 (t, J=7.34 Hz, 3H), 0.89 (t, J=6.97 Hz, 2H)

LC-MS: purity 99% (UV), tR 4.65 min m/z [M+H]+ 847.30

### Stage 5: Compound 11

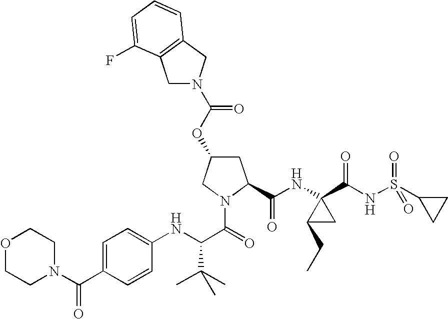

Procedure as described for 1. Yield: 111 mg (67%)

1H NMR (500 MHz, CHLOROFORM-d) δ 10.13 (br. s., 1H), 7.24-7.34 (m, 1H), 7.16 (dd, J=4.77, 8.44 Hz, 2H), 6.90-7.11 (m, 3H), 6.58 (dd, J=5.78, 8.34 Hz, 2H), 5.40 (d, J=1.65 Hz, 1H), 4.67-4.80 (m, 3H), 4.44-4.64 (m, 2H), 4.39 (d, J=7.70 Hz, 1H), 3.92-4.05 (m, 3H), 3.61 (br. s., 8H), 2.90-2.95 (m, 1H), 2.28-2.46 (m, 2H), 1.65-1.72 (m, 1H), 1.49-1.65 (m, 2H), 1.32-1.46 (m, 3H), 1.23-1.30 (m, 1H), 1.10 (s, 9H), 1.01-1.08 (m, 2H), 0.97 (t, J=7.34 Hz, 3H)

LC-MS: purity 98% (UV), tR 4.28 min m/z [M+H]+ 811.45

### Stage 5: Compound 12

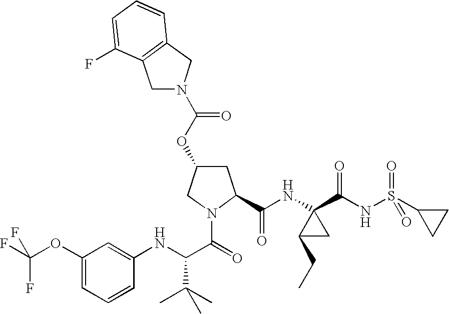

Procedure as described for 1. Yield: 65 mg (40%)

1H NMR (500 MHz, CHLOROFORM-d) δ 10.09 (m, 2H), 7.27-7.34 (m, 1H), 6.83-7.12 (m, 4H), 6.51 (ddd, J=2.75, 2.89, 8.12 Hz, 1H), 6.45 (s, 1H), 6.18-6.32 (m, 1H), 5.41 (s, 1H), 4.72 (d, J=6.05 Hz, 2H), 4.38-4.54 (m, 2H), 4.14-4.24 (m, 1H), 3.83-3.98 (m, 3H), 2.89-3.01 (m, 1H), 2.38-2.49 (m, 1H), 2.26-2.36 (m, 1H), 1.67 (dd, J=5.50, 8.25 Hz, 1H), 1.51-1.64 (m, 2H), 1.33-1.45 (m, 3H), 1.19-1.31 (m, 1H), 1.01-1.15 (m, 12H), 0.98 (t, J=7.34 Hz, 3H)

LC-MS: purity 99% (UV), tR 5.15 min m/z [M+H]+ 782.35

### Stage 5: Compound 13

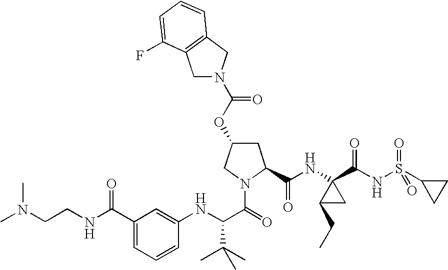

Procedure as described for 1. Yield: 33 mg (19%)

1H NMR (500 MHz, MeOD) δ 7.32-7.42 (m, 1H), 6.87-7.22 (m, 6H), 5.20-5.39 (m, 1H), 4.67 (s, 2H), 4.35-4.61 (m, 2H), 4.12-4.35 (m, 2H), 4.01 (d, J=7.34 Hz, 1H), 3.66-3.81 (m, 2H), 3.55-3.67 (m, 1H), 3.32-3.47 (m, 2H), 2.99-3.07 (m, 1H), 2.93-3.01 (m, 6H), 2.23-2.42 (m, 1H), 1.98-2.12 (m, 1H), 1.48-1.78 (m, 4H), 1.24-1.38 (m, 2H), 1.19 (dd, J=4.77, 8.44 Hz, 1H), 1.15 (s, 9H), 1.05-1.13 (m, 2H), 0.94-1.07 (m, 3H)

LC-MS: purity 100% (UV), tR 3.26 min m/z [M−H]− 810.30

### Stage 5: Compound 14

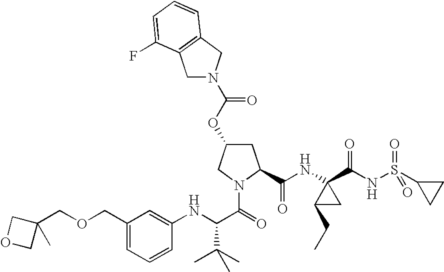

Procedure as described for 1. Yield: 73 mg (44%)

1H NMR (500 MHz, CHLOROFORM-d) δ 10.27-10.40 (m, 1H), 7.30-7.41 (m, 1H), 6.79-7.18 (m, 4H), 6.63-6.73 (m, 1H), 6.07-6.23 (m, 1H), 5.23-5.34 (m, 1H), 4.69-4.84 (m, 3H), 4.31-4.53 (m, 5H), 4.07-4.30 (m, 4H), 3.79-3.89 (m, 3H), 3.49 (d, J=8.80 Hz, 1H), 3.31-3.44 (m, 1H), 2.14-2.32 (m, 2H), 1.55-1.73 (m, 3H), 1.41-1.49 (m, OH), 1.34-1.38 (m, 4H), 1.30-1.34 (m, 3H), 1.12 (s, 9H), 1.02-1.08 (m, 2H), 0.95-1.02 (m, 3H), 0.89 (t, J=6.97 Hz, 2H)

LC-MS: purity 100% (UV), tR 4.86 min m/z [M+H]⁺ 812.45

### Stage 5: Compound 15

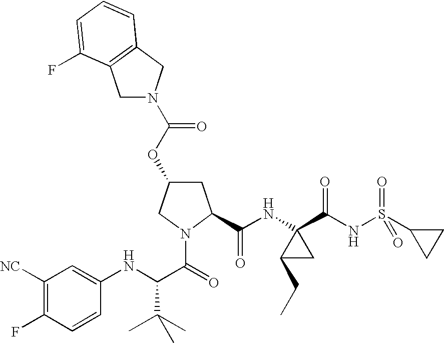

Procedure as described for 1. Yield: 52 mg (34%)

1H NMR (500 MHz, CHLOROFORM-d) δ 10.14 (s, 1H), 7.28-7.34 (m, 1H), 6.93-7.10 (m, 3H), 6.73-6.87 (m, 3H), 5.40 (br. s., 1H), 4.75 (br. s., 2H), 4.51 (t, J=15.31 Hz, 1H), 4.36-4.46 (m, 1H), 4.24-4.35 (m, 1H), 3.89-3.99 (m, 2H), 3.82 (d, J=3.12 Hz, 1H), 2.90-2.98 (m, 1H), 2.45-2.67 (m, 1H), 2.31-2.44 (m, 2H), 1.69 (dd, J=5.59, 8.16 Hz, 1H), 1.53-1.65 (m, 2H), 1.33-1.46 (m, 3H), 1.27 (dd, J=5.41, 9.45 Hz, 1H), 1.02-1.15 (m, 11H), 0.97 (td, J=2.02, 7.34 Hz, 3H)

LC-MS: purity 99% (UV), tR 4.74 min m/z [M+H]+ 741.35

### Stage 5: Compound 16

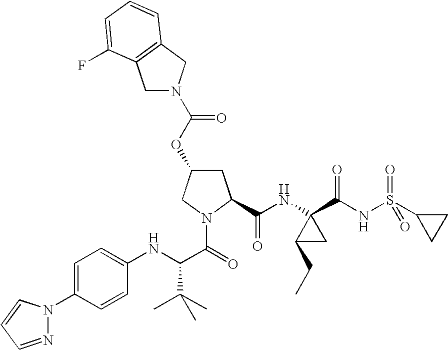

Procedure as described for 1. Yield: 45 mg (29%)

1H NMR (500 MHz, CHLOROFORM-d) δ 10.10 (s, 1H), 7.39-7.54 (m, 2H), 7.32 (d, J=8.80 Hz, 1H), 7.08-7.23 (m, 2H), 6.71-6.95 (m, 2H), 6.67 (dd, J=8.89, 10.36 Hz, 2H), 6.25 (dt, J=1.97, 18.98 Hz, 1H), 5.34-5.41 (m, 1H), 4.62-4.76 (m, 2H), 4.39-4.54 (m, 2H), 4.23-4.30 (m, 1H), 4.01-4.08 (m, 1H), 3.96 (d, J=6.79 Hz, 1H), 3.92 (dd, J=3.48, 11.92 Hz, 1H), 2.91-3.01 (m, 1H), 2.38-2.48 (m, 3H), 2.27-2.36 (m, 1H), 1.68 (dd, J=5.59, 8.16 Hz, 1H), 1.49-1.64 (m, 2H), 1.33-1.44 (m, 3H), 1.24 (dd, J=5.50, 9.35 Hz, 1H), 1.11 (d, J=4.03 Hz, 9H), 1.01-1.09 (m, 2H), 0.97 (td, J=2.84, 7.38 Hz, 3H)

LC-MS: purity 95% (UV), tR 4.63 min m/z [M+H]+ 764.40

### Stage 5: Compound 17

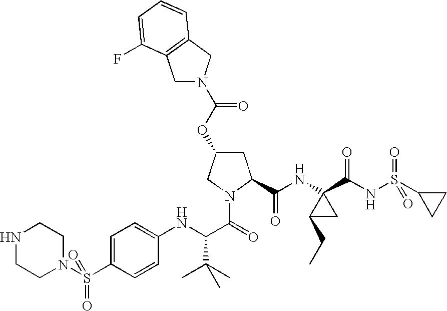

Procedure as described for 1. Yield: 63 mg (94%)

1H NMR (500 MHz, MeOD) δ 7.41-7.52 (m, 2H), 7.28-7.37 (m, 1H), 7.11 (dd, 1H), 6.97-7.05 (m, 1H), 6.85 (dd, J=8.89, 15.68 Hz, 2H), 5.31-5.39 (m, 1H), 4.57-4.76 (m, 3H), 4.41-4.54 (m, 2H), 4.19-4.33 (m, 2H), 3.85-3.96 (m, 1H), 3.19-3.27 (m, 4H), 3.08-3.18 (m, 4H), 3.00 (s, 2H), 2.34-2.44 (m, 1H), 2.08-2.17 (m, 1H), 1.48-1.70 (m, 4H), 1.24-1.33 (m, 4H), 1.16-1.21 (m, 1H), 1.06-1.16 (m, 10H), 0.97-1.02 (m, 3H), 0.78-0.97 (m, 2H)

LC-MS: purity 94% (UV), tR 3.29 min m/z [M+H]+ 846.40

Syntheses of Final Products with 5-Substituted-Isoindoline R¹ Groups:

### General Route

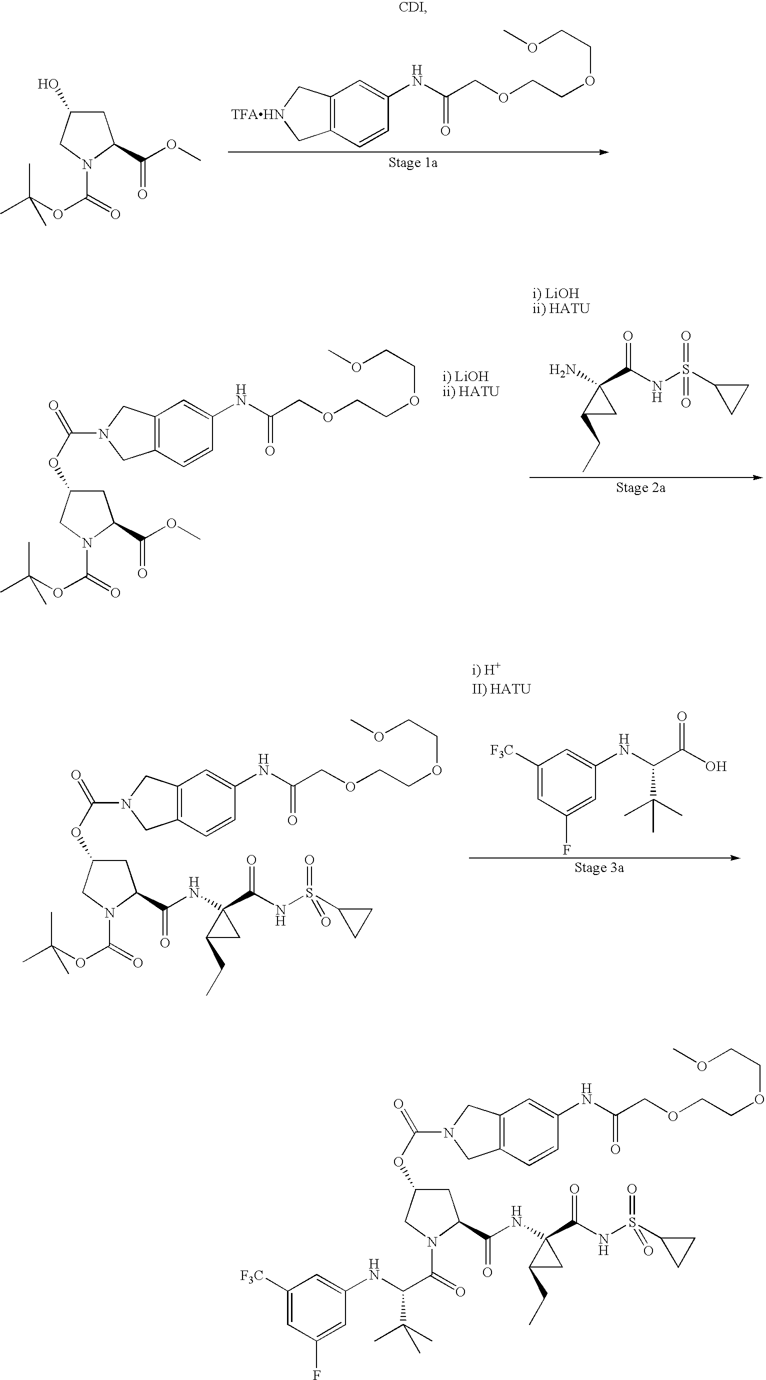

### Stage 3a: Compound 18

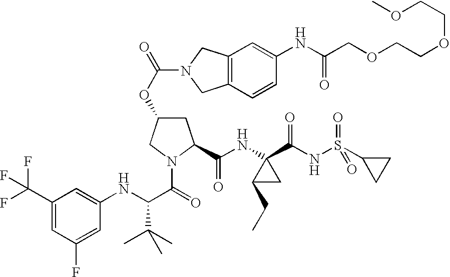

Procedure as described for 1. Yield: 64 mg (23%)

1H NMR (500 MHz, MeOD) δ ppm 7.54-7.72 (1H, m), 7.45-7.54 (1H, m), 7.05-7.32 (1H, m), 6.91 (1H, br. s.), 6.65-6.77 (1H, m), 6.32-6.43 (1H, m), 5.37-5.44 (1H, m), 4.57-4.71 (2H, m), 4.38-4.52 (2H, m), 4.18-4.33 (3H, m), 4.16 (2 H, s), 3.97 (1H, dt, J=12.44, 3.47 Hz), 3.69-3.82 (8H, m), 3.56-3.62 (2H, m), 3.36 (3H, d, J=8.39 Hz), 2.96-3.04 (1H, m), 2.37-2.44 (1H, m), 2.12-2.20 (1H, m), 2.02-2.05 (3H, m), 1.60-1.70 (2H, m), 1.49-1.59 (2H, m), 1.29-1.36 (2H, m), 1.12-1.18 (9H, m), 1.11 (2H, d, J=8.09 Hz), 1.00 (3H, t, J=7.10 Hz)

LC-MS: purity 95% (UV), tR 4.78 min m/z [M+H]+ 941.45

### Alternative Route

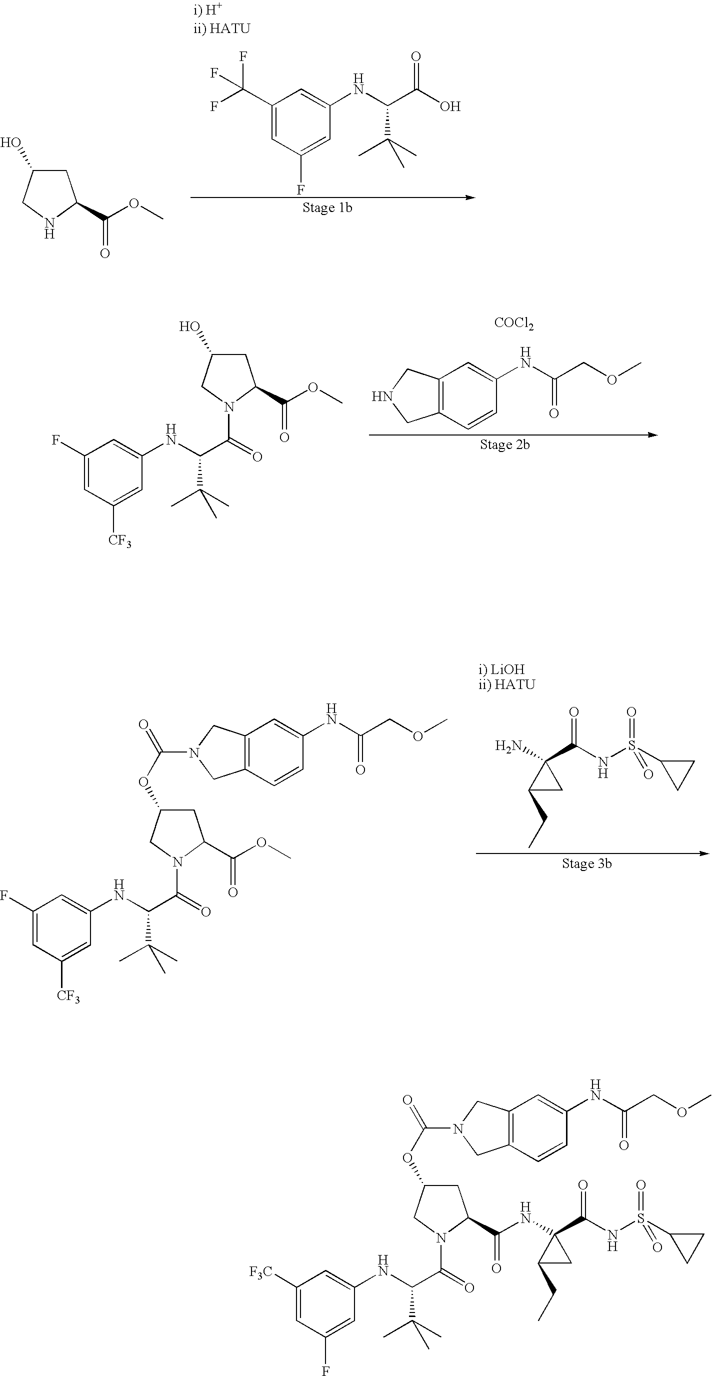

### Stage 1b: I-25

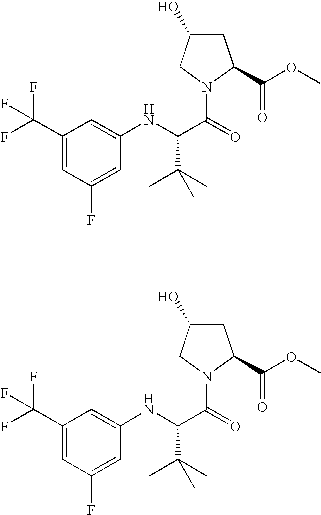

HATU (1.15 mg, 3.02 mmol) was added to a solution of the N-aryl tert-leucine (680 mg, 2.32 mmol) in dimethylformamide (10 mL) at 0° C. and stirred at ambient temperature for 15 minutes. Hydroxy proline methyl ester hydrochloride (505 mg, 2.78 mmol) was then added followed by diisopropylethylamine (1.8 mL, 6.96 mmol). The reaction mixture was allowed to stir overnight whilst warming to ambient temperature. The reaction mixture was concentrated in vacuo, dissolved in ethyl acetate (50 mL), washed with water (50 mL), then brine (50 mL), dried over sodium sulphate, filtered and evaporated. Purification by flash column chromatography, eluting with ethyl acetate:heptanes (4:6) gave the desired product, 700 mg (72%) as a white solid.

1H NMR (500 MHz, CHLOROFORM-d) δ 6.55-6.68 (m, 2H), 6.44 (d, J=11.19 Hz, 1H), 4.79 (d, J=9.72 Hz, 1H), 4.58-4.69 (m, 2H), 3.84-3.95 (m, 2H), 3.67-3.81 (m, 4H), 2.28 (d, J=8.07 Hz, 1H), 2.03-2.20 (m, 1H), 1.67 (br. s., 1H), 1.12 (s, 9H)

### Stage 2b: I-26

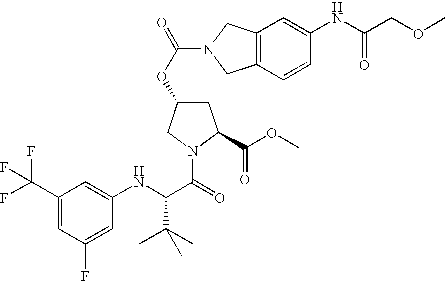

A solution of the hydroxy proline derivative (130 mg, 0.31 mmol) in dichloromethane (2 mL) was added slowly to a stirred solution of phosgene (2M in toluene, 170 μL, 0.34 mmol) and pyridine (50 μL, 0.618 mmol) in dichloromethane at 0° C. and stirred for 5 minutes. The reaction mixture was then stirred for a further 30 minutes whilst allowing to warm to ambient temperature. DMAP was then added to the reaction mixture at 0° C., followed by the isoindoline (99 mg, 0.31 mmol) and then diisopropylethylamine (270 μL, 1.55 mmol). The reaction was allowed to stir for 1 hour then quenched with methanol (5 mL), stirred for 15 minutes then evaporated in vacuo. Purification by flash column chromatography, eluting with a ethyl acetate:heptanes gradient (4:6 to 1:1) gave 147 mg (73%) of the desired product, as a white solid.

1H NMR (250 MHz, CHLOROFORM-d) δ 8.19-8.38 (m, 1H), 7.27-7.83 (m, 2H), 7.01-7.25 (m, 1H), 6.54-6.71 (m, 1H), 6.33-6.45 (m, 1H), 5.31-5.49 (m, 1H), 4.51-4.92 (m, 4H), 4.38-4.51 (m, 1H), 4.24 (d, J=8.68 Hz, 1H), 4.03 (s, 2H), 3.84-3.98 (m, 3H), 3.77 (s, 2H), 3.53 (d, J=1.52 Hz, 3H), 2.41-2.64 (m, 1H), 2.09-2.30 (m, 1H), 1.36-1.53 (m, 2H), 1.06-1.15 (m, 9H)

### Stage 3b: Compound 19

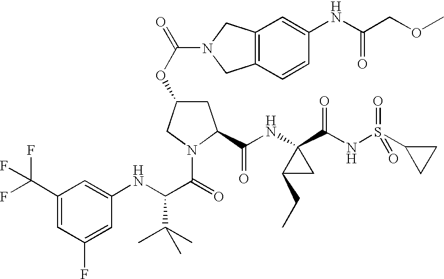

A solution of lithium hydroxide monohydrate (14.2 mg, 0.338 mmol) in water (0.5 mL) was added to a solution of the methyl ester (147 mg, 0.225 mmol) in tetrahydrofuran:methanol (2:1, 1.5 mL) at 0° C. and stirred for 15 minutes. Stirring was continued whilst warming to ambient temperature.

After 2 hours, the solution was neutralised with 1M hydrochloric acid and concentrated in vacuo. The crude product was passed through a small pad of silica gel using a solution of dichloromethane:methanol (90:10) then evaporated to dryness and used in the next step without further purification.

Diisopropylethylamine (235 μL, 1.35 mmol) was added to a solution of the above product, cyclopropanesulfonic acid ((1R,2R)-1-amino-2-ethyl-cyclopropanecarbonyl)-amide (52.3 mg, 0.225 mmol) and HATU (111.2 mg, 0.293 mmol) in dimethyl formamide (2.5 mL) at 0° C. and stirred overnight whilst allowing to warm to ambient temperature. The reaction mixture was then evaporated in vacuo and the residue was purified by flash column chromatography, eluting with ethyl acetate:heptanes (4:6 to 7:3) to give 168 mg (88%) of the desired product.

1H NMR (500 MHz, MeOD) δ 7.54-7.72 (m, 1H), 7.43-7.53 (m, 1H), 7.04-7.34 (m, 1H), 6.85-6.96 (m, 1H), 6.68-6.76 (m, 1H), 6.29-6.44 (m, 1H), 5.40 (br. s., 1H), 4.65 (br. s., 2H), 4.38-4.51 (m, 2H), 4.17-4.31 (m, 3H), 4.06 (s, 2H), 3.92-4.00 (m, 1H), 3.52 (d, J=2.44 Hz, 3H), 2.96-3.06 (m, 1H), 2.35-2.44 (m, 1H), 2.10-2.20 (m, 1H), 1.50-1.73 (m, 4H), 1.39 (dd, J=3.81, 6.71 Hz, 2H), 1.21 (dd, J=4.96, 8.47 Hz, 1H), 1.07-1.18 (m, 11H), 1.00 (t, J=7.02 Hz, 3H)

LC-MS: purity 100% (UV), tR 4.82 min m/z [M+H]+ 853.35

### Stage 3b: Compound 20

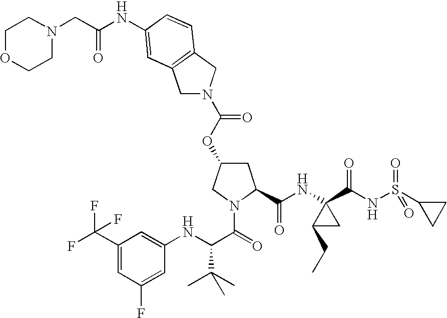

Procedure as described above for 19. Yield: 30 mg (8%)

1H NMR (500 MHz, CHLOROFORM-d) δ 10.14 (br. s., 1H), 8.93-9.43 (m, 1H), 7.62-7.88 (m, 1H), 7.31-7.51 (m, 2H), 6.99-7.24 (m, 2H), 6.58-6.70 (m, 1H), 6.30-6.47 (m, 2H), 5.37-5.46 (m, 1H), 4.79-4.99 (m, 1H), 4.68 (br. s., 3H), 4.42 (br. s., 1H), 4.24 (s, 1H), 3.98 (br. s., 2H), 3.91 (s, 1H), 3.83 (br. s., 5H), 3.65 (br. s., 5H), 2.88-2.99 (m, 2H), 2.81 (s, 1H), 2.56-2.80 (m, 3H), 2.38 (br. s., 2H), 1.10 (d, J=5.95 Hz, 12H), 1.01-1.08 (m, 3H)

LC-MS: purity 100% (UV), tR 3.78 min m/z [M+H]+ 908.45

### Procedure for Preparing I-107

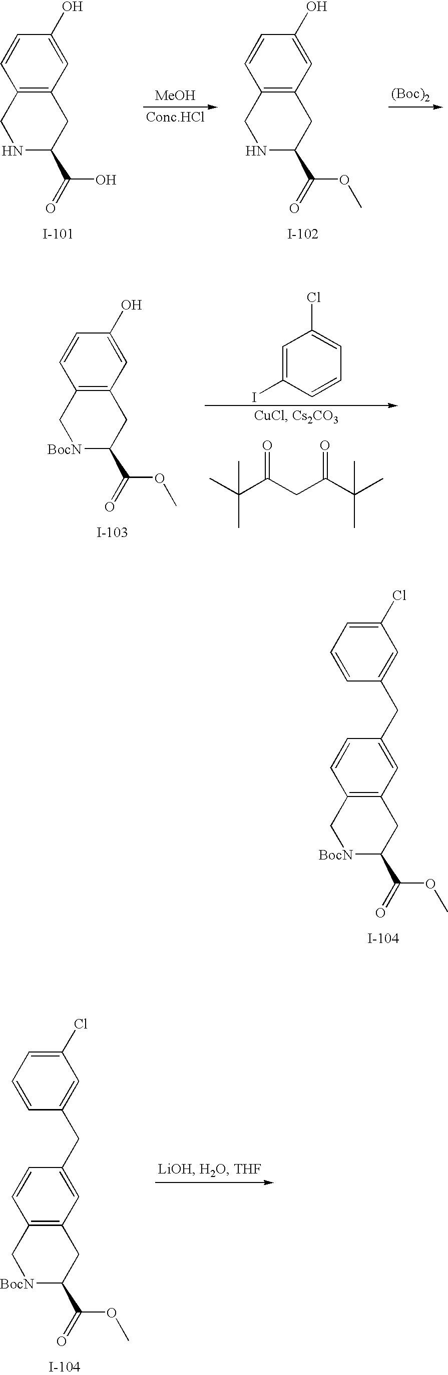

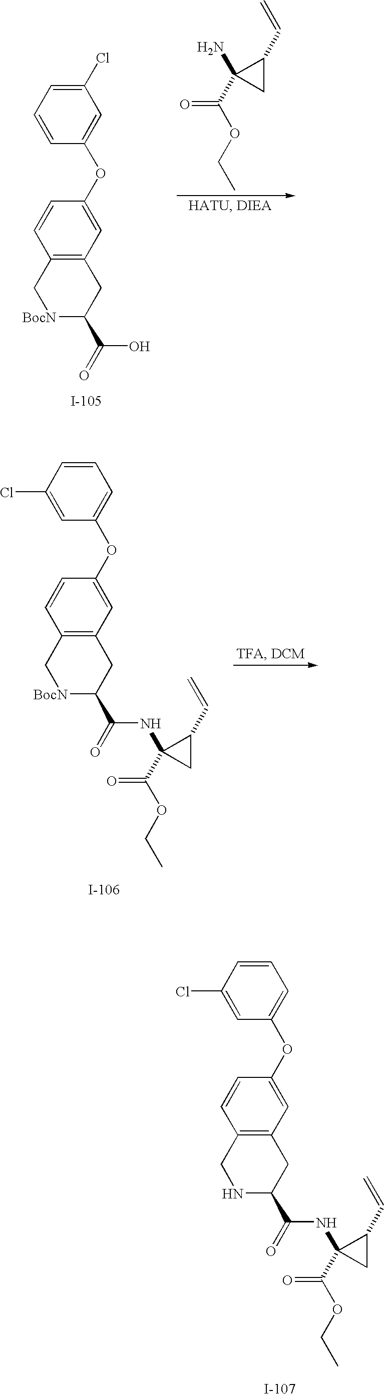

### Procedure for Preparing I-102

To a solution of compound I-101 (2 g, 10.3 mmol) in 50 ml of methanol was added 2 mL of concentrated HCl. The resulting mixture was stirred at reflux temperature for 5 h. evaporated solvent was removed by vacuum to give a residue, which was dissolved in 50 mL of water, adjusted pH=8 with aq. sat. NaHCO₃, extracted by ethyl acetate (50 mL×3), washed with brine, dried over Na2SO4, concentrated to give compound I-102 as an oil (2.1 g, 100%), which was used without further purification. MS (ESI) m/e (M+H+) 207.1

### Procedure for Preparing I-103

To a solution of compound I-102 (4 g, 19.3 mmol) in 100 mL of dry THF was added (Boc)₂₋₀ (5.2 g, 23.2 mmol) and Et₃N (10.7 g, 96.5 mmol) at rt. The resulting mixture was stirred at same temperature for overnight. 50 mL of water was added and extracted by ethyl acetate (100 mL×3), combined organic layers was washed with diluted 1N HCl, water and then brine, filtrated and concentrated to give compound I-103 (1.76 g, 44.5%) as crude product, which was used directly without further purification. MS (ESI) m/e (M+H+) 307.1

### Procedure for Preparing I-104

To a solution of compound I-103 (1.88 g, 6.1 mmol) in 60 mL of dry NMP was added 1-chloro-3-iodobenzene (1.45 g, 6.1 mmol), 2,2,6,6-tetramethylheptane-3,5-dione (1.1 g, 6.1 mmol), Cs₂CO₃ (3.9 g, 12.2 mmol) and CuCl (0.29 g, 3.0 mmol) sequentially. The resulted mixture was stirred for 16 h, then was diluted with MTBE (80 mL), filtered over celite, washed with 1M HCl, 1M NaOH and brine, the organic phase was dried over Na2SO4 and concentrated to give compound I-104 (1.86 g, 73.1%) as an oil, which was used directly without further purification. MS (ESI) m/e (M+H+) 417.1

### Procedure for Preparing I-105

To solution of compound I-104 (1.0 g, 2.4 mmol) in 20 mL of THF: H2O (1:1) was added LiOH (0.39 g, 9.6 mmol), stirred at reflux for 2 h. reaction was cooled down to rt, acidified by diluted 1N HCl to pH=3, extracted by ethyl acetate (40 mL×3), organic phase was washed with brine, concentrated to give compound I-105 (0.9 g, 93.3%) as an oil, which is pure enough for next step. MS (ESI) m/e (M+H+) 403.1

### Procedure for Preparing I-106

To solution of compound I-105 (1.7 g, 4.2 mmol) in 50 mL of acetonitrile was added HATU (1.7 g, 4.6 mmol) and DIEA (2.2 g, 16.8 mmol). This mixture stirred 30 mins before the addition of (1R,2S)-ethyl-1-amino-2-vinylcyclopropanecarboxylate (0.7 g, 4.2 mmol), then stirred overnight. Reaction was diluted by ethyl acetate (50 mL), washed with diluted 1N HCl, water, sat. NaHCO₃ and brine, concentrated to give a residue, which was purified by silica column (petroleum in ethyl acetate 5% to 10% as eluent) to give compound I-106 (1.7 g, 74.5%) as white solid. MS (ESI) m/e (M+H+) 540.2

### Procedure for Preparing I-107

To solution of compound I-106 (0.6 g, 1.1 mmol) in 10 mL of DCM was added TFA (2 mL). solvent was removed under vacuum, water was added, basified with sat.NaHCO₃, extracted by ethyl acetate (20 mL+3), combined organic layer was washed with brine, concentrated to give compound I-107 (0.46 g, 95.0%) as a couple of diastereomers, which was used in the next step without purification. MS (ESI) m/e (M+H+) 440.1

### Procedure for Preparing of Compounds 25, 26, 27, and 28

The configuration of these two pairs of diastereomers in following scheme is referenced from registration, and may not necessarily be the absolute configuration.

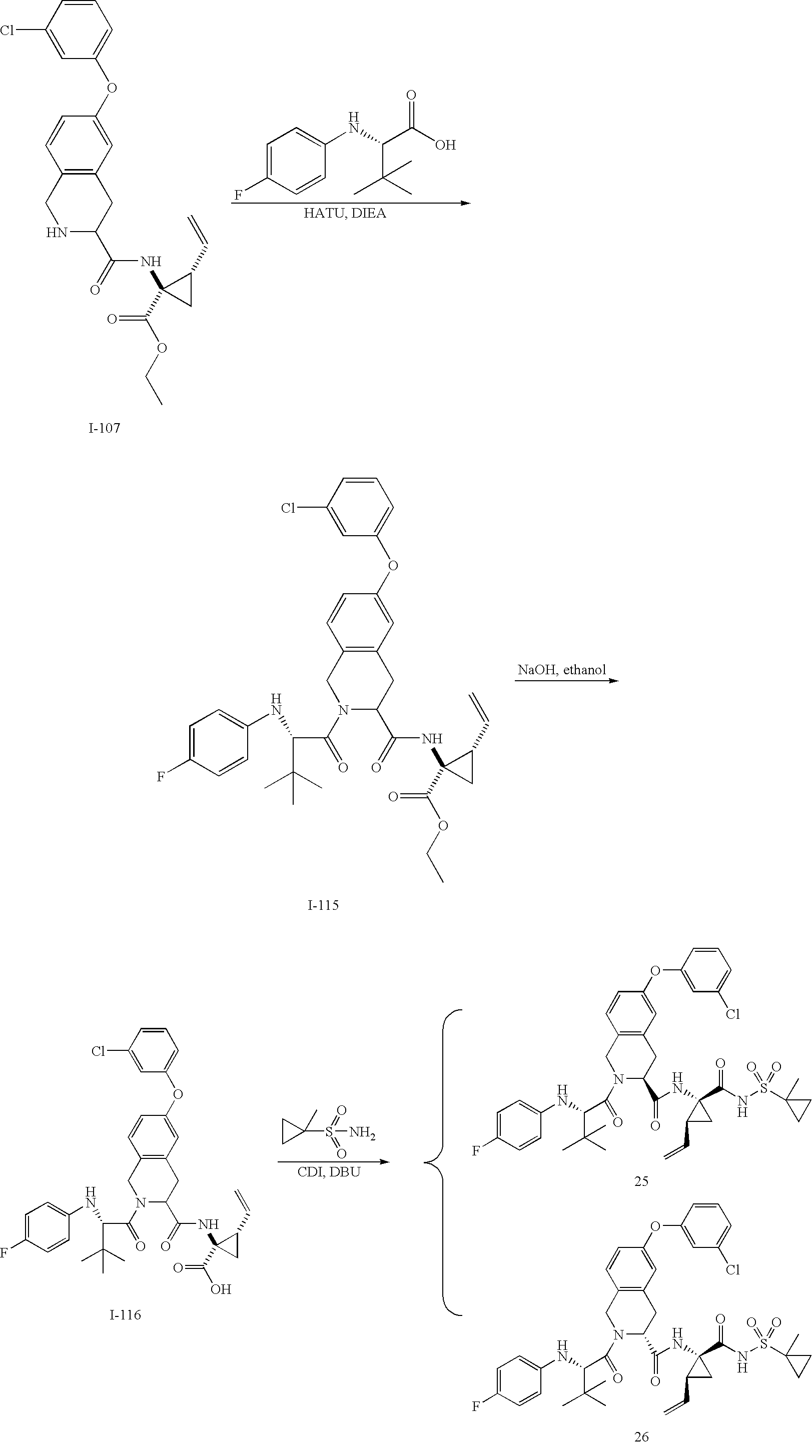

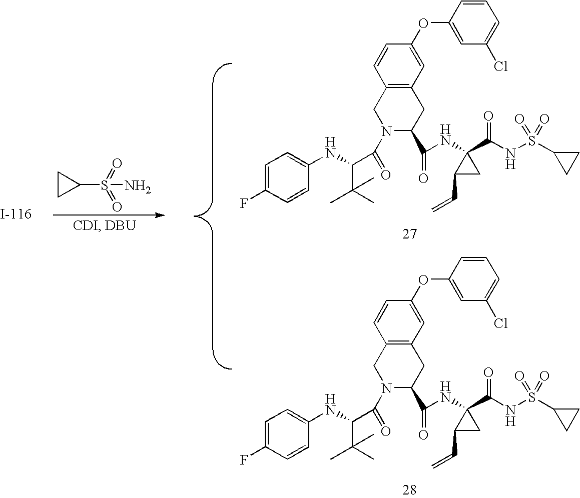

### Procedure for Preparing of I-115

To solution of (S)-2-(4-fluorophenylamino)-3,3-dimethylbutanoic acid (52.0 mg, 0.23 mmol) in 5 mL of DCM was added HATU (105 mg, 0.26 mmol) and DIEA (118 mg, 0.92 mmol). This mixture stirred 30 mins before the addition of compound 7 (100 mg, 0.23 mmol), then stirred overnight. Reaction was diluted by ethyl acetate (10 mL), washed with diluted 1N HCl, water, sat. NaHCO3 and brine, concentrated to give a residue, which was purified by Prep-TLC to give compound I-115 (100 mg, 67.1%) as diastereomer. MS (ESI) m/e (M+H+) 647.3

### Procedure for Preparing of I-116

To solution of compound I-115 (200 mg, 0.31 mmol) in 30 mL of ethanol, was added NaOH (50 mg, 1.3 mmol), stirred at rt for 2 h. reaction was acidified by diluted 1N HCl to pH=3, extracted by ethyl acetate (40 mL×3), organic phase was washed with brine, concentrated to give compound I-116 (180 mg, 93.8%) as solid, which is pure enough for next step. MS (ESI) m/e (M+H+) 619.2

### Procedure for Preparing of Compounds 25 and 26

To solution of compound 16 (180 mg, 0.29 mmol) in 5 mL of DCM was added CDI (140 mg, 0.9 mmol). This mixture stirred 1.5 h before the addition of DBU (220 mg, 1.45 mmol) and 1-methylcyclopropane-1-sulfonamide (202.5 mg, 1.5 mmol), then stirred for another 24 h. Reaction was concentrated to give a residue, which was applied to Prep-HPLC to give each of diastereomer Compound 25 (63.0 mg, 29.5%) and Compound 26 (38.3 mg, 17.9%) as white solid. MS (ESI) m/e (M+H+) 736.2

### Procedure for Preparing of Compounds 27 and 28

The general procedure is same with the preparing of Compound 25 and Compound 26, and the yield for each of the diastereomer are 35.0% for Compound 25 and 22.8% for Compound 28. MS (ESI) m/e (M+H+) 722.2

### Procedure for Preparing of Compounds 21, 22, 23, and 24

The configuration of these two pairs of diastereomers in following scheme is referenced from registration, and may not necessarily be the absolute configuration.

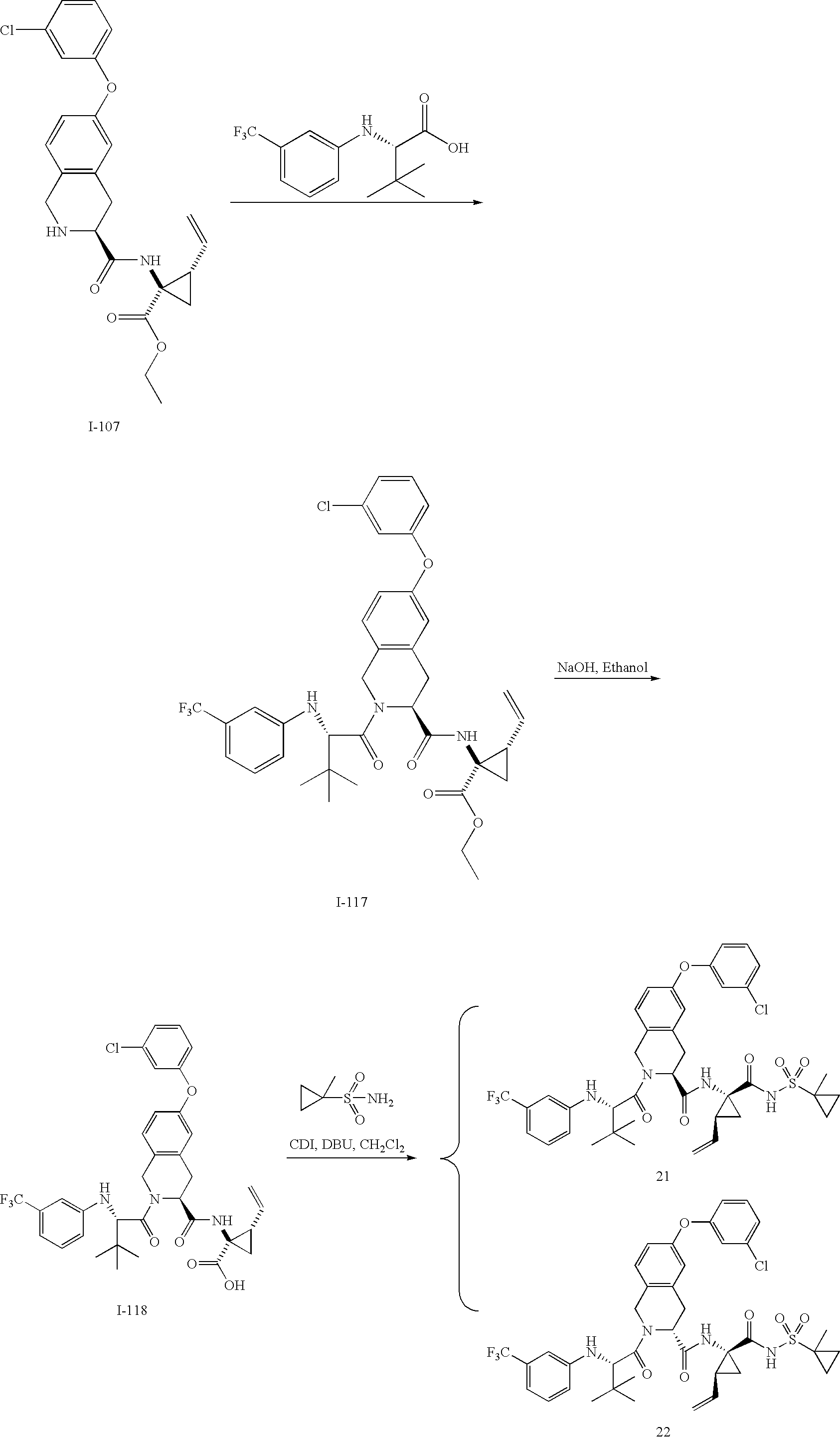

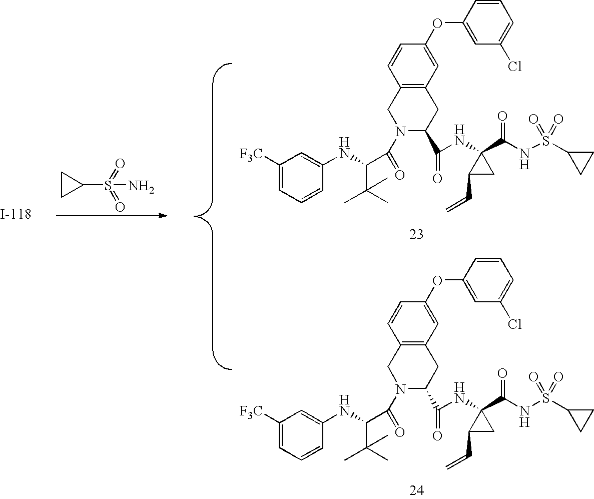

### a) Procedure for Preparing of I-117

The general procedure is same with the preparing of compound I-115, yield is 70.1%. MS (ESI) m/e (M+H+) 697.2

### b) Procedure for Preparing of I-118

The general procedure is same with the preparing of compound I-116, yield is 94.0%. MS (ESI) m/e (M+H+) 669.2

### c) Procedure for Preparing of Compounds 21 and 22

The general procedure is same with the preparing of Compound 25 and Compound 26, and the yield for each of the diastereomer are 15.5% for Compound 22 and 14.3% for Compound 21. MS (ESI) m/e (M+H+) 786.2

### d) Procedure for Preparing of Compounds 23 and 24

The general procedure is same with the preparing of Compound 25 and Compound 26, and the yield for each of the diastereomer are 42.4% for Compound 24 and 40.1% for Compound 23. MS (ESI) m/e (M+H+) 772.2

### Synthesis of Open Chain Protease

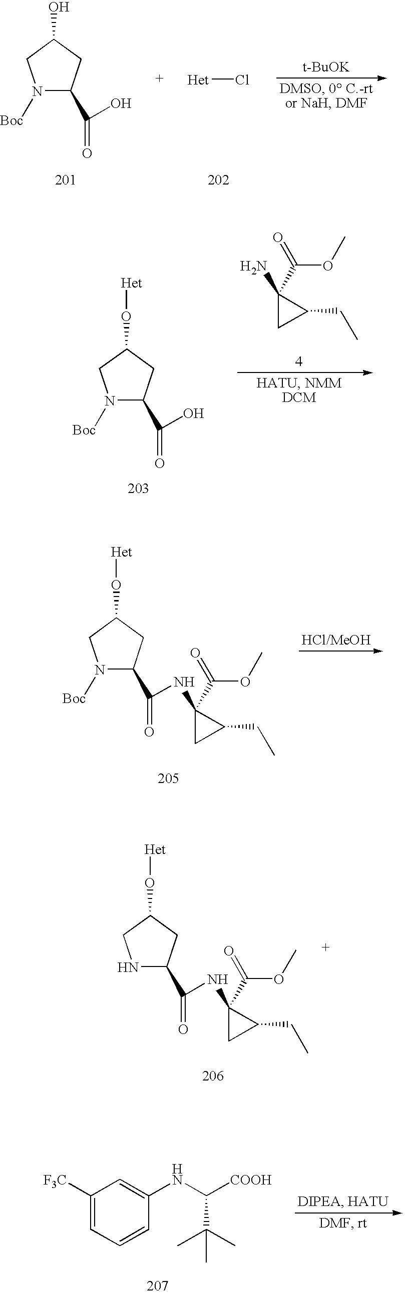

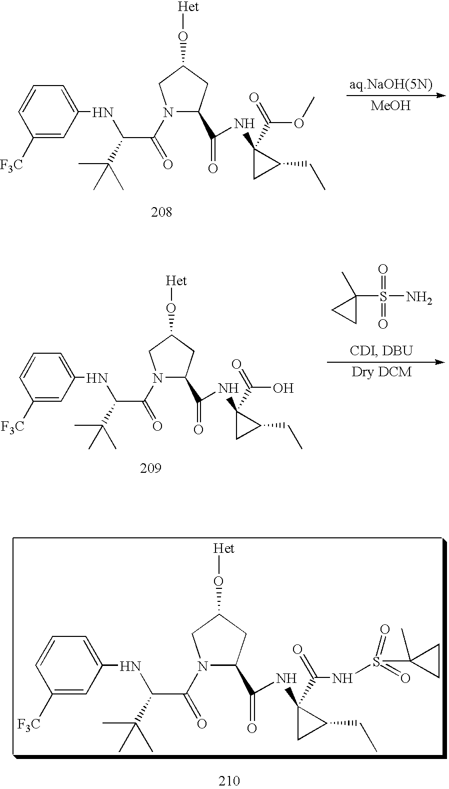

### Preparation of Compound 203a

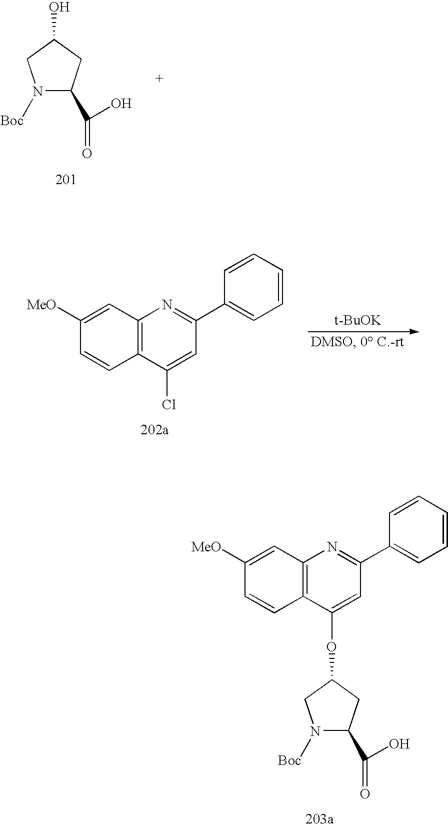

To a suspension of compound 201 (3.0 g, 12.1 mmol) in DMSO (60 ml) was added t-BuOK (3.4 g, 30.25 mmol) at 0° C. The generated mixture was stirred for 1.5 hour and then compound 202 (3.6 g, 13.3 mmol) was added in one portion. The reaction was stirred for one day, and the reaction mixture was then poured into ice-water. The aqueous solution was acidified to pH=4.6, filtered to obtained a white solid, and dried in a freeze drier to give crude compound 203 (3.9 g, 69.6%), which was used directly without purification.

### Preparation of Compound 203b

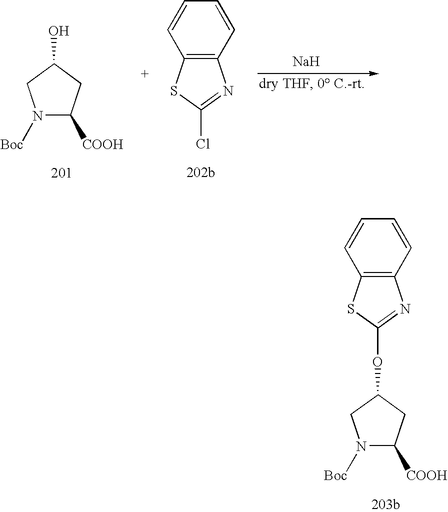

To a solution of compound 201 (2.31 g, 10 mmol) in 80 ml of dry THF, was added to NaH (60%, 2 g, 50 mmol). The reaction mixture was stirred for 10 minutes. To the resulting solution was added compound 202 (2.03 g, 12 mmol) in dry THF. The reaction mixture was stirred overnight. The reaction mixture then was poured into ice-water, and the aqueous phase was washed by petroleum ether to remove raw material compound 202, then acidified to pH=2 with aq. HCl (2 N). The mixture was extracted with ethyl acetate (50 mL×3) and dried over Na₂SO₄. The solvent was removed to give crude product 203b (3.64 g, 100%), which was used directly without purification.

### Preparation of Compound 203c

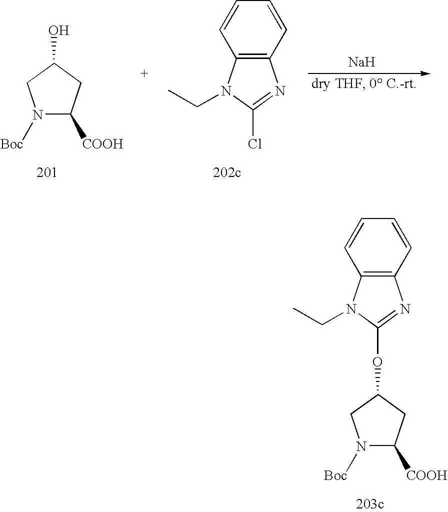

Preparation of compound 203c is similar to that of compound 203b (3.75 g, 100%).

### General Procedure for Preparation of Compound 205:

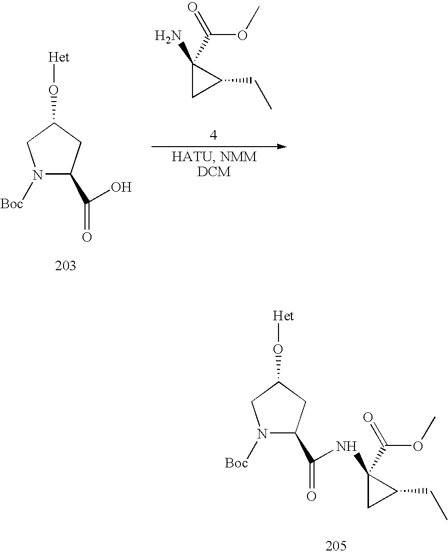

To a solution of compound 204 (2.5 g, 5.4 mmol) in dry DCM (20 mL), then added compound 203 (2 eq.), followed by adding HATU (3.5 g, 9.2 mmol) and 4.7 mL of NMM, the reaction mixture was stirred at room temperature for one day. The resulting mixture was concentrated to remove solvent, diluted with EtOAc, washed with pH=4.0 buffer and saturated aqueous NaHCO₃, dried and concentrated to give residue. The residue was purified by column chromatography to afford compound 205.

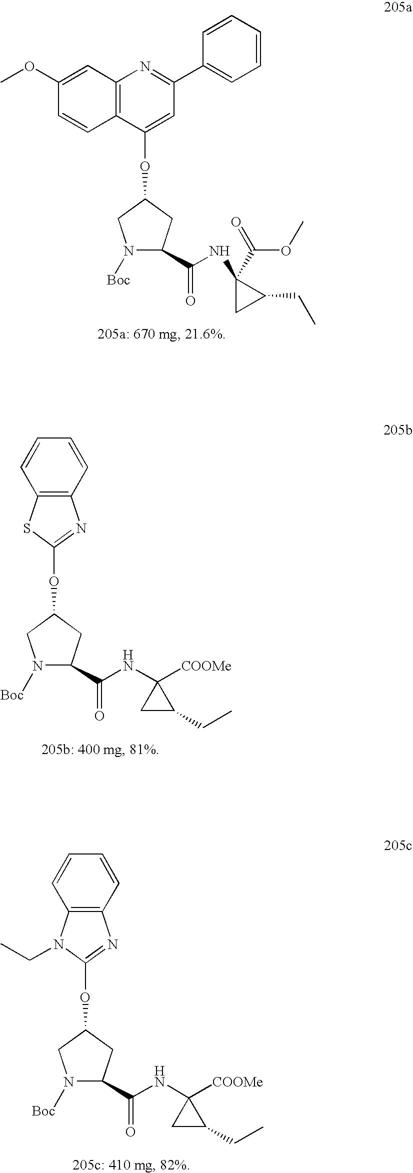

### General Procedure for Preparation of Compound 206:

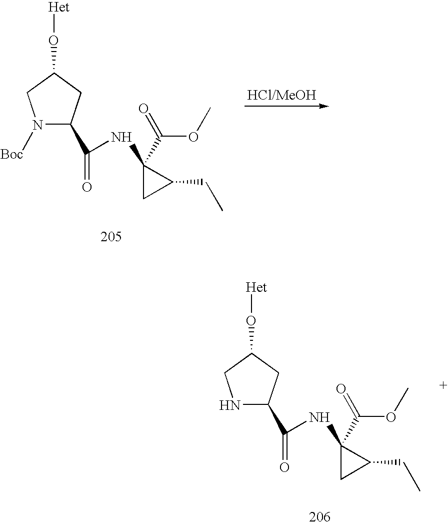

To a solution of compound 205 in dry DCM was added HCl in methanol solution (4N). The reaction mixture was stirred at room temperature for 3 h. LCMS analysis showed the reaction was complete. The reaction mixture was concentrated to give crude compound 206 (90%) used directly without further purification.

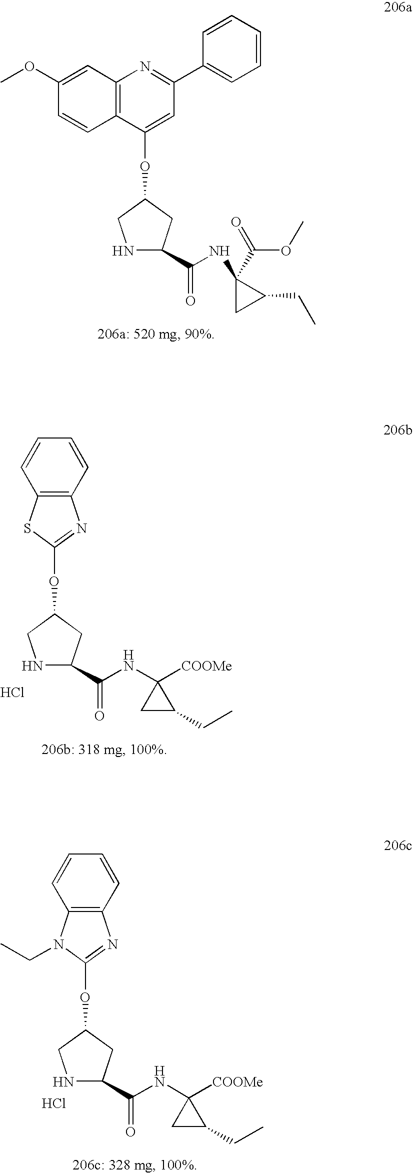

### General Procedure for the Preparation of Compound 208:

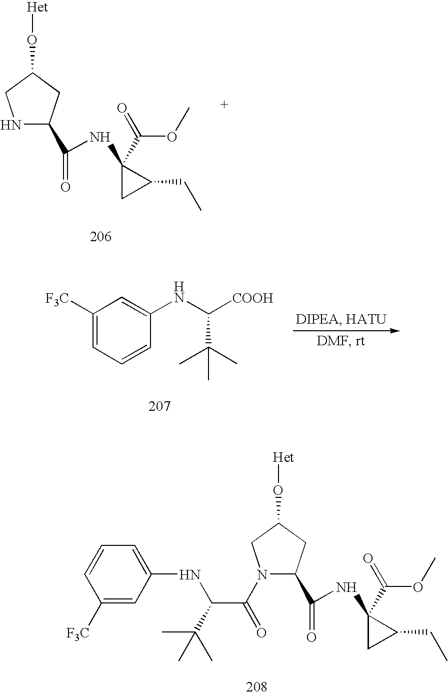

To a solution of compound 206 (1 eq.) in DMF was added DIPEA (8 eq.), then added compound 207 (1 eq.), followed by adding HATU (1.5 eq.). The reaction mixture was stirred overnight. LCMS analysis showed the reaction complete. The mixture was quenched by adding water and extracted with EtOAc (×3), the combined organic layer was dried over Na₂SO₄ and concentrated. The residue was purified by TLC (PE:EA=1:1) to afford compound 208.

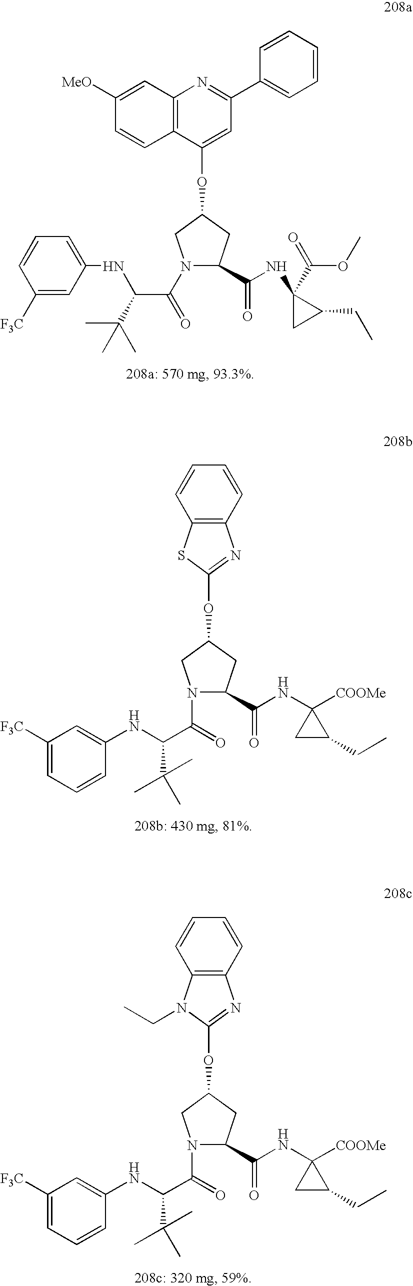

### General Procedure for Preparation of Compound 209:

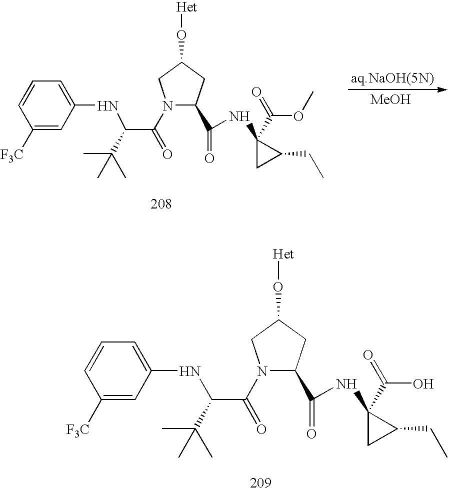

To a solution of compound 208 (1 eq.) in MeOH (5 mL) and was added aq. NaOH solution (6 N, 10 eq.) at room temperature. The reaction mixture was stirred at room temperature for two days. LCMS analysis showed the reaction was complete. The mixture was acidified to pH=4˜5 with 1N HCl solution by ice-water bath. The resulting mixture was extracted with EtOAc (×3). The combined organic layer was dried over Na₂SO₄ and concentrated to give crude compound 209 which was used directly without further purification.

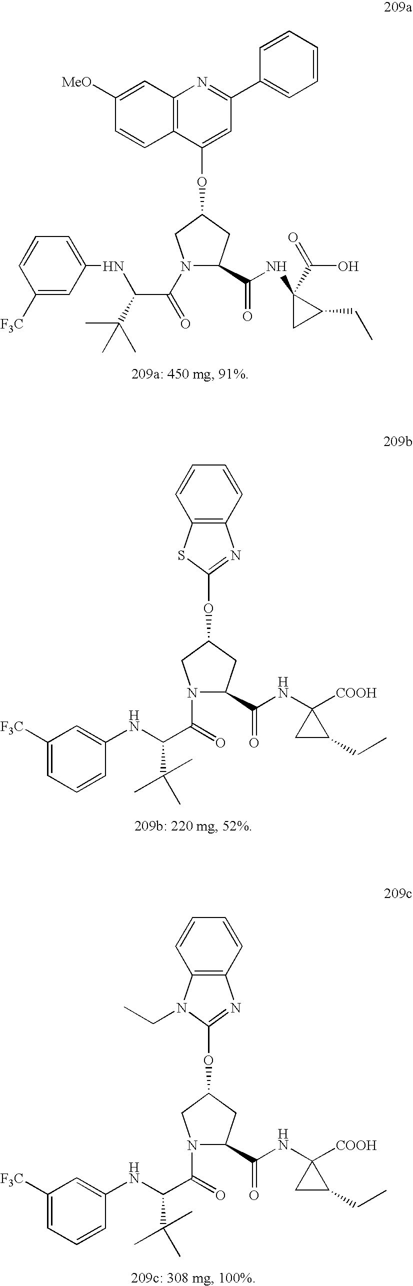

### General Procedure for Preparation of Final Compound 210:

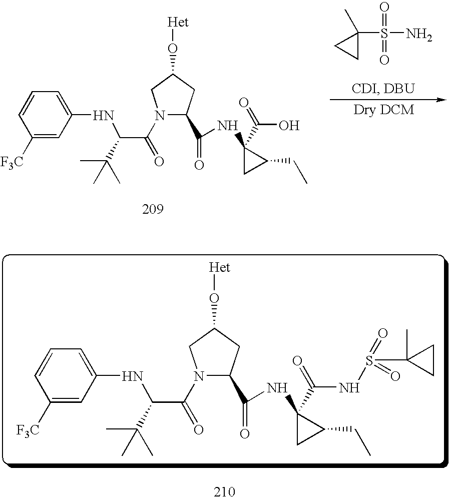

Final compound 210 was prepared by following the general procedure.

The following compounds were prepared using this method:

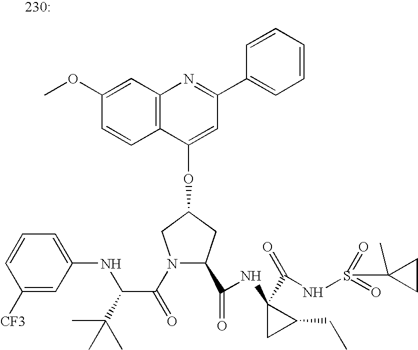

60 mg, 17.2%. MS (ESI) m/z (M+H)⁺850.2.

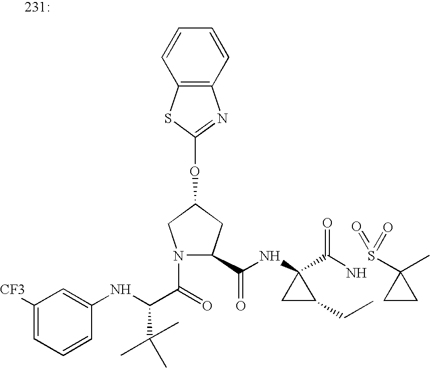

33 mg, 12.6%. MS (ESI) m/z (M+H)⁺750.

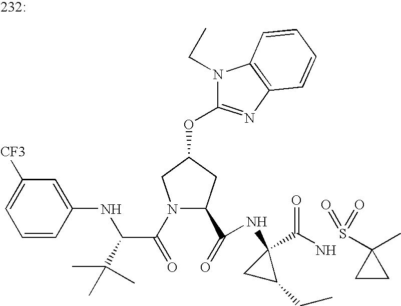

95 mg, 26%. MS (ESI) m/z (M+H)^{±}761.

### Stage 5: Compound 6

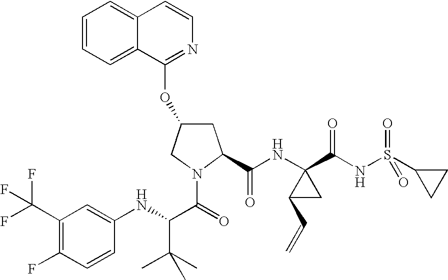

Procedure as described for 230. Yield: 51 mg (56%)

1H NMR (500 MHz, MeOD) δ 7.98 (d, J=5.87 Hz, 1H), 7.83 (dd, J=5.04, 7.98 Hz, 2H), 7.72 (t, J=7.70 Hz, 1H), 7.45 (t, J=7.70 Hz, 1H), 7.36 (d, J=5.87 Hz, 1H), 6.90-7.02 (m, 1H), 6.62 (d, J=9.17 Hz, 1H), 6.28 (t, J=9.63 Hz, 1H), 5.86 (br. s., 1H), 5.70-5.82 (m, 1H), 5.29 (d, J=17.06 Hz, 1H), 5.08-5.20 (m, 1H), 4.59 (s, 1H), 4.51 (dd, J=6.97, 10.27 Hz, 1H), 4.34 (d, J=11.92 Hz, 1H), 4.00-4.14 (m, 2H), 2.93-2.98 (m, 1H), 2.57 (dd, J=7.15, 13.57 Hz, 1H), 2.14-2.35 (m, 2H), 1.88 (dd, J=5.59, 8.16 Hz, 1H), 1.44 (dd, J=5.41, 9.45 Hz, 1H), 1.24-1.33 (m, 3H), 1.12 (s, 9H)

LC-MS: purity 96% (UV), tR 5.23 min m/z [M+H]+ 746.30

### Stage 5: Compound 7

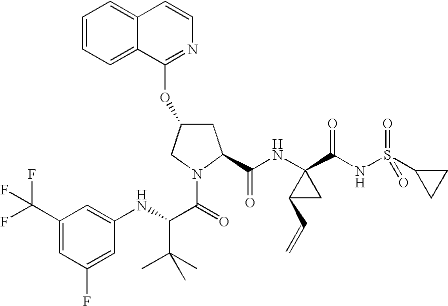

Procedure as described for 230. Yield: 53 mg (58%)

1H NMR (500 MHz, MeOD) δ 7.93 (d, J=5.87 Hz, 1H), 7.85 (d, J=8.25 Hz, 1H), 7.79 (d, J=8.25 Hz, 1H), 7.67 (t, J=7.61 Hz, 1H), 7.40 (t, J=7.70 Hz, 1H), 7.31 (d, J=5.87 Hz, 1H), 6.80 (s, 1H), 6.57 (d, J=11.55 Hz, 1H), 6.39 (d, J=8.80 Hz, 1H), 5.89 (br. s., 1H), 5.71-5.81 (m, 1H), 5.29 (d, J=16.87 Hz, 1H), 5.12 (d, J=10.45 Hz, 1H), 4.87-4.92 (m, 1H), 4.52 (dd, J=7.15, 10.27 Hz, 1H), 4.35 (d, J=12.47 Hz, 1H), 4.17 (s, 1H), 4.11 (dd, J=3.48, 12.10 Hz, 1H), 2.92-2.98 (m, 1H), 2.56-2.62 (m, 1H), 2.24-2.32 (m, 1H), 2.18-2.23 (m, 1H), 1.88 (dd, J=5.50, 7.89 Hz, 1H), 1.44 (dd, J=5.50, 9.54 Hz, 1H), 1.20-1.37 (m, 3H), 1.13 (s, 10H), 1.06-1.11 (m, 2H)

LC-MS: purity 100% (UV), tR 5.20 min m/z [M+H]+ 746.05

### Preparation of N-Aryl Tert-Leucine Amino Acids

General procedure: (2S)-2-(3-Fluoro-5-trifluoromethyl-phenylamino)-3,3-dimethyl-butanoic acid (250)

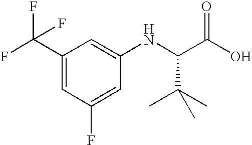

L-tert-leucine (4.0 g, 30.5 mmol, 1.0 eq), lithium chloride (129 mg, 3.05 mmol, 0.1 eq.), copper(I) iodide (289 mg, 1.52 mmol, 0.05 eq) and cesium carbonate (7.5 g, 22.9 mmol, 0.75 eq) were charged into a 250 mL flask. tert-Butanol (100 mL) was added and the resulting mixture stirred at 40° C. for 20 minutes by which time the milky solution had turned blue. 3-Fluoro-5-trifluoromethyl-bromobenzene (7.41 g, 30.5 mmol, 1 eq.) was added dropwise and the reaction mixture heated at 100° C. for 15 hours. LCMS analysis of an aliquot showed around 20% (UV) of unreacted 3-Fluoro-5-trifluoromethyl-bromobenzene. Extra copper(I) iodide (289 mg, 0.05 eq.) was added and the reaction mixture stirred at 100° C. for another 24 hours. LCMS analysis showed ˜16% (UV) of remaining 3-Fluoro-5-trifluoromethyl-bromobenzene. Heating was stopped and the solvent removed under vacuum to give a blue solid. The solid was partitioned between ethyl acetate (100 mL) and water (100 mL). The pH of the aqueous phase was adjusted to pH=1 with 4M Hydrochloric acid (10 mL). The organic phase was collected, washed with 2M hydrochloric acid (2×100 mL) dried over sodium sulfate, filtered and the solvent removed under vacuum to give 6.90 g (77%) of the title compound as an orange solid which was used in the next step without further purification.

¹H NMR (500 MHz, CHLOROFORM-d) δ ppm 6.61-6.75 (m, 2H) 6.49 (dt, J=10.68, 2.14 Hz, 1H) 4.48 (br. s., 1H) 3.79 (s, 1H) 1.11 (s, 9H)

LC-MS: purity 100% (ELS) 90% (UV), t_{R} 2.14 min m/z [M+H]⁺ 294.10

The next amino acids were prepared following the general procedure described for 250.

### (2S)-2-(4-Fluoro-3-trifluoromethyl-phenylamino)-3,3-dimethyl-butanoic acid (251)

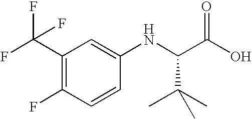

3.86 g (50%) of a brown solid.

¹H NMR (250 MHz, CHLOROFORM-d) δ ppm 6.93-7.06 (m, 1H) 6.84 (dd, J=5.56, 2.97 Hz, 1H) 6.71-6.81 (m, 1H) 6.21 (br. s., 2H) 3.73 (s, 1H) 1.10 (s, 9H)

LC-MS: purity 97% (UV), t_{R} 2.12 min m/z [M+H]⁺294.00 (MET/CR/1278)

### (2S)-2-(3-trifluoromethoxyphenylamino)-3,3-dimethyl-butanoic acid (252)

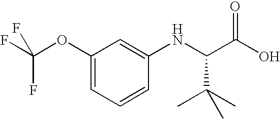

407 mg (11%) of a brown solid.

¹H NMR (500 MHz, CHLOROFORM-d) δ 7.17 (t, J=8.24 Hz, 1H), 6.54-6.66 (m, 2H), 6.50 (s, 1H), 3.78 (s, 1H), 1.04-1.14 (m, 9H)

LC-MS: purity 66% (UV), t_{R} 2.14 min m/z [M+H]⁺292.15 (MET/CR/1278)

### (2S)-2-(4-trifluoromethyl-phenylamino)-3,3-dimethyl-butanoic acid (253)

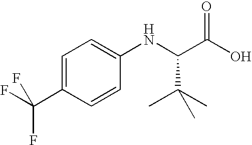

3.6 g (71%) of a dark brown solid.

¹H NMR (250 MHz, CHLOROFORM-d) δ 7.50-7.74 (m, 2H), 7.45 (d, J=8.53 Hz, 2H), 6.78 (d, J=8.53 Hz, 2H), 3.87 (s, 1H), 1.04-1.20 (m, 9H)

LC-MS: purity 86% (UV), t_{R} 2.18 min m/z [M+H]⁺276.10 (MET/CR/1278)

### Preparation of New P1/P1′ Analogs

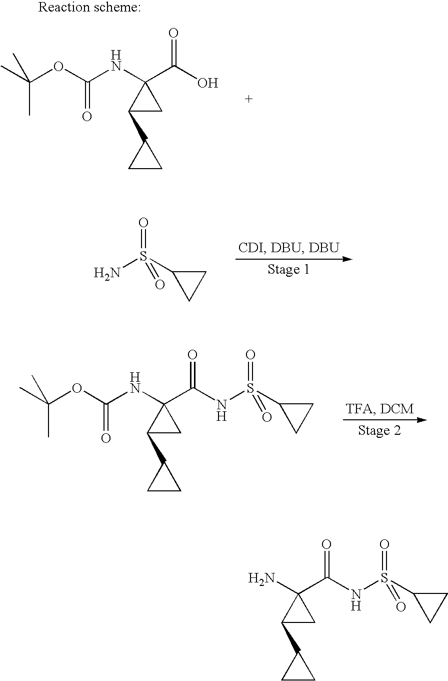

### General Method for Stage 1: 254

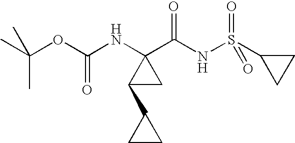

The N-Boc amino acid (3.17 g, 13.14 mmol, 1.0 eq.) was dissolved in dichloroethane (50 mL) and stirred at ambient temperature with molecular sieves (4 g) for 1 hour. After filtration, CDI (2.98 g, 18.39 mmol, 1.4 eq.) was added to the solution. The mixture was stirred during 1.5 hours at 50° C. The solution was then cooled down to ambient temperature and the cyclopropane sulphonamide (2.82 g, 23.26 mmol, 1.77 eq.) and DBU (5.31 mL, 35.48 mmol, 2.7 eq.) were then added. The mixture was stirred at 50° C. for 15 hours. The solvent was removed under vacuum. The residue was dissolved in DCM (40 mL) and washed with 0.5M hydrochloric acid (3×40 mL). The organic phase was dried over sodium sulfate, filtered, and the solvent removed under vacuum to give 2.04 g (45%) of the desired compound as an off white solid.

¹H NMR (250 MHz, CHLOROFORM-d) δ 9.82 (br. s., 1H), 5.02-5.26 (m, 1H), 2.87-3.10 (m, 1H), 1.66-1.80 (m, 1H), 1.37-1.55 (m, 11H), 1.15-1.25 (m, 1H), 1.02-1.15 (m, 3H), 0.73 (br. s., 1H), 0.45-0.63 (m, 2H), 0.25-0.45 (m, 2H)

LC-MS: purity 81% (UV), t_{R}1.82 min m/z [M+Na]⁺367.05 (MET/CR/1278).

The next P1/P1′ derivatives were prepared following the general method for stage 1.

### Stage 1: 255

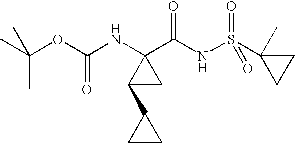

625 mg (21%) of the desired product. Product used in next step without purification

¹H NMR: No spectrum recorded at this stage.

LC-MS: purity 52% (UV), t_{R}1.99 min m/z [M+Na]⁺381.50 (MET/CR/1278).

Stage 1: 256

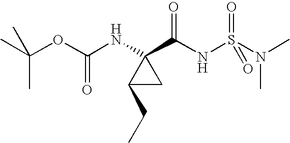

1.36 g (93%) of the desired product.

¹H NMR (500 MHz, CHLOROFORM-d) d 9.50 (br. s., 1H), 5.16 (br. s., 1H), 2.96 (s, 6H), 1.57 (br. s., 2H), 1.36-1.51 (m, 11H), 1.09 (br. s., 1H), 1.02 (s, 3H)

LC-MS: purity 100% (UV), t_{R}1.93 min m/z [M+Na]⁺358.05 (MET/CR/1278).

### General Method for Stage 2: 257

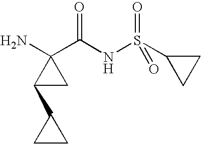

Stage 1 derivative (592 mg, 1.72 mmol, 1 eq.) and dichloromethane (5 mL) were charged into a 25 mL flask. The solution was cooled to 0° C., and a solution of trifluoroacetic acid (1.85 mL) in dichloromethane (5.5 mL) was added slowly and stirring continued for another 30 minutes, then the reaction was allowed to warm to ambient temperature and stirred for 2 hours. The solvent was removed under vacuum to give 420 mg (100%) of the desired product which was used in the next step without further purification.

¹H NMR: No spectrum recorded at this stage.

LC-MS: purity 98% (UV), t_{R} 0.73 min m/z [M+H]⁺245.00 (MET/CR/1278).

The next P1/P1′ derivatives were prepared following the general method for stage 2.

### Stage 2: 258

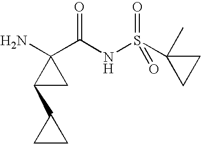

450 mg (99%) of the desired product.

¹H NMR (250 MHz, MeOD) δ 1.59-1.86 (m, 1H), 1.50-1.58 (m, 1H), 1.49 (s, 3H), 1.31-1.47 (m, 2H), 1.08-1.21 (m, 1H), 0.95-1.08 (m, 1H), 0.75-0.95 (m, 1H), 0.60-0.74 (m, 1H), 0.38-0.60 (m, 2H), 0.12-0.36 (m, 2H)

LC-MS: purity 97% (UV), t_{R}1.00 min m/z [M+H]⁺259.10 (MET/CR/1278).

### Stage 2: 259

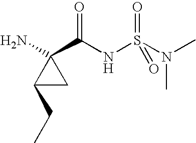

700 mg (99%) of an orange solid.

¹H NMR (500 MHz, CHLOROFORM-d) d 7.97 (br. s., 4H), 2.92 (s, 6H), 1.73-1.82 (m, 1H), 1.58-1.68 (m, 2H), 1.41-1.54 (m, 2H), 1.02 (t, J=7.41 Hz, 3H)

LC-MS: purity 99% (UV), t_{R} 0.67 min m/z [M+H]⁺236.00 (MET/CR/1278).

### Syntheses of Non-Macrocycles Final Products:

### Preparation of Non-Macrocycles Analogues Following Route 1:

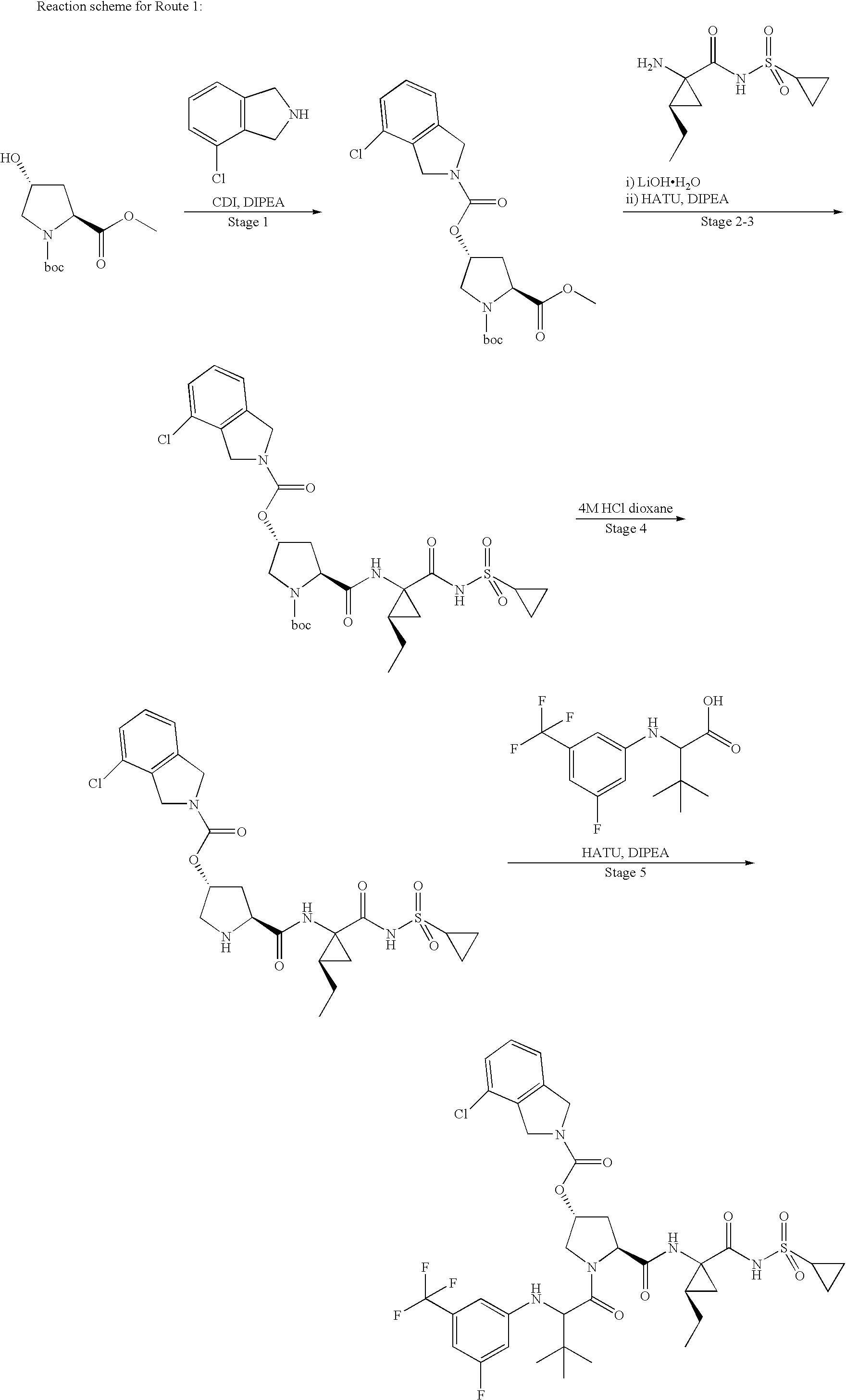

General procedure for Route 1: Synthesis of 260

### Stage 1: 261

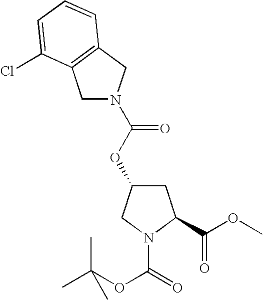

CDI (5.15 g, 31.80 mmol, 1.3 eq) was added to a solution of N—BOC-trans-4-hydroxy-L-proline methyl ester (6.00 g, 24.46 mmol, 1.0 eq) in tetrahydrofuran (100 mL) and stirred for 15 hours whilst allowing to warm to ambient temperature. 4-Chloroisoindoline hydrochloride (4.62 g, 24.46 mmol, 1.0 eq) was then added at 0° C. to the reaction mixture followed by triethylamine (7.1 mL, 50.92 mmol, 2.0 eq) and stirred for 15 hours at ambient temperature. The reaction mixture was concentrated under vacuum to give a pink paste. The residue was dissolved in ethyl acetate (100 mL) and washed with 0.5 M hydrochloric acid twice (2×100 mL). The organic phase was dried over sodium sulfate, filtered, and the solvent removed under vacuum. The residue was purified by flash column chromatography, eluting with ethyl acetate:heptanes (from neat heptanes to 50% EtOAc in heptanes). The relevant fractions were combined and the solvent removed under vacuum to give 9.77 g (94%) of the desired product.

¹H NMR (500 MHz, CHLOROFORM-d) δ 7.20-7.27 (m, 2H), 7.08-7.20 (m, 1H), 5.27-5.38 (m, 1H), 4.78 (br. s., 1H), 4.74 (br. s., 1H), 4.73 (s, 1H), 4.67 (br. s., 1H), 4.33-4.55 (m, 1H), 3.55-3.86 (m, 5H), 2.40-2.55 (m, 1H), 2.25 (ddd, J=5.11, 8.43, 13.77 Hz, 1H), 1.45 (dd, J=3.28, 15.64 Hz, 9H)

LC-MS: purity 87% (UV), t_{R} 2.24 min m/z [M+H]⁺447.15 (MET/CR/1278).

### Stage 2: 262

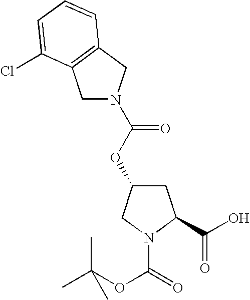

A solution of lithium hydroxide monohydrate (590 mg, 14.05 mmol, 1.5 eq) in water (20 mL) was added to a solution of the methyl ester (3.98 g, 9.37 mmol, 1.0 eq) in tetrahydrofuran:methanol (2:1, 60 mL) at 0° C. and stirred for 15 minutes before continuing at ambient temperature for a further 15 hours. The reaction mixture was then concentrated in vacuo. Ethyl acetate (50 mL) and brine (50 mL) were added and the mixture was acidified to pH 3 with 1M hydrochloric acid. The organic layer was separated and the aqueous layer was further extracted with ethyl acetate (50 mL). The combined organic layers were dried over sodium sulphate, filtered and evaporated in vacuo, to give 3.71 g (96%) of the desired product.

3.71 g (96%) of the desired product

¹H NMR (250 MHz, CHLOROFORM-d) d 7.10-7.27 (m, 3H), 5.34 (br. s., 1H), 4.62-4.86 (m, 4H), 4.49-4.62 (m, 1H), 4.32-4.50 (m, 1H), 3.65-3.83 (m, 2H), 2.43-2.63 (m, 1H), 2.21-2.43 (m, OH), 1.38-1.53 (m, 9H)

LC-MS: purity 98% (UV), t_{R}1.97 min m/z [M+Na]⁺433.10(MET/CR/1278).

Stage 3: 263

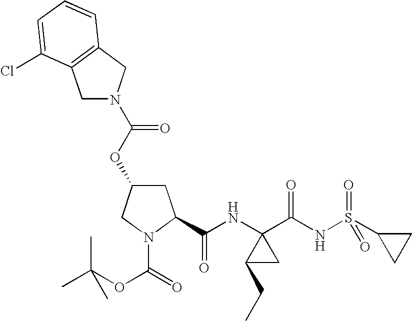

Diisopropylethylamine (4.7 mL, 27.12 mmol, 3.0 eq) was added to a stirred suspension of the above proline (3.73 g, 9.04 mmol, 1.0 eq) and HATU (5.16 g, 13.56 mmol, 1.5 eq) in dichloromethane (100 mL) at 0° C. After 1 hour cyclopropanesulfonic acid ((1R,2R)-1-amino-2-ethyl-cyclopropanecarbonyl)-amide (2.43 g, 9.04 mmol, 1.0 eq) was added and this was stirred for 15 hours whilst allowing to warm to ambient temperature. The reaction mixture was washed with brine (100 mL) then the aqueous phase was extracted with dichloromethane (100 mL). The combined organic layers were dried over sodium sulfate, filtered and evaporated under vacuum. The residue was purified by flash column chromatography, eluting with ethyl acetate:heptanes (from 6:4 to 7:3) to give partial purification. The relevant mixed fractions were combined and the solvent removed under vacuum. The residue was purified a second time by flash column chromatography, eluting with a methanol:dichloromethane gradient (1% MeOH in DCM to 3% MeOH in DCM). The relevant fractions were combined and the solvent removed under vacuum to give 4.10 g (72%) of the desired product.

¹H NMR (500 MHz, CHLOROFORM-d) δ 7.27 (s, 1H), 7.09-7.22 (m, 1H), 6.95 (br. s., 1H), 5.34 (br. s., 1H), 4.61-4.85 (m, 4H), 4.22-4.31 (m, 1H), 3.70-3.77 (m, 1H), 3.63-3.70 (m, 1H), 2.94-3.02 (m, 1H), 2.40 (br. s., 1H), 2.34 (br. s., 1H), 2.18 (s, 2H), 1.69 (br. s., 1H), 1.60-1.66 (m, 2H), 1.50 (d, J=3.30 Hz, 9H), 1.36-1.46 (m, 2H), 1.34 (br. s., 1H), 1.21 (br. s., 1H), 1.07 (br. s., 2H), 0.96-1.03 (m, 3H)

LC-MS: purity 96% (UV), t_{R} 2.25 min m/z [M+Na]⁺647.25(MET/CR/1278).

### Stage 4: 264

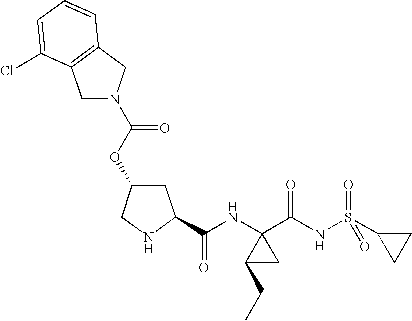

4M HCl in Dioxane (16 mL) was added to a solution of the N-Boc derivative (668 mg, 1.06 mmol, 1.0 eq) at 0° C. and stirred for 15 minutes then for a further 2 hours at ambient temperature. The reaction mixture was allowed to stand for 15 hours then evaporated to dryness. The residue was then evaporated from dichloromethane (2×25 mL) and used in the next stage without any further purification.

LC-MS: purity 99% (UV), t_{R}1.40 min m/z [M+H]⁺525.00

### Stage 5-260

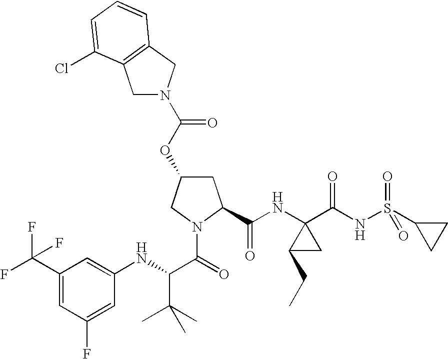

(S)-3-fluoro-5-trifluoromethyl-2-(benzenylamino)-butyric acid (155 mg, 0.53 mmol, 1.1 eq.) and dimethylformamide (5 mL) were charged into a 7 mL vial and the reaction mixture cooled on top of an ice bath. HATU (237 mg, 0.62 mmol, 1.3 eq) and diisopropylethylamine (0.585 mL, 3.36 mmol, 7.0 eq) were added each as a single portion and the reaction mixture stirred at 0° C. for a further 30 minutes. Stage 4 intermediate (269 mg, 0.48 mmol, 1 eq.) was added and the reaction mixture stirred at ambient temperature for a further 15 hours. The reaction mixture was diluted with ethyl acetate (30 mL) and washed with water (2×25 mL). The organic layer was dried over sodium sulphate, filtered and the solvent removed under vacuum. Purification by flash column chromatography eluting with a methanol:dichloromethane gradient (from 1% MeOH in DCM to 5% MeOH in DCM) gave 210 mg (55%) of the desired product as an off white solid.

LC-MS: purity 100% (UV), t_{R} 5.28 min m/z [M+H]⁺800.35 (MET/CR/1416).

¹H NMR (500 MHz, CHLOROFORM-d) δ ppm 9.99-10.19 (m, 1H) 7.22-7.27 (m, 2H) 6.97-7.20 (m, 1H) 6.91-6.96 (m, 1H) 6.63 (d, 1H) 6.38 (t, 2H) 5.40-5.47 (m, 1H) 4.83 (d, 1H) 4.77 (s, 1H) 4.69-4.74 (m, 1H) 4.48-4.55 (m, 1H) 4.38-4.47 (m, 1H) 4.25-4.34 (m, 1H) 3.95-4.02 (m, 2H) 3.89 (dd, 1H) 2.90-2.98 (m, 1H) 2.31-2.49 (m, 2H) 1.67-1.72 (m, 1H) 1.52-1.64 (m, 2H) 1.34-1.46 (m, 3H) 1.28-1.34 (m, 1H) 1.10 (s, 9H) 1.01-1.08 (m, 2H) 0.97 (t, J=7.40 Hz, 3H)

The following 4-Fluoro and 5-methoxy-isoindoline derivatives were prepared following the method described for 260

### Stage 1: 265

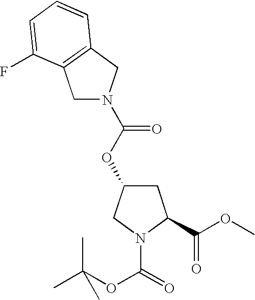

8.30 g (83%) of the desired product

¹H NMR (250 MHz, CHLOROFORM-d) δ 7.27 (s, 1H), 6.90-7.12 (m, 2H), 5.31-5.40 (m, 1H), 4.74 (d, J=14.92 Hz, 4H), 4.32-4.57 (m, 1H), 3.77 (s, 5H), 2.36-2.59 (m, 1H), 2.25 (ddd, J=4.95, 8.41, 13.74 Hz, 1H), 1.38-1.54 (m, 9H)

LC-MS: purity 99% (UV), t_{R}1.38 min m/z [M+H]⁺431.15 (MET/CR/1278).

### Stage 1: 266

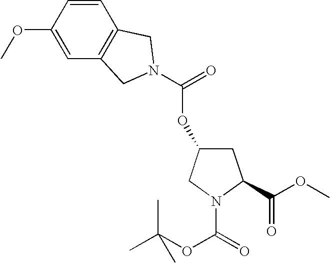

730 mg (65%) of the desired product

¹H NMR (250 MHz, CHLOROFORM-d) d 7.15 (dd, J=8.53, 10.96 Hz, 1H), 6.63-6.95 (m, 2H), 5.33 (br. s., 1H), 4.65 (dd, J=9.44, 15.38 Hz, 4H), 4.22-4.56 (m, 1H), 3.70-3.91 (m, 8H), 2.35-2.65 (m, 1H), 2.12-2.35 (m, 1H), 1.45 (d, J=7.16 Hz, 9H)

LC-MS: purity 99% (UV), t_{R}1.54 min m/z [M+Na]⁺443.15 (MET/CR/1278).

### Stage 2: 267

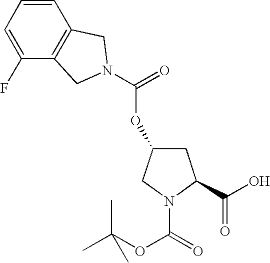

8.1 g (99%) of off white/beige foam

¹H NMR (500 MHz, CHLOROFORM-d) δ 7.26-7.36 (m, 1H), 6.89-7.15 (m, 2H), 5.25-5.45 (m, 1H), 4.64-4.88 (m, 4H), 4.35-4.62 (m, 1H), 3.57-3.90 (m, 2H), 2.23-2.68 (m, 2H), 1.43-1.56 (m, 9H)

LC-MS: purity 96% (UV), t_{R}1.27 min m/z [M+H]⁺295.05 (MET/CR/1278).

### Stage 3: 268

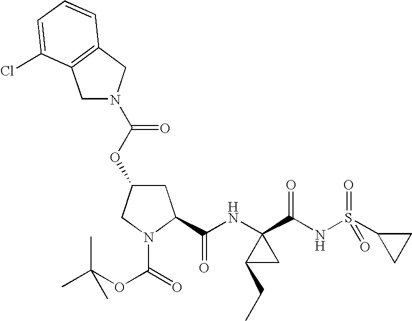

4.1 g (70%) of the desired product

¹H NMR (500 MHz, CHLOROFORM-d) δ 7.27 (s, 1H), 7.09-7.22 (m, 1H), 6.95 (br. s., 1H), 5.34 (br. s., 1H), 4.61-4.85 (m, 4H), 4.22-4.31 (m, 1H), 3.70-3.77 (m, 1H), 3.63-3.70 (m, 1H), 2.94-3.02 (m, 1H), 2.40 (br. s., 1H), 2.34 (br. s., 1H), 2.18 (s, 2H), 1.69 (br. s., 1H), 1.60-1.66 (m, 2H), 1.50 (d, J=3.30 Hz, 9H), 1.36-1.46 (m, 2H), 1.34 (br. s., 1H), 1.21 (br. s., 1H), 1.07 (br. s., 2H), 0.96-1.03 (m, 3H)

LC-MS: purity 96% (UV), t_{R} 2.25 min m/z [M+Na]⁺647.25 (MET/CR/1278)

### Stage 3: 269

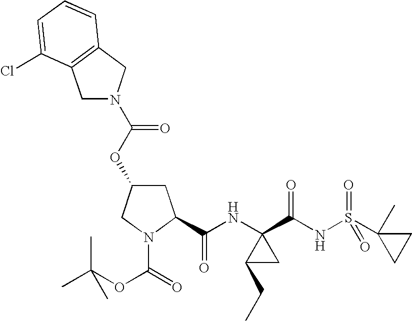

1.26 g (84%) of an off white solid

¹H NMR (500 MHz, CHLOROFORM-d) δ 9.74 (br. s., 1H), 7.22-7.27 (m, 1H), 7.09-7.20 (m, 1H), 6.98-7.07 (m, 1H), 5.34 (br. s., 1H), 4.60-4.85 (m, 4H), 4.29 (d, J=7.48 Hz, 1H), 3.59-3.80 (m, 2H), 2.25-2.54 (m, 2H), 1.69-1.77 (m, 1H), 1.64-1.69 (m, 2H), 1.62 (s, 2H), 1.60 (br. s., 1H), 1.53-1.56 (m, 3H), 1.51 (d, J=4.12 Hz, 9H), 1.35-1.46 (m, 1H), 1.18 (br. s., 1H), 1.01 (t, J=6.87 Hz, 3H), 0.87-0.93 (m, 1H), 0.80-0.87 (m, 1H)

LC-MS: purity 100% (UV), t_{R}1.54 min m/z [M+Na]⁺661.25 (MET/CR/1278).

### Stage 3: 270

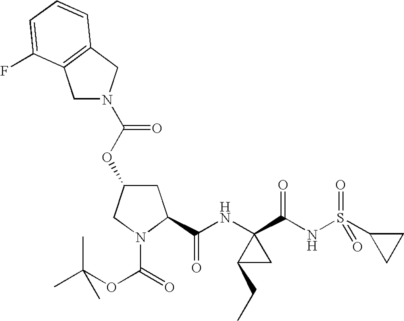

543 mg (70%) of the desired product

¹H NMR (500 MHz, CHLOROFORM-d) δ 10.04 (br. s., 1H), 6.88-7.18 (m, 3H), 5.33 (br. s., 1H), 4.76 (d, J=6.87 Hz, 2H), 4.68 (d, J=8.09 Hz, 2H), 4.26 (t, J=7.63 Hz, 1H), 3.58-3.80 (m, 2H), 2.91-3.03 (m, 1H), 2.22-2.46 (m, 2H), 1.84 (s, 1H), 1.55-1.74 (m, 3H), 1.41-1.53 (m, 11H), 1.33-1.41 (m, 1H), 1.28-1.34 (m, 1H), 1.02-1.13 (m, 2H), 0.94-1.01 (m, 3H)

LC-MS: purity 100% (UV), t_{R}1.58 min m/z [M+Na]⁺631.35 (MET/CR/1278).

### Stage 3: 271

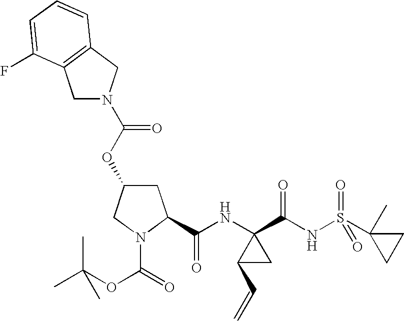

2.85 g (54%) of an off white solid

¹H NMR (500 MHz, DMSO-d₆) δ 10.09-10.94 (m, 1H), 8.33-9.13 (m, 1H), 7.31-7.42 (m, 1H), 7.08-7.24 (m, 2H), 5.36-5.67 (m, 1H), 5.07-5.33 (m, 3H), 4.69 (br. s., 4H), 4.15-4.26 (m, 1H), 3.45-3.76 (m, 2H), 2.89 (s, 1H), 2.05-2.47 (m, 2H), 1.72 (br. s., 1H), 1.29-1.45 (m, 14H), 1.14-1.28 (m, 1H), 0.89 (br. s., 2H)

LC-MS: purity 98% (UV), t_{R} 2.19 min m/z [M+Na]⁺643.25 (MET/CR/1278).

### Stage 3: 272

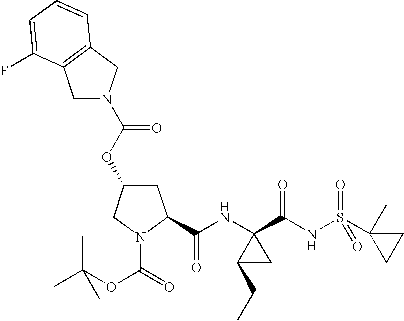

640 mg (83%) of the desired product

¹H NMR (250 MHz, CHLOROFORM-d) δ 9.75 (s, 1H), 6.87-7.15 (m, 3H), 5.33 (br. s., 1H), 4.74 (d, J=19.80 Hz, 4H), 4.28 (br. s., 1H), 3.70 (br. s., 2H), 2.81 (s, 1H), 1.67 (br. s., 4H), 1.54 (s, 4H), 1.50 (s, 9H), 1.49 (br. s., 1H), 1.17 (br. s., 1H), 0.96-1.08 (m, 3H), 0.89 (br. s., 2H)

LC-MS: purity 57% (UV), t_{R}1.49 min m/z [M+Na]⁺645.25 (MET/CR/1278).

### Stages 2-3: 273/274

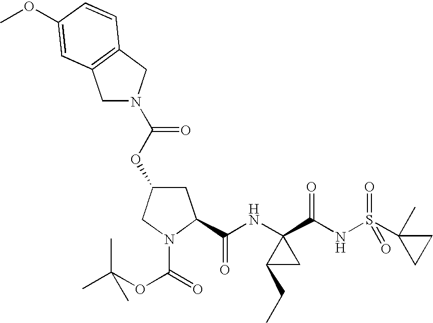

Note: Stage 2 intermediate was used crude (assume quantitative yield) for stage 3 and was not fully characterised.

600 mg (56%) of the white solid

¹H NMR (250 MHz, CHLOROFORM-d) d 9.77 (s, 1H), 7.16 (dd, J=8.38, 13.40 Hz, 1H), 7.03 (s, 1H), 6.70-6.91 (m, 2H), 5.27-5.40 (m, 1H), 4.53-4.76 (m, 4H), 4.28 (t, J=7.84 Hz, 1H), 3.82 (s, 3H), 3.60-3.77 (m, 2H), 2.20-2.52 (m, 2H), 1.57-1.76 (m, 2H), 1.54 (s, 3H), 1.50 (s, 9H), 1.33-1.45 (m, 1H), 1.10-1.32 (m, 3H), 1.00 (t, J=7.23 Hz, 3H), 0.78-0.94 (m, 3H)

LC-MS: purity 99% (UV), t_{R}1.47 min m/z [M+Na]⁺657.30 (MET/CR/1278).

### Stage 3: 275

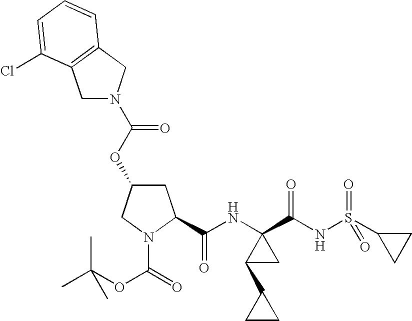

117 mg (99%) of the desired product

¹H NMR (250 MHz, CHLOROFORM-d) δ 10.03 (br. s., 1H), 7.26 (br. s., 1H), 7.14 (d, J=16.90 Hz, 2H), 4.61-4.85 (m, 4H), 4.29 (br. s., 1H), 3.68 (br. s., 1H), 2.36 (br. s., 2H), 1.84 (br. s., 1H), 1.62 (d, J=1.98 Hz, 3H), 1.42-1.55 (m, 10H), 1.31-1.42 (m, 2H), 1.21 (br. s., 1H), 1.08 (br. s., 3H), 0.81-0.97 (m, 1H), 0.58 (br. s., 2H), 0.34 (br. s., 2H)

LC-MS: purity 76% (UV), t_{R} 5.33 min m/z [M+Na]⁺659.20 (MET/CR/1278).

### Stage 3: 276

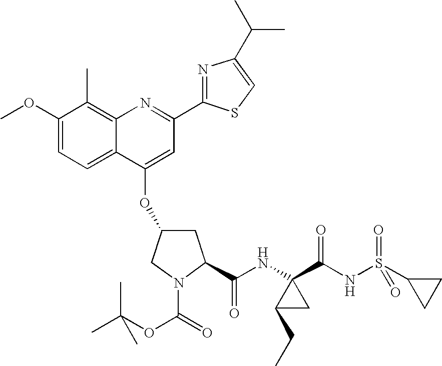

200 mg (71%) of the desired product

H NMR (250 MHz, CHLOROFORM-d) d 10.03 (s, 1H), 7.92 (d, J=9.14 Hz, 1H), 7.50 (s, 1H), 7.23 (d, J=9.29 Hz, 1H), 7.05 (d, J=0.76 Hz, 1H), 6.91 (s, 1H), 5.42 (br. s., 1H), 4.35 (t, J=7.54 Hz, 1H), 4.00 (s, 3H), 3.86-3.94 (m, 2H), 3.20 (spt, 1H), 2.92-3.05 (m, 1H), 2.71 (s, 3H), 2.52-2.64 (m, 1H), 1.64-1.78 (m, 2H), 1.47 (s, 10H), 1.39 (d, J=6.85 Hz, 9H), 1.07 (d, J=8.22 Hz, 3H), 0.99 (t, J=7.39 Hz, 4H)

LC-MS: purity 100% (UV), t_{R} 2.50 min m/z [M+H]⁺742.30 (MET/CR/1278).

### Stage 3: 277

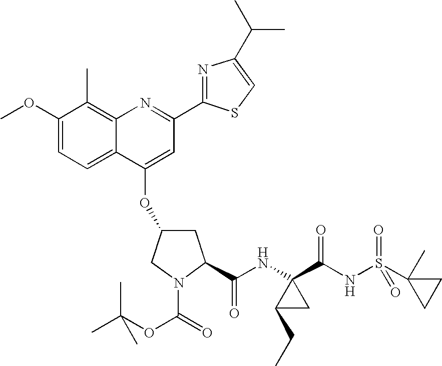

1.7 g (76%) of an off white solid

¹H NMR (250 MHz, CHLOROFORM-d) d 9.76 (br. s., 1H), 7.93 (d, J=9.14 Hz, 1H), 7.50 (s, 1H), 7.24 (d, J=9.14 Hz, 1H), 7.04 (s, 2H), 5.41 (br. s., 1H), 4.39 (t, J=7.92 Hz, 1H), 4.00 (s, 3H), 3.80-3.94 (m, 2H), 3.07-3.31 (m, 1H), 2.70 (s, 3H), 2.51-2.65 (m, 2H), 1.56-1.82 (m, 6H), 1.43-1.52 (m, 13H), 1.39 (d, J=7.01 Hz, 8H), 0.99 (t, J=7.31 Hz, 3H)

LC-MS: purity 100% (ELS), t_{R} 2.98 min m/z [M+H]⁺756.22 (MET/CR/1278).

### Stage 3: 278

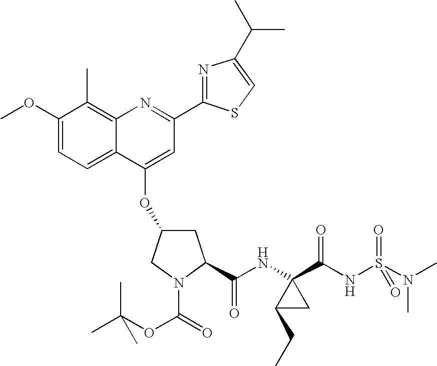

865 mg (68%) of the desired product

¹H NMR (250 MHz, CHLOROFORM-d) δ 9.76 (br. s., 1H), 7.92 (d, J=9.14 Hz, 1H), 7.50 (s, 1H), 7.23 (d, J=9.29 Hz, 1H), 7.05 (d, J=0.91 Hz, 1H), 6.93 (s, 1H), 5.41 (br. s., 1H), 4.37 (s, 1H), 4.00 (s, 3H), 3.81-3.94 (m, 2H), 3.11-3.30 (m, 1H), 2.95 (s, 6H), 2.71 (s, 3H), 2.48-2.64 (m, 2H), 1.67 (br. s., 1H), 1.58 (d, J=7.16 Hz, 2H), 1.48 (s, 9H), 1.39 (d, J=6.85 Hz, 7H), 1.19 (br. s., 1H), 1.00 (t, J=7.23 Hz, 3H)

LC-MS: purity 95% (UV), t_{R} 2.51 min m/z [M+H]⁺745.35 (MET/CR/1981).

### Stage 3: 279

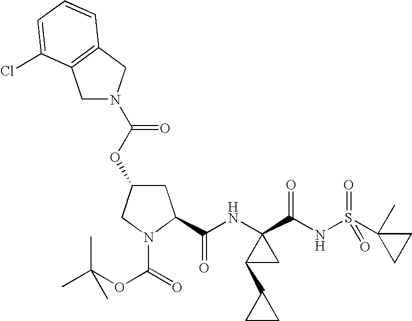

720 mg (63%) of the desired product

¹H NMR (250 MHz, MeOD) δ 7.11-7.41 (m, 3H), 5.28 (br. s., 1H), 4.59-4.81 (m, 4H), 4.19-4.45 (m, 1H), 3.59-3.83 (m, 2H), 2.81 (s, 2H), 2.29-2.47 (m, 1H), 2.05-2.23 (m, 1H), 1.74 (br. s., 1H), 1.38-1.66 (m, 14H), 1.30-1.35 (m, 1H), 1.01-1.18 (m, 1H), 0.92-0.99 (m, 1H), 0.44-0.69 (m, 2H), 0.22-0.43 (m, 2H)

LC-MS: purity 82% (UV), t_{R} 2.41 min m/z [M+Na]⁺673.30 (MET/CR/1278).

### Stages 4/5: 280

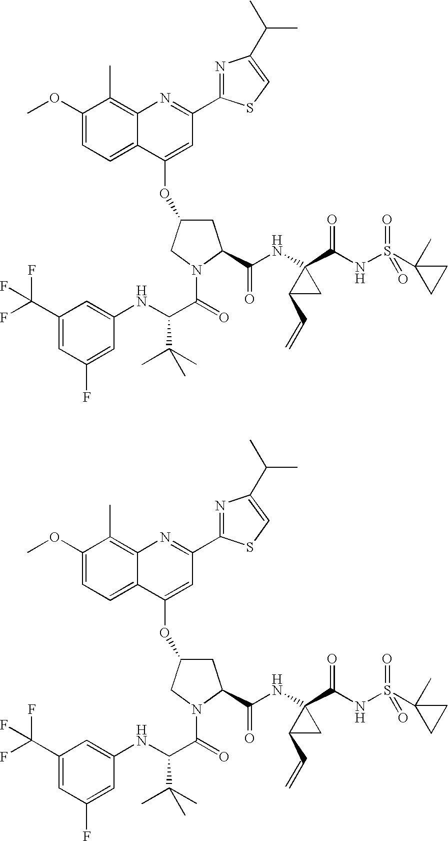

84 mg (35%) of a yellow solid

¹H NMR (250 MHz, CHLOROFORM-d) δ 9.80 (br. s., 1H), 7.34-7.71 (m, 2H), 6.85-7.26 (m, 3H), 6.17-6.82 (m, 3H), 5.60-5.88 (m, 1H), 5.50 (br. s., 1H), 5.01-5.36 (m, 2H), 4.88 (d, J=11.12 Hz, 1H), 4.55 (t, J=8.15 Hz, 1H), 4.00-4.34 (m, 2H), 3.95 (s, 3H), 2.98-3.45 (m, 1H), 2.41-2.85 (m, 5H), 2.00-2.14 (m, 1H), 1.92 (dd, J=5.94, 7.92 Hz, 1H), 1.57-1.71 (m, 2H), 1.47 (s, 3H), 1.39 (d, J=6.55 Hz, 7H), 1.02-1.17 (m, 10H), 0.73-0.95 (m, 2H)

LC-MS: purity 100% (UV), t_{R} 5.28 min m/z [M+H]⁺929.68 (MET/CR/1426).

### Stages 4/5: 281

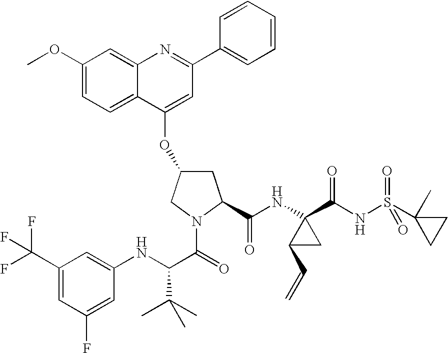

78 mg (26%) of a yellow solid

¹H NMR (500 MHz, CHLOROFORM-d) δ ppm 9.84 (br. s., 1H) 8.04 (d, J=7.17 Hz, 2H) 7.37-7.60 (m, 5H) 7.21 (br. s., 1H) 6.91-7.01 (m, 2H) 6.64 (s, 1H) 6.59 (d, J=8.24 Hz, 1H) 6.40 (d, J=10.68 Hz, 1H) 5.61-5.73 (m, 1H) 5.40 (br. s., 1H) 5.24 (d, J=17.24 Hz, 1H) 5.14 (d, J=10.53 Hz, 1H) 4.84 (d, J=10.07 Hz, 1H) 4.54 (t, J=8.09 Hz, 1H) 4.08-4.21 (m, 2H) 3.92-3.98 (m, 4H) 2.60 (d, J=7.02 Hz, 2H) 2.09 (d, J=8.85 Hz, 1H) 1.93 (dd, J=7.93, 6.10 Hz, 1H) 1.55-1.64 (m, 3H) 1.48 (s, 3H) 1.40 (dd, J=9.38, 6.03 Hz, 1H) 1.12 (s, 9H) 0.81-0.92 (m, 2H)

LC-MS: purity 100% (UV), t_{R} 4.30 min m/z [M+H]⁺866.40 (MET/CR/1416).

### Stages 4/5: 282

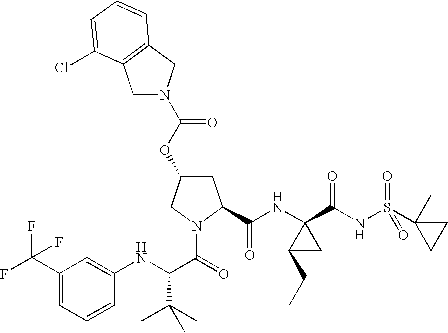

200 mg (66%) of an off white solid

¹H NMR (500 MHz, CHLOROFORM-d) δ 9.76 (d, J=16.48 Hz, 1H), 7.21-7.31 (m, 1H), 6.91-7.21 (m, 3H), 6.82 (d, J=5.80 Hz, 1H), 6.66-6.77 (m, 1H), 6.60 (t, J=8.39 Hz, 1H), 5.40 (br. s., 1H), 4.65-4.88 (m, 2H), 4.56-4.65 (m, 1H), 4.40-4.56 (m, 2H), 4.12 (t, J=15.03 Hz, 1H), 3.75-4.04 (m, 3H), 2.42-2.71 (m, 1H), 2.16-2.42 (m, 1H), 1.71-1.78 (m, 1H), 1.61-1.71 (m, 2H), 1.45-1.57 (m, 5H), 1.32-1.43 (m, 1H), 1.14-1.24 (m, 1H), 1.05-1.13 (m, 9H), 1.01 (t, J=7.32 Hz, 3H), 0.83-0.94 (m, 2H)

LC-MS: purity 100% (UV), t_{R} 5.34 min m/z [M+H]⁺796.1 (MET/CR/1416).

### Stages 4/5: 283

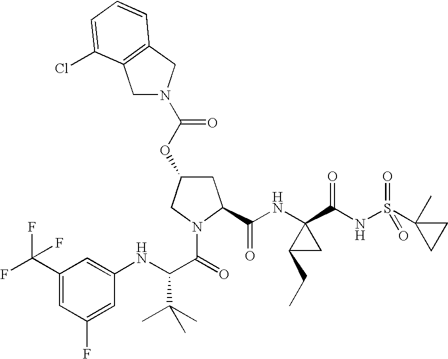

235 mg (76%) of an off white solid

¹H NMR (500 MHz, CHLOROFORM-d) δ 9.77 (d, J=13.89 Hz, 1H), 7.21-7.31 (m, 2H), 6.93-7.21 (m, 2H), 6.63 (d, J=10.99 Hz, 1H), 6.18-6.51 (m, 2H), 5.42 (br. s., 1H), 4.60-4.94 (m, 3H), 4.44-4.61 (m, 2H), 4.24 (d, J=14.80 Hz, 1H), 3.77-4.03 (m, 3H), 2.44-2.70 (m, 1H), 2.35 (dd, J=7.93, 14.19 Hz, 1H), 1.71-1.78 (m, 1H), 1.62-1.71 (m, 2H), 1.48-1.56 (m, 5H), 1.32-1.45 (m, 1H), 1.15-1.24 (m, 1H), 1.09 (s, 9H), 1.01 (t, J=7.32 Hz, 3H), 0.79-0.95 (m, 2H)

LC-MS: purity 100% (UV), t_{R} 5.39 min m/z [M+H]⁺814.0 (MET/CR/1416).

### Stages 4/5: 284

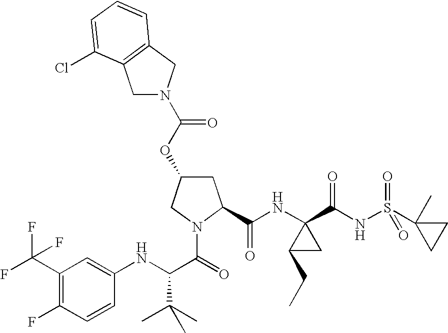

187 mg (60%) of an off white solid

¹H NMR (500 MHz, CHLOROFORM-d) δ 9.79 (d, J=6.87 Hz, 1H), 6.93-7.30 (m, 4H), 6.55-6.94 (m, 3H), 5.39 (br. s., 1H), 4.59-4.85 (m, 2H), 4.41-4.57 (m, 3H), 4.24 (t, J=14.65 Hz, 1H), 3.88-3.99 (m, 2H), 3.85 (dd, J=5.57, 10.45 Hz, 1H), 2.51 (ddd, J=4.88, 9.00, 13.89 Hz, 1H), 2.16-2.42 (m, 1H), 1.70-1.80 (m, 1H), 1.63-1.71 (m, 2H), 1.48-1.60 (m, 5H), 1.32-1.44 (m, 1H), 1.15-1.24 (m, 1H), 1.04-1.15 (m, 9H), 1.00 (d, J=14.65 Hz, 3H), 0.82-0.93 (m, 2H)

LC-MS: purity 95% (UV), t_{R} 5.31 min m/z [M+H]⁺814.0 (MET/CR/1416).

Stages 4/5: 285

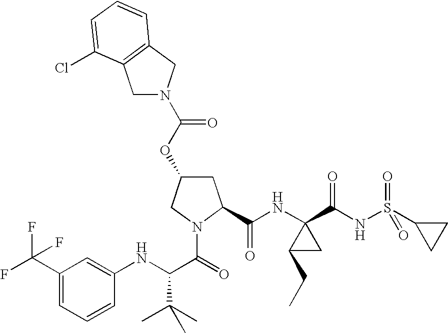

122 mg (37%) of an off white solid

¹H NMR (500 MHz, CHLOROFORM-d) δ 10.11 (d, J=14.50 Hz, 1H), 7.26-7.36 (m, 1H), 6.85-7.13 (m, 4H), 6.82 (s, 1H), 6.75 (d, J=7.63 Hz, 1H), 6.65 (dd, J=7.71, 14.11 Hz, 1H), 5.41 (br. s., 1H), 4.72 (d, J=6.71 Hz, 2H), 4.62 (dd, J=8.09, 10.38 Hz, 1H), 4.29-4.55 (m, 2H), 4.09-4.24 (m, 1H), 3.78-4.06 (m, 3H), 2.80-3.10 (m, 1H), 2.39-2.62 (m, 1H), 2.23-2.39 (m, 1H), 1.67-1.84 (m, 1H), 1.57-1.67 (m, 1H), 1.19-1.47 (m, 3H), 1.01-1.16 (m, 11H), 0.98 (t, J=7.40 Hz, 3H), 0.89 (t, J=6.87 Hz, 2H)

LC-MS: purity 99% (UV), t_{R} 5.13 min m/z [M+H]⁺766.0 (MET/CR/1416).

### Stages 4/5: 286

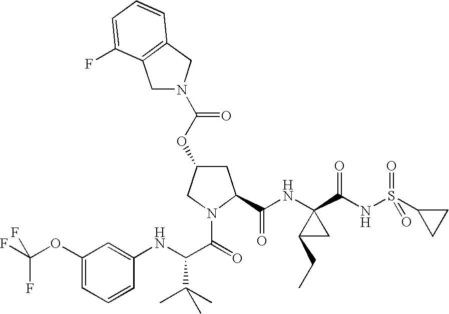

101 mg (30%) of an off white solid

¹H NMR (500 MHz, CHLOROFORM-d) δ 10.09 (d, J=14.50 Hz, 1H), 7.25-7.38 (m, 1H), 6.84-7.12 (m, 4H), 6.52 (d, J=8.09 Hz, 1H), 6.45 (s, 1H), 6.15-6.32 (m, 1H), 5.41 (br. s., 1H), 4.72 (d, J=6.26 Hz, 2H), 4.51-4.66 (m, 1H), 4.34-4.52 (m, 2H), 4.12-4.28 (m, 1H), 3.82-4.04 (m, 3H), 2.87-3.08 (m, 1H), 2.38-2.59 (m, 1H), 2.21-2.40 (m, 1H), 1.66-1.79 (m, 1H), 1.57-1.66 (m, 1.5H), 1.32-1.49 (m, 3H), 1.18-1.31 (m, 1.5H), 1.02-1.19 (m, 11H), 0.98 (t, J=7.32 Hz, 3H)

LC-MS: purity 100% (UV), t_{R} 5.17 min m/z [M+H]⁺782.0 (MET/CR/1416).

### Stages 4/5: 287

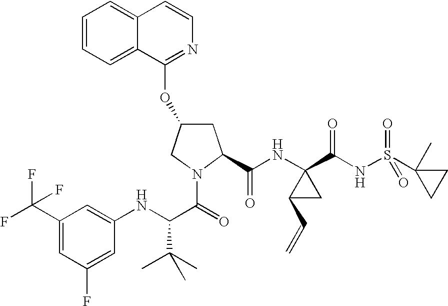

70 mg (23%) of an off white solid

¹H NMR (500 MHz, CHLOROFORM-d) δ ppm 7.96 (d, J=5.95 Hz, 1H) 7.82 (d, J=8.39 Hz, 1H) 7.73 (d, J=8.24 Hz, 1H) 7.62-7.67 (m, 1H) 7.37-7.44 (m, 1H) 7.09 (br. s., 1H) 6.55 (s, 1H) 6.40 (d, 1H) 6.27 (d, 1H) 5.89 (br. s., 1H) 5.67-5.77 (m, 1H) 5.24-5.33 (m, 1H) 5.17 (d, J=10.53 Hz, 1H) 4.78 (d, J=10.38 Hz, 1H) 4.60 (t, J=8.39 Hz, 1H) 4.07-4.19 (m, 2H) 3.90 (d, J=10.22 Hz, 1H) 2.55-2.63 (m, 2H) 2.05-2.14 (m, 1H) 1.94-2.01 (m, 1H) 1.70-1.78 (m, 1H) 1.66 (d, J=6.56 Hz, 2H) 1.52 (s, 3H) 1.43 (dd, J=9.46, 5.95 Hz, 1H) 1.26 (br. s., 2H) 1.07-1.14 (m, 7H) 0.82-0.93 (m, 3H)

LC-MS: purity 98% (UV), t_{R} 5.44 min m/z [M+H]⁺760.4 (MET/CR/1416).

### Stages 4/5: 288

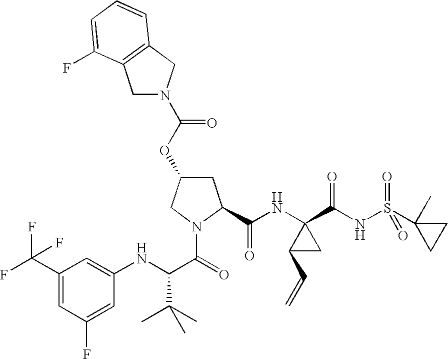

125 mg (40%) of an off white solid

¹H NMR (500 MHz, CHLOROFORM-d) δ ppm 9.86 (br. s., 1H) 7.17-7.33 (m, 1H) 6.82-7.09 (m, 2H) 6.64 (d, J=13.28 Hz, 1H) 6.28-6.45 (m, 2H) 5.64-5.75 (m, 1H) 5.43 (br. s., 1H) 5.22-5.33 (m, 1H) 5.17 (d, J=10.68 Hz, 1H) 4.66-4.91 (m, 3H) 4.44-4.57 (m, 2H) 4.18-4.30 (m, 1H) 3.82-4.04 (m, 3H) 2.31-2.54 (m, 2H) 2.04-2.19 (m, 1H) 1.95 (ddd, J=8.13, 5.84, 2.82 Hz, 1H) 1.57-1.68 (m, 4H) 1.46-1.54 (m, 4H) 1.40 (ddd, J=9.12, 6.22, 2.37 Hz, 1H) 1.02-1.14 (m, 7H) 0.77-0.91 (m, 2H)

LC-MS: purity 95% (UV), t_{R} 5.25 min m/z [M+H]⁺796.4 (MET/CR/1416).

Stages 4/5: 289

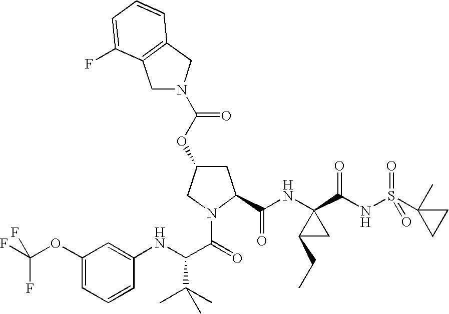

120 mg (34%) of a beige solid

¹H NMR (500 MHz, CHLOROFORM-d) δ ppm 9.76 (br. s., 1H) 7.23-7.37 (m, 1H) 7.18 (d, J=9.77 Hz, 1H) 6.82-7.09 (m, 3H) 6.51 (d, J=8.24 Hz, 1H) 6.45 (s, 1H) 6.07-6.28 (m, 1H) 5.39 (br. s., 1H) 4.71 (d, J=6.10 Hz, 2H) 4.56 (dd, J=10.38, 6.10 Hz, 1H) 4.34-4.53 (m, 2H) 4.04-4.19 (m, 1H) 3.80-4.00 (m, 3H) 2.42-2.63 (m, 1H) 2.31 (dd, J=14.34, 7.63 Hz, 1H) 1.64-1.80 (m, 2H) 1.42-1.64 (m, 6H) 1.30-1.42 (m, 1H) 1.17 (dt, J=9.46, 5.95 Hz, 1H) 0.93-1.13 (m, 12H) 0.73-0.93 (m, 2H)

LC-MS: purity 98% (UV), t_{R} 5.25 min m/z [M+H]⁺796.4 (MET/CR/1416).

### Stages 4/5: 290

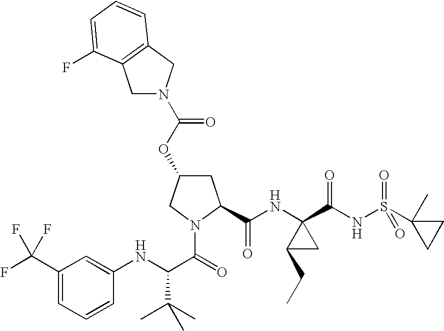

165 mg (45%) of an off white solid

¹H NMR (500 MHz, CHLOROFORM-d) δ ppm 9.67-9.85 (m, 1H) 7.28-7.35 (m, 1H) 6.91-7.17 (m, 3H) 6.79-6.90 (m, 1H) 6.74 (d, J=8.24 Hz, 1H) 6.61 (dd, J=16.56, 7.55 Hz, 1H) 5.39 (br. s., 1H) 4.70 (d, J=7.02 Hz, 2H) 4.61 (dd, J=10.22, 7.02 Hz, 1H) 4.47-4.54 (m, 1H) 4.36-4.46 (m, 1H) 4.10 (t, J=15.49 Hz, 1H) 3.80-4.00 (m, 3H) 2.46-2.61 (m, 1H) 2.30 (dd, J=14.11, 7.71 Hz, 1H) 1.71-1.80 (m, 1H) 1.61-1.70 (m, 3H) 1.55 (s, 5H) 1.32-1.44 (m, 1H) 1.24-1.32 (m, 1H) 1.14-1.23 (m, 1H) 1.09 (s, 8H) 1.01 (t, J=7.40 Hz, 3H) 0.81-0.96 (m, 2H)

LC-MS: purity 100% (UV), t_{R} 5.22 min m/z [M+H]⁺780.4 (MET/CR/1416).

### Stages 4/5: 291

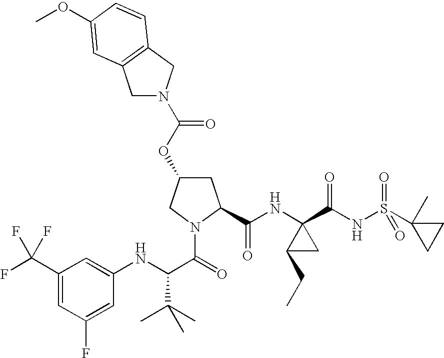

150 mg (56%) of an off white solid

¹H NMR (500 MHz, CHLOROFORM-d) δ ppm 9.77 (d, J=0.92 Hz, 1H) 6.94-7.19 (m, 2H) 6.54-6.92 (m, 3H) 6.23-6.52 (m, 2H) 5.41 (br. s., 1H) 4.80 (d, J=9.92 Hz, 1H) 4.57-4.72 (m, 2H) 4.45-4.56 (m, 1H) 4.32-4.44 (m, 1H) 4.13 (dd, J=14.50, 7.32 Hz, 1H) 3.87-4.01 (m, 3H) 3.82 (d, J=8.54 Hz, 3H) 2.51 (ddd, J=13.58, 9.16, 4.12 Hz, 1H) 2.35 (dd, J=14.27, 7.40 Hz, 1H) 1.71-1.77 (m, 1H) 1.62-1.70 (m, 2H) 1.57 (d, J=7.32 Hz, 1H) 1.47-1.55 (m, 4H) 1.37 (quin, J=8.13 Hz, 1H) 1.15-1.30 (m, 1H) 1.05-1.14 (m, 9H) 1.00 (t, J=7.32 Hz, 3H) 0.80-0.94 (m, 2H)

LC-MS: purity 100% (UV), t_{R} 5.26 min m/z [M+H]⁺810.25 (MET/CR/1416).

### Stages 4/5: 292

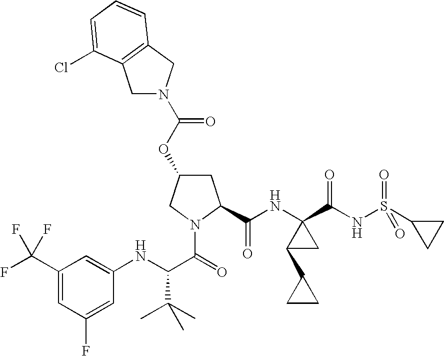

140 mg (29%) of an off white solid

¹H NMR (500 MHz, CHLOROFORM-d) δ ppm 10.02-10.14 (m, 1H) 7.21-7.29 (m, 2H) 6.95-7.19 (m, 2H) 6.62 (d, J=12.82 Hz, 1H) 6.32-6.42 (m, 2H) 5.43 (br. s., 1H) 4.79 (d, J=10.07 Hz, 1H) 4.76 (s, 1H) 4.71 (br. s., 1H) 4.51 (d, J=14.95 Hz, 1H) 4.41-4.47 (m, 1H) 4.27 (dd, J=14.65, 6.26 Hz, 1H) 3.91-4.03 (m, 2H) 3.88 (dd, J=10.22, 1.68 Hz, 1H) 2.92-2.99 (m, 1H) 2.46 (ddd, J=13.85, 9.27, 4.65 Hz, 1H) 2.35 (dd, J=14.11, 7.40 Hz, 1H) 1.79-1.86 (m, 1H) 1.33-1.46 (m, 3H) 1.05-1.15 (m, 10H) 0.97-1.05 (m, 3H) 0.57 (dd, J=13.35, 8.62 Hz, 2H) 0.29 (d, J=4.27 Hz, 2H)

LC-MS: purity 100% (UV), t_{R} 5.33 min m/z [M+H]⁺812.4 (MET/CR/1416).

### Stages 4/5: 293

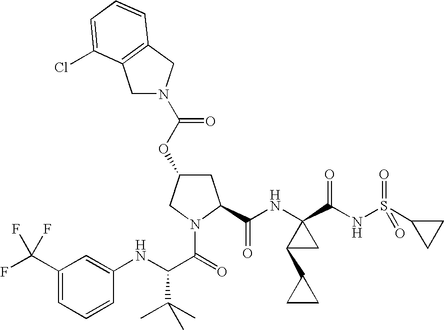

47 mg (10%) of an off white solid

¹H NMR (500 MHz, MeOD) δ 6.99-7.38 (m, 5H), 6.92 (dd, J=8.32, 15.95 Hz, 1H), 6.43-6.60 (m, 1H), 5.38 (d, J=12.21 Hz, 1H), 4.69-4.80 (m, 1H), 4.56-4.69 (m, 1H), 4.47 (d, J=14.80 Hz, 1H), 4.37 (ddd, J=7.10, 10.38, 13.50 Hz, 1H), 4.12-4.20 (m, 3H), 3.89-3.99 (m, 1H), 2.96-3.03 (m, 1H), 2.34 (dd, J=6.94, 13.81 Hz, 1H), 2.07-2.20 (m, 1H), 1.77 (dd, J=5.72, 7.71 Hz, 1H), 1.29 (br. s., 2H), 1.05-1.19 (m, 13H), 0.78-0.91 (m, 1H), 0.48-0.65 (m, 2H), 0.27-0.38 (m, 2H)

LC-MS: purity 100% (UV), t_{R} 5.17 min m/z [M+H]⁺794.30 (MET/CR/1416).

### Stages 4/5: 294

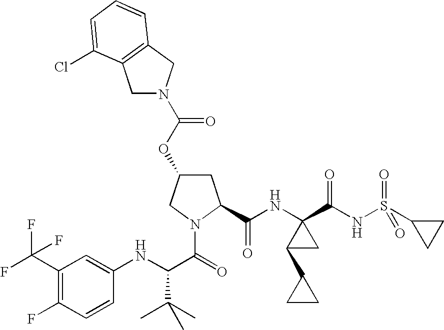

32 mg (7%) of an off white solid

¹H NMR (500 MHz, MeOD) δ ppm 0.32 (d, J=4.12 Hz, 2H) 0.59 (s, 2H) 0.81-0.90 (m, 1H) 1.13 (s, 12H) 1.30 (br. s., 2H) 1.73-1.80 (m, 1H) 2.16 (s, 1H) 2.29-2.39 (m, 1H) 3.00 (s, 1H) 3.90-4.00 (m, 1H) 4.06-4.15 (m, 3H) 4.20-4.42 (m, 2H) 4.46-4.56 (m, 1H) 4.58-4.78 (m, 2H) 5.33-5.42 (m, 1H) 6.78-7.39 (m, 6H)

LC-MS: purity 91% (UV), t_{R} 5.14 min m/z [M+H]⁺812.25 (MET/CR/1416).

### Stages 4/5: 295

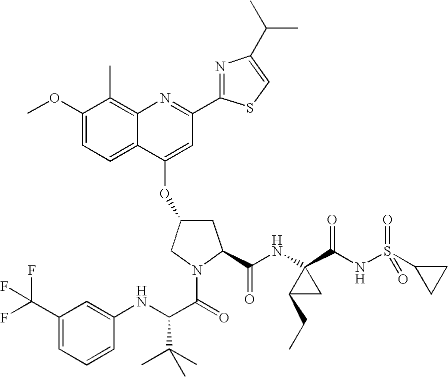

112 mg (62%) of an off white solid

¹H NMR (500 MHz, CHLOROFORM-d) δ ppm 10.11 (s, 1H) 7.50 (s, 1H) 7.43-7.48 (m, 1H) 7.06 (s, 1H) 6.99 (d, J=9.16 Hz, 1H) 6.69-6.90 (m, 4H) 6.57 (d, J=7.32 Hz, 1H) 5.51 (d, J=2.14 Hz, 1H) 4.61 (d, J=10.53 Hz, 1H) 4.46 (t, J=8.32 Hz, 1H) 4.22 (d, J=11.75 Hz, 1H) 4.08-4.15 (m, 1H) 3.99 (d, J=10.53 Hz, 1H) 3.97 (s, 3H) 3.07-3.36 (m, 1H) 2.78-3.08 (m, 1H) 2.70 (s, 3H) 2.60 (d, J=8.39 Hz, 2H) 1.71 (dd, J=8.24, 5.49 Hz, 1H) 1.32-1.44 (m, 9H) 1.19-1.30 (m, 2H) 1.14 (s, 9H) 1.06 (t, J=8.77 Hz, 2H) 0.96 (t, J=7.32 Hz, 3H)

LC-MS: purity 100% (UV), t_{R} 5.29 min m/z [M+H]⁺899.40 (MET/CR/1426).

### Stages 4/5: 296

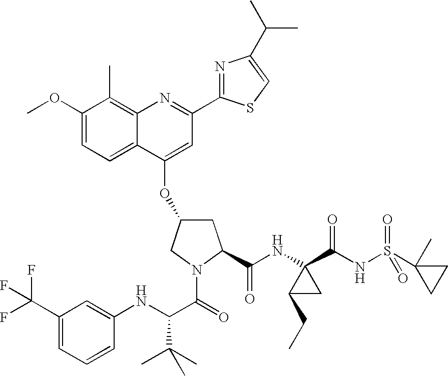

251 mg (35%) of an off white solid

¹H NMR (500 MHz, DMSO-d6) δ ppm 10.30 (br. s., 1H) 8.69 (s, 1H) 7.58 (d, J=9.31 Hz, 1H) 7.54 (s, 1H) 7.48 (s, 1H) 7.10-7.28 (m, 2H) 6.64-6.82 (m, 3H) 5.80 (d, J=9.92 Hz, 1H) 5.66 (br. s., 1H) 4.32-4.52 (m, 2H) 4.23 (d, J=9.92 Hz, 1H) 3.87-4.09 (m, 4H) 3.10-3.23 (m, 1H) 2.59 (s, 3H) 2.19 (t, J=10.30 Hz, 1H) 1.39-1.51 (m, 6H) 1.30-1.38 (m, 9H) 1.24 (br. s., 1H) 1.07 (s, 9H) 0.81-0.99 (m, 6H)

LC-MS: purity 100% (UV), t_{R} 5.33 min m/z [M+H]⁺913.33 (MET/CR/1426).

### Stages 4/5: 297

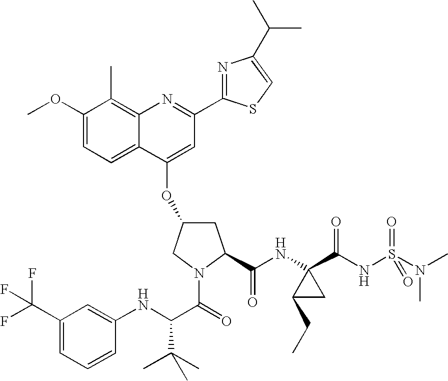

65 mg (45%) of an off white solid

¹H NMR (500 MHz, CHLOROFORM-d) δ ppm 9.81 (br. s., 1H) 7.50 (br. s., 1H) 7.43 (d, J=9.16 Hz, 1H) 7.06 (s, 1H) 6.97 (d, J=9.31 Hz, 1H) 6.91 (s, 1H) 6.85 (s, 1H) 6.74-6.84 (m, 2H) 6.57 (d, J=7.78 Hz, 1H) 5.50 (br. s., 1H) 4.61 (d, J=10.53 Hz, 1H) 4.50 (t, J=8.24 Hz, 1H) 4.22 (d, J=11.75 Hz, 1H) 4.05-4.13 (m, 1H) 4.00 (d, J=10.38 Hz, 1H) 3.96 (s, 3H) 3.21 (ddd, J=13.35, 6.56, 6.33 Hz, 1H) 2.94 (s, 6H) 2.69 (s, 3H) 2.55-2.65 (m, 2H) 1.50-1.59 (m, 2H) 1.40 (dd, J=6.87, 1.83 Hz, 6H) 1.34 (dt, J=16.14, 8.03 Hz, 1H) 1.27 (t, J=7.17 Hz, 1H) 1.19 (dd, J=9.54, 5.57 Hz, 1H) 1.13 (s, 9H) 0.98 (t, J=7.32 Hz, 3H)

LC-MS: purity 100% (UV), t_{R} 5.29 min m/z [M+H]⁺902.42 (MET/CR/1426).

### Stages 4/5: 298

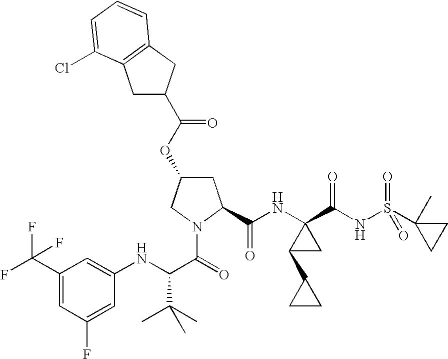

73 mg (24%) of a beige solid

¹H NMR (500 MHz, MeOD) δ ppm 7.01-7.37 (m, 3H) 6.88 (d, J=9.61 Hz, 1H) 6.64-6.72 (m, 1H) 6.15-6.31 (m, 1H) 5.41 (d, J=16.94 Hz, 1H) 4.70-4.77 (m, 1H) 4.60-4.70 (m, 1H) 4.47-4.56 (m, 1H) 4.37-4.47 (m, 1H) 4.13-4.31 (m, 3H) 3.93 (dt, J=12.36, 3.13 Hz, 1H) 2.81 (s, 1H) 2.37 (dt, J=13.77, 6.77 Hz, 1H) 2.10-2.20 (m, 1H) 1.73 (dd, J=8.09, 5.34 Hz, 1H) 1.51-1.63 (m, 5H) 1.17-1.23 (m, 1H) 1.04-1.17 (m, 11H) 0.81 (d, J=7.48 Hz, 1H) 0.48-0.64 (m, 2H) 0.27-0.37 (m, 2H)

LC-MS: purity 100% (UV), t_{R} 5.45 min m/z [M+H]⁺826.35 (MET/CR/1416).

### Stages 4/5: 299

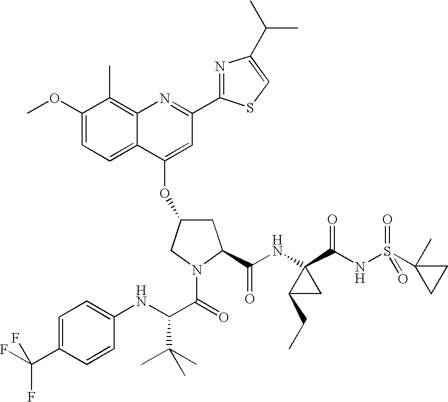

151 mg (37%) of a beige solid

¹H NMR (500 MHz, CHLOROFORM-d) δ ppm 9.78 (s, 1H) 7.62 (d, J=9.16 Hz, 1H) 7.53 (s, 1H) 7.11 (d, J=8.54 Hz, 2H) 7.03-7.08 (m, 2H) 6.91 (s, 1H) 6.52 (d, J=8.54 Hz, 2H) 5.50-5.56 (m, 1H) 4.69-4.75 (m, 1H) 4.52 (s, 1H) 4.30-4.38 (m, 1H) 4.12 (s, 1H) 4.02 (d, J=10.38 Hz, 1H) 3.96 (s, 3H) 3.16-3.26 (m, 1H) 2.70 (s, 3H) 2.61-2.67 (m, 2H) 1.63 (br. s., 3H) 1.62 (s, 5H) 1.48-1.59 (m, 0H) 1.41 (d, J=7.02 Hz, 6H) 1.32-1.39 (m, 1H) 1.17-1.22 (m, 1H) 1.14 (s, 9H) 0.97 (t, J=7.32 Hz, 3H) 0.81-0.93 (m, 2H)

LC-MS: purity 100% (UV), t_{R} 5.27 min m/z [M+H]⁺913.42 (MET/CR/1426).

### Stages 4/5: 300

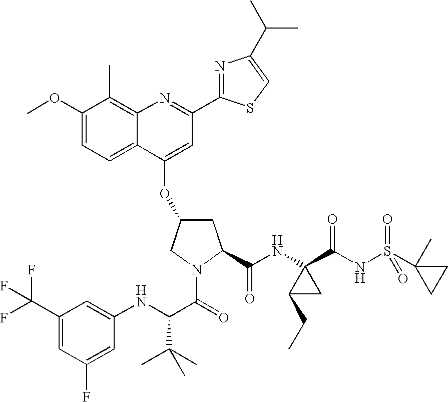

201 mg (49%) of a beige solid

¹H NMR (500 MHz, CHLOROFORM-d) δ ppm 9.58-9.93 (m, 1H) 7.50 (s, 1H) 7.47 (d, J=9.00 Hz, 1H) 7.05 (s, 1H) 6.95-7.02 (m, 2H) 6.64 (s, 1H) 6.50-6.57 (m, 1H) 6.31-6.40 (m, 1H) 5.45-5.55 (m, 1H) 4.79-4.88 (m, 1H) 4.50-4.60 (m, 1H) 4.19 (s, 1H) 4.06-4.12 (m, 1H) 3.90-4.00 (m, 4H) 3.15-3.26 (m, 1H) 2.68 (s, 3H) 2.65 (br. s., 2H) 1.66-1.75 (m, 2H) 1.64 (br. s., 5H) 1.53 (s, 1H) 1.40 (dd, J=6.87, 1.83 Hz, 6H) 1.33-1.37 (m, 1H) 1.16-1.22 (m, 1H) 1.11 (s, 9H) 0.98 (t, J=7.40 Hz, 3H) 0.80-0.93 (m, 2H)

LC-MS: purity 100% (UV), t_{R} 5.36 min m/z [M+H]⁺931.40 (MET/CR/1426).

### Stages 4/5: 301

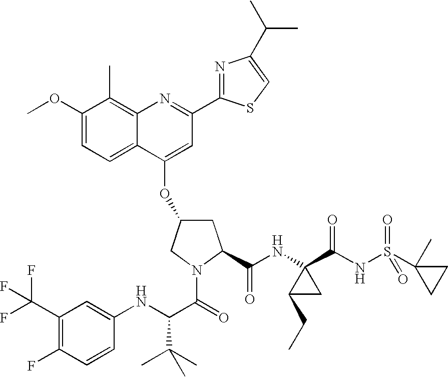

159 mg (39%) of a beige solid

¹H NMR (500 MHz, CHLOROFORM-d) δ ppm 9.72-9.84 (m, 1H) 7.49 (s, 1H) 7.40 (d, J=9.16 Hz, 1H) 7.06 (s, 1H) 7.03 (d, J=9.31 Hz, 1H) 6.96 (s, 1H) 6.81-6.88 (m, 1H) 6.45 (s, 2H) 5.45-5.53 (m, 1H) 4.43-4.57 (m, 2H) 4.16-4.24 (m, 1H) 4.02-4.09 (m, 1H) 3.98 (s, 3H) 3.88 (d, J=10.68 Hz, 1H) 3.15-3.26 (m, 1H) 2.70 (s, 3H) 2.56-2.67 (m, 2H) 1.64-1.76 (m, 3H) 1.60-1.64 (m, 4H) 1.54 (s, 1H) 1.40 (dd, J=6.87, 1.98 Hz, 6H) 1.32-1.38 (m, 1H) 1.17-1.23 (m, 1H) 1.12 (s, 9H) 0.99 (t, J=7.40 Hz, 3H) 0.82-0.94 (m, 2H))

LC-MS: purity 100% (UV), t_{R} 5.31 min m/z [M+H]⁺931.40 (MET/CR/1426).

### Stages 4/5: 302

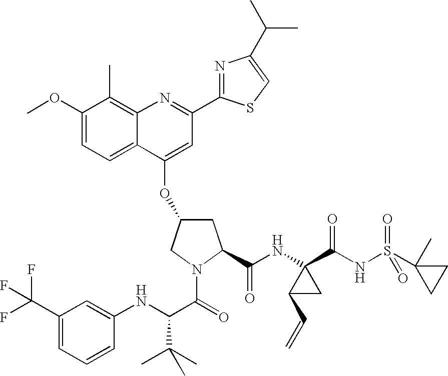

86 mg (31%) of a beige solid

¹H NMR (500 MHz, CHLOROFORM-d) δ 9.80 (br. s., 1H), 7.36-7.65 (m, 3H), 7.01-7.10 (m, 1H), 6.96 (d, J=9.16 Hz, 1H), 6.86 (br. s., 1H), 6.67-6.82 (m, 2H), 6.56 (d, J=7.17 Hz, 1H), 5.55-5.70 (m, 1H), 5.44 (br. s., 1H), 5.22 (d, J=17.09 Hz, 1H), 5.11 (d, J=10.38 Hz, 1H), 4.72 (d, J=9.92 Hz, 1H), 4.57 (t, J=8.16 Hz, 1H), 4.21 (d, J=11.75 Hz, 1H), 4.04-4.14 (m, 1H), 3.99 (d, J=10.07 Hz, 1H), 3.94 (s, 3H), 3.21 (spt, J=6.84 Hz, 1H), 2.59-2.76 (m, 4H), 2.47-2.59 (m, 1H), 2.10 (q, J=8.60 Hz, 1H), 1.81 (t, J=6.79 Hz, 1H), 1.53 (br. s., 1H), 1.36-1.49 (m, 9H), 1.33 (dd, J=5.95, 9.16 Hz, 1H), 1.18-1.30 (m, 1H), 0.98-1.16 (m, 9H), 0.64-0.86 (m, 2H)

LC-MS: purity 100% (UV), t_{R} 5.31 min m/z [M+H]⁺911.34 (MET/CR/1426).

### Stages 4/5: 303

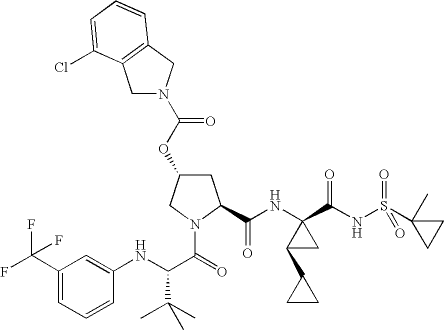

32 mg (11%) of a beige solid

¹H NMR (500 MHz, MeOD) δ 7.21-7.37 (m, 2H), 6.83-7.11 (m, 4H), 6.41-6.59 (m, 1H), 5.37 (d, J=12.97 Hz, 1H), 4.67-4.78 (m, 1H), 4.57-4.67 (m, 1H), 4.45 (d, J=14.80 Hz, 1H), 4.33-4.42 (m, 1H), 4.12-4.24 (m, 2H), 3.88-3.98 (m, 1H), 2.30-2.39 (m, 1H), 2.13 (qd, J=4.50, 9.74 Hz, 1H), 1.73 (t, J=6.71 Hz, 1H), 1.51-1.65 (m, 5H), 1.05-1.18 (m, 12H), 0.85-0.96 (m, 2H), 0.74-0.85 (m, 1H), 0.45-0.64 (m, 2H), 0.27-0.36 (m, 2H)

LC-MS: purity 94% (UV), t_{R} 5.38 min m/z [M+H]⁺808.35 (MET/CR/1416).

### Stages 4/5: 304

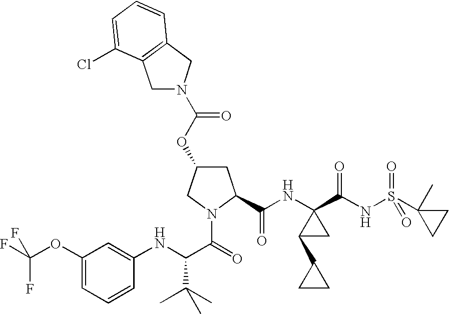

34 mg (11%) of a beige solid

¹H NMR (500 MHz, MeOD) δ 6.77-7.47 (m, 4H), 6.55-6.79 (m, 2H), 5.89-6.34 (m, 1H), 5.37 (d, J=14.34 Hz, 1H), 4.68-4.78 (m, 1H), 4.58-4.68 (m, 1H), 4.47 (d, J=14.65 Hz, 1H), 4.40 (ddd, J=7.17, 10.26, 12.78 Hz, 1H), 4.05-4.15 (m, 3H), 3.86-3.95 (m, 1H), 2.30-2.40 (m, 1H), 2.07-2.18 (m, 1H), 1.73 (dd, J=6.03, 7.25 Hz, 1H), 1.50-1.63 (m, 5H), 1.26-1.33 (m, 1H), 1.18-1.21 (m, 1H), 1.15 (br. s., 11H), 0.76-0.85 (m, 1H), 0.45-0.64 (m, 2H), 0.26-0.37 (m, 2H)

LC-MS: purity 100% (UV), t_{R} 5.50 min m/z [M+H]⁺824.20 (MET/CR/1416).

### Stages 4/5: 305

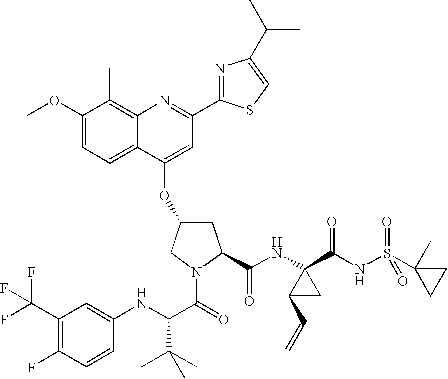

117 mg (41%) of a yellow solid

¹H NMR (500 MHz, CHLOROFORM-d) δ 9.87 (s, 1H), 7.50 (s, 1H), 7.41 (d, J=9.16 Hz, 1H), 7.01-7.10 (m, 3H), 6.84 (dd, J=2.67, 5.42 Hz, 1H), 6.41-6.54 (m, 2H), 5.65-5.76 (m, 1H), 5.50 (d, J=2.29 Hz, 1H), 5.26 (d, J=17.09 Hz, 1H), 5.16 (d, J=10.38 Hz, 1H), 4.52 (t, J=8.39 Hz, 1H), 4.19 (d, J=11.75 Hz, 1H), 4.06 (dd, J=3.13, 11.67 Hz, 1H), 3.95-4.01 (m, 3H), 3.87 (s, 1H), 3.21 (spt, J=6.82 Hz, 1H), 2.71 (s, 3H), 2.64 (d, J=8.39 Hz, 2H), 2.03-2.11 (m, 1H), 1.96 (dd, J=6.03, 8.01 Hz, 1H), 1.69-1.76 (m, 1H), 1.59-1.69 (m, 3H), 1.51 (s, 3H), 1.36-1.46 (m, 8H), 1.12 (s, 9H)

LC-MS: purity 100% (UV), t_{R} 5.25 min m/z [M+H]⁺928.00 (MET/CR/1426).

### Stages 4/5: 306

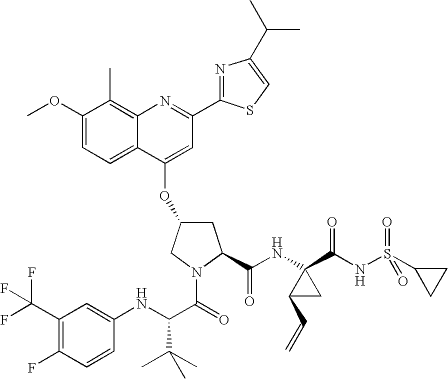

98 mg (34%) of a yellow solid

¹H NMR (500 MHz, CHLOROFORM-d) δ 10.21 (s, 1H), 7.50 (s, 1H), 7.42 (d, J=9.16 Hz, 1H), 7.02-7.09 (m, 2H), 6.87-6.93 (m, 1H), 6.85 (dd, J=2.59, 5.49 Hz, 1H), 6.42-6.56 (m, 2H), 5.71-5.83 (m, 1H), 5.51 (br. s., 1H), 4.47 (d, J=10.38 Hz, 1H), 4.43 (dd, J=7.25, 9.69 Hz, 1H), 4.14-4.22 (m, 1H), 4.06-4.13 (m, 1H), 3.98 (s, 3H), 3.86 (d, J=10.07 Hz, 1H), 3.21 (spt, J=6.92 Hz, 1H), 2.85-2.93 (m, 1H), 2.71 (s, 3H), 2.60-2.68 (m, 1H), 2.49-2.58 (m, 1H), 2.06 (q, J=8.54 Hz, 1H), 1.98 (d, J=6.41 Hz, 1H), 1.68 (br. s., 2H), 1.51 (dd, J=5.95, 9.31 Hz, 1H), 1.40 (dd, J=1.98, 6.87 Hz, 6H), 1.32-1.38 (m, 2H), 1.13 (s, 9H), 0.97-1.07 (m, 2H)

LC-MS: purity 100% (UV), t_{R} 5.21 min m/z [M+H]⁺915.00 (MET/CR/1426).

### Stages 4/5: 307

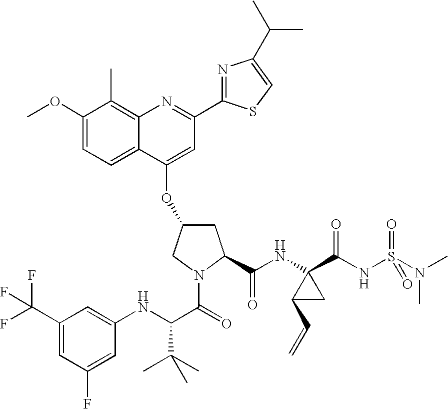

140 mg (34%) of a beige solid

¹H NMR (500 MHz, CHLOROFORM-d) d 9.82 (s, 1H), 7.50 (s, 1H), 7.48 (d, J=9.14 Hz, 1H), 7.05 (s, 1H), 6.99 (d, J=9.14 Hz, 1H), 6.87 (s, 1H), 6.64 (s, 1H), 6.54 (dd, 1H), 6.36 (d, 1H), 5.50 (br. s., 1H), 4.84 (dd, 1H), 4.50 (t, 1H), 4.19 (d, 1H), 4.08-4.15 (m, 1H), 3.96 (s, 4H), 3.20 (spt, 1H), 2.94 (s, 6H), 2.68 (s, 3H), 2.59-2.64 (m, 2H), 1.64-1.67 (m, 1H), 1.51-1.59 (m, 2H), 1.40 (dd, J=1.66, 6.86 Hz, 6H), 1.29-1.37 (m, 1H), 1.17-1.23 (m, 1H), 1.12 (s, 9H), 0.97 (t, J=7.33 Hz, 3H)

LC-MS: purity 100% (UV), t_{R} 5.30 min m/z [M+H]⁺919.00 (MET/CR/1426).

### Stages 4/5: 308

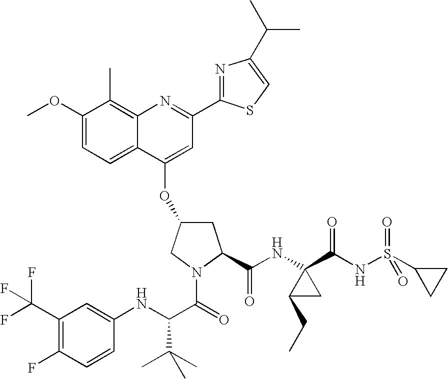

66 mg (27%) of a yellow solid

¹H NMR (500 MHz, CHLOROFORM-d) δ 10.13 (s, 1H), 7.50 (s, 1H), 7.42 (d, J=9.16 Hz, 1H), 7.02-7.09 (m, 2H), 6.84 (dd, J=2.44, 5.34 Hz, 1H), 6.78 (s, 1H), 6.42-6.53 (m, 2H), 5.51 (br. s., 1H), 4.40-4.50 (m, 1H), 4.15-4.22 (m, 1H), 4.05-4.14 (m, 1H), 3.98 (s, 3H), 3.87 (s, 1H), 3.21 (dt, J=6.85, 13.62 Hz, 1H), 2.91-2.97 (m, 1H), 2.71 (s, 3H), 2.52-2.67 (m, 2H), 1.71 (dd, J=5.34, 7.93 Hz, 1H), 1.51-1.68 (m, 4H), 1.40 (dd, J=1.83, 6.87 Hz, 8H), 1.28 (dd, J=5.49, 9.46 Hz, 1H), 1.13 (s, 9H), 1.06 (t, J=8.62 Hz, 2H), 0.95 (t, J=7.32 Hz, 3H)

LC-MS: purity 100% (UV), t_{R} 5.28 min m/z [M+H]⁺916.00 (MET/CR/1426).

### Stages 4/5: 309

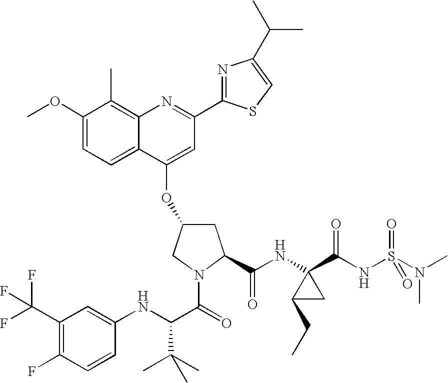

230 mg (56%) of a yellow solid

¹H NMR (500 MHz, CHLOROFORM-d) δ ppm 9.66-10.01 (m, 1H) 7.50 (s, 1H) 7.40 (d, J=9.14 Hz, 1H) 7.06 (s, 1H) 7.04 (d, J=9.30 Hz, 1H) 6.84 (s, 2H) 6.41-6.53 (m, 2H) 5.50 (br. s., 1H) 4.48 (s, 2H) 4.18 (s, 1H) 4.04-4.10 (m, 1H) 3.98 (s, 3H) 3.88 (d, J=10.72 Hz, 1H) 3.21 (spt, 1H) 2.95 (s, 6H) 2.71 (s, 3H) 2.57-2.65 (m, 2H) 1.63-1.68 (m, 1H) 1.56 (quin, 2H) 1.40 (dd, J=6.86, 1.81 Hz, 6H) 1.30-1.38 (m, 1H) 1.19-1.24 (m, 1H) 1.13 (s, 9H) 0.97 (t, J=7.41 Hz, 3H)

LC-MS: purity 100% (UV), t_{R} 5.24 min m/z [M+H]⁺919.00 (MET/CR/1426).

### Preparation of Non-Macrocycles Analogues Following Route 2:

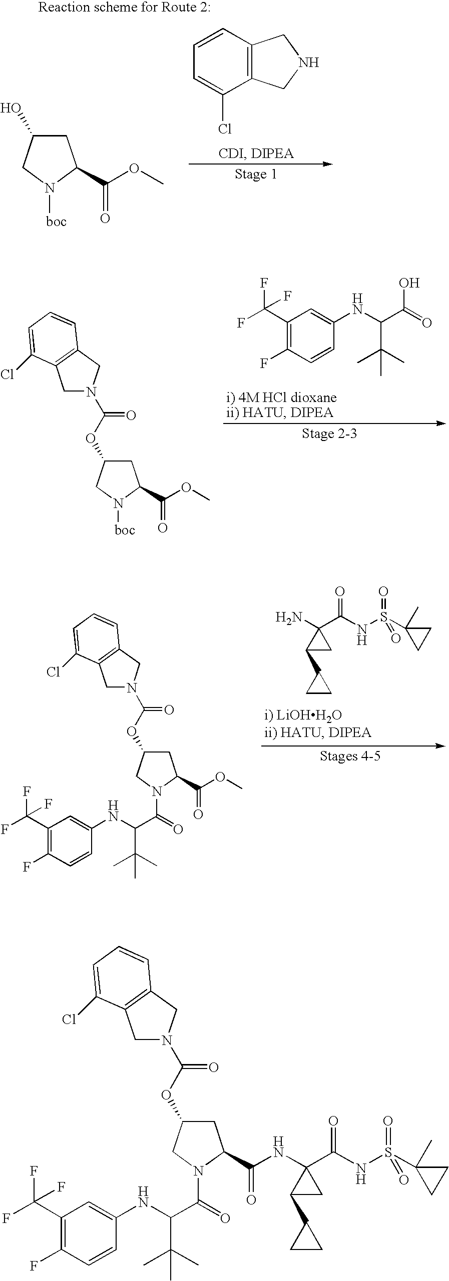

### Synthesis of 310

### Stage 1: 311

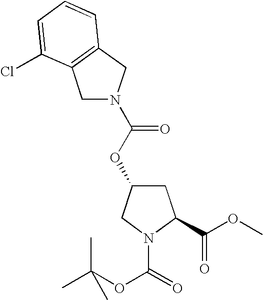

9.77 g (94%) of the desired product

¹H NMR (500 MHz, CHLOROFORM-d) δ 7.20-7.27 (m, 2H), 7.08-7.20 (m, 1H), 5.27-5.38 (m, 1H), 4.78 (br. s., 1H), 4.74 (br. s., 1H), 4.73 (s, 1H), 4.67 (br. s., 1H), 4.33-4.55 (m, 1H), 3.55-3.86 (m, 5H), 2.40-2.55 (m, 1H), 2.25 (ddd, J=5.11, 8.43, 13.77 Hz, 1H), 1.45 (dd, J=3.28, 15.64 Hz, 9H)

LC-MS: purity 87% (UV), t_{R} 2.24 min m/z [M+H]⁺447.15 (MET/CR/1278).

### Stages 2-3: 312

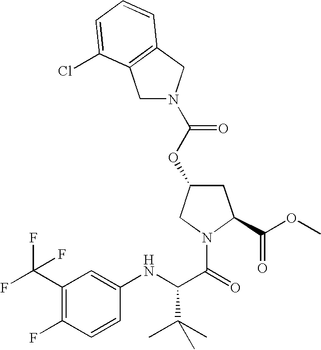

502 mg (71%) of the desired product

¹H NMR (250 MHz, CHLOROFORM-d) δ 7.25-7.35 (m, 3H), 6.99-7.25 (m, 1H), 6.66-6.91 (m, 3H), 5.40 (br. s., 1H), 4.71-4.81 (m, 2H), 4.59-4.71 (m, 1H), 4.43-4.58 (m, 2H), 4.21-4.38 (m, 1H), 3.82-4.07 (m, 3H), 2.45-2.59 (m, 1H), 2.14-2.31 (m, J=4.89, 4.89, 9.31, 13.95 Hz, 1H), 1.07-1.21 (m, 9H), 0.84-0.98 (m, 2H)

LC-MS: purity 92% (UV), t_{R} 2.60 min m/z [M+H]⁺600.30 (MET/CR/1278).

### Stage 4: 313

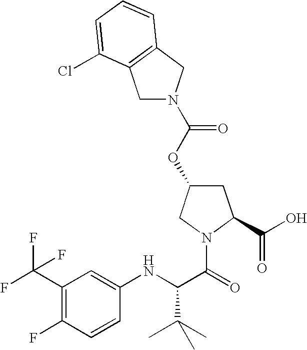

482 mg (98%) of the desired product

¹H NMR (250 MHz, MeOD) δ 7.18-7.34 (m, 3H), 6.74-7.11 (m, 3H), 5.29-5.39 (m, 1H), 4.59-4.73 (m, 2H), 4.45-4.59 (m, 2H), 4.17-4.28 (m, 1H), 4.06-4.17 (m, 2H), 3.87 (ddd, J=3.43, 5.52, 12.37 Hz, 1H), 3.52-3.76 (m, OH), 2.41-2.61 (m, 1H), 2.10-2.31 (m, 1H), 1.01-1.15 (m, 9H)

LC-MS: purity 90% (UV), t_{R} 2.341 min m/z [M+H]⁺586.15 (MET/CR/1278).

### Stage 5: 310

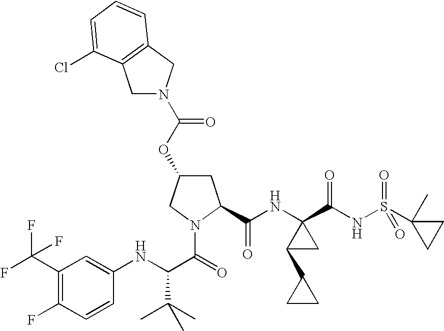

50 mg (15%) of an off white solid

¹H NMR (500 MHz, CHLOROFORM-d) δ ppm 9.83 (br. s., 1H) 7.21-7.33 (m, 1H) 6.98-7.22 (m, 2H) 6.74-6.89 (m, 2H) 6.64-6.74 (m, 1H) 5.33-5.44 (m, 1H) 4.64-4.88 (m, 2H) 4.39-4.57 (m, 3H) 4.17-4.32 (m, 1H) 3.91 (dd, J=10.38, 2.14 Hz, 2H) 3.83 (dd, J=10.53, 5.80 Hz, 1H) 2.47-2.59 (m, 1H) 2.28-2.40 (m, 1H) 1.72-1.87 (m, 2H) 1.62-1.70 (m, 1H) 1.56 (s, 3H) 1.23-1.32 (m, 1H) 1.09-1.23 (m, 2H) 1.04-1.10 (m, 9H) 0.83-0.93 (m, 2H) 0.73-0.83 (m, 1H) 0.48-0.66 (m, 2H) 0.25-0.39 (m, 2H)

LC-MS: purity 100% (UV), t_{R} 5.33 min m/z [M+H]⁺826.30 (MET/CR/1416).

### Preparation of Non-Macrocycles Analogues Following Route 3:

Preparation of 5-(1-morpholinylethylamino)-isoindoline

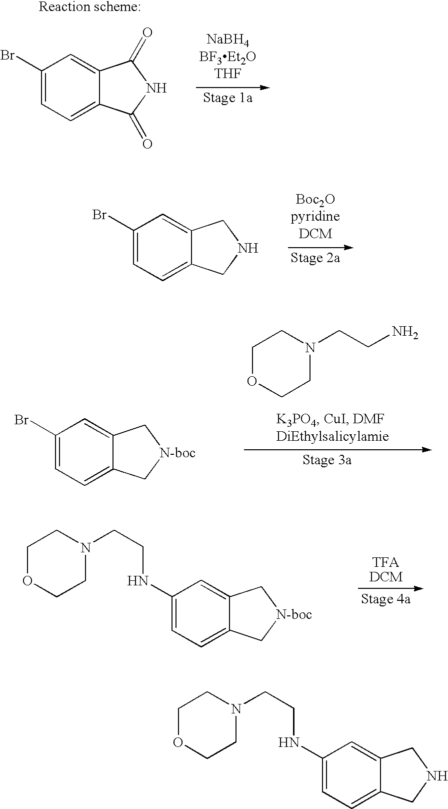

### Stage 1a: 5-Bromo-isoindoline HCl salt

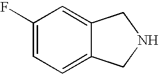

5-Bromo-phtalamide (5.35 g, 23.7 mmol, 1 eq.) and tetrahydrofuran (230 mL) were charged into a 1 L flask. Sodium borohydride (9.30 g, 244 mmol, 10 eq.) was added portionwise and the reaction mixture cooled to −40° C. Borontrifluoride etherate (39.4 g, 278 mmol, 1.2 eq.) was added dropwise over 10 minutes while the temperature increase to −25° C. The reaction mixture was left to warm up to ambient temperature and then the white suspension was heated at 70° C. for 15 hours. The reaction mixture was cooled to 0° C. and water (50 mL) added dropwise (large amount of frothing noticed). Ethylacetate (400 mL) was added. The organic layer was collected, washed with brine (4×50 mL), dried over sodium sulfate, and the solvent removed under vacuum. The residue was partitioned between tert-butylmethyl ether (150 mL) and 5M hydrochloric acid (75 mL) and the resulting mixture stirred at ambient temperature for 4 hours until no more gas evolution was noticed. The aqueous layer was collected and the solvent removed under vacuum. The residue was triturated with warm isopropanol (40 mL) to give a crystalline solid which was collected by filtration. The cake was rinsed with cold isopropanol (3×5 mL) and the dried under high vacuum for 2 hours to give 2.80 g (50%) of the title compound as an off-white crystalline solid.

¹H NMR (250 MHz, DEUTERIUM OXIDE) δ ppm 7.48-7.61 (m, 2H) 7.29 (d, J=8.07 Hz, 1H) 4.62 (s, 2H) 4.58 (s, 2H)

LC-MS: purity 90% (UV), t_{R} 0.68 min m/z [M+H]⁺ 198/200 (MET/CR/1278)

### Stage 2a: N-Boc-5-Bromo-isoindoline

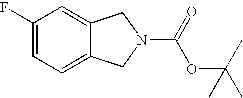

5-Bromo-isoindoline HCl salt (3.85 g, 16.4 mmol) was partitioned between tert-butylmethylether (100 mL) and 0.5 M aqueous sodium hydroxide (50 mL). The aqueous layer was extracted further with tert-butylmethylether (2×50 mL). The organic phases were combined, dried over anhydrous potassium carbonate, filtered, and the solvent removed under vacuum to give 1.55 g of a beige solid.

The solid (1.55 g, 7.83 mmol, 1 eq) was dissolved in pyridine (2.7 mL). Di-tertbutyldicarbonate (1.78 g, 8.15 mmol, 1.05 eq.), previously dissolved in dichloromethane (6 mL) was added dropwise over 5 minutes. Stirring was continued for 15 hours at ambient temperature and the reaction mixture was concentrated to dryness. The residue was portioned between tert-butylmethylether (25 mL) and 5% aqueous citric acid solution (20 mL). The aqueous phase was discarded and the organic phase dried over sodium sulfate, filtered and the solvent removed under vacuum to give 2.31 g (99%) of a yellow oil which solidified on standing.

¹H NMR (250 MHz, CHLOROFORM-d) δ ppm 7.33-7.48 (m, 2H) 7.04-7.21 (m, 1H) 4.53-4.73 (m, 4H) 1.52 (s, 9H)

LC-MS: purity 95% (UV), t_{R} 2.39 min m/z [M+H-tBu]⁺242.80 (MET/CR/1278)

Stage 3a: N-Boc-5-(1-morpholinylethylamino-isoincloline

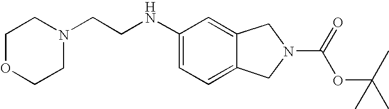

N-Boc-5-Bromo-isoindoline (298 mg, 1 mmol, 1 eq.), tripotassium phosphate (425 mg, 2 mmol, 2 eq.), copper (I) iodide (10 mg, 0.05 mmol, 0.05 eq.), diethyl salicylamide (39 mg, 0.2 mmol, 0.2 eq.) and N,N-dimethylformamide (3 mL) were charged in a pressure tube. 2-morpholinylethylamine (195 mg, 1.5 mmol, 1.5 eq.) was added as a single portion and the atmosphere on top of tube replaced with nitrogen. The tube was sealed and the reaction mixture heated at 100° C. for 15 hours. The reaction mixture was left to cool to ˜30° C. and was partitioned between water (10 mL) and ethyl acetate (15 mL). 0.88% aqueous ammonia (0.5 mL) was added and the 2 phase mixture stirred for a further 5 min. The organic phase was collected and the aqueous phase extracted with ethyl acetate (10 mL). The organic phases were combined, dried over anhydrous potassium carbonate, filtered and the solvent removed under vacuum. The residue was purified by chromatography using a tert-butylmethylether/methanol gradient (up to 1% MeOH in TBME). After combining the relevant fractions and removing the solvent under vacuum, 81 mg (23%) of the title compound was isolated as a colourless gum.

¹H NMR (500 MHz, CHLOROFORM-d) δ ppm 6.99-7.12 (m, 1H) 6.55-6.61 (m, 1H) 6.45-6.56 (m, 1H) 4.60 (d, J=17.42 Hz, 2H) 4.56 (d, J=15.04 Hz, 2H) 4.33 (br. s., 1H) 3.73 (t, J=4.40 Hz, 4H) 3.12-3.21 (m, 2H) 2.64 (t, J=5.87 Hz, 2H) 2.48 (br. s., 4H) 1.52 (s, 9H)

LC-MS: purity 91% (ELS), t_{R}1.40 min m/z [M+H]⁺348.10 (MET/CR/1278)

Stage 4a: 5-1-morpholinylethylamino-isoindoline

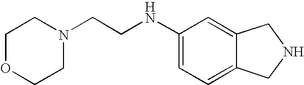

N-Boc-5-(1-morpholinylethylamino)-isoindoline (410 mg, 1.180 mmol, 1 eq.) and dichloromethane (7 mL) were charged into a 25 mL flask. The reaction mixture was cooled to 0° C. and trifluoroacetic acid (0.125 mL) was added dropwise. The reaction mixture was left to warm to ambient temperature and stirring was continued for a further 2 hours. The solvent was removed under vacuum to give 292 mg (100%) of a solid which was used in the next step without further purification.

¹H NMR: not submitted

LC-MS: purity 98% (ELS), t_{R} 0.29 min m/z [M+H]⁺248.15 (MET/CR/1278)

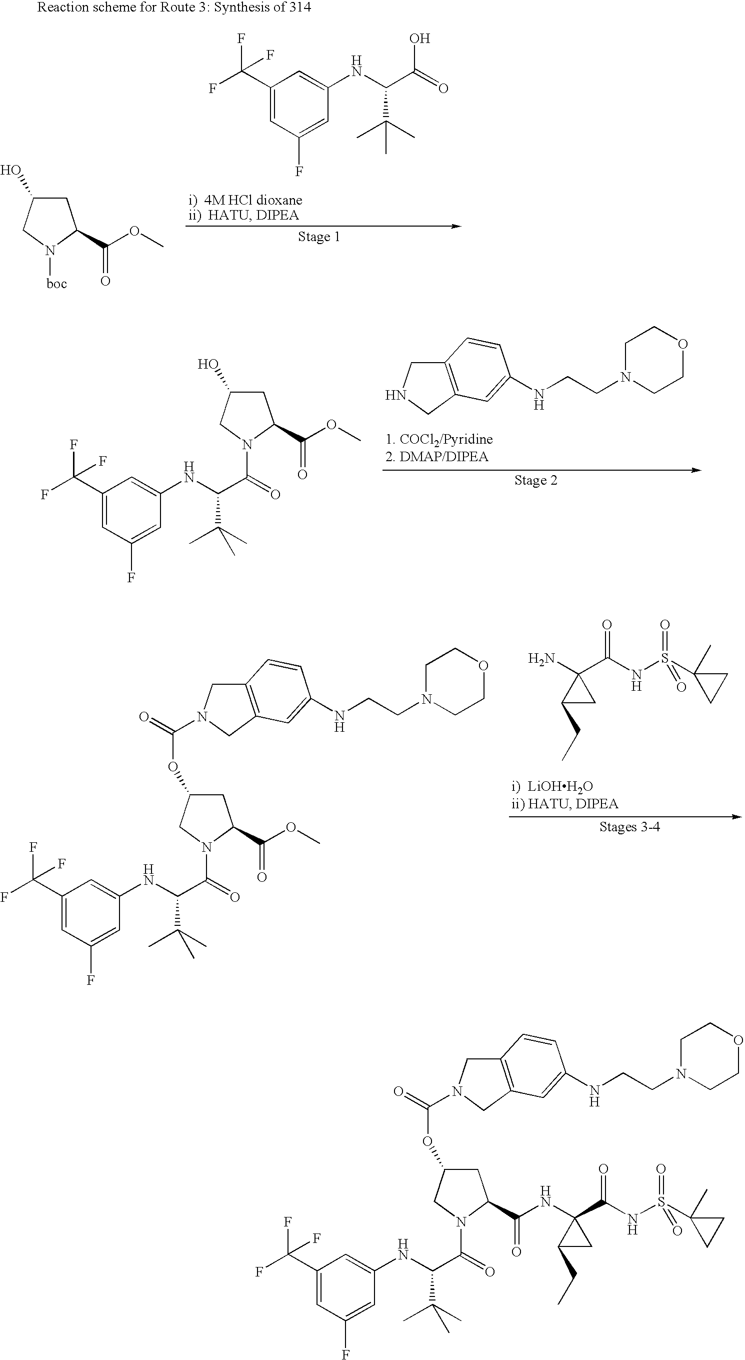

### Synthesis of 314

### Stage 1: 315

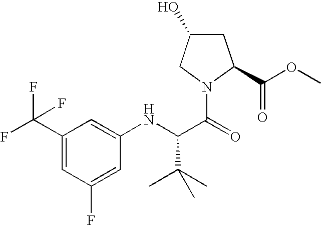

(2S)-2-(3-Fluoro-5-trifluoromethyl-phenylamino)-3,3-dimethyl-butanoic acid (480 mg, 1.637 mmol, 1.0 eq.), (2S,4R)—N-boc-4-hydroxy-hydroxyproline methyl ester (357 mg, 1.964 mmol, 1.2 eq.) and HATU (809 mg, 2.128 mmol, 1.13 eq.) and N,N-dimethylformamide (6.5 mL) were charged into a 25 mL flask and the reaction mixture cooled to 0° C. Diisopropylethylamine (0.856 mL, 4.911 mmol, 3.0 eq.) was added dropwise and the reaction mixture stirred for 15 hours at ambient temperature. The reaction mixture was diluted with ethyl acetate (35 mL) and washed with brine (2×35 mL). The aqueous phase was back extracted with ethyl acetate (35 mL). The organic phases were combined, dried over sodium sulfate, filtered and the solvent removed under vacuum. The residue was purified by flash column chromatography using ethyl acetate/heptanes (6:4) as eluent. After combining the relevant fractions and removing the solvent under vacuum, 578 mg (84%) of the desired product was isolated.

¹H NMR (500 MHz, CHLOROFORM-d) δ 6.55-6.68 (m, 2H), 6.44 (d, J=11.19 Hz, 1H), 4.79 (d, J=9.72 Hz, 1H), 4.58-4.69 (m, 2H), 3.84-3.95 (m, 2H), 3.67-3.81 (m, 4H), 2.28 (d, J=8.07 Hz, 1H), 2.03-2.20 (m, 1H), 1.67 (br. s., 1H), 1.12 (s, 9H)

LC-MS: purity 100% (UV), t_{R} 2.07 min m/z [M+H]⁺421.15(MET/CR/1278).

### Stage 2: 316

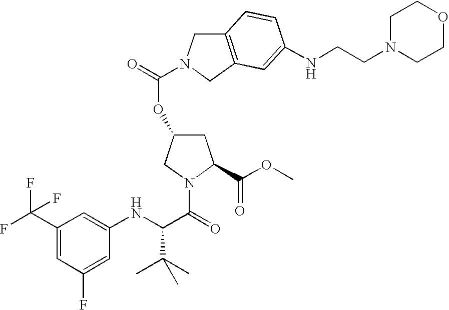

Phosgene (20% in toluene, 0.687 mL, 1.3 mmol, 1.1 eq.) and dichloromethane (12 mL) were charged into a 50 mL flask and the solution cooled to 0° C. Stage 1 intermediate (496 mg, 1.18 mmol, 1.0 eq.) was dissolved in dichloromethane (8 mL) and the resulting solution was added dropwise to the reaction flask over 5 min. The reaction mixture was left to warm up to ambient temperature and stirring was continued for a further 30 minutes. LCMS analysis of an aliquot showed around 80% conversion to the desired chloroformate intermediate. Extra phosgene (0.2 eq.) was added and stirring continued for a further 30 minutes. The reaction mixture was cooled to 0° C. N,N-dimethylaminopyridine (288 mg, 2.36 mmol, 2.0 eq.), 5-(1-morpholinylethylamino)-isoindoline (292 mg, 1.18 mmol, 1.0 eq.) and diisopropylethylamine (1.03 mL, 5.90 mmol, 5.0 eq.) were added sequentially dropwise. The reaction mixture was then stirred at ambient temperature for 15 hours. The reaction mixture was quenched with methanol (20 mL) and stirring was continued for 15 minutes. The solvent was removed under vacuum and the residue purified by flash column chromatography using an ethyl acetate/heptanes gradient (from 3:7 to neat EtOAc) as eluent. As no product was identified in the fraction the column was flushed with 10% methanol in dichloromethane. After combining the relevant fractions and removing the solvent under vacuum, 175 mg (21%) of the desired product was isolated.

¹H NMR (250 MHz, CHLOROFORM-d) δ 6.84-7.10 (m, 1H), 6.27-6.68 (m, 5H), 5.32-5.43 (m, 1H), 4.80 (br. s., 1H), 4.60 (t, J=8.22 Hz, 3H), 4.29-4.41 (m, 1H), 4.04-4.19 (m, 1H), 3.83-4.02 (m, 3H), 3.73-3.83 (m, 8H), 1.49 (s, 4H), 1.21-1.28 (m, 2H), 1.03-1.16 (m, 11H), 0.80-0.92 (m, 2H)

LC-MS: purity 82% (UV), t_{R}1.81 min m/z [M+H]⁺694.50(MET/CR/1278).

### Stage 3: 317

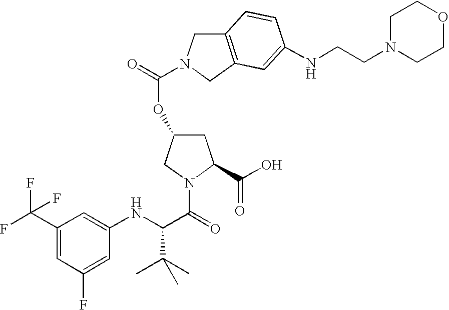

Stage 2 intermediate (175 mg, 0.252 mmol, 1.0 eq.), tetrahydrofuran (1 mL), water (0.5 mL) and methanol (0.5 mL) were charged into a 7 mL vial and the reaction mixture cooled down to 0° C. Lithium hydroxide monohydrate (16 mg, 0.378 mmol, 1.5 eq) previously dissolved in water (0.5 mL) was added dropwise and stirring was continued at 0° C. for another 20 min. Stirring was then continued at ambient temperature for a further 2 hours by when LCMS analysis of an aliquot showed 10% remaining of the starting material. Extra lithium hydroxide (0.5 eq.) was added and the reaction mixture left to stir at ambient temperature for 15 hours. The reaction mixture pH was adjusted to pH=7 by slow addition of 1M hydrochloric acid and the solvent removed under vacuum. The residue was purified by flash column chromatography using a methanol/dichloromethane gradient (from neat DCM to 4% MeOH in DCM) as eluent. After combining the relevant fractions and removing the solvent under vacuum, 78 mg (42%) of the desired product was isolated.

¹H NMR (250 MHz, MeOD) δ 6.74-7.14 (m, 2H), 6.42-6.74 (m, 3H), 6.26-6.38 (m, 1H), 5.36 (br. s., 1H), 4.53 (d, J=10.66 Hz, 4H), 4.02-4.40 (m, 5H), 3.95 (t, J=4.57 Hz, 5H), 3.37 (d, J=14.16 Hz, 5H), 2.46-2.60 (m, 1H), 2.24 (d, J=12.33 Hz, 1H), 1.03-1.21 (m, 9H), 0.88 (d, J=6.70 Hz, 1H)

LC-MS: purity 100% (UV), t_{R}1.69 min m/z [M+H]⁺690.40 (MET/CR/1278).

### Stage 4: 314

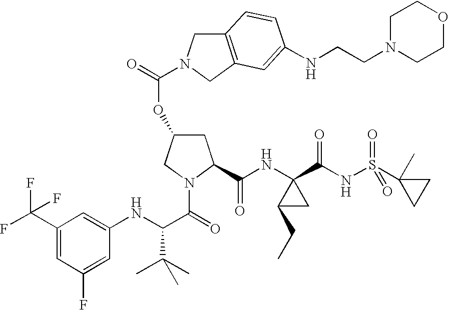

Stage 3 intermediate (73 mg, 0.107 mmol, 1.0 eq.), HATU (53 mg, 0.139 mmol, 1.3 eq.), (1R,2S)-1-amino-2-ethyl-cyclopropane-1-carbonyl-(1′-methyl)-cyclopropane-sulfon-amide (26 mg, 0.107 mmol, 1.0 eq.) and N,N-dimethylformamide (1.5 mL) were charged into a 7 mL vial and the reaction mixture cooled to 0° C. Diisopropylethylamine (0.112 mL, 0.642 mmol, 6 eq.) was added dropwise. The reaction mixture was left to warm to ambient temperature and stirred for a further 15 hours. The solvent was removed under vacuum and the residue purified by flash column chromatography using a methanol/dichloromethane gradient (from neat DCM to 4% MeOH in DCM) as eluent. After combining the relevant fractions and removing the solvent under vacuum, 30 mg (31%) of the desired product was isolated as an off white solid

¹H NMR (500 MHz, MeOD) δ 6.83-7.08 (m, 2H), 6.69-6.76 (m, 1H), 6.30-6.67 (m, 3H), 5.37 (br. s., 1H), 4.54 (dd, J=8.39, 16.33 Hz, 2H), 4.43 (t, J=8.55 Hz, 1H), 4.33 (t, J=16.02 Hz, 1H), 4.17-4.23 (m, 2H), 4.04-4.16 (m, 2H), 3.93 (dt, J=3.49, 12.40 Hz, 1H), 3.75 (t, J=4.50 Hz, 4H), 3.24-3.31 (m, 2H), 2.66-2.75 (m, 2H), 2.57-2.66 (m, 4H), 2.35-2.43 (m, 1H), 2.16 (d, J=8.09 Hz, 1H), 1.49-1.68 (m, 9H), 1.10-1.20 (m, 11H), 1.00 (t, J=7.17 Hz, 3H)

LC-MS: purity 100% (UV), t_{R} 3.86 min m/z [M+H]⁺908.50 (MET/CR/1416).

334, 335, and 336 were prepared following procedures described above for the preparation of compound 210.

The sodium salt formation for 335 and 336 is new and the procedure for this stage is presented in paragraph 2.

The analytical data for the 3 free NH compounds and 2 sodium salts compounds is presented in paragraph 3.

### Sodium Salt Formation Procedure: 335 Example

Compound 335 (110 mg, 0.122 mmol, 1 eq., free NH) was charged in a 10 mL flask. Water 2 mL was added to give a white slurry. 0.1 N aqueous sodium hydroxide solution (1.16 mL, 0.116 mmol, 0.95 eq.) was added dropwise. The obtained slurry was further diluted with water (4 mL) but full dissolution was not observed. The slurry was stirred at ambient temperature for 15 hours. The reaction mixture remained a white slurry after this time. The pH of the supernatant was measured at 6.5 (special 6-8 pH paper range).

A 0.25 mL aliquot was taken and the solvent removed under vacuum. ¹H NMR analysis showed disappearance of the sulfonamide proton (messy spectrum), while LCMS analysis showed a 100% UV peak with identical retention time to ITMN-8083 free NH (no decomposition noticed). The solvent for the remaining of the reaction mixture was removed under vacuum to give 95 mg (84%) of a white solid.

### Analytical Data:

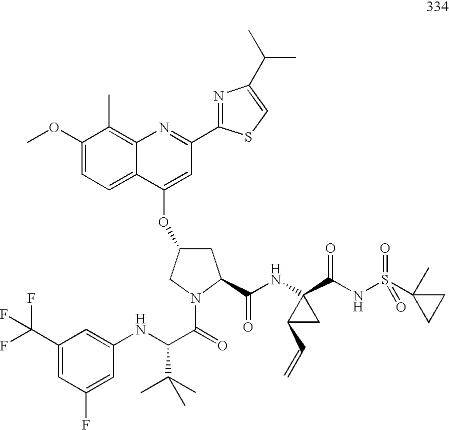

84 mg (35%), yellow solid.

¹H NMR (250 MHz, CHLOROFORM-d) δ 9.80 (br. s., 1H), 7.34-7.71 (m, 2H), 6.85-7.26 (m, 3H), 6.17-6.82 (m, 3H), 5.60-5.88 (m, 1H), 5.50 (br. s., 1H), 5.01-5.36 (m, 2H), 4.88 (d, J=11.12 Hz, 1H), 4.55 (t, J=8.15 Hz, 1H), 4.00-4.34 (m, 2H), 3.95 (s, 3H), 2.98-3.45 (m, 1H), 2.41-2.85 (m, 5H), 2.00-2.14 (m, 1H), 1.92 (dd, J=5.94, 7.92 Hz, 1H), 1.57-1.71 (m, 2H), 1.47 (s, 3H), 1.39 (d, J=6.55 Hz, 7H), 1.02-1.17 (m, 10H), 0.73-0.95 (m, 2H)

LC-MS: purity 100% (UV), t_{R} 5.28 min m/z [M+H]⁺929.68 (MET/CR/1426)

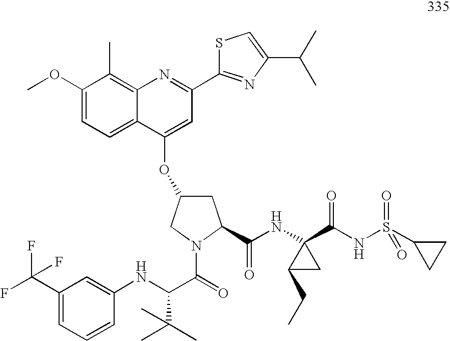

112 mg (62%), white solid.

¹H NMR (500 MHz, CHLOROFORM-d) δ ppm 10.11 (s, 1H) 7.50 (s, 1H) 7.43-7.48 (m, 1H) 7.06 (s, 1H) 6.99 (d, J=9.16 Hz, 1H) 6.69-6.90 (m, 4H) 6.57 (d, J=7.32 Hz, 1H) 5.51 (d, J=2.14 Hz, 1H) 4.61 (d, J=10.53 Hz, 1H) 4.46 (t, J=8.32 Hz, 1H) 4.22 (d, J=11.75 Hz, 1H) 4.08-4.15 (m, 1H) 3.99 (d, J=10.53 Hz, 1H) 3.97 (s, 3H) 3.07-3.36 (m, 1H) 2.78-3.08 (m, 1H) 2.70 (s, 3H) 2.60 (d, J=8.39 Hz, 2H) 1.71 (dd, J=8.24, 5.49 Hz, 1H) 1.32-1.44 (m, 9H) 1.19-1.30 (m, 2H) 1.14 (s, 9H) 1.06 (t, J=8.77 Hz, 2H) 0.96 (t, J=7.32 Hz, 3H)

LC-MS: purity 100% (UV), t_{R} 5.29 min m/z [M+H]⁺899.40 (MET/CR/1426)

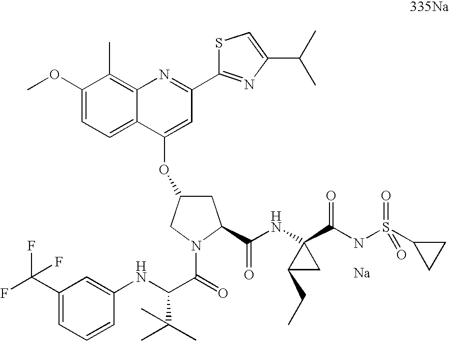

95 mg (84%), white solid.

LC-MS: purity 100% (UV), t_{R} 5.28 min m/z [M+H]⁺899.41 (MET/CR/1426)

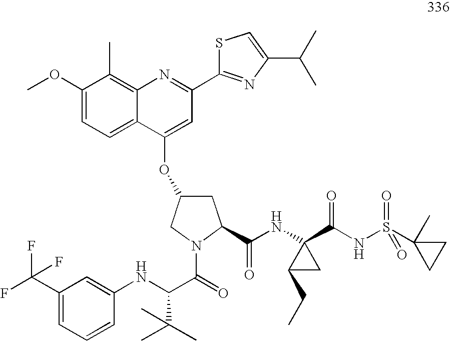

410 mg (55%), white solid.

¹H NMR (500 MHz, CHLOROFORM-d) δ ppm 9.76 (s, 1H) 7.49 (s, 1H) 7.42 (d, J=9.16 Hz, 1H) 7.06 (s, 1H) 7.03 (s, 1H) 6.96 (d, J=9.31 Hz, 1H) 6.85 (s, 1H) 6.74-6.83 (m, 2H) 6.56 (d, J=7.63 Hz, 1H) 5.49 (br. s., 1H) 4.60 (d, J=10.22 Hz, 1H) 4.55 (t, J=8.24 Hz, 1H) 4.23 (d, J=11.75 Hz, 1H) 4.07 (dd, J=11.83, 3.43 Hz, 1H) 4.00 (d, J=10.38 Hz, 1H) 3.96 (s, 3H) 3.21 (quin, J=6.87 Hz, 1H) 2.69-2.71 (m, 3H) 2.64-2.69 (m, 1H) 2.56-2.63 (m, 1H) 1.63-1.77 (m, 1H) 1.56-1.59 (m, 1H) 1.54 (s, 3H) 1.47-1.53 (m, 1H) 1.40 (dd, J=6.87, 1.98 Hz, 6H) 1.36 (d, J=7.78 Hz, 1H) 1.23-1.33 (m, 2H) 1.19 (dd, J=9.54, 5.57 Hz, 1H) 1.12 (s, 9H) 1.00 (t, J=7.40 Hz, 3H) 0.83-0.94 (m, 2H)

LC-MS: purity 100% (UV), t_{R} 2.42 min m/z [M+H]⁺913.45 (MET/CR/1981)

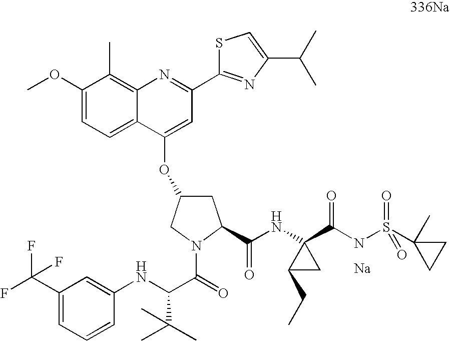

396 mg (94%), white solid.

LC-MS: purity 100% (UV), t_{R} 5.34 min m/z [M+H]⁺913.39 (MET/CR/1426)

### Preparation of 350 & 351

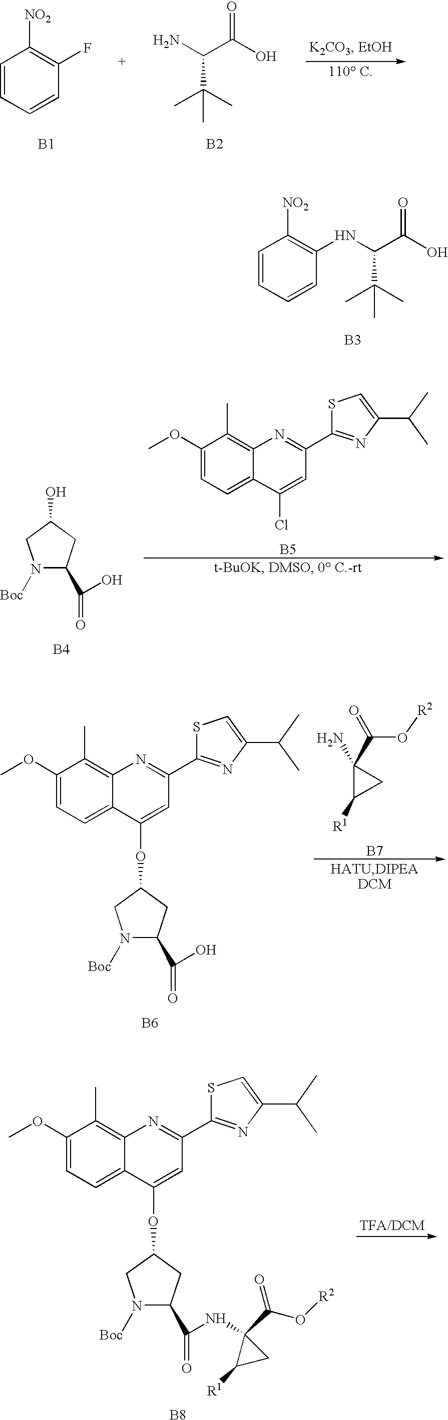

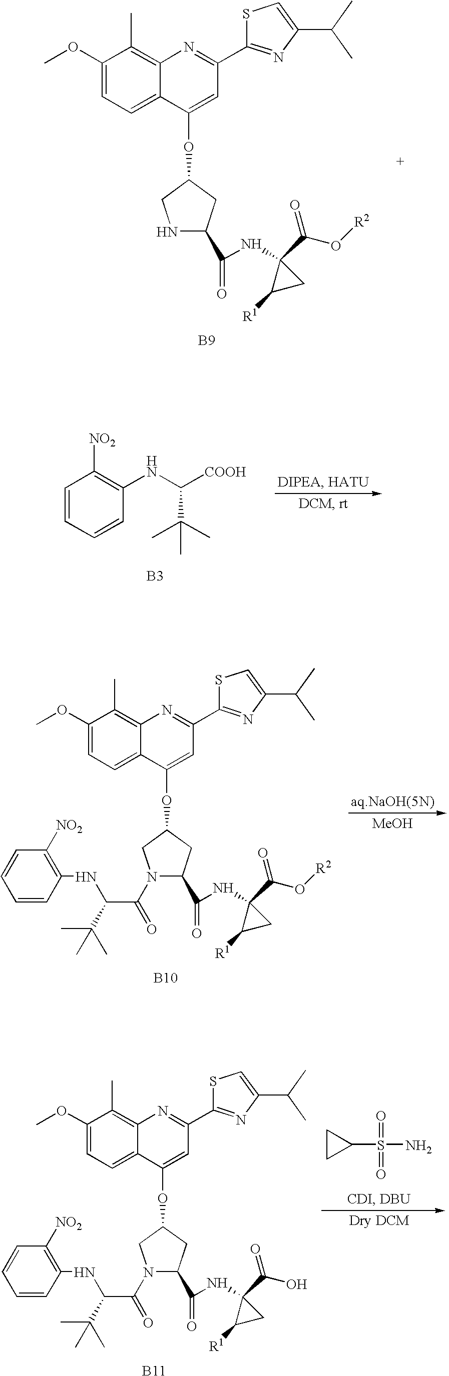

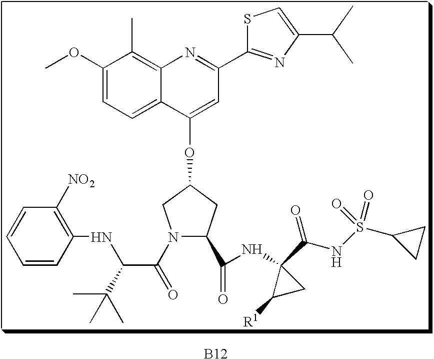

### Preparation of Compound B3

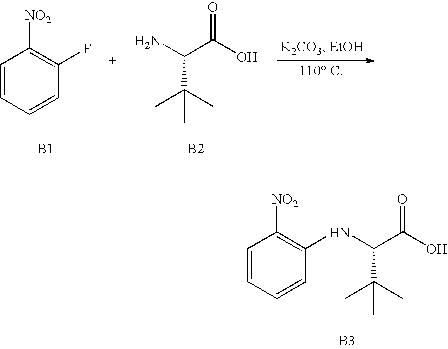

To a slurry of L-tert-leucine (1.0 g 7.7 mmol) in EtOH (20 mL) in a sealed tube was added 1-fluoro-2-nitrobenzene (812 μL, 7.7 mmol) and K₂CO₃ (2.3 g, 15.4 mmol). After heating to 110° C. for 2 h, the resulting red slurry was filtered to remove excess K₂CO₃ and washed with DCM. Solvent was dried by vacuum and re-crystallized with CH₃OH/Et₂O (V/V= 1/10). The title compound was obtained (1.7 g, 87%) as a red solid. NMR: (400 MHz, CD₃OD) δ 1.09 (s, 1H), 1.16 (s, 9H), 3.82 (s, 3H), 6.62 (ddd, 1H, J=8.6, 7.0, 1.2 Hz, 1H), 7.01 (d, J=8.5 Hz, 1H), 7.43 (ddd, J=8.9, 7.0, 1.8 Hz, 1H), 8.13 (dd, J=8.6, 1.5 Hz, 1H)

### Preparation of Compound B6

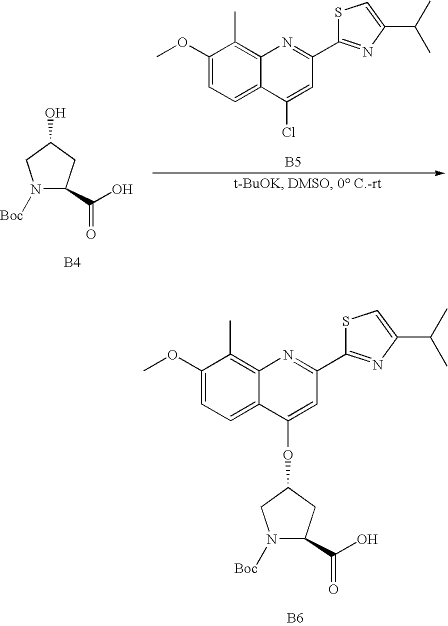

To a suspension of compound B4 (3.0 g, 12.1 mmol) in DMSO (60 ml) was added t-BuOK (3.4 g, 30.2 mmol) at 0° C. The generated mixture was stirred for 1.5 hour and then the compound B5 (4.4 g, 13.3 mmol) was added in one portion. The reaction was stirred for one day, and the reaction mixture was poured into ice-water. The aqueous solution was acidified to pH=4.6, filtered to obtained a white solid, and dried in freeze drier to give crude compound B6 (4.1 g, 65.2%), which was used directly without purification.

### General Procedure for Preparation of Compound B8:

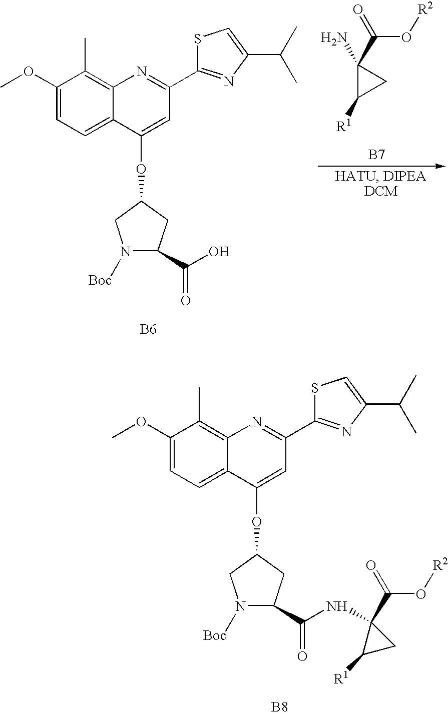

To a solution of compound B6 (200 mg, 1 eq) in dry DCM (10 mL) was added amine B7 (2 eq.), followed by adding HATU (1.5 eq) and DIPEA (4 eq), and the reaction mixture was stirred at room temperature for one day. The resulting mixture was concentrated to remove solvent, diluted with EtOAc, washed with pH=4.0 buffer and saturated aqueous NaHCO₃, dried and concentrated to give a residue. The residue was purified by flash column chromatography to afford compound B8.

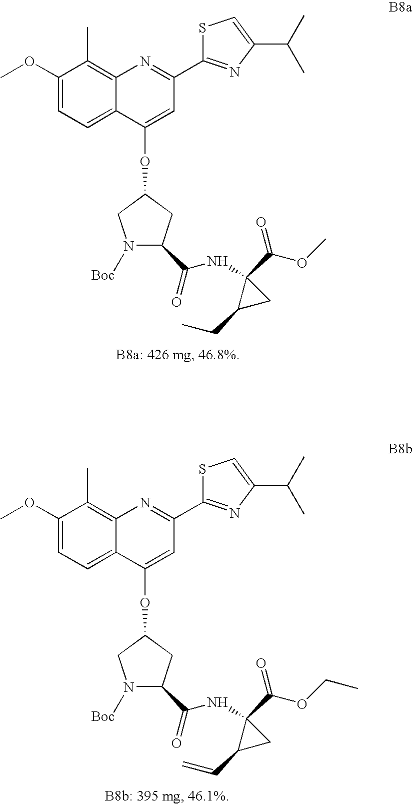

### General Procedure for Preparation of Compound 9:

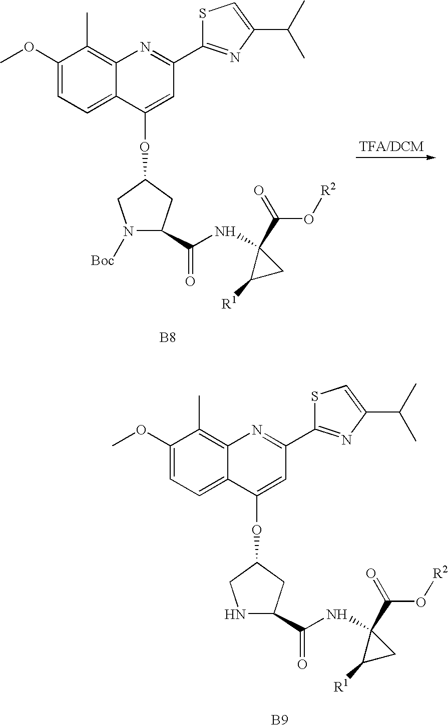

To a solution of compound B8 (400 mg) in dry DCM (5 mL) was added TFA (2.5 mL). The reaction mixture was stirred at room temperature for 2 h, at which time LC-MS analysis showed the reaction to be complete. The reaction mixture was concentrated, diluted with EtOAc, washed with saturated aqueous NaHCO₃, dried, and concentrated to give crude compound B9, which used directly without further purification.

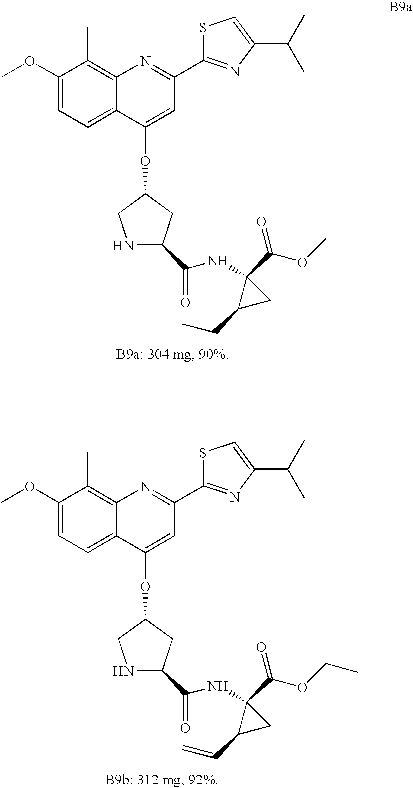

### General Procedure for the Preparation of Compound B10:

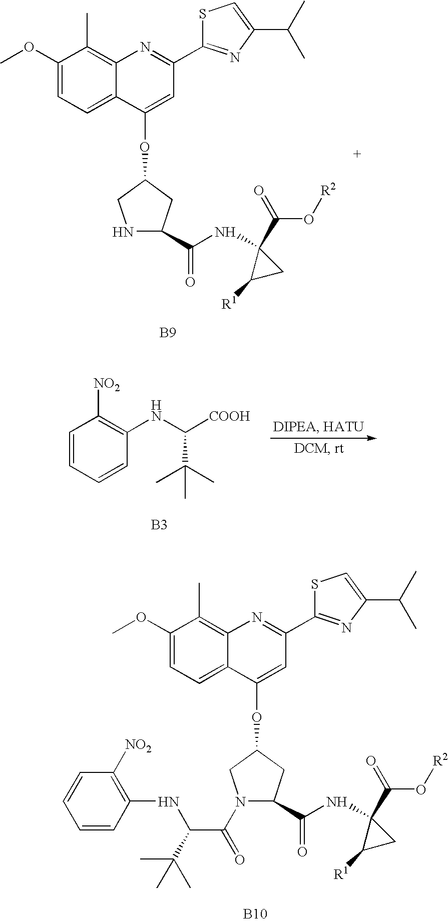

To a solution of compound B9 (300 mg, 1 eq) in DCM was added DIPEA (8 eq.), then added compound 3 (1.1 eq.), followed by HATU (1.5 eq.). The reaction mixture was stirred overnight, at which time LC-MS analysis showed the reaction to be complete. The mixture was quenched by adding water and extracted with EtOAc. The combined organic layer was then dried over Na₂SO₄ and concentrated. The residue was purified by prep-TLC (PE:EA=1:1) to afford compound B10.

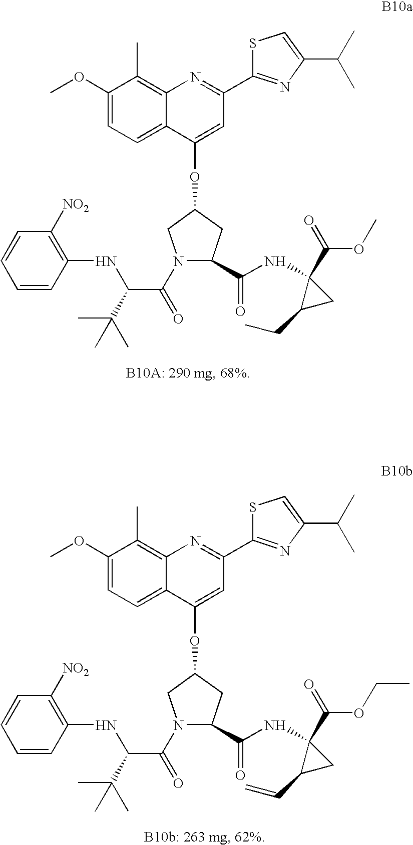

### General Procedure for Preparation of Compound B11:

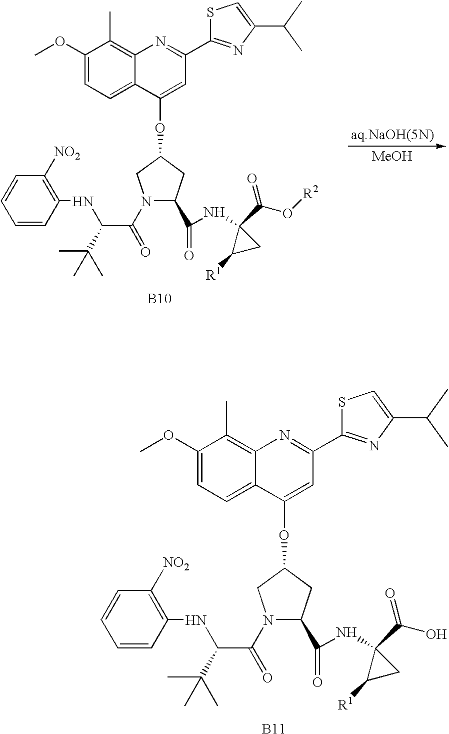

To a solution of compound B10 (150 mg, 1 eq.) in MeOH (5 mL) and was added aqueous NaOH (5 N, 10 eq) at room temperature. The reaction mixture was stirred at room temperature for two days, at which time LC-MS analysis showed the reaction complete. The reaction vessel was placed in an ice-water bath and the mixture was acidified to a pH of about 6-7 with aqueous HCl solution (1N). The resulting mixture was extracted with EtOAc, and the combined organic layer was dried over Na₂SO₄ and concentrated to give crude compound B11 which was used directly without further purification.

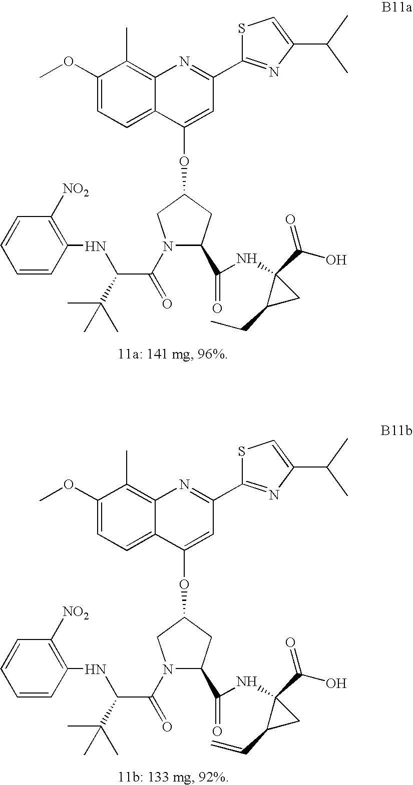

### General Procedure for Preparation of Final Compound 12:

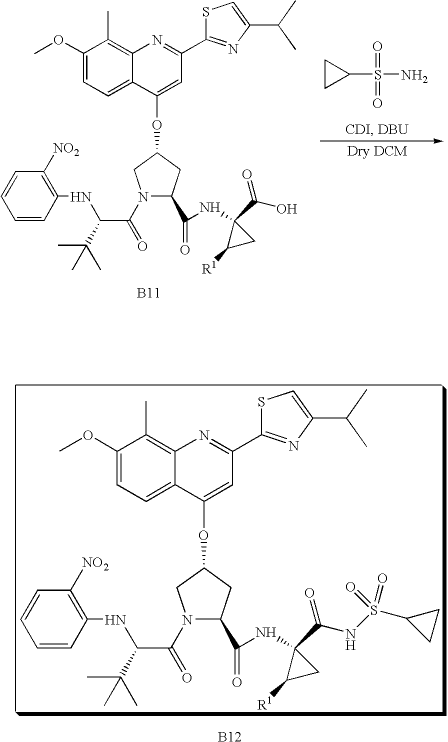

The final compounds B12 (350 and 351) are prepared following the general procedure mentioned above

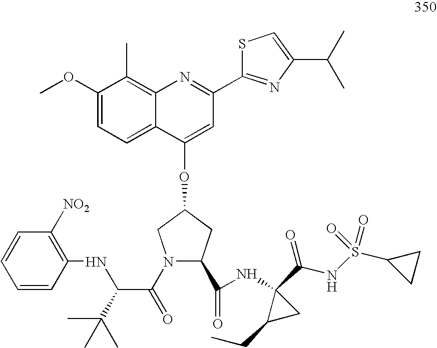

50 mg, 48%. MS (ESI) m/z (M+H)⁺876.2.

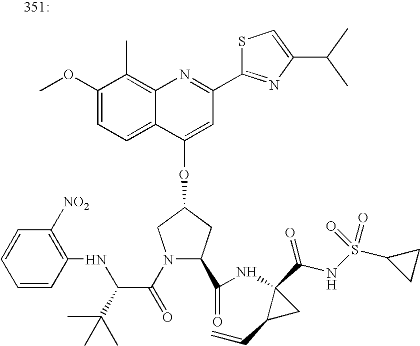

25 mg, 42%. MS (ESI) m/z (M+H)^{±}874.3.

### Preparation of 352 & 353

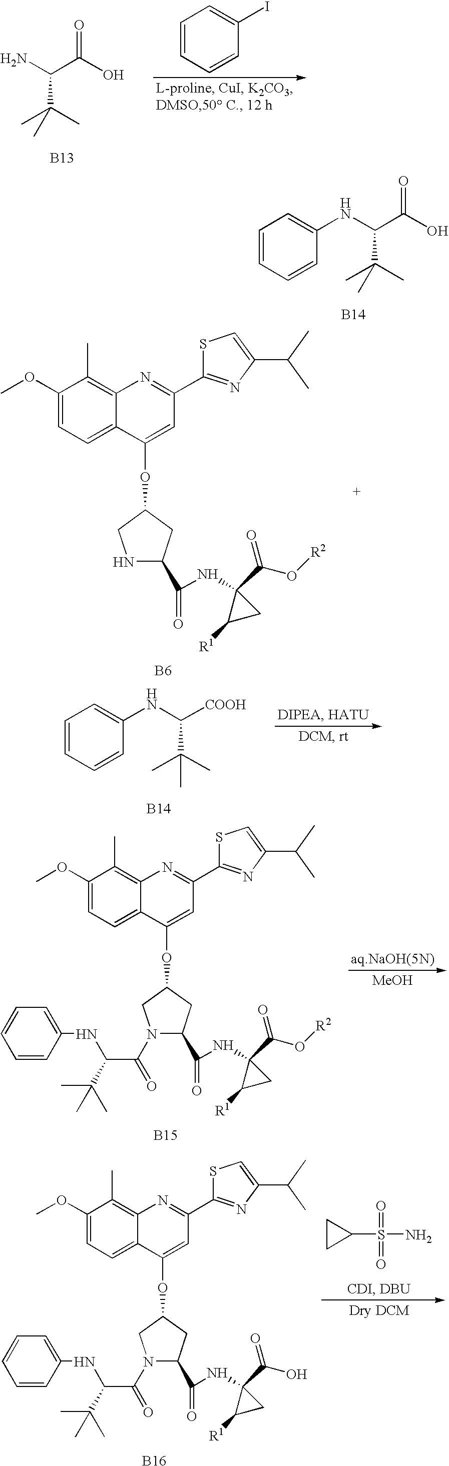

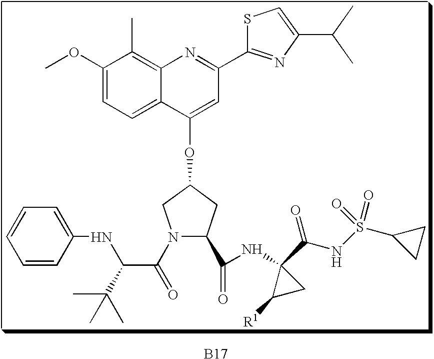

### Preparation of Compound B14

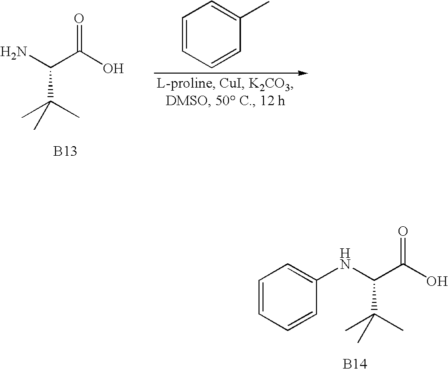

Compound B14 was prepared by followed the general procedure (Yield 15%) used for preparing B3. ¹H NMR: (400 MHz, DMSO-d₆) δ 7.04 (t, J=8.0 Hz, 2H), 6.66 (d, J=8.0 Hz, 2H), 6.52 (t, J=7.2 Hz, 1H), 5.42 (brs, 1H), 3.60 (s, 1H), 1.01 (s, 9H).

### Preparation of Compound B15

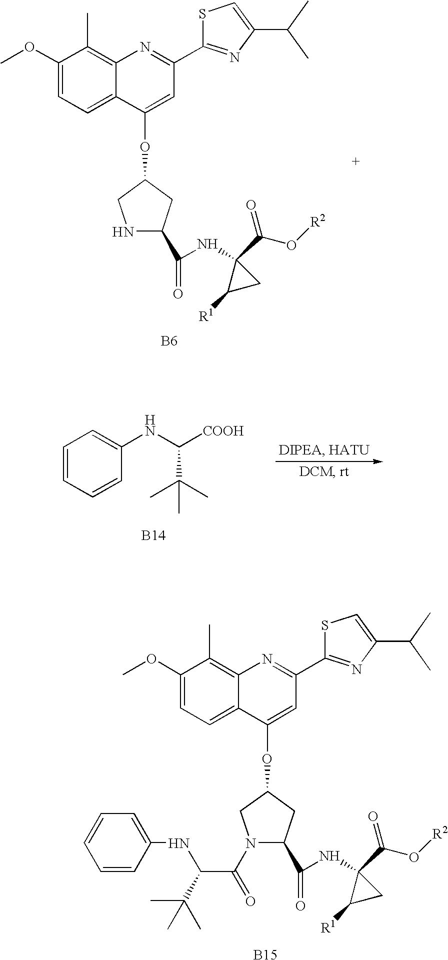

Final compound 15 is prepared using the general procedure.

### Preparation of Compound B16

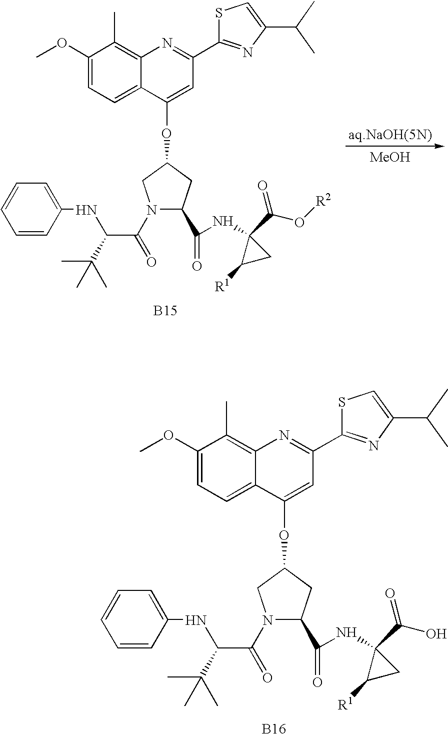

Final compound B16 is prepared using the general procedure.

### Preparation of Final Compound B17

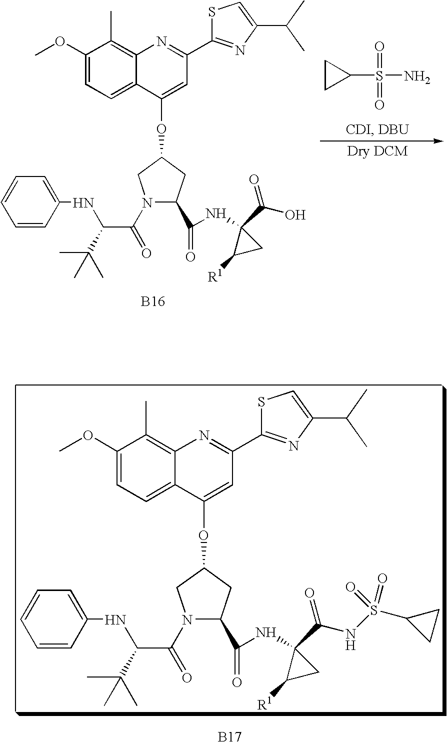

Final compound B17 is prepared using the general procedure. The following compounds were prepared using this method:

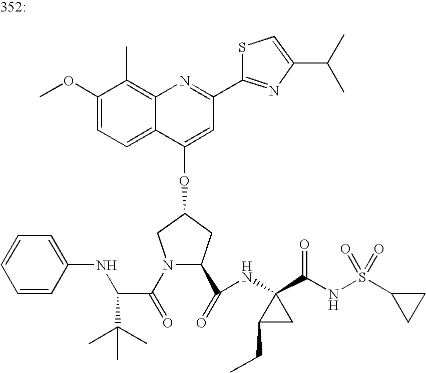

120 mg, 75.4%. MS (ESI) m/z (M+H)^{±}831.5

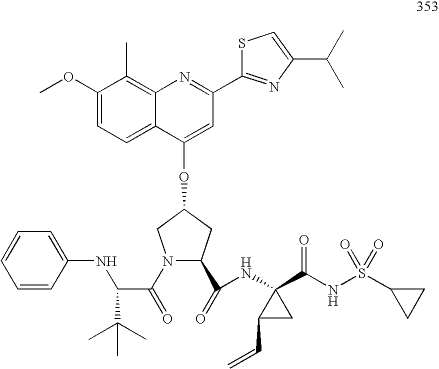

300 mg, 67%. MS (ESI) m/z (M+H)^{±}829.5

### Preparation of 354

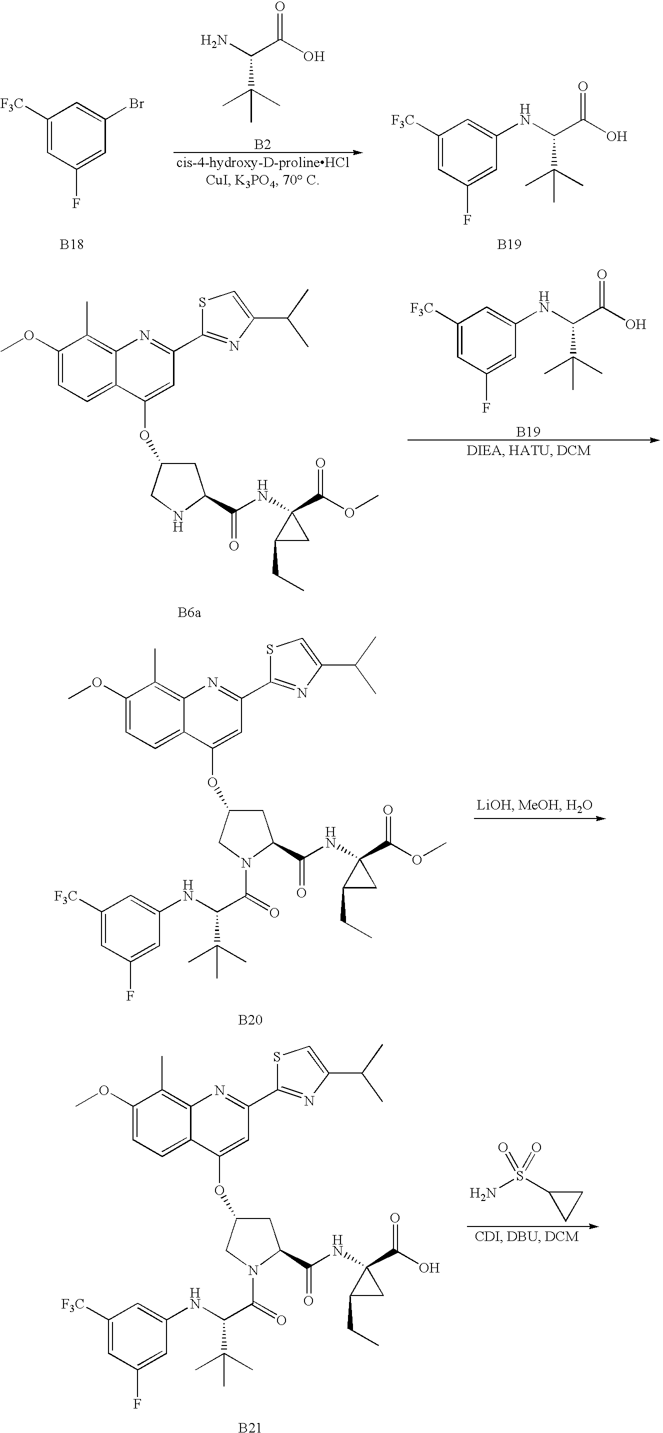

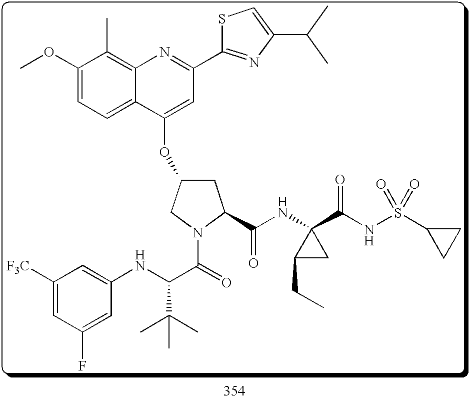

### Preparation of Compound B19:

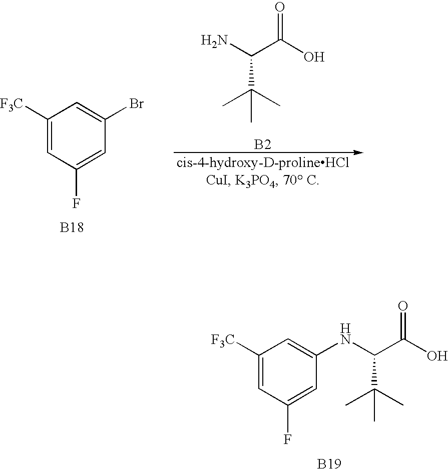

Final compound B19 is prepared by followed the general procedure. Yield 50%. ¹H NMR (400 MHz, DMSO-d₆) δ 12.79 (brs, 1H), 6.91 (s, 1H), 6.73-6.63 (m, 2H), 6.45 (d, J=9.2 Hz, 1H), 3.74 (d, J=9.2 Hz, 1H), 1.02 (s, 9H).

Preparation of compound B17

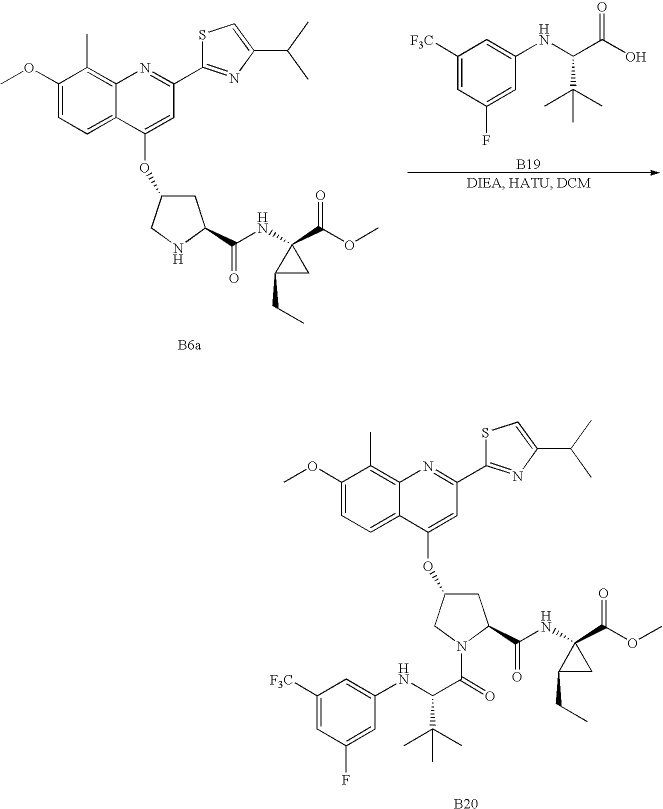

To a solution of compound B6a (1.1 g, 2 mmol) in dry DCM (30 ml) was added DIEA (1.29 g, 10 mmol), then compound B19 (879 mg, 3 mmol), followed by HATU (1.52 g, 4 mmol). The reaction mixture was stirred overnight, at which time TLC analysis showed the reaction was complete. The mixture was quenched by adding water and extracted with DCM, and the combined organic layers were dried and concentrated. The residue was purified by silica gel (PE:EA=3:1) to afford compound B20 (1.31 g, 79%).

### Preparation of Compound 21

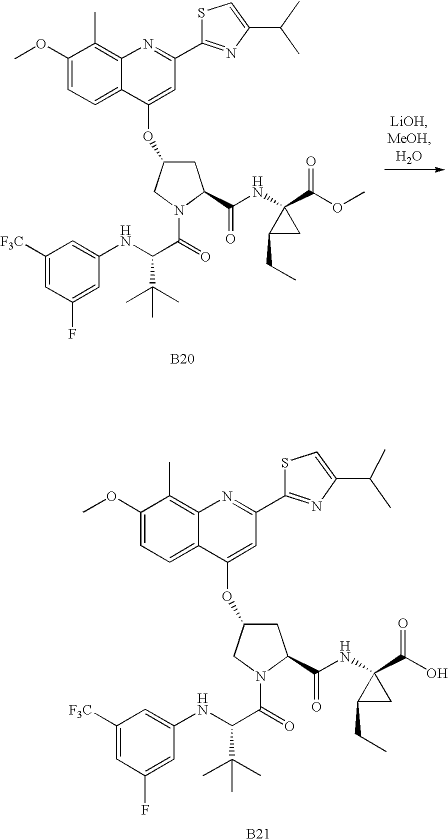

To a solution of compound B20 (1.31 g, 1.58 mmol) in methanol (30 ml) and water (10 ml) was added LiOH.H₂O (2.33 g, 55.3 mmol). The reaction mixture was stirred at room temperature overnight. TLC analysis showed the reaction was complete. The mixture was acidified to pH=3 with 2M aq. HCl solution under ice bath. The result mixture was extracted with ethyl acetate. The combined organic layers were dried and concentrated to afford compound B21 (1.28 g, 100%) used directly without further purification.

### Preparation of 354

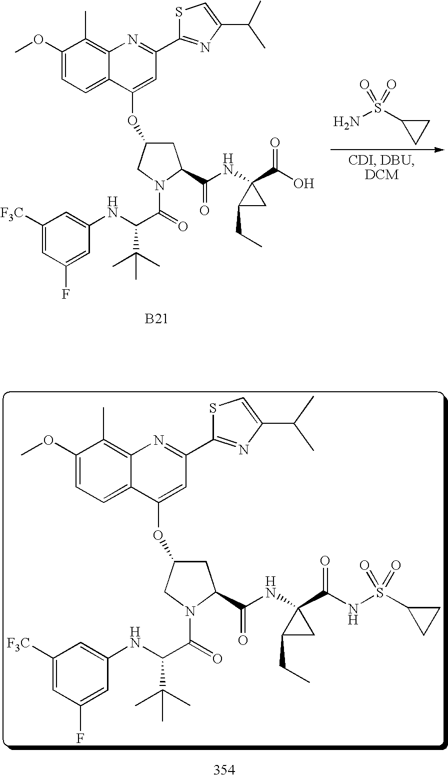

To a solution of compound B21 (1.28 g, 1.58 mmol) in dry DCM (30 ml) was added CDI (1.03 g, 6.36 mmol), and the mixture was stirred at 30° C. for 2 h. DBU (2.4 g, 15.8 mmol) was then added to the mixture, followed by cyclopropylsulfonamide (765 mg, 6.32 mmol). The reaction mixture was then stirred at 30° C. overnight, at which time TLC analysis showed the reaction was complete. The mixture was quenched by adding water, extracted with ethyl acetate, and the combined organic layers were dried and concentrated. The residue was purified by prep-HPLC to afford 354 (95 mg, 7%). MS (ESI) m/z (M+H)^{±}917.3

### Preparation of 400 and 401

### Preparation of N-Aryl Tert-Leucine Amino Acids

### General Procedure: (2S)-2-(3-Fluoro-5-trifluoromethyl-phenylamino)-3,3-dimethyl-butanoic acid (450)

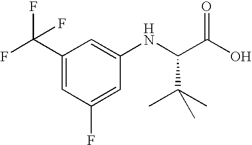

L-tert-leucine (4.0 g, 30.5 mmol, 1.0 eq.), lithium chloride (129 mg, 3.05 mmol, 0.1 eq.), copper(I) iodide (289 mg, 1.52 mmol, 0.05 eq.) and cesium carbonate (7.5 g, 22.9 mmol, 0.75 eq.) were charged into a 250 mL flask. tert-Butanol (100 mL) was added and the resulting mixture was stirred at 40° C. for 20 minutes, by which time the milky solution had turned blue. 3-Fluoro-5-trifluoromethyl-bromobenzene (7.41 g, 30.5 mmol, 1 eq.) was added dropwise, and the reaction mixture was heated at 100° C. for 15 hours. LCMS analysis of an aliquot showed around 20% (UV) of unreacted 3-Fluoro-5-trifluoromethyl-bromobenzene. Extra copper(I) iodide (289 mg, 0.05 eq.) was added and the reaction mixture was stirred at 100° C. for another 24 hours. LCMS analysis showed ˜16% (UV) of remaining 3-Fluoro-5-trifluoromethyl-bromobenzene. Heating was stopped and the solvent removed under vacuum to give a blue solid. The solid was partitioned between ethyl acetate (100 mL) and water (100 mL). The pH of the aqueous phase was adjusted to pH=1 with 4M Hydrochloric acid (10 mL). The organic phase was collected, washed with 2M hydrochloric acid (2×100 mL) dried over sodium sulfate, filtered and the solvent removed under vacuum to give 6.90 g (77%) of the title compound as an orange solid which was used in the next step without further purification.

¹H NMR (500 MHz, CHLOROFORM-d) δ ppm 6.61-6.75 (m, 2H) 6.49 (dt, J=10.68, 2.14 Hz, 1H) 4.48 (br. s., 1H) 3.79 (s, 1H) 1.11 (s, 9H)

LC-MS: purity 100% (ELS) 90% (UV), t_{R} 2.14 min m/z [M+H]⁺294.10

The next amino acids were prepared following the general procedure described for 450.

### (2S)-2-(4-Fluoro-3-trifluoromethyl-phenylamino)-3,3-dimethyl-butanoic acid (451)

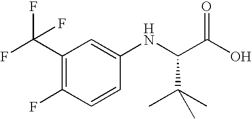

451 was prepared in the same fashion as 450.

3.86 g (50%) of a brown solid.

¹H NMR (250 MHz, CHLOROFORM-d) δ ppm 6.93-7.06 (m, 1H) 6.84 (dd, J=5.56, 2.97 Hz, 1H) 6.71-6.81 (m, 1H) 6.21 (br. s., 2H) 3.73 (s, 1H) 1.10 (s, 9H)

LC-MS: purity 97% (UV), t_{R} 2.12 min m/z [M+H]⁺ 294.00 (MET/CR/1278)

Preparation of di-methyl-sulfamide P1/P1′ Intermediate

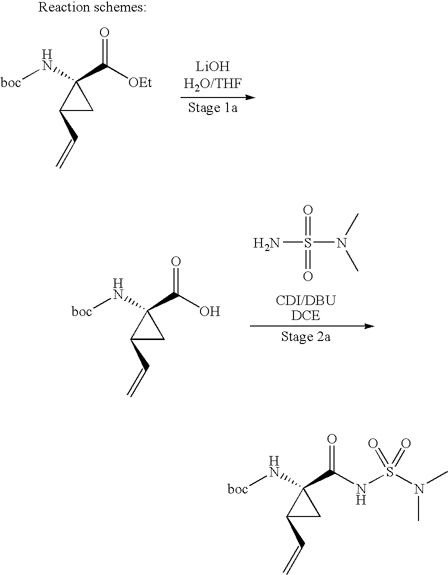

### Procedure

### Stage 1a: (1R,2S)-1-(tert-butoxycarbonylamino)-2-vinyl-cyclopropane-1-carboxylic acid (452)

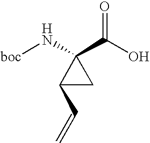

Ethyl (1R,2S)-1-(tert-butoxycarbonylamino)-2-vinyl-cyclopropane-1-carboxylate (61 g, 0.239 mol, 1.0 eq.) and tetrahydrofuran (700 mL) were charged into a 2 L round bottom flask placed in ice/water bath. Lithium hydroxide monohydrate (30 g, 0.714 mol, 3.0 eq.) was dissolved in water (800 mL) and added slowly to the mixture. The reaction mixture was heated at 50° C. for 18 hours. Monitoring the reaction conversion by LCMS showed some residual starting material so lithium hydroxide (20 g, 0.476 mol, 2 eq.) was added. The reaction was stirred further for 5 hours and then stirred at room temperature for 2 days. Monitoring the reaction conversion by LCMS showed complete conversion. The reaction mixture was acidified to pH 3 by slow addition of 1M hydrochloric acid then extracted with ethyl acetate (4×900 mL). The organic extracts were pooled, washed with brine (600 mL), dried over sodium sulfate, filtered and concentrated to dryness. Cyclohexane (100 mL) was added to the dried crude material and concentrated to give 71.44 g (54.0 g, 100%, corrected for residual solvent) of the title compound as a pale yellow solid which contained residual cyclohexane (24.5% w/was calculated from ¹HNMR). The compound was used in the next step without further purification.

¹H NMR (500 MHz, CHLOROFORM-d) δ ppm 5.79 (dt, J=17.01, 9.65 Hz, 1H) 5.27 (br. s., 1H) 5.30 (d, J=17.09 Hz, 1H) 5.14 (d, J=10.38 Hz, 1H) 2.20 (q, J=8.85 Hz, 1H) 1.70-1.90 (m, 1H) 1.52-1.63 (m, 1H) 1.45 (s, 9H)

LC-MS: purity 100% (UV), m/z [M+Na]⁺250.00, 1.60 min (MET/CR/1278).

### Stage 2a: (1R,2-1-(tert-butoxcarbonlyamino)-2-vinyl-cyclopropane-1-carbonyl-(1′-methyl)-cyclopropanesulfonamide (453)

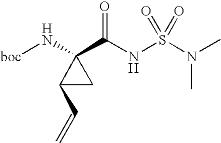

(1R,2S)-1-(tert-Butoxycarbonylamino)-2-vinyl-cyclopropane-1-carboxylic acid (1.3 g, 5.72 mmol, 1.0 eq.), dichloroethane (30 mL) and molecular sieves were charged into a 100 nth round bottom flask. The mixture was stirred at room temperature for 15 minutes. The molecular sieves were filtered off and washed with dichloroethane (2×5 mL). 1,1′-Carbonyldiimidazole (1.29 g, 8.01 mmol, 1.4 eq.) was added portionwise and the reaction mixture stirred vigorously at 50° C. for 1 hour until no more gas evolution was noticed. Dimethylsulfamide (1.70 g, 13.62 mmol, 1.7 eq.) was added portionwise followed by dropwise addition of DBU (3.2 mL, 21.63 mmol, 2.7 eq.). Stirring was continued at 50° C. for a further 15 hours by which time LCMS analysis of the reaction mixture showed full consumption of the starting material. The reaction mixture was washed with 0.5 M hydrochloric acid (3×50 mL) and brine (50 mL), dried over sodium sulfate and filtered. The residue was purified by flash column chromatography, using a methanol:dichloromethane gradient (from neat dichloromethane to 2% methanol in dichloromethane). After combining the relevant fractions and solvent removal, 1.5 g (78%) of the title compound was isolated as a white solid.

¹H NMR (500 MHz, CHLOROFORM-d) δ ppm 8.90-9.88 (m, 1H) 5.46-5.73 (m, 2H) 5.14 (d, J=10.38 Hz, 1H) 2.90 (s, 6H) 2.12 (q, J=8.70 Hz, 1H) 1.87 (dd, J=7.93, 5.80 Hz, 1H) 1.45 (br. s., 9H) 1.23-1.38 (m, 1H). LC-MS: purity 99% (UV), m/z [M+Na]⁺356.35, 1.32 min (MET/CR/1278)

### Preparation of 400 and 401

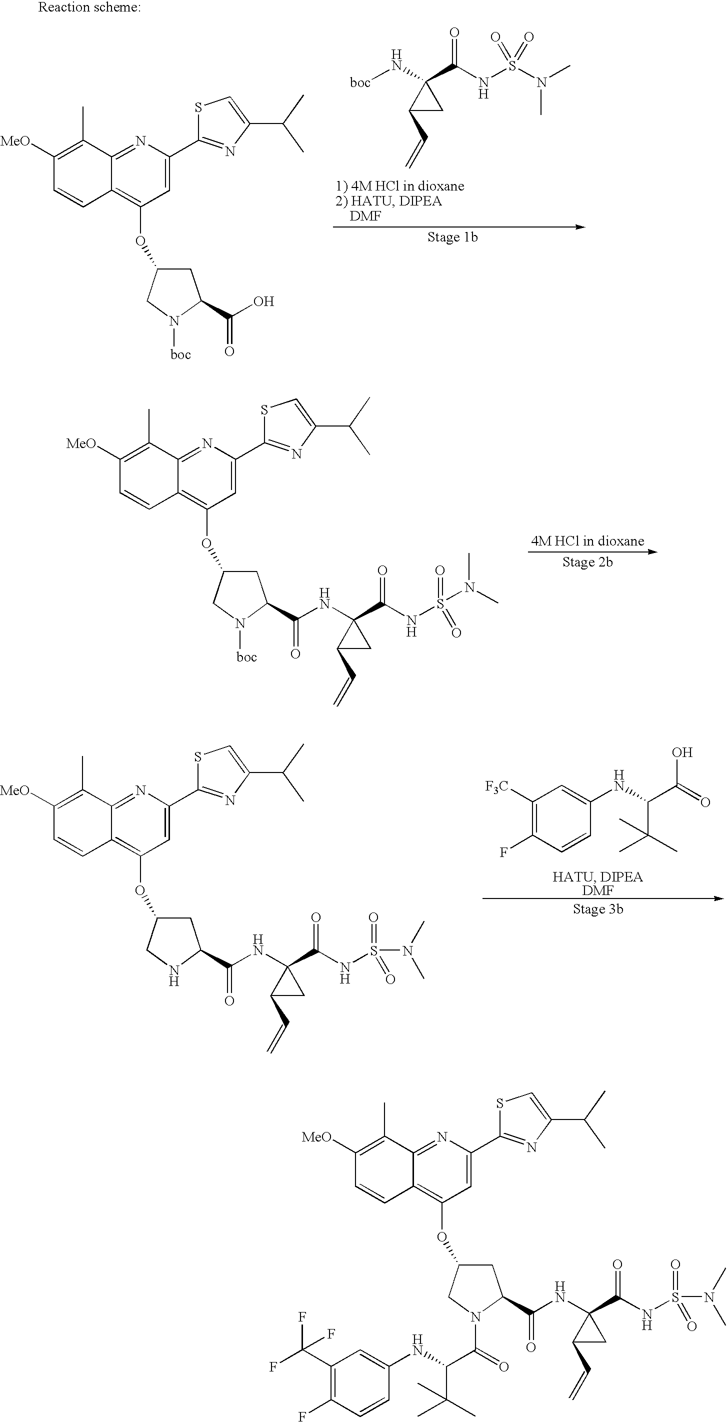

### Stage 1b: Synthesis of N-boc-α1-α1′/P2Intermediate (454)

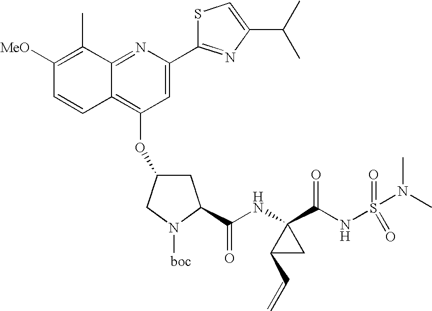

Compound 453 (1.5 g, 4.50 mmol, 1.0 eq.) and dioxane (3 mL) were charged into a 50 mL round bottom flask and the reaction mixture cooled on top of an ice bath. 4M HCl in dioxane (15 mL) was added and the reaction mixture stirred at ambient temperature for 1 hour. After this time, LCMS analysis of an aliquot showed the reaction to be complete. The solvent was removed under vacuum and the residue azeotroped with dichloromethane (2×30 mL) twice. The residue was used in the next step without further purification.

MMQ-proline derivative (2.05 g, 4.05 mmol, 0.9 eq.) and N,N-dimethylformamide (20 mL) were charged into a 50 mL round bottom flask and the reaction mixture cooled to 0° C. HATU (2.2 g, 5.85 mmol, 1.3 eq.) was added portion wise followed by diisopropylethylamine (4 mL, 22.5 mmol, 5.0 eq.). Stirring was continued at 0° C. for a further 15 minutes. A solution of the amino acid residue in N,N-dimethylformamide (5 mL) was then added to the reaction mixture. The reaction mixture was stirred at ambient temperature for a further 2 hours by which time LCMS analysis of an aliquot showed the reaction to be complete. The solvent was removed under vacuum and the residue dissolved in ethyl acetate (100 mL). The organic phase was washed with water (2×100 mL), dried over sodium sulfate, filtered and the solvent removed under vacuum. The residue was purified by flash column chromatography, using a ethyl acetate:heptanes gradient (from 1:9 to 7:3 ethyl acetate:heptanes). After combining the relevant fractions and solvent removal, 2.40 g (83%) of the title compound was isolated as a pale yellow solid.

¹H NMR (500 MHz, CHLOROFORM-d) δ ppm 9.82 (s, 1H) 7.92 (d, J=9.16 Hz, 1H) 7.51 (s, 1H) 7.24 (d, J=9.16 Hz, 1H) 7.07 (br. s., 1H) 7.05 (s, 1H) 5.71-5.85 (m, 1H) 5.43 (br. s., 1H) 5.30 (d, J=17.09 Hz, 1H) 5.17 (d, J=10.38 Hz, 1H) 4.38 (t, J=7.93 Hz, 1H) 4.00 (s, 3H) 3.82-3.96 (m, 2H) 3.20 (spt, J=6.82 Hz, 1H) 2.93 (s, 6H) 2.70 (s, 3H) 2.60 (d, J=6.10 Hz, 2H) 2.11 (q, J=8.65 Hz, 1H) 1.97 (dd, J=8.01, 5.87 Hz, 1H) 1.47 (s, 9H) 1.40-1.44 (m, 1H) 1.39 (d, J=7.78 Hz, 6H)

LC-MS: purity 100% (UV), t_{R} 2.48 min m/z [M+H]⁺ 743.30 (MET/CR/1981)

### Stage 1b: Synthesis P1/P1′/P2 intermediate (455)

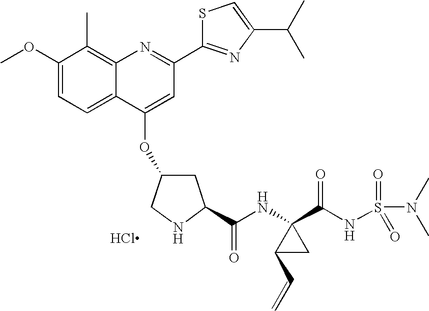

Stage 1b intermediate (1.4 g, 1.884 mmol, 1 eq.) and dioxane (3 mL) were charged into a 50 mL round bottom flask and the reaction mixture cooled on top of an ice bath. 4M HCl in dioxane (15 mL) was added and the reaction mixture stirred at ambient temperature for 1.5 hour. After this time, LCMS analysis of an aliquot showed the reaction to be complete. The solvent was removed under vacuum and the residue azeotroped with dichloromethane (2×30 mL) twice to give 1.41 g (99%) of the desired product as a beige solid which was used in the next step without further purification.

¹H NMR (250 MHz, MeOD) δ ppm 9.19 (s, 1H) 8.41 (d, J=9.44 Hz, 1H) 7.77 (s, 1H) 7.67 (s, 1H) 7.58 (d, J=9.44 Hz, 1H) 5.86 (br. s., 1H) 5.49-5.71 (m, 1H) 5.22-5.37 (m, 1H) 5.14 (dd, J=10.36, 1.22 Hz, 1H) 4.70-4.83 (m, 1H) 4.05 (s, 3H) 3.96 (s, 2H) 3.03 (br. s., 1H) 2.78-2.93 (m, 6H) 2.60 (s, 4H) 2.31 (s, 1H) 1.84-1.98 (m, 1H) 1.42 (d, J=6.85 Hz, 6H) 1.34 (dd, J=9.44, 5.63 Hz, 1H)

LC-MS: purity 100% (UV), t_{R}1.55 min m/z [M+H]⁺ 643.25 (MET/CR/1981)

### Stage 3b: Synthesis of 400:

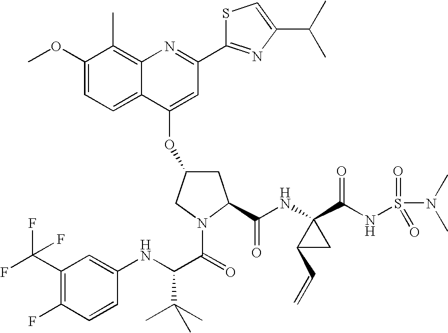

(2S)-2-(4-Fluoro-3-trifluoromethyl-phenylamino)-3,3-dimethyl-butanoic acid (118 mg, 0.404 mmol, 1.0 eq.) was dissolved in N,N-dimethylformamide (4 mL) and HATU (200 mg, 0.525 mmol, 1.3 eq.) was added portionwise. The reaction mixture was stirred at ambient temperature for 10 minutes then cooled to 0° C. Diisopropylethylamine (0.422 mL, 2.424 mmol, 6.0 eq.) was added as a single portion followed by stage 2b intermediate (274 mg, 0.404 mmol, 1.0 eq.). The reaction was left to stir at ambient temperature for 15 hours by when LCMS analysis of an aliquot showed the reaction to be complete. The solvent was removed under vacuum and the residue partitioned between water (20 mL) and ethyl acetate (15 mL). The organic phase was further washed with water (3×15 mL), dried over sodium sulfate, filtered and concentrated to dryness. The residue was purified by flash column chromatography, using a ethyl acetate:heptanes gradient (from neat heptanes to 1:1 ethyl acetate:heptanes). After combining the relevant fractions and solvent removal, 106 mg (29%) of the title compound was isolated as a beige solid.

¹H NMR (500 MHz, CHLOROFORM-d) δ ppm 9.93 (br. s., 1H) 7.45-7.54 (m, 1H) 7.34-7.44 (m, 1H) 7.22-7.26 (m, 1H) 6.98-7.08 (m, 2H) 6.78-6.91 (m, 2H) 6.38-6.52 (m, 2H) 5.63-5.81 (m, 1H) 5.43-5.55 (m, 1H) 5.19-5.28 (m, 1H) 5.09-5.19 (m, 1H) 4.39-4.52 (m, 2H) 4.12-4.22 (m, 1H) 4.02-4.11 (m, 1H) 3.92-4.02 (m, 3H) 3.80-3.90 (m, 1H) 3.12-3.26 (m, 1H) 2.84-2.98 (m, 6H) 2.65-2.74 (m, 3H) 2.52-2.64 (m, 2H) 1.99-2.11 (m, 1H) 1.88-1.99 (m, 1H) 1.34-1.42 (m, 6H) 1.07-1.16 (m, 9H). LC-MS: purity 100% (UV), t_{R} 5.22 min m/z [M+H]⁺918.29 (MET/CR/1426)

### Stage 1b: Synthesis of 401:

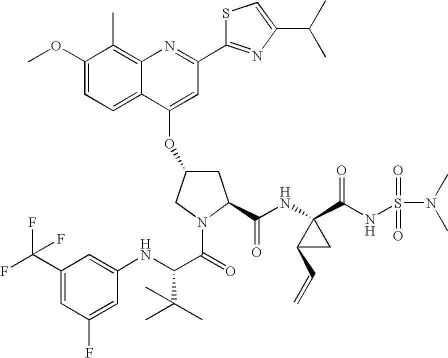

401 was prepared following the same method as 400.

120 mg (32%), beige solid.

¹H NMR (500 MHz, CHLOROFORM-d) δ ppm 9.89 (br. s., 1H) 7.46-7.54 (m, 2H) 7.05 (s, 1H) 7.01 (d, J=9.16 Hz, 1H) 6.91 (br. s., 1H) 6.65 (s, 1H) 6.55 (d, J=8.24 Hz, 1H) 6.37 (d, J=10.83 Hz, 1H) 5.68-5.79 (m, 1H) 5.24 (d, J=17.24 Hz, 1H) 5.16 (d, J=10.53 Hz, 1H) 4.84 (d, J=10.07 Hz, 1H) 4.48 (t, J=8.32 Hz, 1H) 4.17-4.24 (m, 1H) 4.09-4.17 (m, 1H) 3.92-3.98 (m, 4H) 3.20 (spt, J=6.69 Hz, 1H) 2.90 (d, J=2.29 Hz, 6H) 2.68 (s, 3H) 2.55-2.66 (m, 2H) 1.99-2.04 (m, 1H) 1.91-1.98 (m, 1H) 1.40 (d, J=6.87 Hz, 6H) 1.26-1.34 (m, 2H) 1.13 (s, 9H)

LC-MS: purity 100% (UV), t_{R} 5.28 min m/z [M+H]⁺918.30 (MET/CR/1426)

### Preparation of 402 and New Derivatives

### Preparation of N-Aryl Tent-Leucine Amino Acids

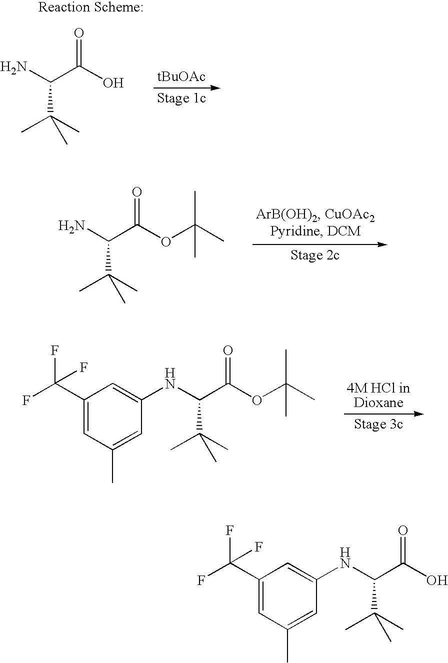

### Stage 1c: (2S)-2-Amino-3,3-dimethyl-butanoic acid tert-butyl ester (456)

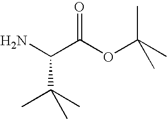

Tert-Leucine (1.5 g, 11.43 mmol, 1.0 eq.) and tert-butyl acetate (30 mL) were charged into a 100 mL round bottom flask and the reaction mixture cooled to 0° C. Perchloric acid (1.72 g, 1 mL, 17.2 mmol, 1.5 eq.) was added dropwise and the reaction mixture was left to warm up to ambient temperature and stirred for a further 48 hours. The organic phase was washed with water (50 mL) and then 1M hydrochloric acid (30 mL). The aqueous phases were combined and the pH adjusted to 9 with 1M aqueous potassium carbonate solution. The aqueous phase was extracted with dichloromethane (3×40 mL). The first organic phase and the dichloromethane extracts were combined, dried over sodium sulfate, filtered and the solvent removed under vacuum (caution: desired product has a low boiling point keep Buchi batch cold and pressure around 100 mbars). The residue was purified by flash column chromatography, using ethyl acetate:heptanes (1:1) as eluent. After combining the relevant fractions and solvent removal, 1.20 g (56%) of the title compound was isolated as a colorless oil.

¹H NMR (250 MHz, CHLOROFORM-d) δ ppm 3.03 (s, 1H) 1.56 (s, 2H) 1.48 (s, 9H) 0.97 (s, 9H)

### Stage 2c: (2S)-2-(3-Methyl-5-trifluoromethylphenylamino)-3,3-dimethyl-butanoic acid tert-butyl ester (457)

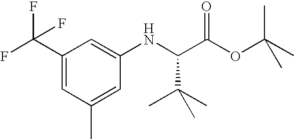

Copper (II) acetate (250 mg, 1.37 mmol, 1.1 eq.) and 4 Å molecular sieves (200 mg) were charged in a 50 mL round bottom flask. Dichloromethane (10 mL, previously saturated with air) was added as a single portion. (2S)-2-Amino-3,3-dimethyl-butanoic acid tert-butyl ester (233 mg, 1.25 mmol, 1.0 eq.) was added and the reaction mixture was stirred for a further 5 min. 3-Methyl-5-trifluoromethylbenzene boronic acid (500 mg, 2.49 mmol, 2 eq.) was added followed by pyridine (0.200 mL, 2.49 mmol, 2 eq.). The reaction mixture was stirred overnight under an air atmosphere. 1M hydrochloric acid (20 mL) was added. The organic layer was collected and the aqueous phase extracted twice with dichloromethane (20 mL). The organic extracts were combined, dried over sodium sulfate, filtered, and the solvent removed under vacuum. The residue was purified by flash column chromatography, using a ethyl acetate:heptanes gradient (from neat heptane to 2.5% ethyl acetate in heptanes). After combining the relevant fractions and solvent removal, 260 mg (73%) of the title compound was isolated as a pale yellow oil.

¹H NMR (500 MHz, CHLOROFORM-d) δ ppm 6.79 (s, 1H) 6.69 (s, 1H) 6.64 (s, 1H) 3.67 (s, 1H) 2.31 (s, 3H) 1.43 (s, 9H) 1.08 (s, 9H)

LC-MS: purity 96% (UV), t_{R} 2.78 min m/z [M+H]⁺346.15 (MET/CR/1278)

### Stage 2c: (2S)-2-(3-Chloro-5-trifluoromethylphenylamino)-3,3-dimethyl-butanoic acid tert-butyl ester (458)

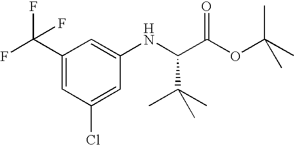

458 was prepared following the same method as 457.

132 mg (36%) as a yellow gum

¹H NMR (250 MHz, CHLOROFORM-d) δ ppm 6.93 (s, 1H) 6.71-6.83 (m, 2H) 4.45 (d, J=9.75 Hz, 1H) 3.65 (d, J=9.90 Hz, 1H) 1.39-1.51 (m, 9H) 1.01-1.14 (m, 9H)

LC-MS: purity 97% (UV), t_{R} 5.90 min m/z [M-tBu+H]⁺309.90 (MET/CR/1416)

### Stage 2c: (2S)-2-(3-Fluoro-5-trifluoromethoxyphenylamino)-3,3-dimethyl-butanoic acid tert-butyl ester (459)

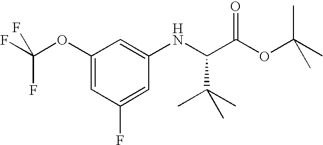

459 was prepared following the same method as 457.

194 mg (50%) as a yellow gum

¹H NMR (500 MHz, CHLOROFORM-d) δ ppm 6.23-6.34 (m, 3H) 4.39-4.46 (m, 1H) 3.60 (d, J=10.07 Hz, 1H) 1.44 (s, 9H) 1.06 (s, 9H)

LC-MS: purity 100% (UV), t_{R} 5.73 min m/z [M-tBu+H]⁺309.95 (MET/CR/1416)

### Stage 3c: (2S)-2-(3-Methyl-5-trifluoromethylphenylamino)-3,3-dimethyl-butanoic acid (460)

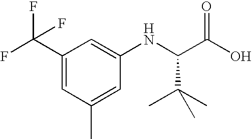

(2S)-2-(3-Methyl-5-trifluoromethylphenylamino)-3,3-dimethyl-butanoic acid tert-butyl ester (260 mg, 0.858 mmol, 1 eq.) was dissolved in 4M HCl in dioxane (4.2 mL). The reaction was heated in a sealed tube at 60° C. for 15 hours. LCMS analysis of a reaction aliquot showed the ester cleavage to be complete. The solvent was removed under vacuum and the residue further dried under vacuum to give 219 mg (88%) of the title compound as a pale yellow gum.

H NMR (500 MHz, CHLOROFORM-d) δ ppm 6.82 (s, 1H) 6.69 (s, 1H) 6.63 (s, 1H) 3.81 (s, 1H) 2.32 (s, 3H) 1.11 (s, 9H)

LC-MS: purity 95% (UV), t_{R} 2.19 min m/z [M+H]⁺290.05 (MET/CR/1278)

### Stage 3c: (2S)-2-(3-Chloro-5-trifluoromethylphenylamino)-3,3-dimethyl-butanoic acid (461)

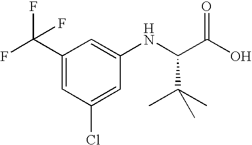

461 was prepared following the same method as 460.

166 mg (98%) as a beige solid

¹H NMR (500 MHz, CHLOROFORM-d) δ ppm 6.97 (s, 1H) 6.79 (s, 1H) 6.76 (s, 1H) 4.42 (br. s., 1H) 3.83 (s, 1H) 1.21-1.31 (m, 1H) 1.11 (s, 9H)

LC-MS: purity 87% (UV), t_{R} 4.81 min m/z [M+H]⁺309.95 (MET/CR/1416)

### Stage 3c: (2S)-2-(3-Fluoro-5-trifluoromethoxyphenylamino)-3,3-dimethyl-butanoic acid (462)

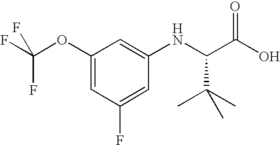

462 was prepared following the same method as 460.

159 mg (97%) as a beige solid

¹H NMR (500 MHz, CHLOROFORM-d) δ ppm 6.33 (d, J=9.16 Hz, 1H) 6.27-6.31 (m, 2H) 4.40 (br. s., 1H) 3.55-3.85 (m, 2H) 1.10 (s, 9H)

LC-MS: purity 95% (UV), t_{R} 4.67 min m/z [M+H]⁺310.00 (MET/CR/1416)

Preparation of P2/P1/P1′ building block (507)

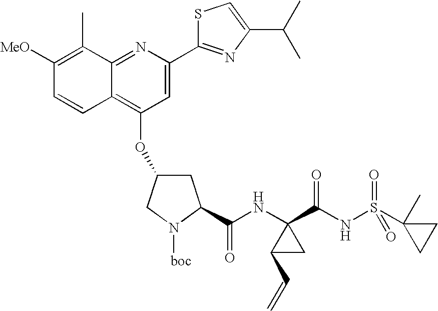

### (2S,4R)-1-(tert-butoxycarbonylamino)-4-[2-(3′-isopropyl-thiazol-2-yl)-7-methoxy-8-methyl-quinoline-4-oxy]-proline

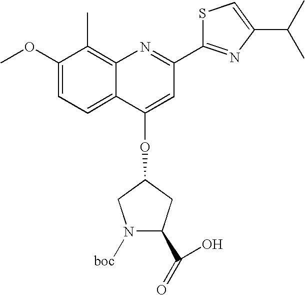

(2S,4R)-1-(tert-Butoxycarbonylamino)-4-hydroxy-proline (24.25 g, 105 mmol, 1.0 eq.) and dimethylsulfoxide (350 mL) were charged into a 2 L round bottom flask. Potassium tert-butoxide (23.56 g, 210 mmol, 2.0 eq.) was added portionwise over 10 minutes at ambient temperature. The reaction mixture was stirred for 1 hour at ambient temperature while the color changed from pale yellow to dark orange. 2-(4-isopropyl-thiazol-2-yl)-4-chloro-7-methoxy-8-methyl-quinoline (35.00 g, 105 mmol, 1.0 eq.) was added portionwise leading to the formation of a brown sticky residue. Further dimethylsulfoxide (150 mL) was added to help solubilize the reagents and the stirring was continued at 35° C. for a further 20 min. As the reaction mixture remained very thick more dimethylsulfoxide (300 mL) was added. The resulting mixture was stirred at 28° C. for 15 hours by which time LCMS analysis of the reaction mixture showed the reaction to be complete. The reaction mixture was diluted with methanol (300 mL) and stirred for 30 min. The reaction mixture was left to cool to ambient temperature and split into two portions to ease the work up. Both fractions were treated in the same way as follows. The mixture was diluted with ethyl acetate (500 mL) and water (300 mL). The aqueous phase was acidified to pH 3 with 1M hydrochloric acid (80 mL) and extracted with ethyl acetate (3×200 mL). The organic extracts were combined, washed with water (5×350 mL) and brine (300 mL), dried over sodium sulfate, filtered and the solvent removed under vacuum to give 24 g and 25 g of crude product respectively. Each solid was purified separately by dry flash chromatography onto 500 g of silica and eluting with a dichloromethane:methanol gradient (from neat dichloromethane to 5% methanol in dichloromethane). After combining the relevant fractions and solvent removal 20.6 g (37%) and 21.7 g (39%) of the desired product were isolated as a yellow solid. The combined yield was 42.3 g (76%).

¹H NMR (500 MHz, CHLOROFORM-d) δ ppm 7.89-8.03 (m, 1H) 7.44-7.56 (m, 1H) 7.24 (d, J=9.16 Hz, 1H) 7.04 (br. s., 1H) 5.39 (br. s., 1H) 4.69 (s, 1H) 4.47-4.60 (m, 1H) 4.00 (s, 3H) 3.98 (br. s., 1H) 3.78-3.88 (m, 1H) 3.18-3.25 (m, 1H) 2.71 (s, 3H) 1.47 (s, 9H) 1.42-1.45 (m, 1H) 1.40 (d, J=6.71 Hz, 6H) 1.36-1.38 (m, 1H)

LC-MS: purity 100% (UV), m/z [M+Na]⁺550.20, 2.65 min (MET/CR/1981).

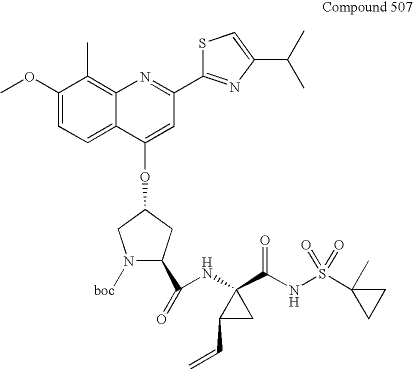

(2S,4R)-1-(tert-Butoxycarbonylamino)-4-[2-(3′-isopropyl-thiazol-2-yl)-7-methoxy-8-methyl-quinoline-4-oxy]-proline (25.00 g, 47.38 mmol, 1.0 eq.) and N,N-dimethylformamide (200 mL) were charged into a 1 L round bottom flask under nitrogen. HATU (21.62 g, 56.86 mmol, 1.2 eq.) and diisopropylethylamine (50 mL, 284.3 mmol, 6.0 eq.) were added at 0° C. and the reaction mixture stirred at ambient temperature for a further 30 minutes. (1R,2S)-1-Amino-2-vinyl-cyclopropane-1-carbonyl-(1′-methyl)cyclopropane-sulfonamide hydrochloride salt (13.98 g, 49.75 mmol, 1.05 eq.), previously dissolved in N,N-dimethylformamide (50 mL) was added dropwise over 15 minutes at 0° C. and stirring was continued for 2 hours ambient temperature. Monitoring the reaction conversion by LCMS showed complete consumption of the starting material. The solvent was removed under vacuum and the residue partitioned between water (0.5 L) and ethyl acetate (0.5 L) leading to the precipitation of a solid. The phases were separated and the solid partitioned between ethyl acetate (1.5 L) and water (3 L). The organic phases were combined, washed with water (2×1 L), dried over sodium sulfate, filtered and the solvent removed under vacuum. The residue was purified by dry flash chromatography, using a heptanes:ethyl acetate gradient (from 4:1 to neat EtOAc). After combining the relevant fractions and solvent removal, 21.0 g (59%) of the title compound was isolated as a yellow solid.

¹H NMR (500 MHz, CHLOROFORM-d) δ ppm 9.79 (br. s., 1H) 7.93 (d, J=9.00 Hz, 1H) 7.51 (br. s., 1H) 7.24 (d, J=9.16 Hz, 1H) 7.16 (br. s., 1H) 7.05 (s, 1H) 5.65-5.88 (m, 1H) 5.37-5.48 (m, 1H) 5.30 (d, J=17.09 Hz, 1H) 5.17 (d, J=10.38 Hz, 1H) 4.40 (t, J=7.78 Hz, 1H) 4.00 (s, 3H) 3.92 (br. s., 2H) 3.12-3.30 (m, 1H) 2.71 (s, 3H) 2.54-2.68 (m, 2H) 2.12 (q, J=8.70 Hz, 1H) 1.99 (dd, J=8.09, 5.80 Hz, 1H) 1.61-1.78 (m, 3H) 1.52 (s, 2H) 1.44-1.50 (m, 9H) 1.33-1.43 (m, 7H) 0.76-0.95 (m, 2H)

LC-MS: purity 98% (UV), m/z [M+H]⁺754.45, 2.50 min (MET/CR/1981).

### Preparation of New Derivatives

### Preparation of 402

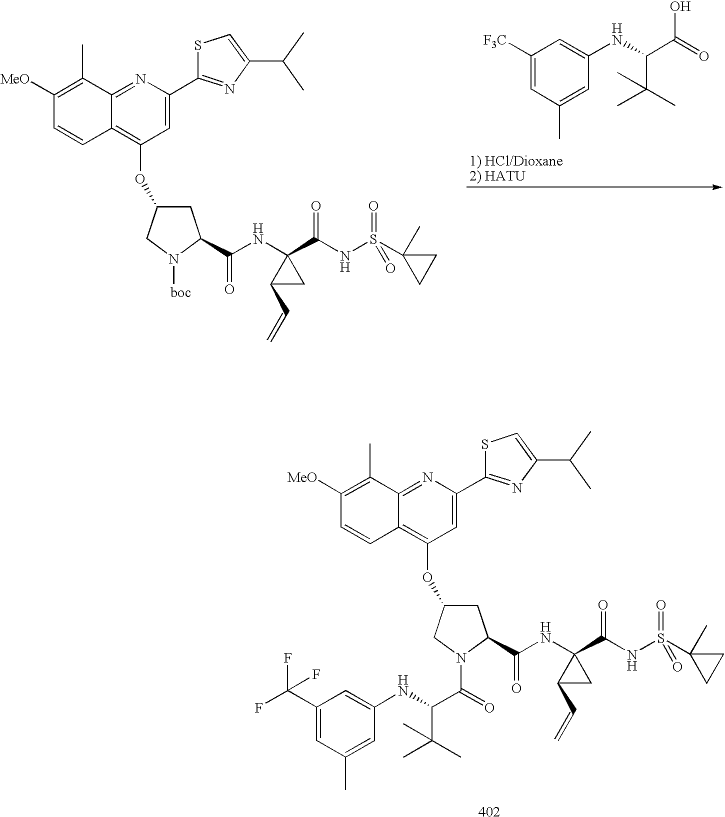

MMQ-proline intermediate (5.404 g, 72.0 mmol, 1 eq.) was dissolved in dioxane (10 mL), then 4M HCl in dioxane (50 mL) was added portion wise. The reaction mixture was stirred at ambient temperature for 15 hours by which time LCMS analysis of an aliquot showed the reaction to be complete. The solvent was removed under vacuum and the solid (463) used in the next step without purification.

The amino acid (460, 160 mg, 0.554 mmol, 1.1 eq.) was dissolved in N,N-dimethylformamide (7 mL) and HATU (214 mg, 0.565 mmol, 1.1 eq.) was added portion wise. The reaction mixture was stirred at ambient temperature for 10 minutes then cooled to 0° C. Diisopropylethylamine (390 mg, 3.03 mmol, 6 eq.) was added as a single portion followed by the MMQ-proline intermediate (463, 348 mg, 0.504 mmol, 1.0 eq.). The reaction was left to stir at ambient temperature for 15 hours, at which time LCMS analysis of an aliquot showed the reaction to be complete. The solvent was removed under vacuum and the residue partitioned between water (20 mL) and ethyl acetate (20 mL). The organic phase was further washed with water (20 mL), dried over sodium sulfate, filtered and concentrated to dryness. The residue was purified by flash column chromatography, using a methanol:dichloromethane gradient (from neat dichloromethane to 3% methanol in dichloromethane). The column had to be repeated a second time as the initial product purity was still contaminated with HATU by-product. The second column was repeated using the same gradient. After combining the relevant fractions and solvent removal, 199 mg (43%) of the title compound was isolated as a yellow solid.

¹H NMR (500 MHz, CHLOROFORM-d) δ ppm 9.87 (br. s., 1H) 7.50 (br. s., 1H) 7.40 (d, J=9.16 Hz, 1H) 7.24 (br. s., 1H) 7.06 (s, 1H) 6.96 (d, J=9.31 Hz, 1H) 6.66 (d, J=9.16 Hz, 2H) 6.48 (s, 1H) 5.61-5.75 (m, 1H) 5.50 (br. s., 1H) 5.26 (d, J=17.09 Hz, 1H) 5.15 (d, J=10.83 Hz, 1H) 4.61 (d, J=9.16 Hz, 1H) 4.55 (t, J=8.24 Hz, 1H) 4.07-4.23 (m, 2H) 4.00 (br. s., 1H) 3.95 (s, 3H) 3.16-3.29 (m, 1H) 2.67 (s, 3H) 2.56-2.65 (m, 1H) 2.05-2.13 (m, 1H) 2.03 (s, 3H) 1.93 (dd, J=7.93, 6.10 Hz, 1H) 1.68 (dt, J=10.80, 5.36 Hz, 2H) 1.58-1.65 (m, 2H) 1.49 (s, 3H) 1.40 (dd, J=6.71, 1.83 Hz, 6H) 1.12 (s, 9H) 0.77-0.92 (m, 2H)

LC-MS: purity 92% (UV), t_{R} 5.40 min m/z [M+H]⁺925.29 (MET/CR/1426)

### Preparation of 403

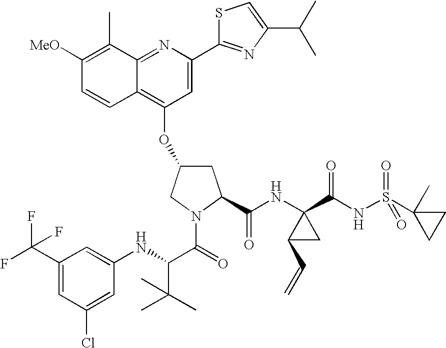

403 was prepared following the same method as 402.

224 mg (48%) as a white solid.

¹H NMR (500 MHz, CHLOROFORM-d) δ ppm 9.82 (br. s., 1H) 7.50 (s, 1H) 7.46 (d, J=9.16 Hz, 1H) 7.12 (br. s., 1H) 7.05 (s, 1H) 6.99 (d, J=9.16 Hz, 1H) 6.82 (s, 1H) 6.71 (s, 1H) 6.67 (s, 1H) 5.66-5.74 (m, 1H) 5.52 (br. s., 1H) 5.26 (d, J=17.24 Hz, 1H) 5.15 (d, J=10.53 Hz, 1H) 4.82 (d, J=10.22 Hz, 1H) 4.54 (t, J=8.32 Hz, 1H) 4.16-4.21 (m, 1H) 4.12 (dd, 1H) 3.98 (s, 1H) 3.96 (s, 3H) 3.20 (spt, J=6.92 Hz, 1H) 2.68 (s, 3H) 2.64 (d, J=8.39 Hz, 2H) 2.08 (q, J=8.70 Hz, 1H) 1.94 (dd, J=7.86, 6.18 Hz, 1H) 1.66-1.72 (m, 1H) 1.59-1.65 (m, 1H) 1.49 (s, 3H) 1.41-1.44 (m, 1H) 1.40 (d, J=8.24 Hz, 6H) 1.12 (s, 9H) 0.77-0.91 (m, 2H)

LC-MS: purity 100% (UV), t_{R} 5.49 min m/z [M+H]⁺945.25 (MET/CR/1426)

### Preparation of 404

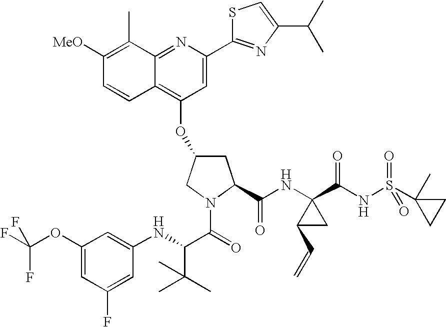

404 was prepared following the same method as 402.

243 mg (55%) as a white solid.

¹H NMR (500 MHz, CHLOROFORM-d) δ ppm 9.82 (br. s., 1H) 7.53 (d, J=9.16 Hz, 1H) 7.50 (s, 1H) 7.12 (br. s., 1H) 7.01-7.07 (m, 2H) 6.24 (s, 1H) 6.16-6.22 (m, 2H) 5.65-5.74 (m, 1H) 5.51 (br. s., 1H) 5.26 (d, J=17.09 Hz, 1H) 5.15 (d, J=10.38 Hz, 1H) 4.77 (d, J=10.07 Hz, 1H) 4.55 (t, J=8.24 Hz, 1H) 4.19 (d, 1H) 4.10 (dd, 1H) 3.97 (s, 3H) 3.92 (d, J=10.22 Hz, 1H) 3.20 (spt, J=6.94 Hz, 1H) 2.69 (s, 3H) 2.65 (d, J=8.09 Hz, 2H) 2.08 (q, J=8.65 Hz, 1H) 1.94 (t, 1H) 1.66-1.72 (m, 1H) 1.59-1.64 (m, 1H) 1.49 (s, 3H) 1.41-1.44 (m, 1H) 1.40 (d, J=7.02 Hz, 6H) 1.11 (s, 9H) 0.79-0.90 (m, 2H)

LC-MS: purity 97% (UV), t_{R} 5.37 min m/z [M+H]⁺945.25 (MET/CR/1426)

### Preparation of 405

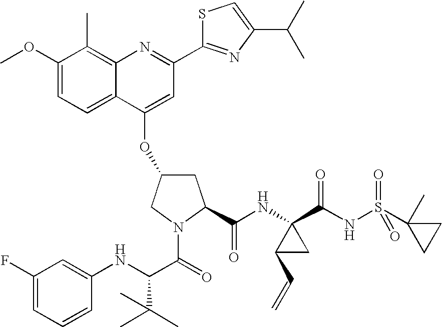

405 was prepared following the same method as 402.

¹H-NMR (DMSO-d₆), δ: 10.37 (s, 1H), 8.73 (s, 1H), 7.62 (d, 1H), 7.51 (s, 1H), 7.46 (d, 1H), 7.21 (d, 1H), 6.68-6.72 (m, 2H), 6.58 (ddd, 1H), 6.42 (dd, 1H), 6.22 (ddd, 1H), 5.64 (m, 1H), 5.48-5.58 (m, 2H), 5.16 (dd, 1H), 5.05 (dd, 1H), 4.43 (d, 1H), 4.34 (dd, 1H), 4.14 (d, 1H), 3.97 (m, 1H), 3.93 (s, 3H), 3.15 (m, 1H), 2.57 (s, 3H), 2.50-2.56 (m, 1H), 2.10-2.22 (m, 2H), 1.64 (dd, 1H), 1.28-1.41 (m, 11H), 1.05 (s, 9H), 0.86-0.90 (m, 2H).

### Preparation of 406

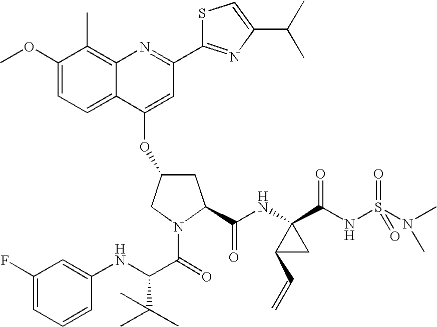

406 was prepared following the same method as 402

¹H-NMR (DMSO-d₆), δ: 10.37 (s, 1H), 8.73 (s, 1H), 7.65 (d, 1H), 7.53 (s, 1H), 7.48 (d, 1H), 7.21 (d, 1H), 6.65-6.73 (m, 2H), 6.60 (ddd, 1H), 6.43 (ddd, 1H), 6.23 (ddd, 1H), 5.65 (m, 1H), 5.51-5.60 (m, 2H), 5.20 (dd, 1H), 5.09 (dd, 1H), 4.45 (d, 1H), 4.36 (dd, 1H), 4.16 (d, 1H), 3.97-4.00 (m, 1H), 3.96 (s, 3H), 3.18 (m, 1H), 2.78 (s, 6H), 2.50-2.56 (m, 1H), 2.09-2.24 (m, 2H), 1.67 (dd, 1H), 1.35 (d, 3H), 1.33 (d, 3H), 1.26-1.30 (m, 2H), 1.07 (s, 9H).

### HPLC Methods:

| | MET/CR/1426 | | MET/CR/1981 | |
|---|---|---|---|---|
| | Method for strongly | | Method for strongly | |
| | retained non-polar | | retained non-polar | |
| | compounds | | compounds | |
| Column | Symmetry Shield RP8 | | Symmetry Shield RP8 | |
| | 2.1 × 50 mm, 3.5 μm | | 2.1 × 50 mm, 3.5 μm | |
| | column | | column | |
| | 40° C. | | 40° C. | |
| | A = Formic acid | | A = Formic acid | |
| | (aq) 0.1% | | (aq) 0.1% | |
| | B = Formic acid | | B = Formic acid | |
| | (acetonitrile) 0.1% | | (acetonitrile) 0.1% | |
| Flow rate | 0.6 ml/min | | 1.0 ml/min | |
| Injection | 3 μl | | 3 μl | |
| volume | | | | |
| Detector | 215 nm (nominal) | | 215 nm (nominal) | |
| | Time (mins) | % Organic | Time (mins) | % Organic |
| Gradient | 0 | 5 | 0 | 5 |
| | 5.0 | 100 | 2.20 | 100 |
| | 7.00 | 100 | 2.70 | 100 |
| | 7.10 | 5 | 2.71 | 5 |
| | MET/CR/1278 | | | |
| | Standard 3.5 minute | | MET/CR/1416 | |
| | method | | High resolution method’ | |
| Column | Atlantis dC18 | | Waters Atlantis dC18 | |
| | 2.1 × 50 mm, 5 μm column | | 100 × 2.1 mm, | |
| | 40° C. | | 3 μm column | |
| | | | 40° C. | |
| | A = Formic acid (aq) 0.1% | | A - 0.1% Formic acid | |
| | B = Formic acid | | (water) | |
| | (acetonitrile) 0.1% | | B - 0.1% Formic acid | |
| | | | (acetonitrile) | |
| Flow rate | 1 ml/min | | 0.6 ml/min | |
| Injection | 3 μl | | 3 μl | |
| volume | | | | |
| Detector | 215 nm (nominal) | | 215 nm (nominal) | |
| | Time (mins) | Time (mins) | Time (mins) | % Organic |
| Gradient | 0 | 0.00 | 0.00 | 5 |
| | 2.5 | 5.00 | 5.00 | 100 |
| | 2.7 | 5.40 | 5.40 | 100 |
| | 2.71 | 5.42 | 5.42 | 5 |

The compounds below may be prepared in a manner analogous to that used to prepare compound 295.

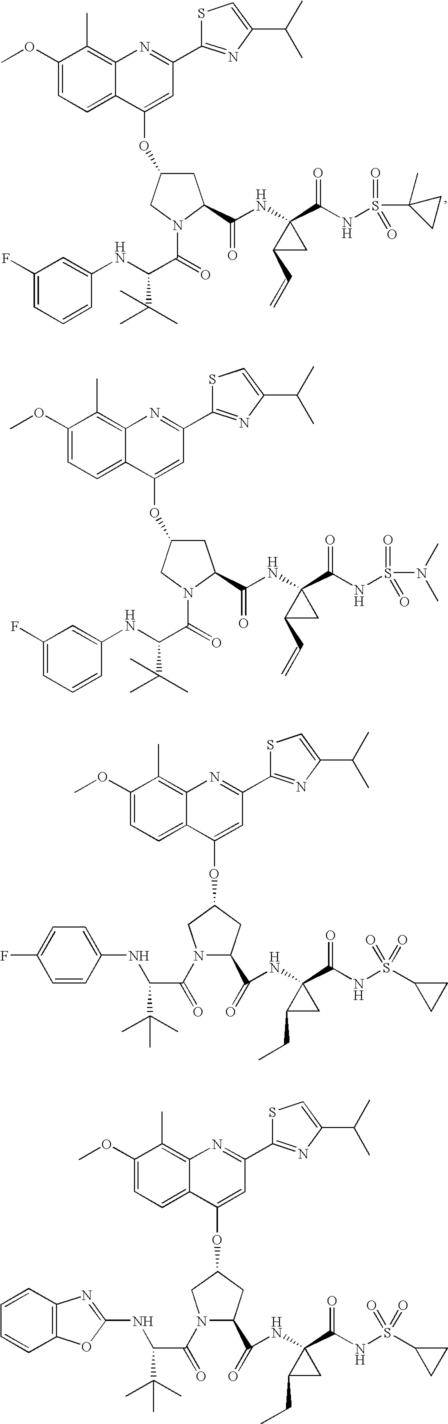

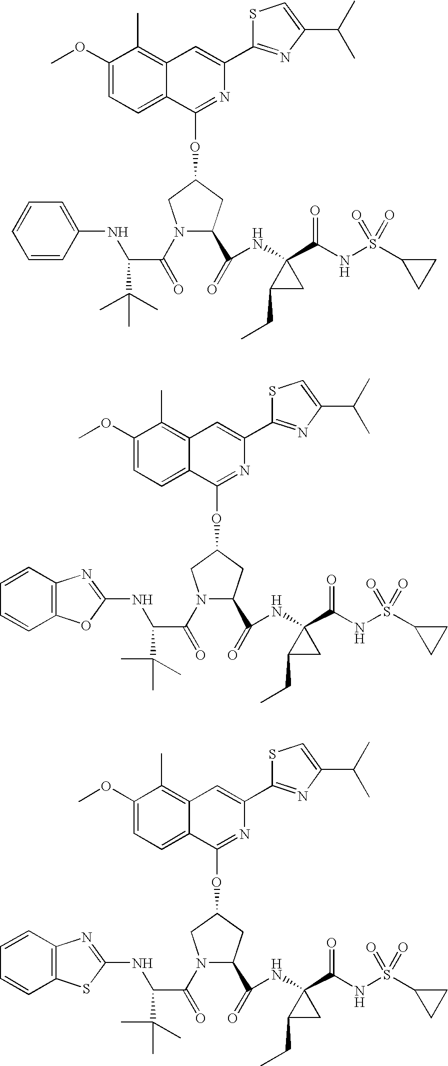

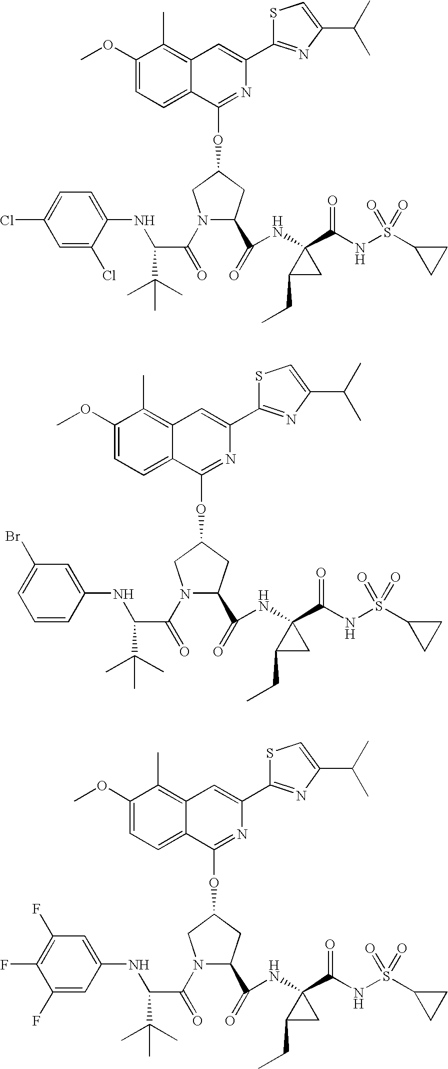

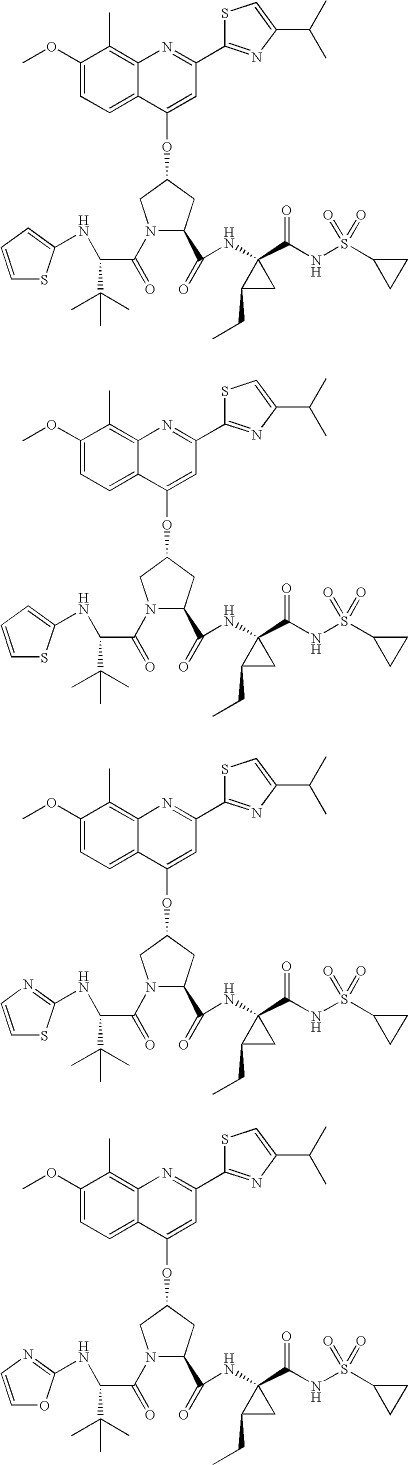

The compounds below may be prepared in a manner analogous to that used to prepare compound 295.

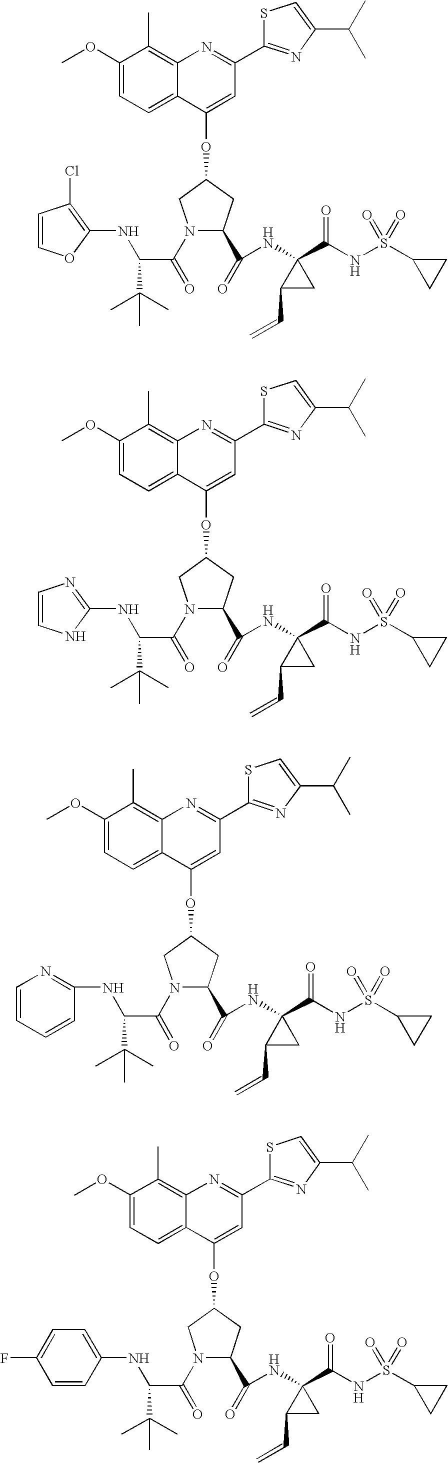

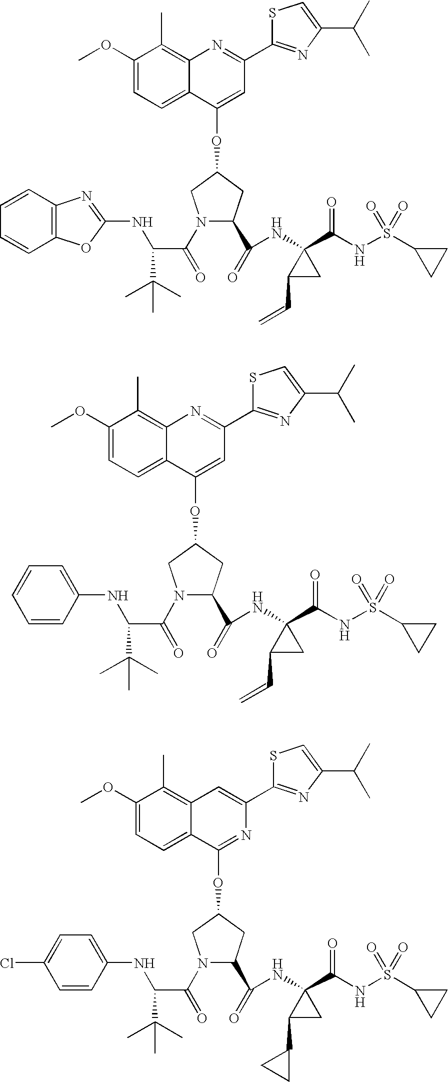

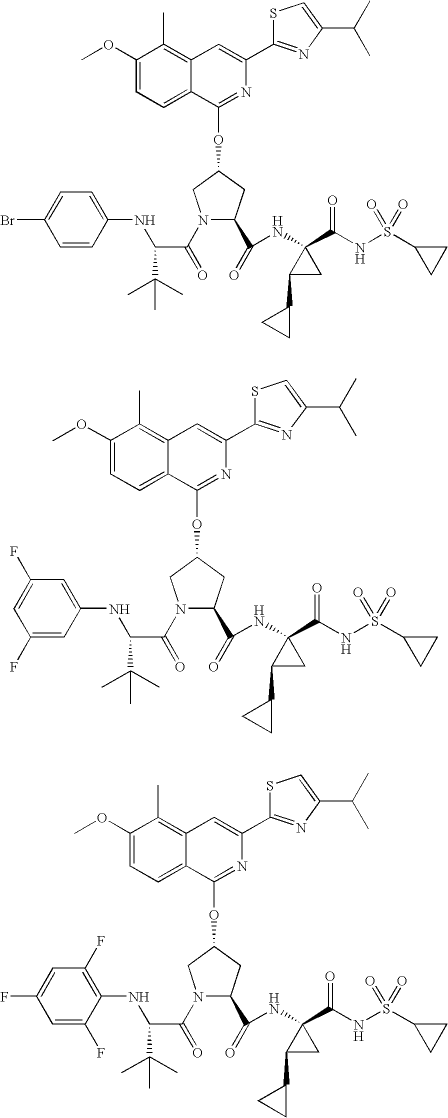

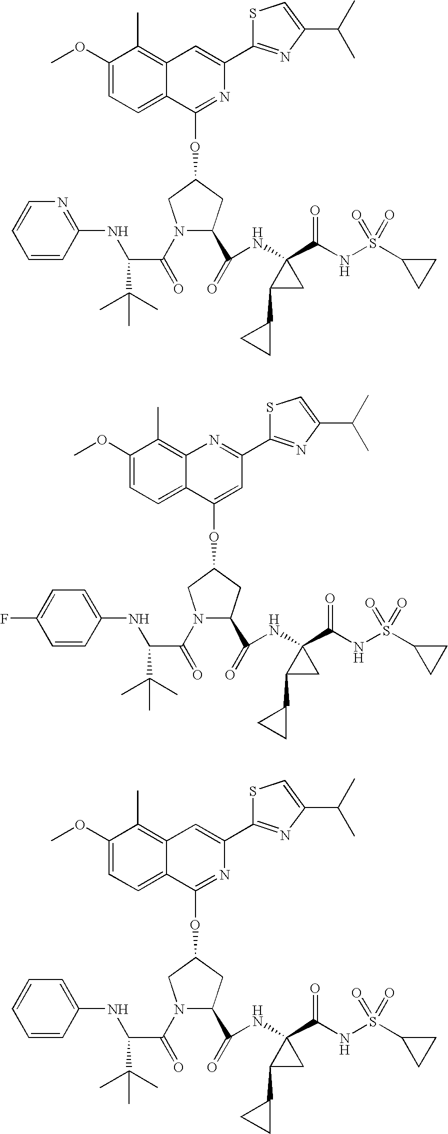

### Example A

### NS3-NS4 Protease Assay

NS3 Complex Formation with NS4A-2.

Recombinant E. coli or Baculovirus full-length NS3 was diluted to 3.33 μM with assay buffer and transferred material to an eppendorf tube and placed in a water bath in a 4° C. refrigerator. The appropriate amount of NS4A-2 diluted to 8.3 mM in assay buffer was added to equal the volume of NS3 above (conversion factor—3.8 mg/272 μL assay buffer). The material was transferred to an eppendorf tube and placed in water bath in a 4° C. refrigerator.

After equilibration to 4° C., equal volumes of NS3 and NS4A-2 solutions were combined in an eppendorf tube, mix gently with a manual pipettor, and incubate mixture for 15 minutes in the 4° C. water bath. Final concentrations in the mixture are 1.67 μM NS3, 4.15 mM NS4A-2 (2485-fold molar excess NS4A-2).

After 15 minutes at 4° C., the NS3/NS4A-2 eppendorf tube was removed and place it in a room temperature water bath for 10 minutes. NS3/NS4A-2 was aliquoted at appropriate volumes and store at −80° C. (E. coli NS3 run at 2 nM in assay, aliquot at 25 μL. BV NS3 run at 3 nM in assay, aliquot at 30 μL).

### Example B

### NS3 Inhibition Assay

Step a. Sample compounds were dissolved to 10 mM in DMSO then diluted to 2.5 mM (1:4) in DMSO. Typically, compounds were added to an assay plate at 2.5 mM concentration, yielding upon dilution a starting concentration of 50 microM in the assay inhibition curve. Compounds were serial diluted in assay buffer to provide test solutions at lower concentrations.

Step b. The E. coli. NS3/NS4A-2 was diluted to 4 nM NS3 (1:417.5 of 1.67 μM stock−18 μL1.67 μM stock+7497 μL assay buffer). The BV NS3/NS4A-2 was diluted to 6 nM NS3 (1:278.3 of 1.67 μM stock−24 μL1.67 μM stock+6655 μL assay buffer).

Step c. Using the manual multichannel pipettor, and being careful not to introduce bubbles into the plate, 50 μL assay buffer were added to wells A01-H01 of a black Costar 96-well polypropylene storage plate.

Step d. Using the manual multichannel pipettor, and being careful not to introduce bubbles into the plate, 50 μL of diluted NS3/NS4A-2 from Step b were added to wells A02-H12 of the plate in Step c.

Step e. Using the manual multichannel pipettor, and being careful not to introduce bubbles into the plate, 25 μL of the wells in drug dilution plate in Step a were transferred to corresponding wells in assay plate in Step d. The tips on the multichannel pipettor were changed for each row of compounds transferred.

Step f Using the manual multichannel pipettor, and being careful not to introduce bubbles into the plate, the contentes of the wells from the assay plate in Step e were mixed by aspirating and dispensing 35 μL of the 75 μL in each well five times. The tips on multichannel pipettor were changed for each row of wells mixed.

Step g. The plate was covered with a polystyrene plate lid, and the plate from Step f containing NS3 protease and sample compounds was pre-incubated 10 minutes at room temperature.

While plate from Step g is pre-incubating, the RETS1 substrate was diluted in a 15 mL polypropylene centrifuge tube. The RETS1 substrate was diluted to 8 μM (1:80.75 of 646 μM stock—65 μL 646 μM stock+5184 μL assay buffer).

After the plate in Step g was done pre-incubating, and using the manual multichannel, 25 μL of substrate were added to all wells on the plate. The contents of the wells of the plate were quickly mixed, as in Step f, mixing 65 μL of the 100 μL in the wells.

The plate was read in kinetic mode on the Molecular Devices SpectraMax Gemini XS plate reader. Reader settings: Read time: 30 minutes, Interval: 36 seconds, Reads: 51, Excitation λ: 335 nm, Emission λ: 495 nm, cutoff: 475 nm, Automix: off, Calibrate: once, PMT: high, Reads/well: 6, Vmax pts: 21 or 28/51 depending on length of linearity of reaction

IC₅₀s are determined using a four parameter curve fit equation, and converted to Ki's using the following Km's:

Full-length E. coli NS3−2.03 μM

Full-length BV NS3−1.74 μM

where Ki=IC₅₀/(1+[S]/Km))

### Quantitation by ELISA of the Selectable Marker Protein, Neomycin Phosphotransferase II (NPTII) in the HCV Sub-Genomic Replicon, GS4.3

The HCV sub-genomic replicon (1377/NS3-3′, accession No. AJ242652), stably maintained in HuH-7 hepatoma cells, was created by Lohmann et al. Science 285: 110-113 (1999). The replicon-containing cell culture, designated GS4.3, was obtained from Dr. Christoph Seeger of the Institute for Cancer Research, Fox Chase Cancer Center, Philadelphia, Pa.

GS4.3 cells were maintained at 37° C., 5% CO₂, in DMEM (Gibco 11965-092) supplemented with L-glutamine 200 mM (100×) (Gibco25030-081), non-essential amino acids (NEAA)(Biowhittaker 13-114E), heat-inactivated (HI) Fetal Bovine Serum(FBS)(Hyclone SH3007.03) and 750 μg/mL geneticin (G418)(Gibco 10131-035). Cells were sub-divided 1:3 or 4 every 2-3 days.

24 h prior to the assay, GS4.3 cells were collected, counted, and plated in 96-well plates (Costar 3585) at 7500 cells/well in 100 μg standard maintenance medium (above) and incubated in the conditions above. To initiate the assay, culture medium was removed, cells were washed once with PBS (Gibco 10010-023) and 90 μl Assay Medium (DMEM, L-glutamine, NEAA, 10% HI FBS, no G418) was added. Inhibitors were made as a 10× stock in Assay Medium, (3-fold dilutions from 10 μM to 56 μM final concentration, final DMSO concentration 1%), 10 μg were added to duplicate wells, plates were rocked to mix, and incubated as above for 72 h.

An NPTII Elisa kit was obtained from AGDIA, Inc. (Compound direct ELISA test system for Neomycin Phosphotransferase II, PSP 73000/4800). Manufacturer's instructions were followed, with some modifications. 10×PEB-1 lysis buffer was made up to include 500 μM PMSF (Sigma P7626, 50 mM stock in isopropanol). After 72 h incubation, cells were washed once with PBS and 150 μg PEB-1 with PMSF was added per well. Plates were agitated vigorously for 15 minutes, room temperature, then frozen at −70° C. Plates were thawed, lysates were mixed thoroughly, and 100 μl were applied to an NPTII Elisa plate. A standard curve was made. Lysate from DMSO-treated control cells was pooled, serially diluted with PEB-1 with PMSF, and applied to duplicate wells of the ELISA plate, in a range of initial lysate amount of 150 μL-2.5 μL. In addition, 100 μL buffer alone was applied in duplicate as a blank. Plates were sealed and gently agitated at room temperature for 2 h. Following capture incubation, the plates were washed 5×300 μL with PBS-T (0.5% Tween-20, PBS-T was supplied in the ELISA kit). For detection, a 1× dilution of enzyme conjugate diluent MRS-2 (5×) was made in PBS-T, into which 1:100 dilutions of enzyme conjugates A and B were added, as per instructions. Plates were resealed, and incubated with agitation, covered, room temperature, for 2 h. The washing was then repeated and 100 μL, of room temperature TMB substrate was added. After approximately 30 minutes incubation (room temperature, agitation, covered), the reaction was stopped with 50 μL, 3M sulfuric acid. Plates were read at 450 nm on a Molecular Devices Versamax plate reader.

Inhibitor effect was expressed as a percentage of DMSO-treated control signal, and inhibition curves were calculated using a 4-parameter equation: y=A+((B−A)/(1+((C/x)̂D))), where C is half-maximal activity or EC₅₀.

### Examples of Activity

The table below shows examples of active compounds.

| Compound # | Structure | EC₅₀ (nM) | IC₅₀ (nM) |
|---|---|---|---|
| 1 | 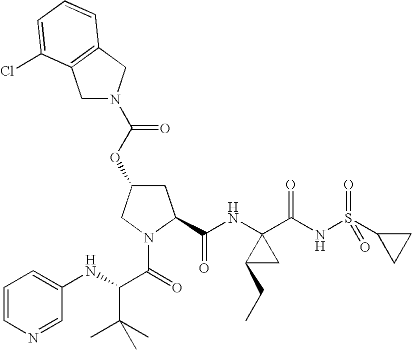 | D | D |
| 2 | 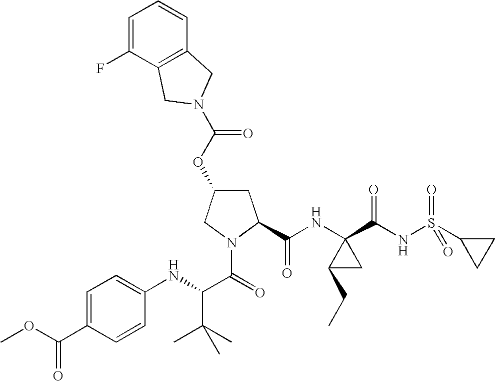 | C | D |
| 3 | 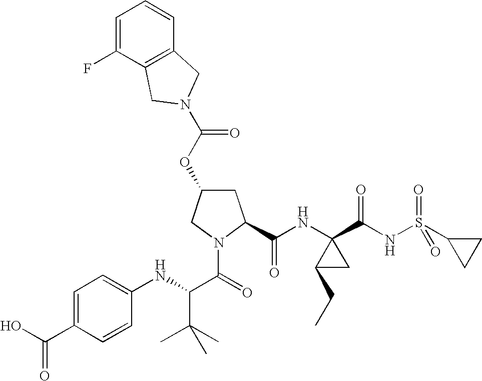 | B | D |
| 4 | 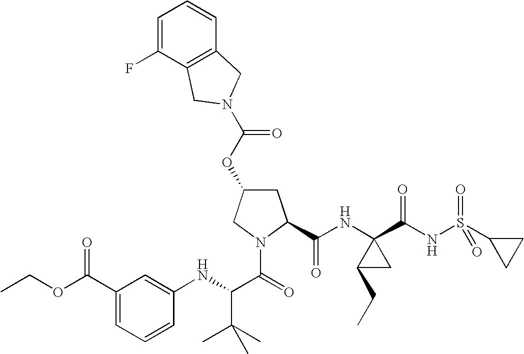 | C | D |
| 5 | 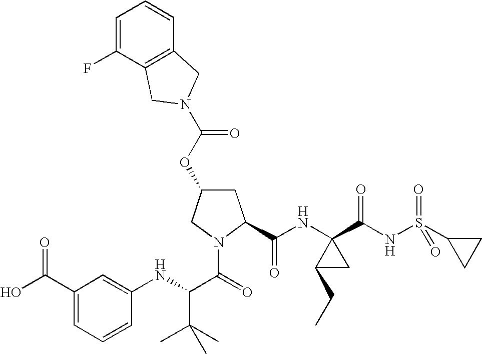 | B | D |
| 6 | 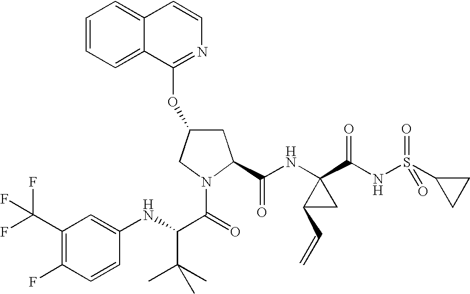 | D | D |
| 7 | 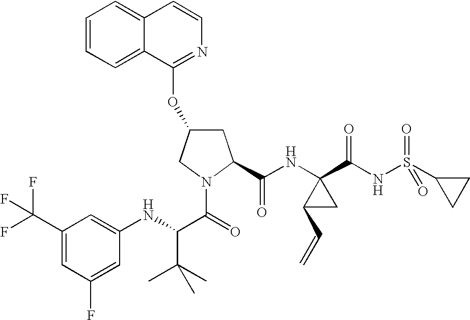 | D | D |
| 8 | 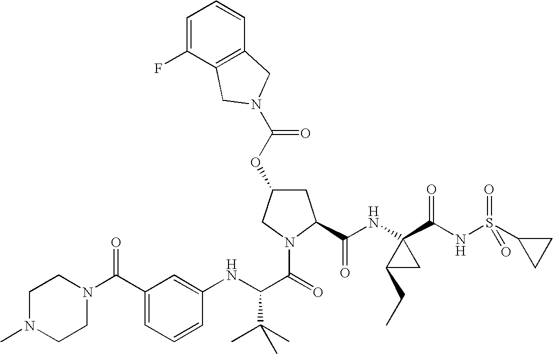 | B | D |
| 9 | 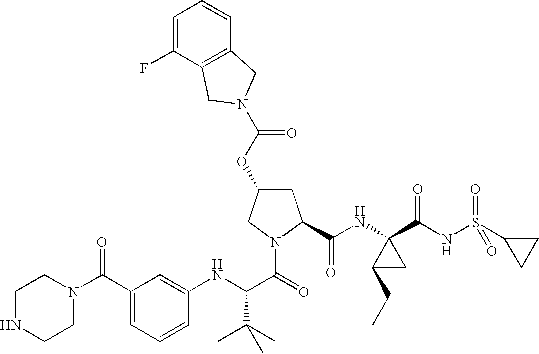 | B | D |
| 10 | 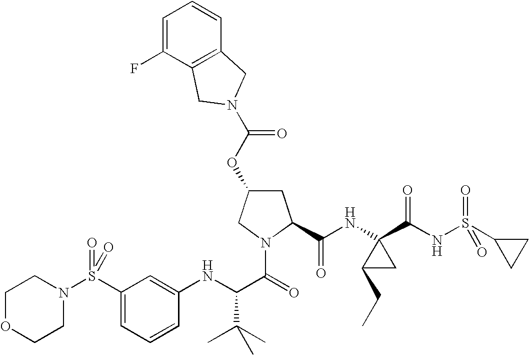 | B | D |
| 11 | 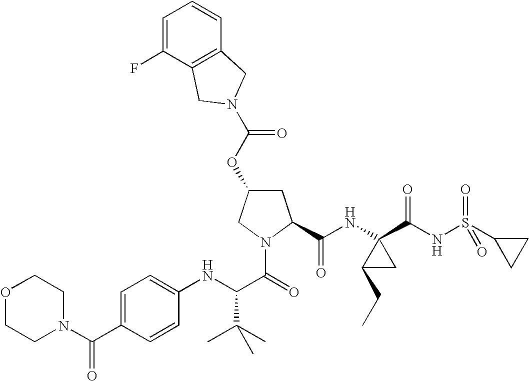 | B | D |
| 12 | 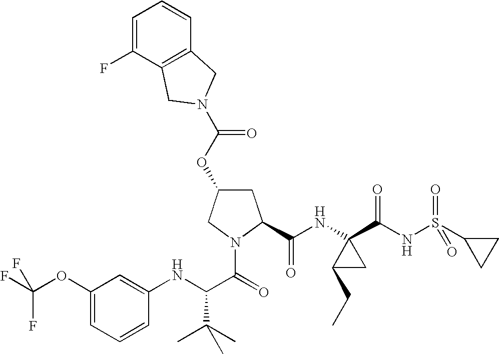 | D | D |
| 13 | 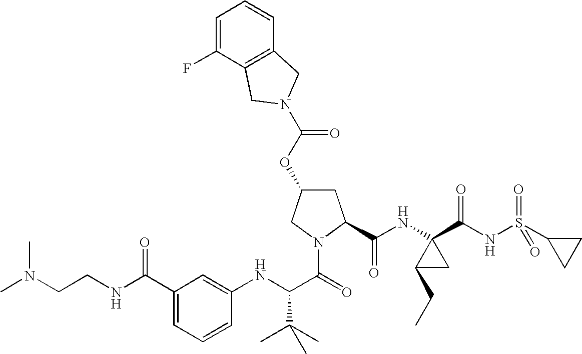 | B | D |
| 14 | 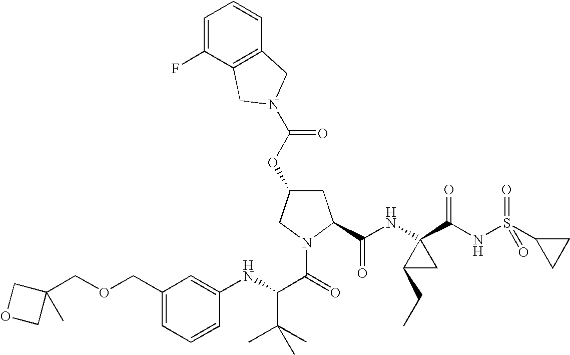 | C | D |
| 15 | 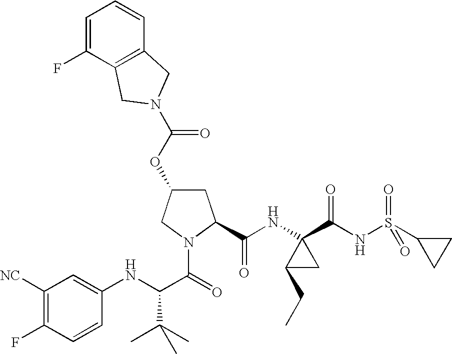 | C | D |
| 16 | 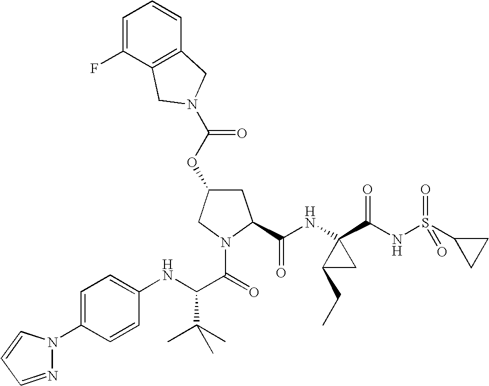 | C | D |
| 17 | 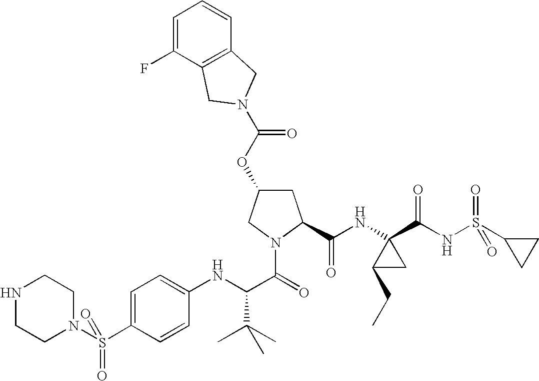 | B | D |
| 18 | 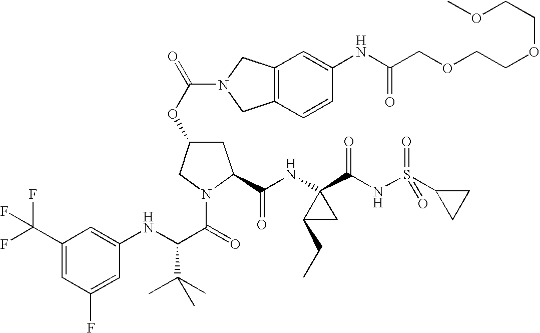 | C | C |
| 19 | 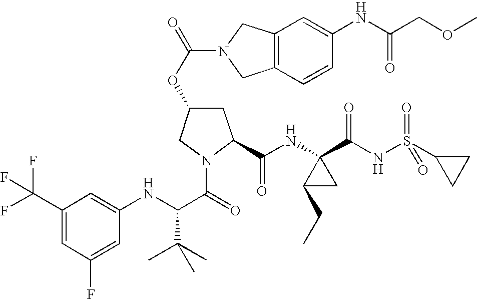 | D | D |
| 20 | 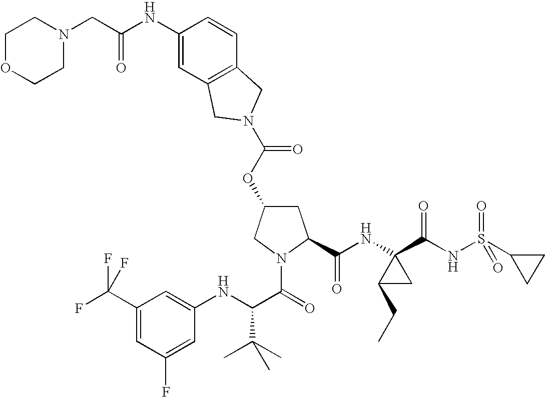 | D | D |
| 21 | 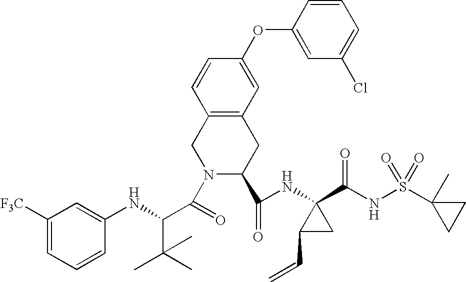 | NA | D |
| 22 | 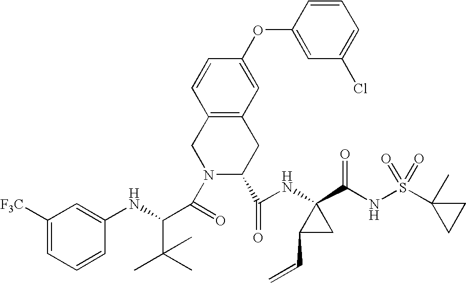 | B | D |
| 23 | 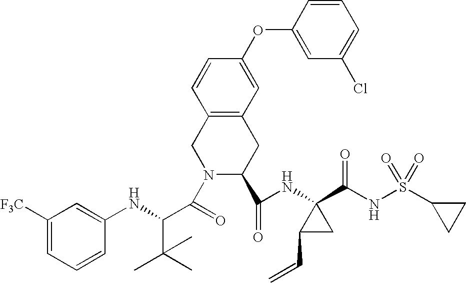 | B | D |
| 24 | 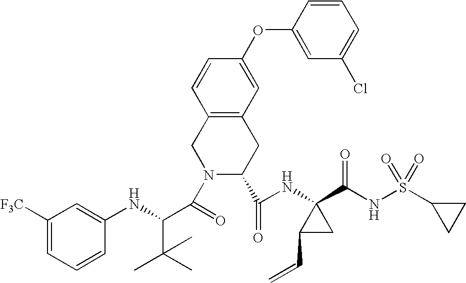 | B | D |
| 25 | 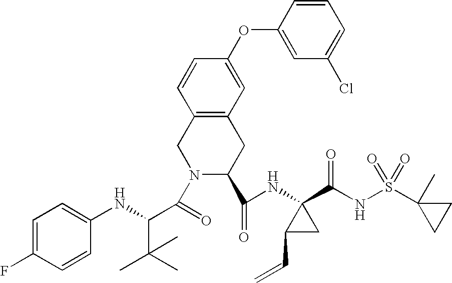 | NA | D |
| 26 | 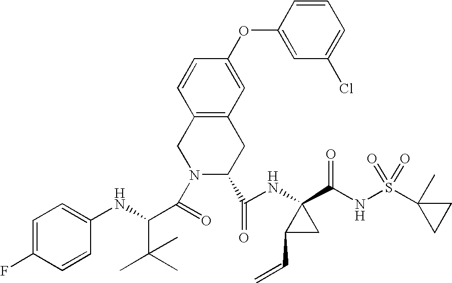 | NA | C |
| 27 | 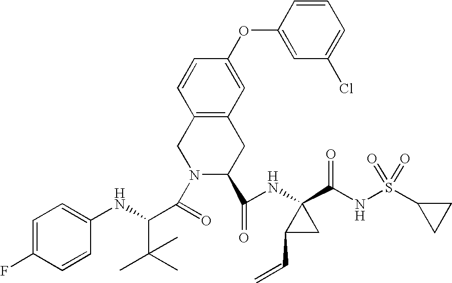 | NA | D |
| 28 | 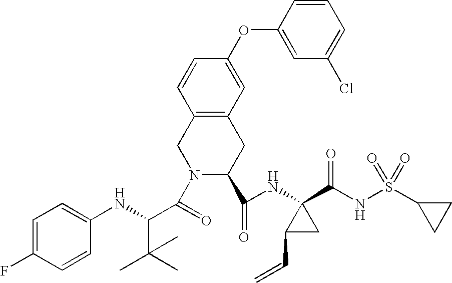 | NA | C |

| Compound | Structure | EC₅₀ (nM) | IC₅₀ (nM) |
|---|---|---|---|
| 230 | 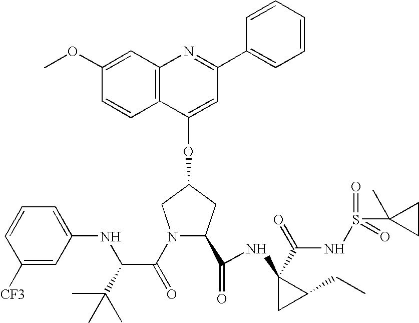 | D | D |
| 231 | 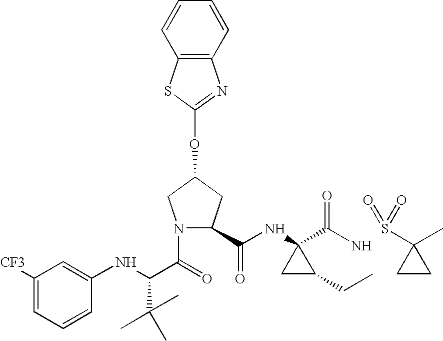 | D | D |
| 232 | 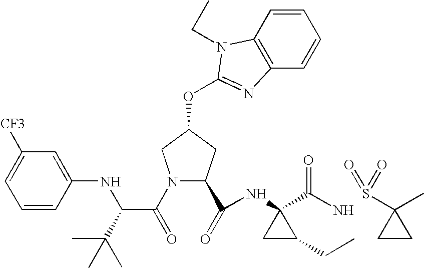 | D | D |
| 261 | 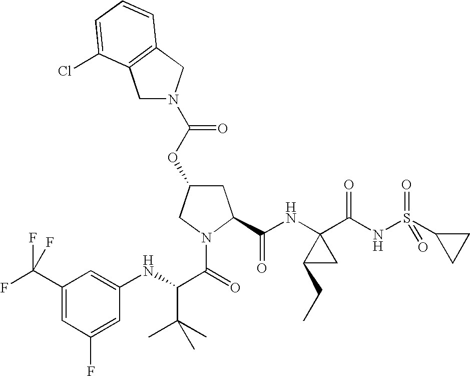 | D | D |
| 281 | 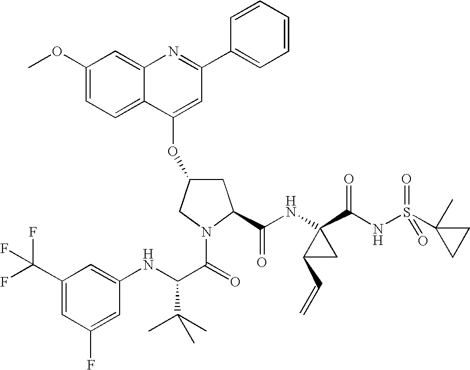 | D | D |
| 282 | 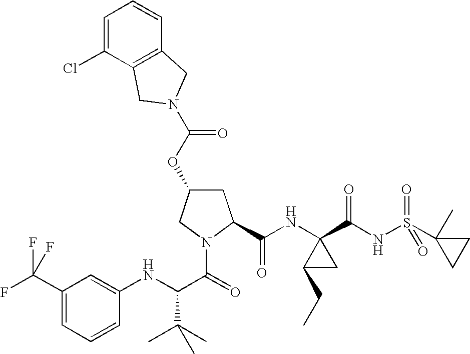 | D | D |
| 283 | 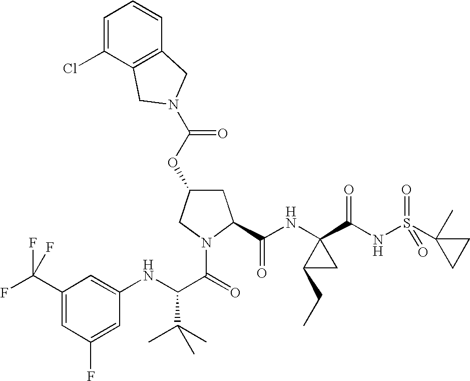 | D | D |
| 284 | 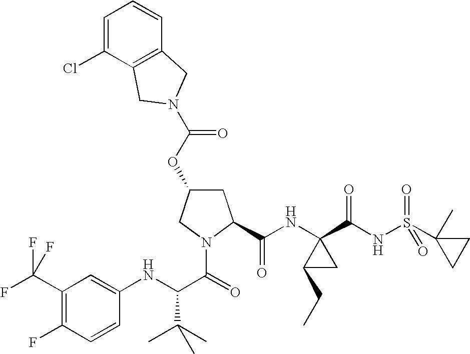 | D | D |
| 285 | 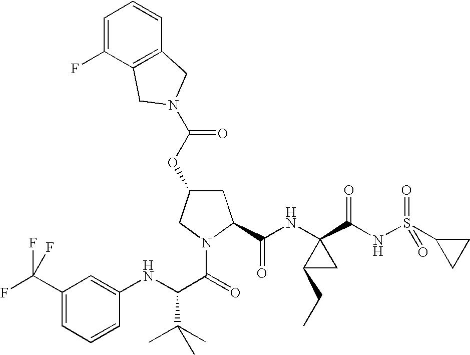 | D | D |
| 286 | 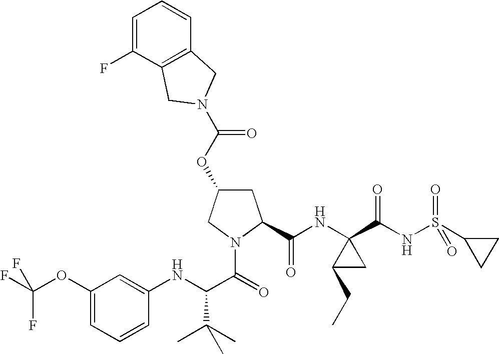 | D | D |
| 287 | 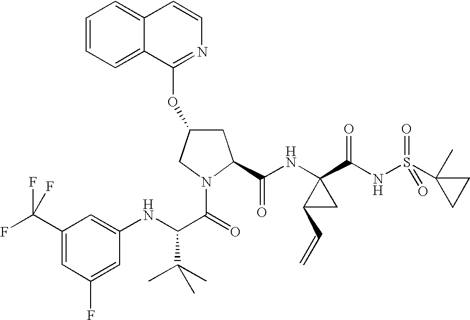 | D | D |
| 288 | 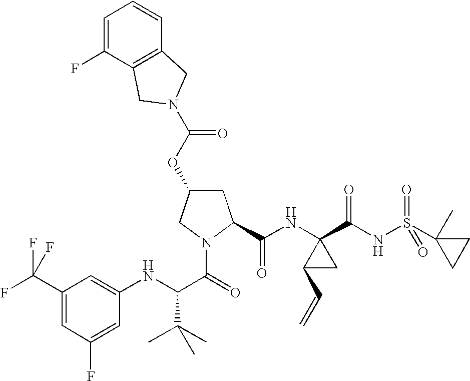 | D | D |
| 289 | 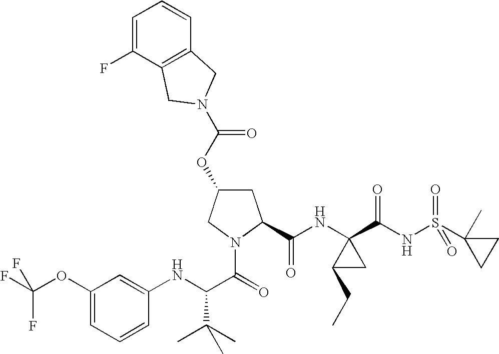 | D | D |
| 290 | 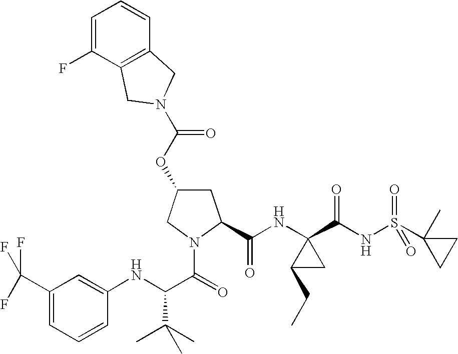 | D | D |
| 291 | 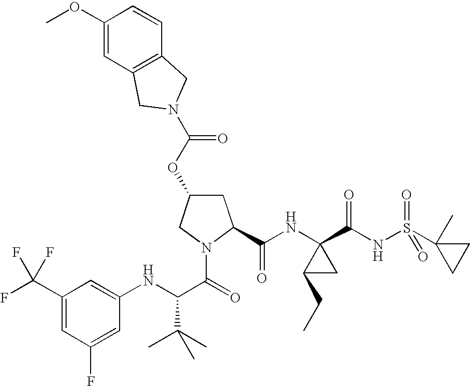 | D | D |
| 292 | 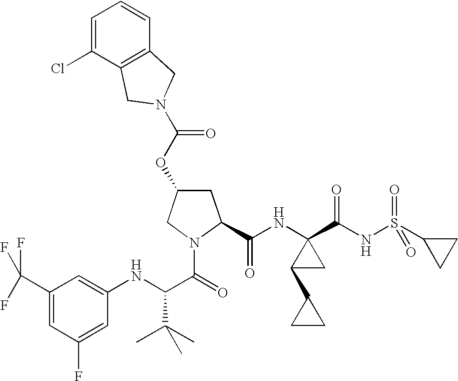 | D | D |
| 293 | 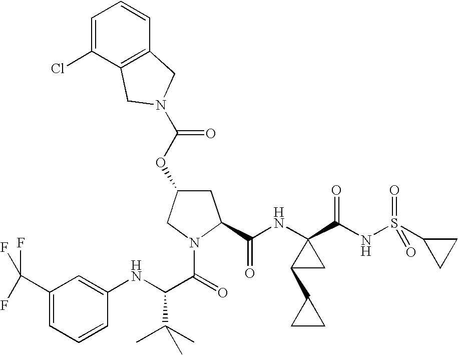 | D | D |
| 294 | 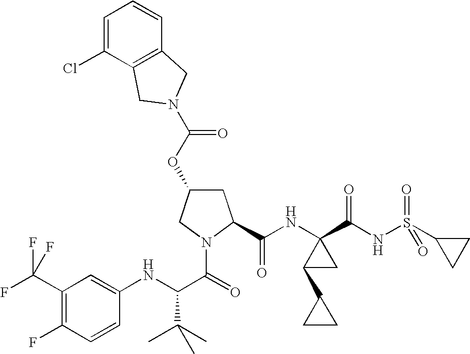 | D | D |
| 297 | 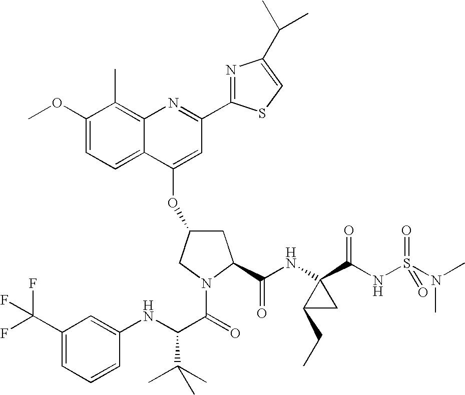 | D | D |
| 298 | 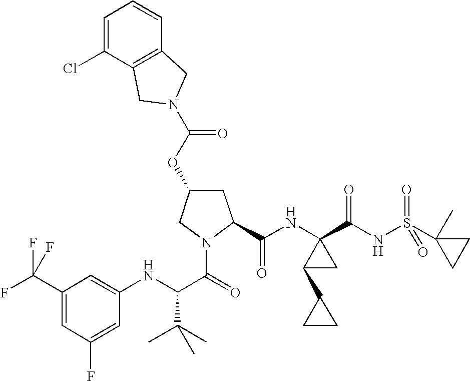 | D | D |
| 299 | 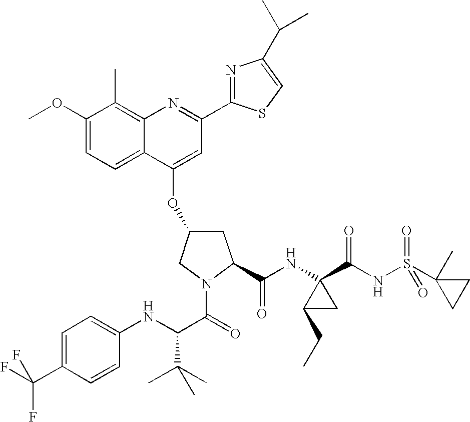 | D | D |
| 300 | 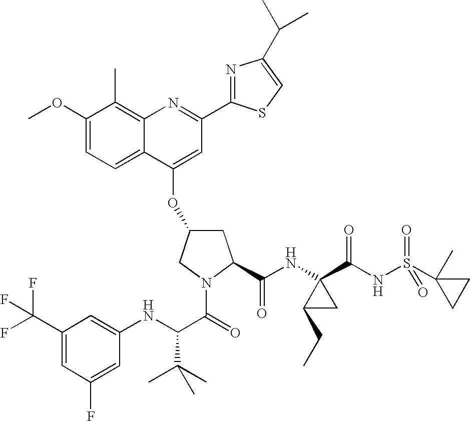 | D | NA |
| 301 | 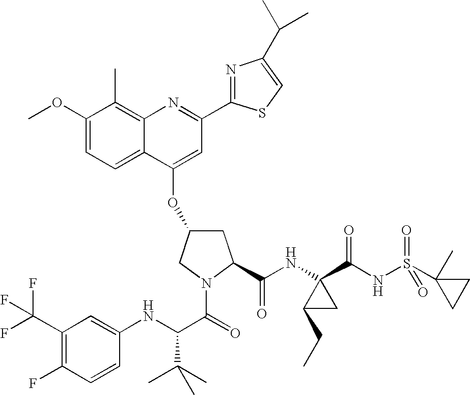 | D | D |
| 302 | 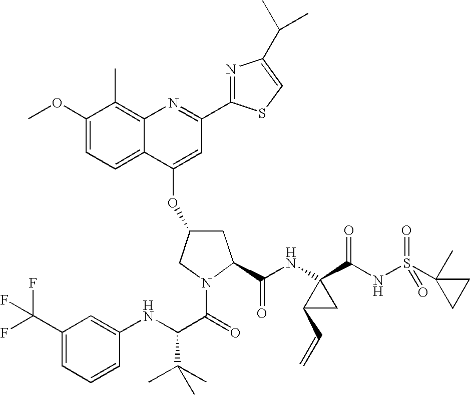 | D | D |
| 303 | 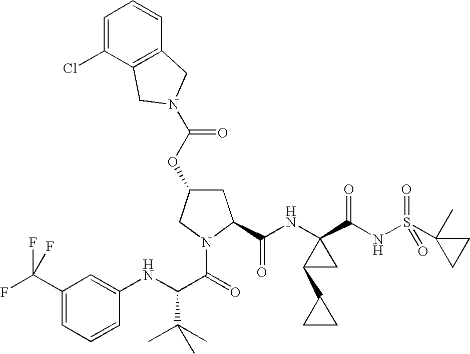 | D | D |
| 304 | 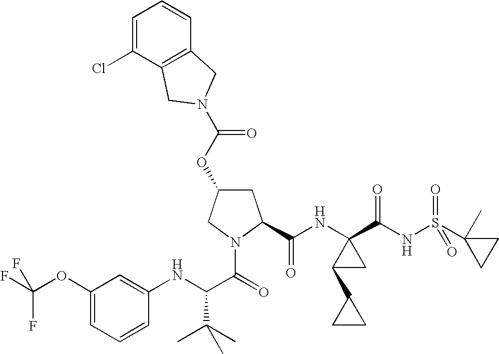 | D | D |
| 305 | 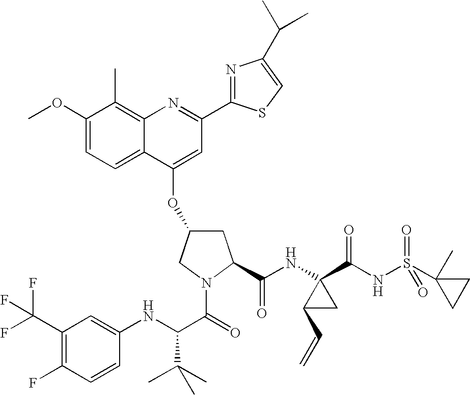 | D | D |
| 306 | 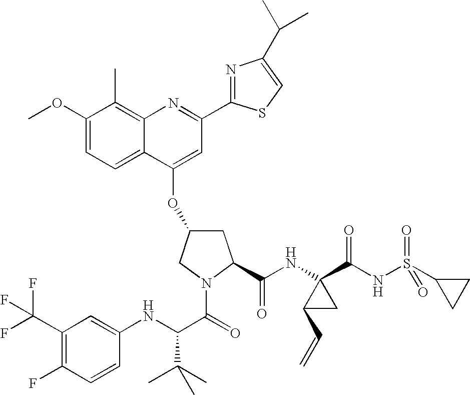 | D | D |
| 307 | 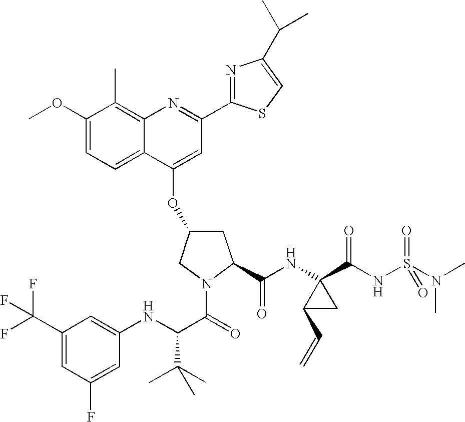 | D | D |
| 308 | 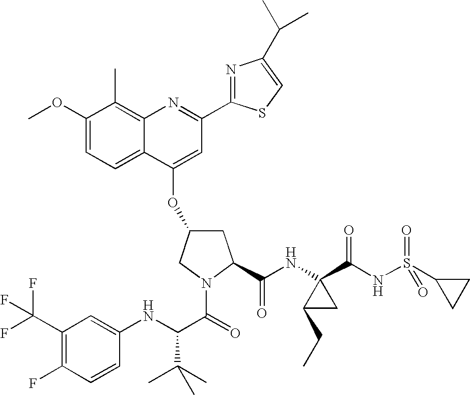 | D | D |
| 309 | 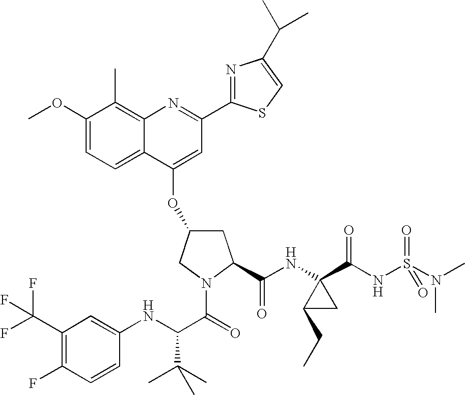 | D | D |
| 310 | 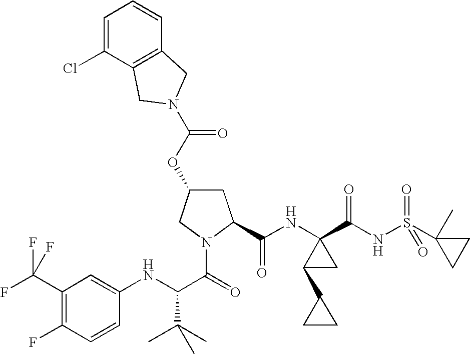 | D | D |
| 314 | 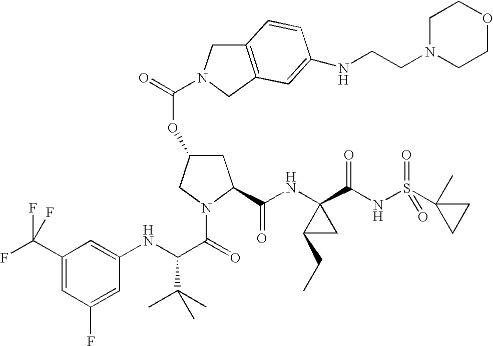 | D | D |
| 334 | 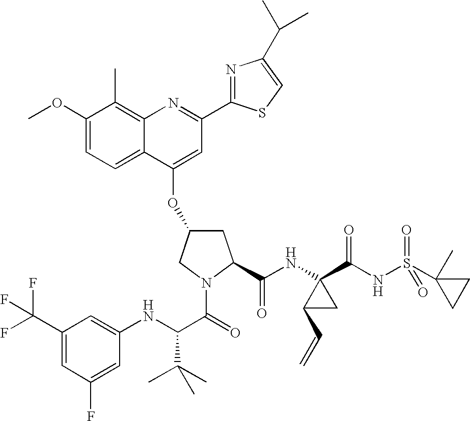 | D | D |
| 335 | 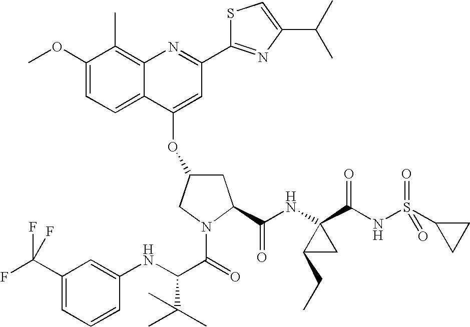 | D | D |
| 335Na | 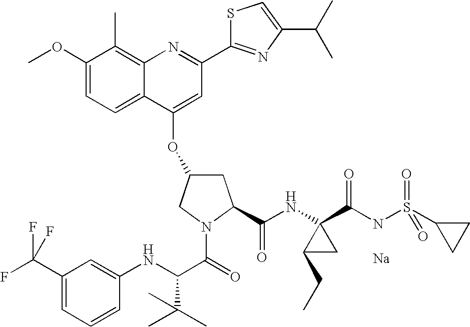 | | |
| 336 | 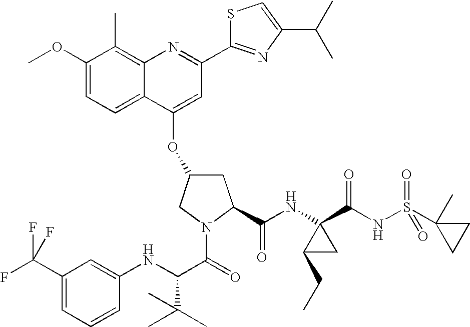 | D | D |
| 336Na | 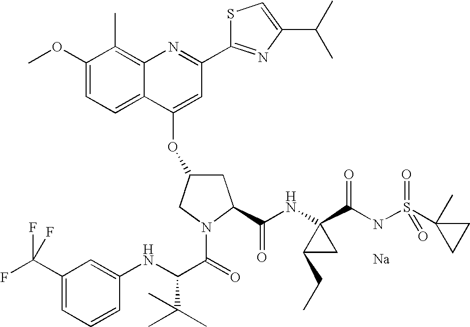 | | |
| 350 | 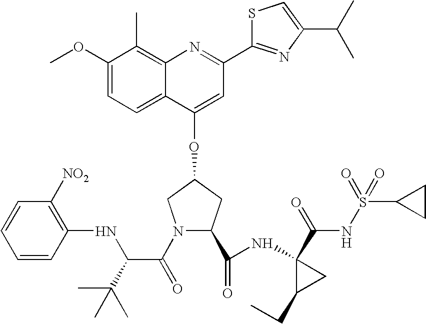 | D | B |
| 351 | 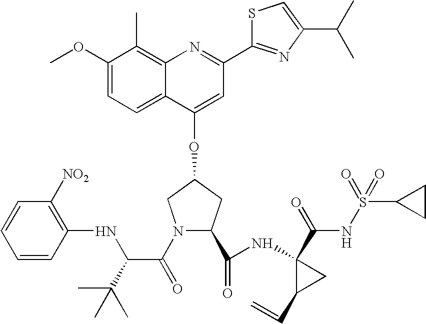 | D | C |
| 352 | 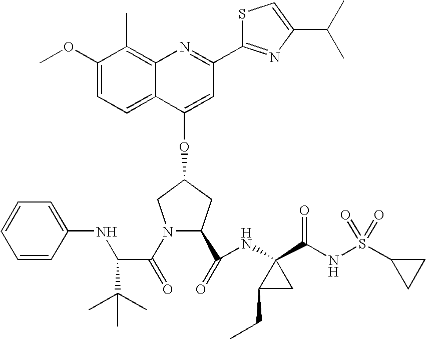 | D | D |
| 353 | 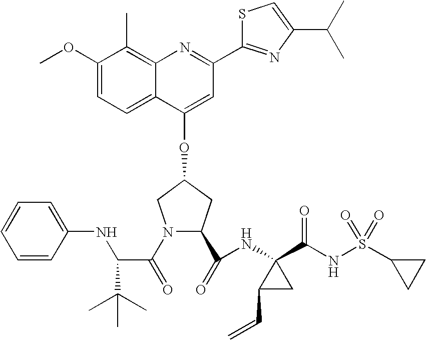 | D | D |
| 354 | 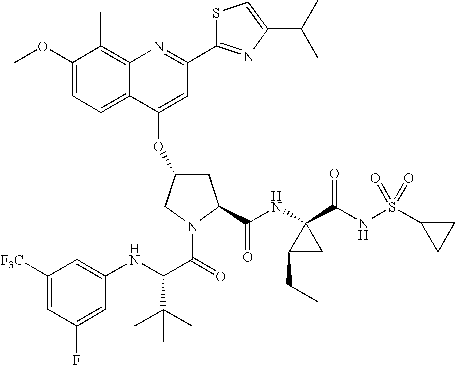 | D | D |
| 400 | 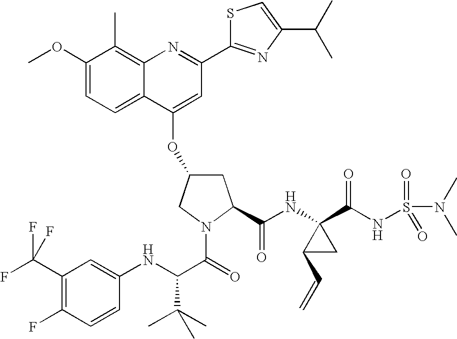 | D | D |
| 401 | 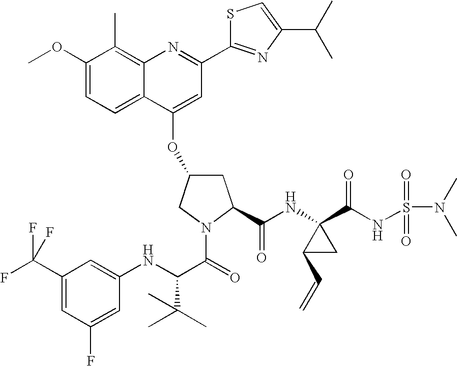 | D | D |
| 402 | 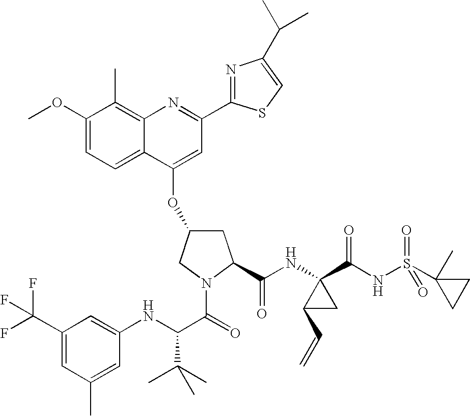 | D | D |
| 403 | 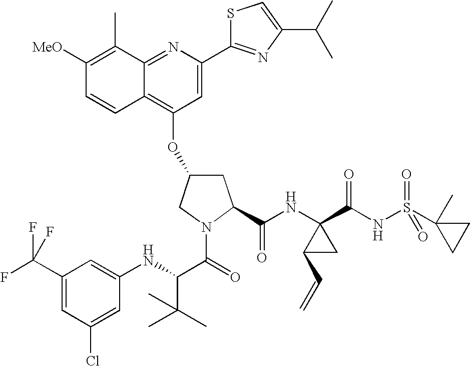 | D | D |
| 404 | 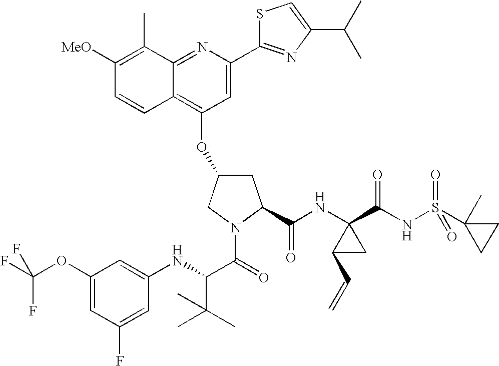 | D | D |

B indicates an EC₅₀ or IC₅₀ > 1 μM
C indicates an EC₅₀ or IC₅₀ between 0.1 and 1 μM
D indicates an EC₅₀ or IC₅₀ of less than 0.1 μM
NA means not available.

### CONCLUSION

Potent small molecule inhibitors of the HCV NS3 protease have been developed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A compound represented by a formula: 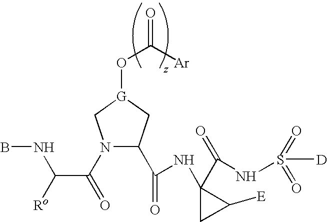
or a pharmaceutically acceptable salt thereof,
wherein Ar is optionally substituted fused bicyclic heteroaryl, optionally substituted C₆₋₁₀ aryl, or optionally substituted isoindolinyl;
z is 0 or 1;
G is 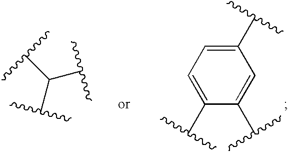
B is optionally substituted C₆₋₁₀ aryl or optionally substituted heteroaryl;
R^{o} is H or C₁₋₁₂ hydrocarbyl;
D is C₁₋₁₀ alkyl or NR¹¹R¹², wherein R¹¹ and R¹² are independently H or C₁₋₅ alkyl and wherein R¹¹ and R¹² may be connected to form one or more rings; and
E is C₁₋₆ hydrocarbyl;
provided that the compound is not:

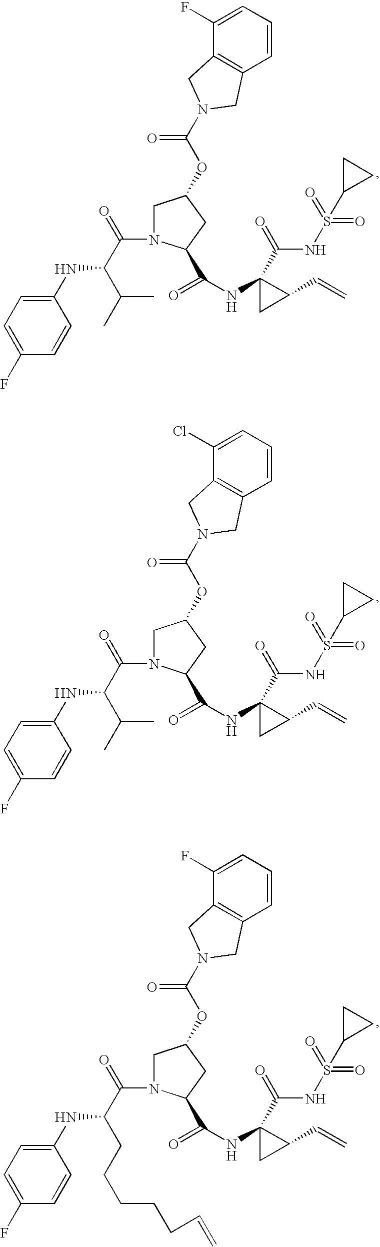

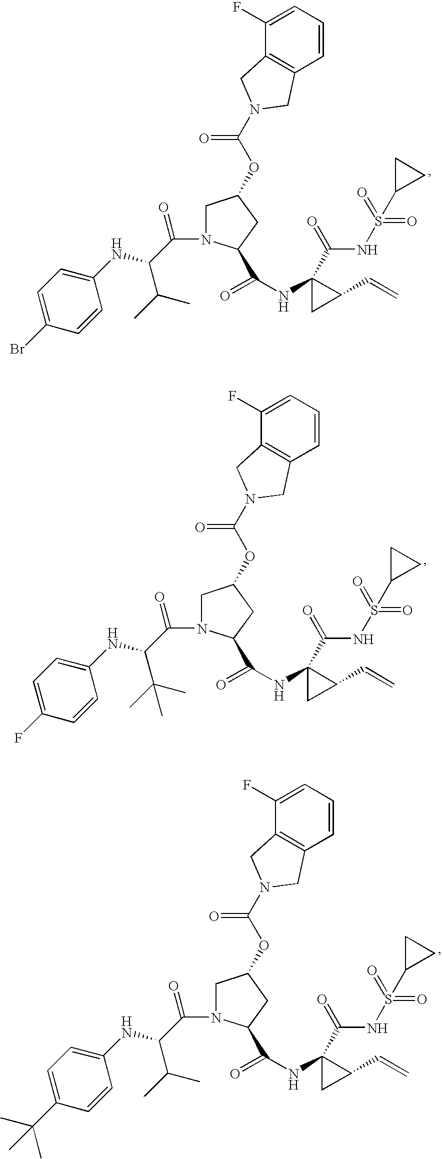

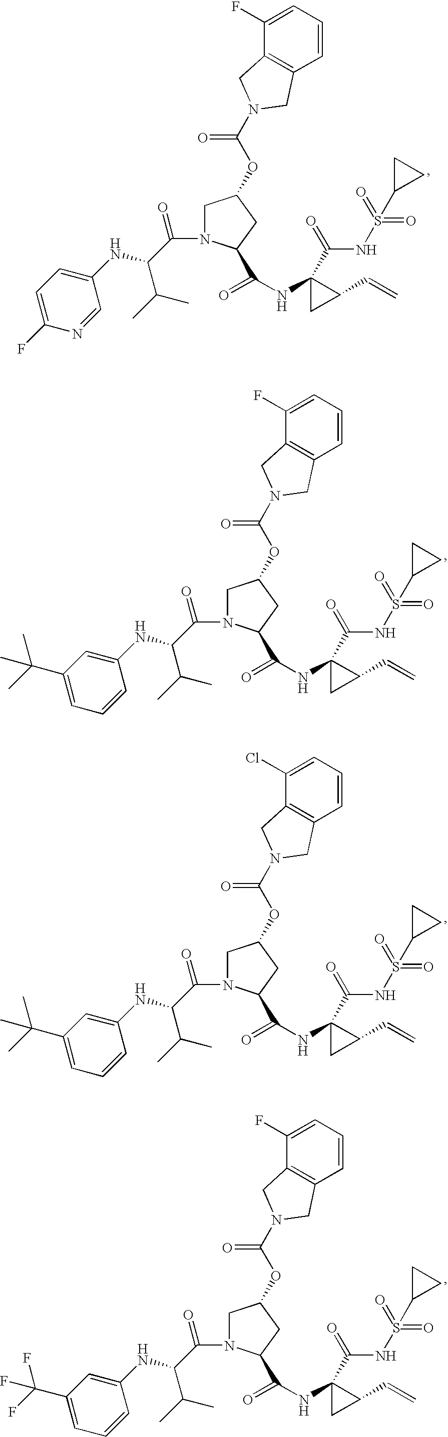

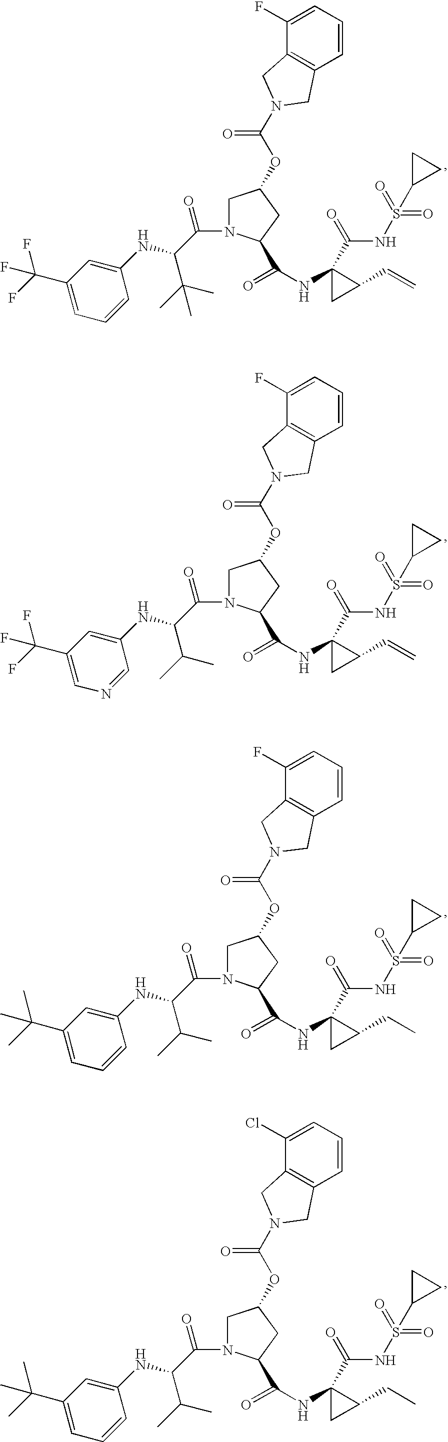

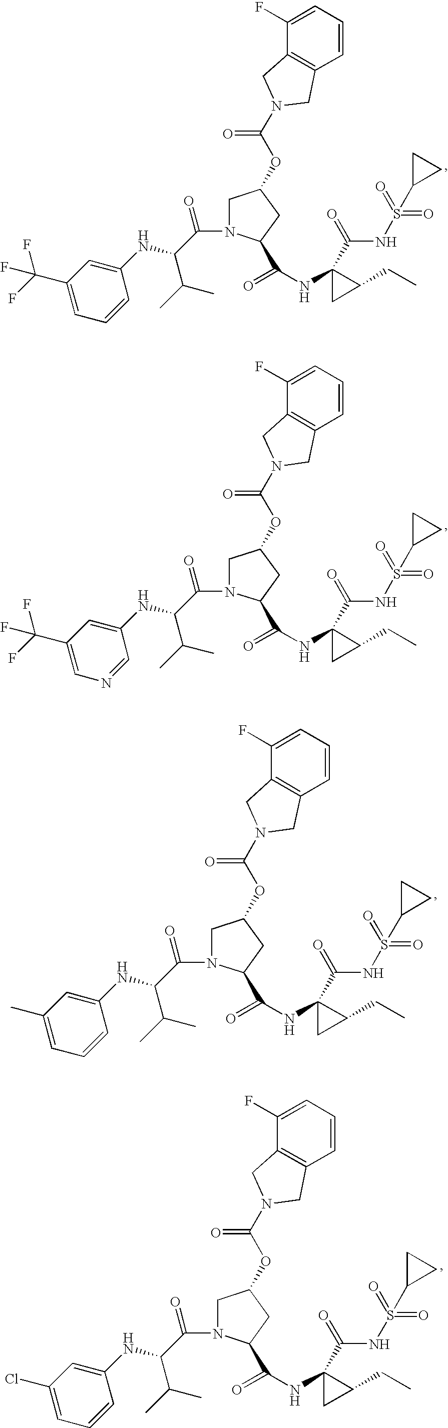

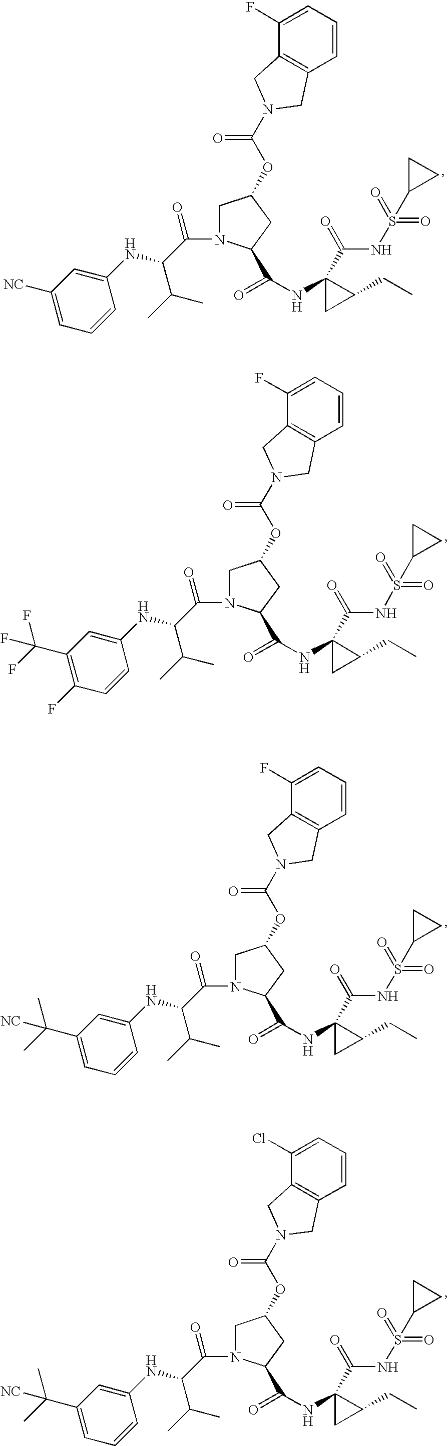

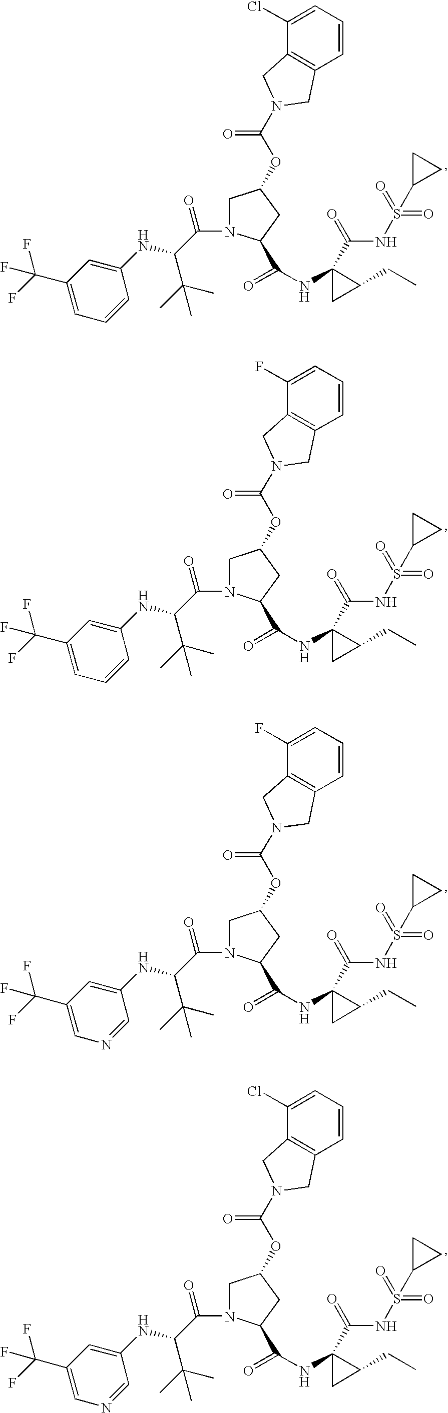

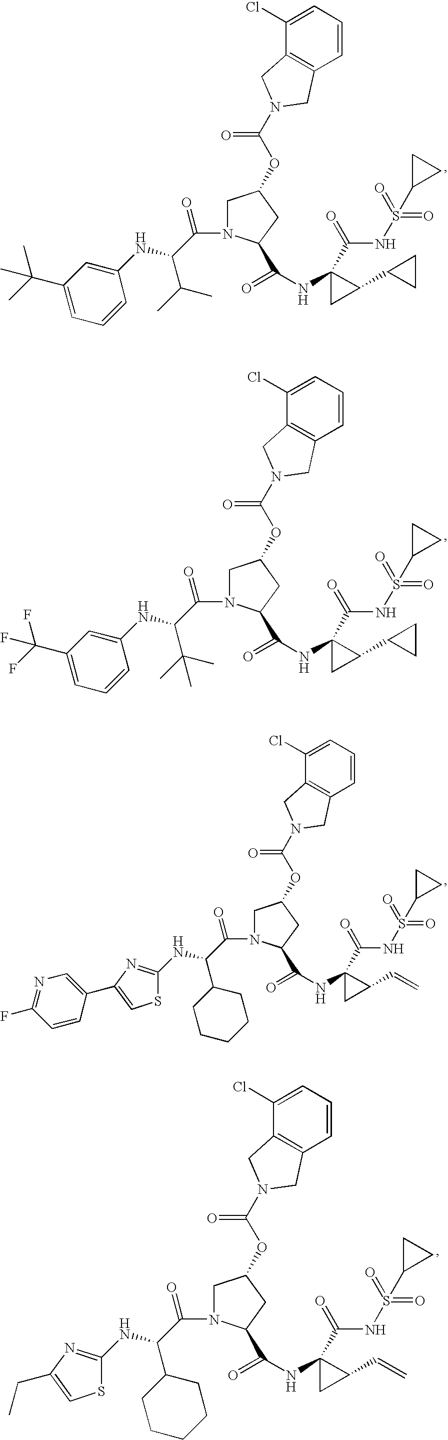

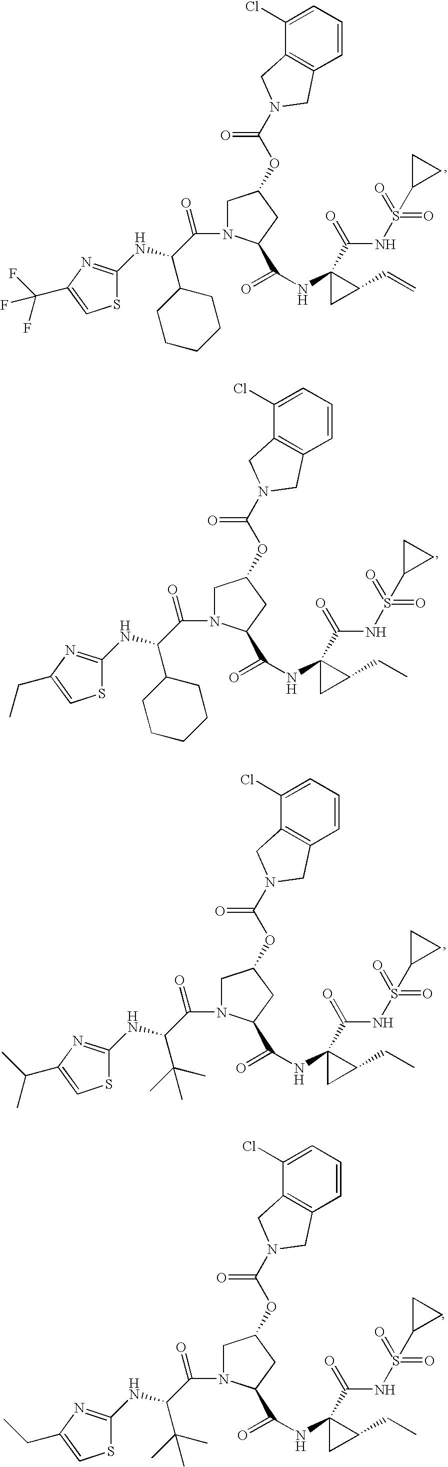

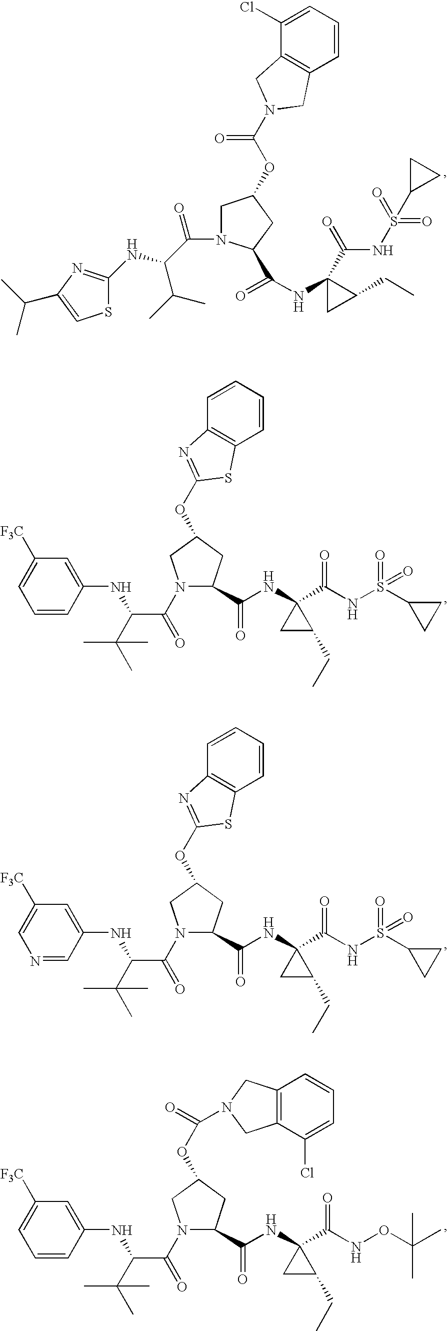

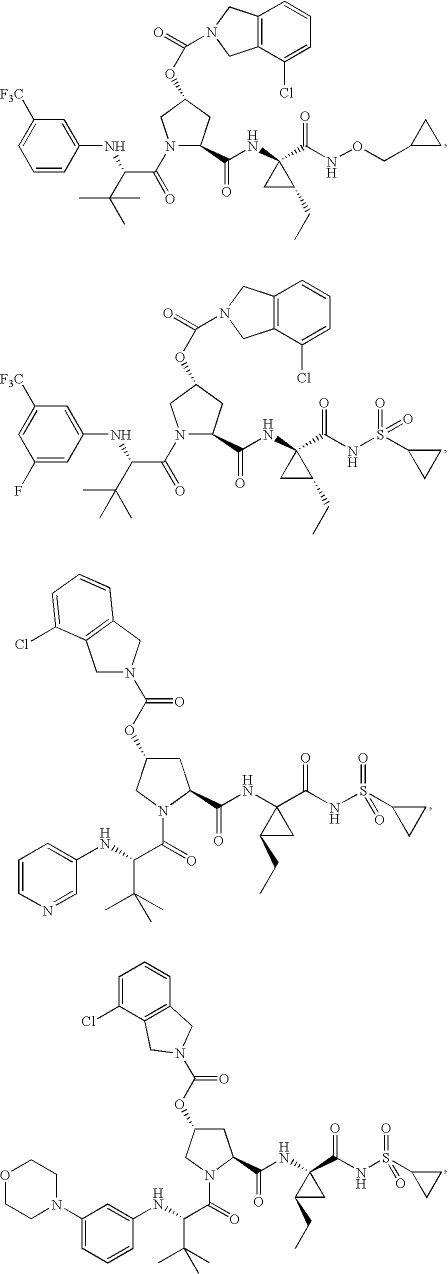

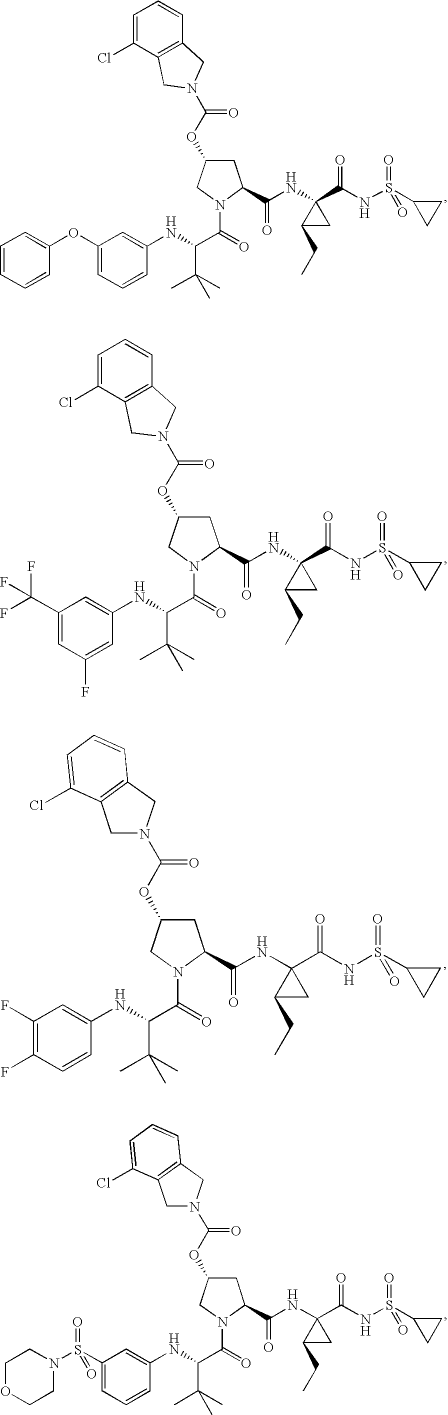

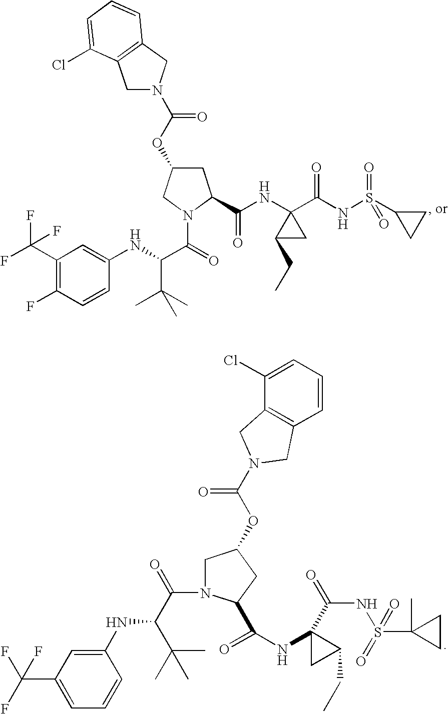

2. The compound of claim 1, wherein z is 0.

3. The compound of claim 2, wherein G is

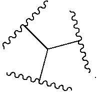

4. The compound of claim 3, wherein Ar is optionally substituted quinolinyl.

5. The compound of claim 4, wherein Ar is optionally substituted quinolin-4-yl.

6. The compound of claim 5, wherein B is optionally substituted phenyl.

7. The compound of claim 6, wherein B is phenyl having from 1 to 3 substituents independently selected from: CF₃, F, Cl, Br, I, C₁₋₃ alkyl, OCH₃, and OCF₃.

8. The compound of claim 5, wherein B is optionally substituted benzooxazol-2-yl.

9. The compound of claim 8, wherein B is benzooxazol-2-yl having from 1 to 3 substituents independently selected from: CF₃, F, Cl, Br, I, C₁₋₃ alkyl, OCH₃, and OCF₃.

10. The compound of claim 5, wherein B is optionally substituted benzothiazol-2-yl.

11. The compound of claim 10, wherein B is benzothiazol-2-yl having from 1 to 3 substituents independently selected from: CF₃, F, Cl, Br, I, C₁₋₃ alkyl, OCH₃, and OCF₃.

12. The compound of claim 5, wherein B is an optionally substituted 5- or 6-membered heteroaryl.

13. The compound of claim 12, wherein B is pyridinyl, imidazolyl, thiazolyl, oxazolyl, thienyl, or furyl; and B has from 1 to 3 substituents independently selected from: CF₃, F, Cl, Br, I, C₁₋₃ alkyl, OCH₃, and OCF₃.

14. The compound of claim 5, wherein D is 1-methylcyclopropyl.

15. The compound of claim 5, wherein D is cyclopropyl.

16. The compound of claim 5, wherein D is N(CH₃)₂.

17. The compound of claim 5, wherein E is C₁₋₆ alkyl.

18. The compound of claim 5, wherein E is ethyl.

19. The compound of claim 5, wherein E is vinyl.

20. The compound of claim 5, wherein E is cyclopropyl.

21. The compound of claim 5, wherein Ar is:

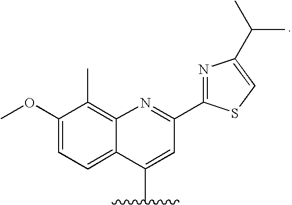

22. The compound of claim 3 further represented by a formula: 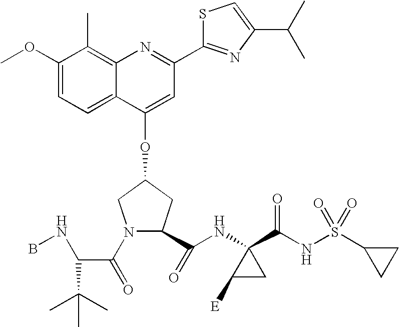
wherein B is optionally substituted benzothiazolyl, optionally substituted benzooxazolyl, optionally substituted phenyl, or an optionally substituted 5- or 6-membered heteroaryl; and
E is ethyl, vinyl, or cyclopropyl.

23. The compound of claim 3, wherein Ar is:

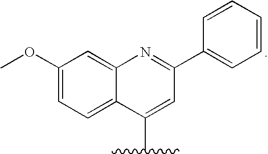

24. The compound of claim 3, wherein Ar is optionally substituted 3-(thiazol-2-yl)isoquinolinyl.

25. The compound of claim 3, wherein Ar is optionally substituted benzothiazol-2-yl, B is optionally substituted phenyl, and D is C₄₋₆ hydrocarbyl.

26. The compound of claim 25, wherein Ar is benzothiazol-2-yl having from 0 to 3 substituents independently selected from: CF₃, F, Cl, Br, I, CH₃, CH₂CH₃, CH₂CH₂CH₃, CH(CH₃)₂, OCH₃, OCF₃, and 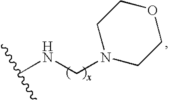
wherein x is 1, 2, or 3.

27. The compound of claims 3, wherein Ar is optionally substituted benzoimidazol-2-yl and B is optionally substituted phenyl.

28. The compound of claim 27, wherein Ar is benzoimidazol-2-yl having from 0 to 3 substituents independently selected from: CF₃, F, Cl, Br, I, CH₃, CH₂CH₃, CH₂CH₂CH₃, CH(CH₃)₂, OCH₃, OCF₃, and 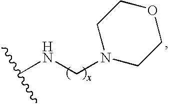
wherein x is 1, 2, or 3.

29. The compound of claim 1, wherein:
Ar is optionally substituted isoindolin-2-yl;
z is 1; and
B is optionally substituted phenyl;
with the proviso that if D is cyclopropyl, then: B is fluorotrifluoro-methylphenyl and E is cyclopropyl.

30. The compound of claim 29, wherein Ar is isoindolin-2-yl having from 0 to 3 substituents independently selected from: CF₃, F, Cl, Br, I, CH₃, CH₂CH₃, CH₂CH₂CH₃, CH(CH₃)₂, OCH₃, OCF₃, and 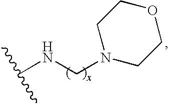
wherein x is 1, 2, or 3.

31. The compound of claim 3, wherein Ar is unsubstituted isoquinolinyl.

32. The compound of claim 1 selected from:

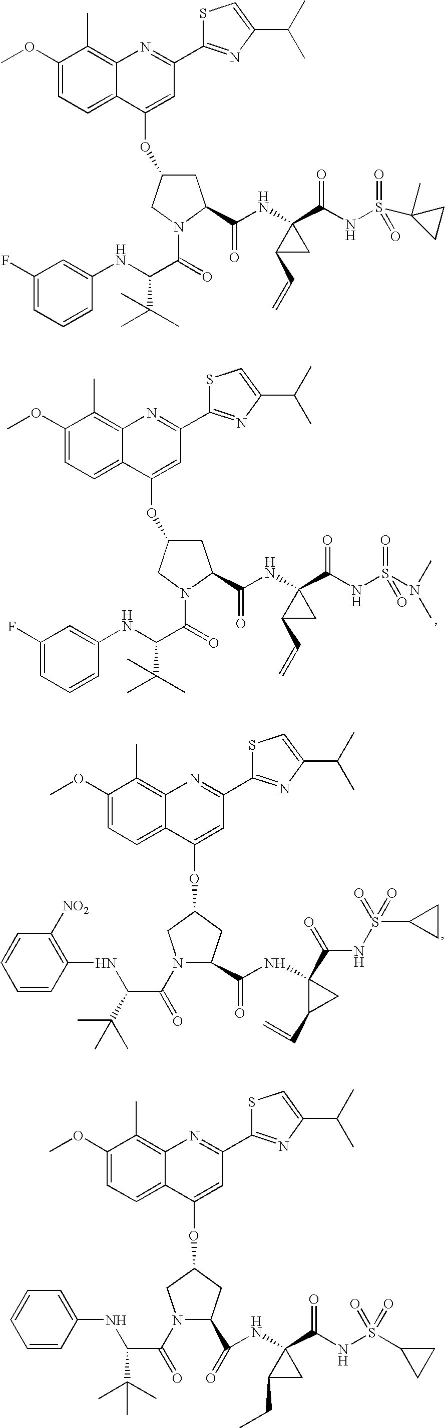

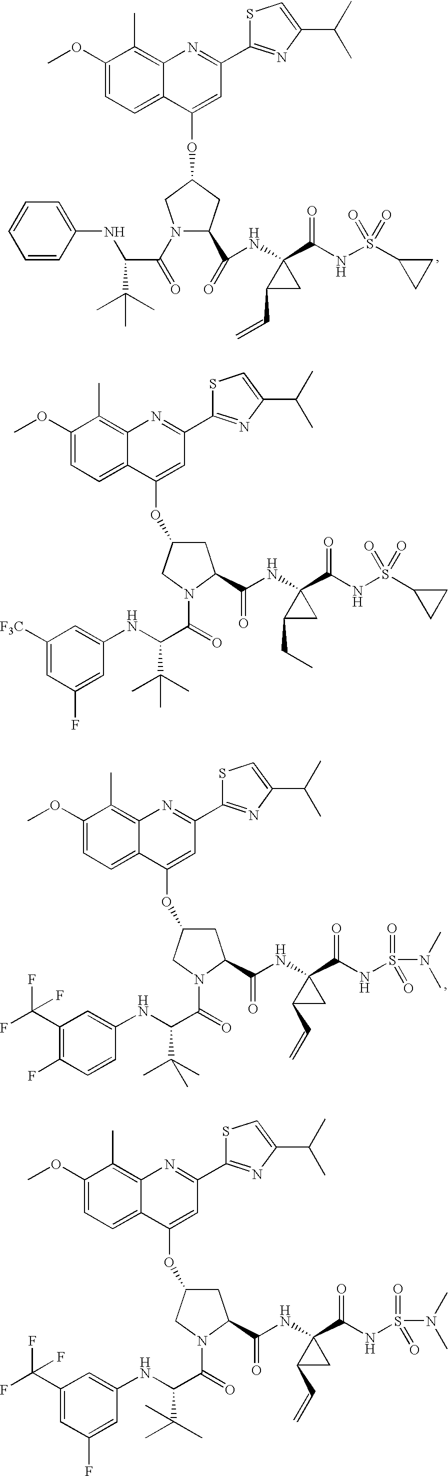

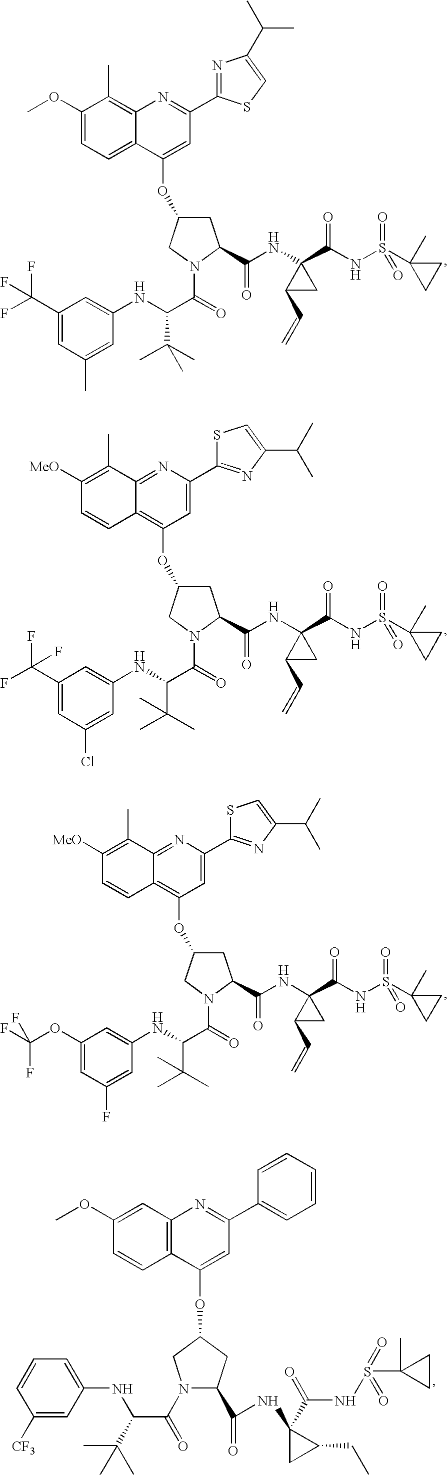

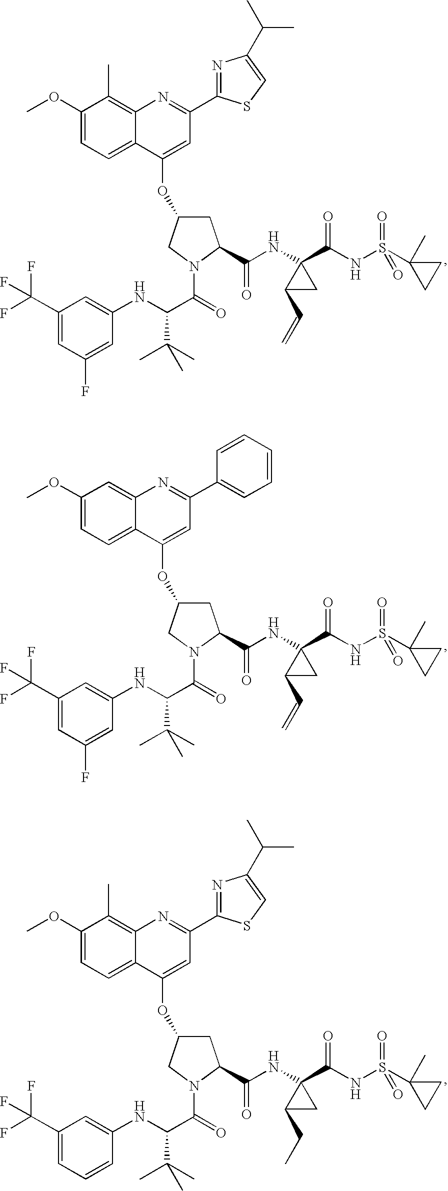

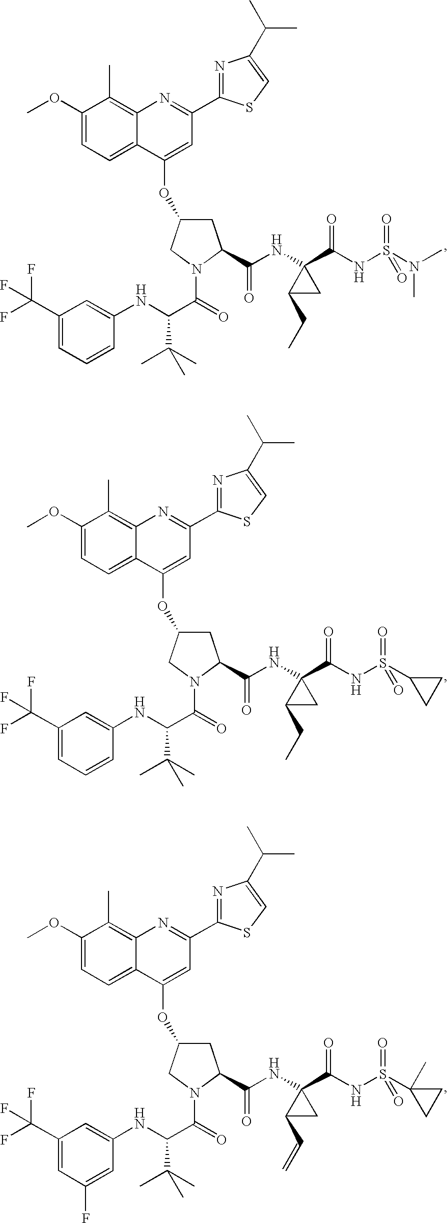

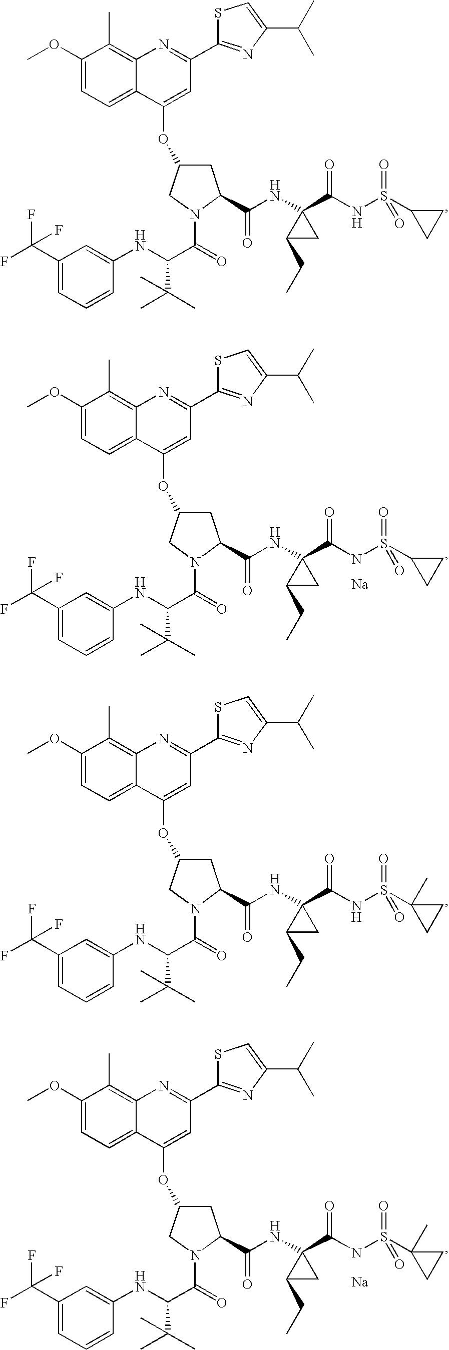

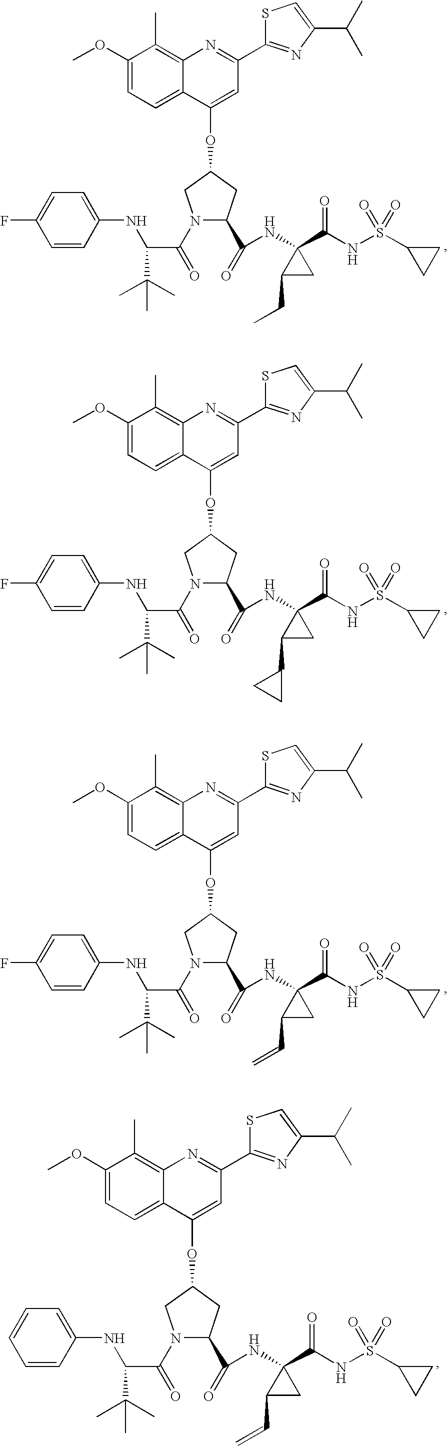

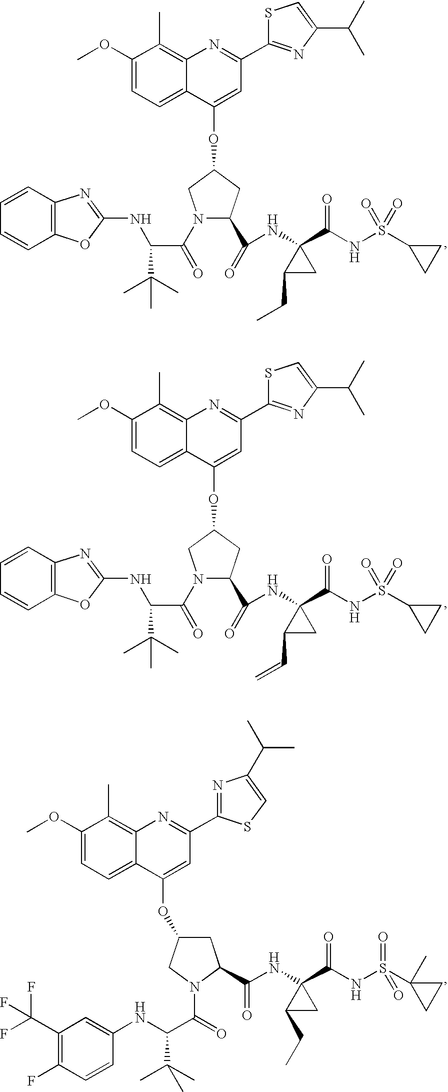

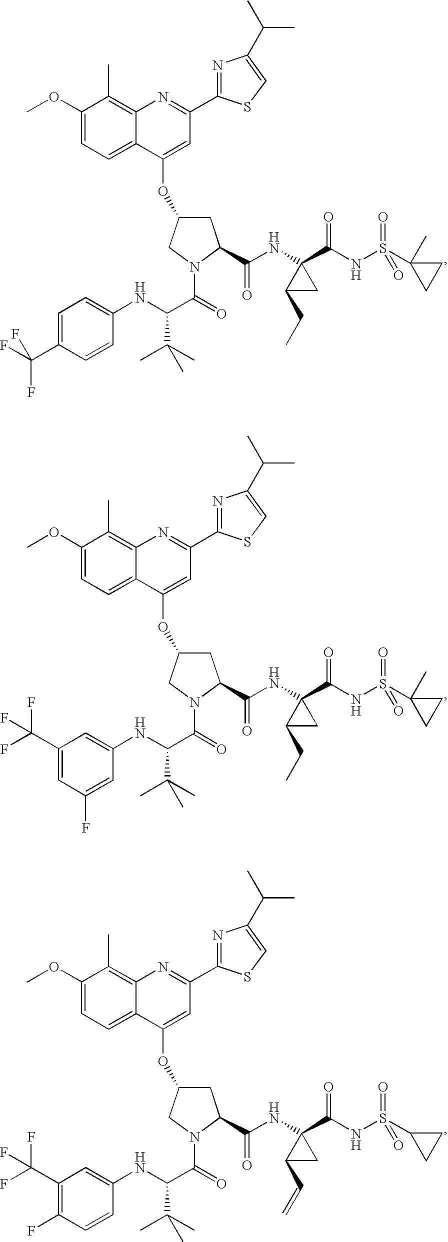

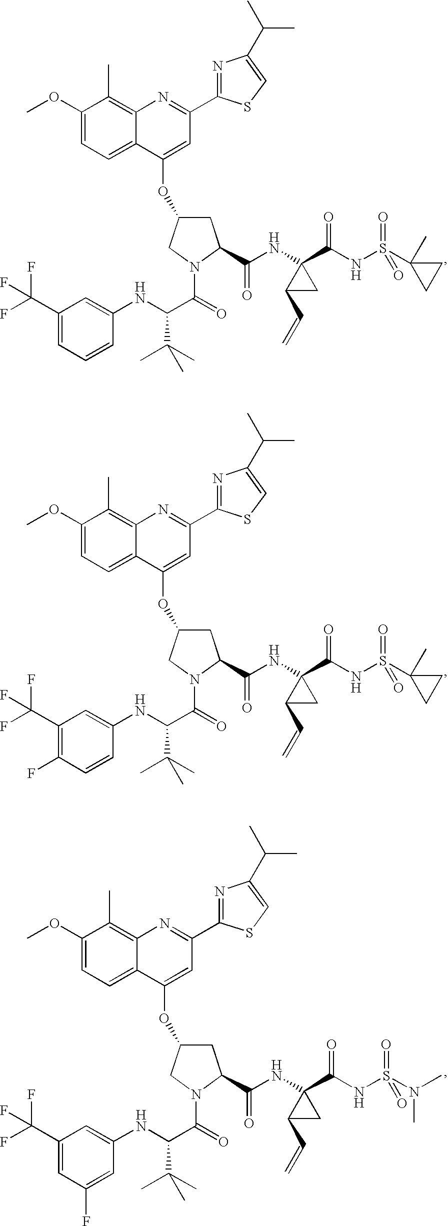

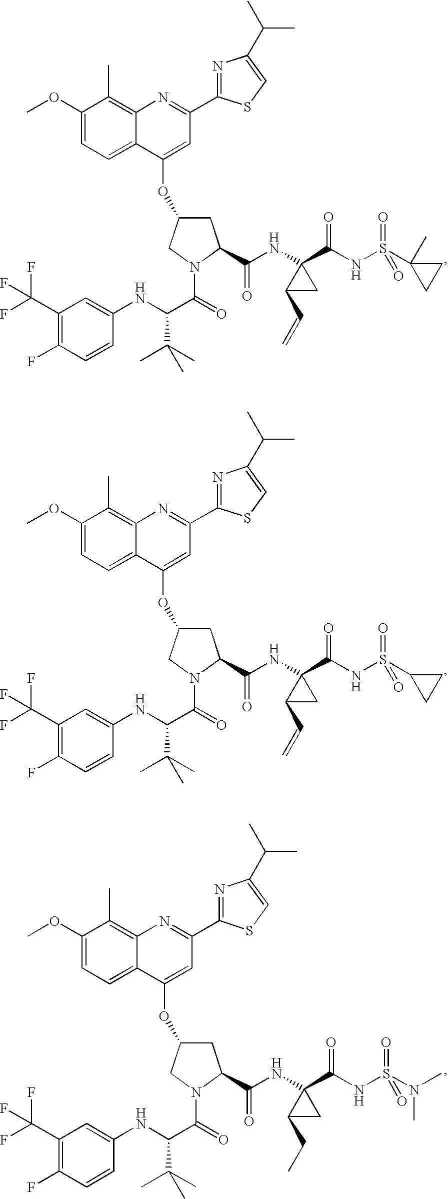

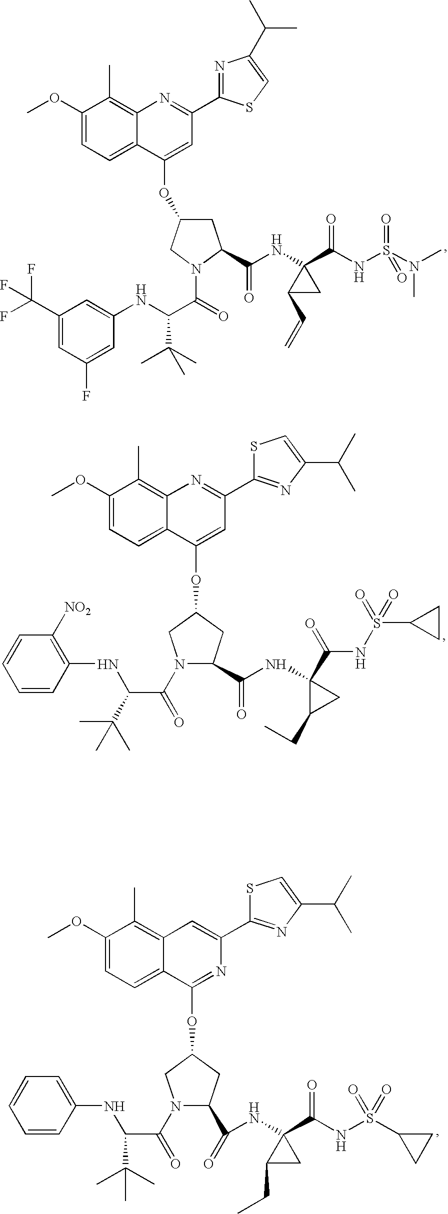

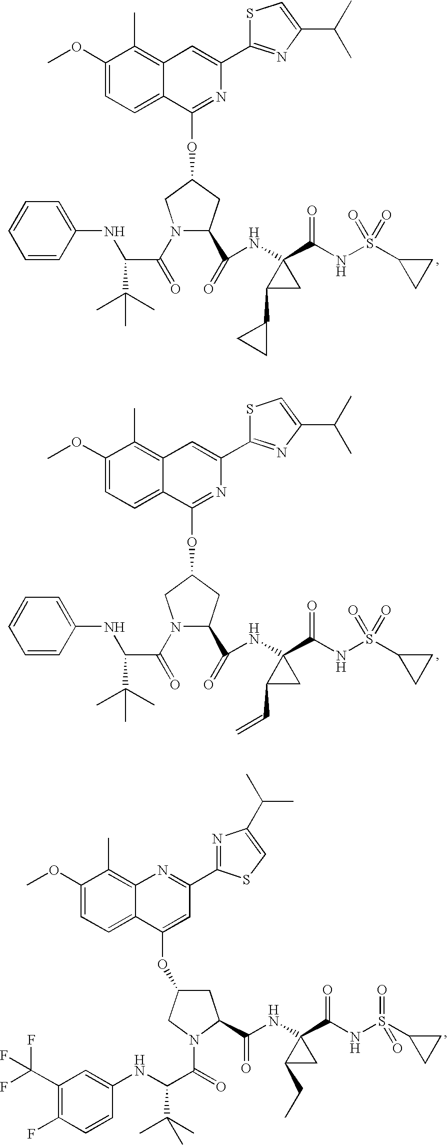

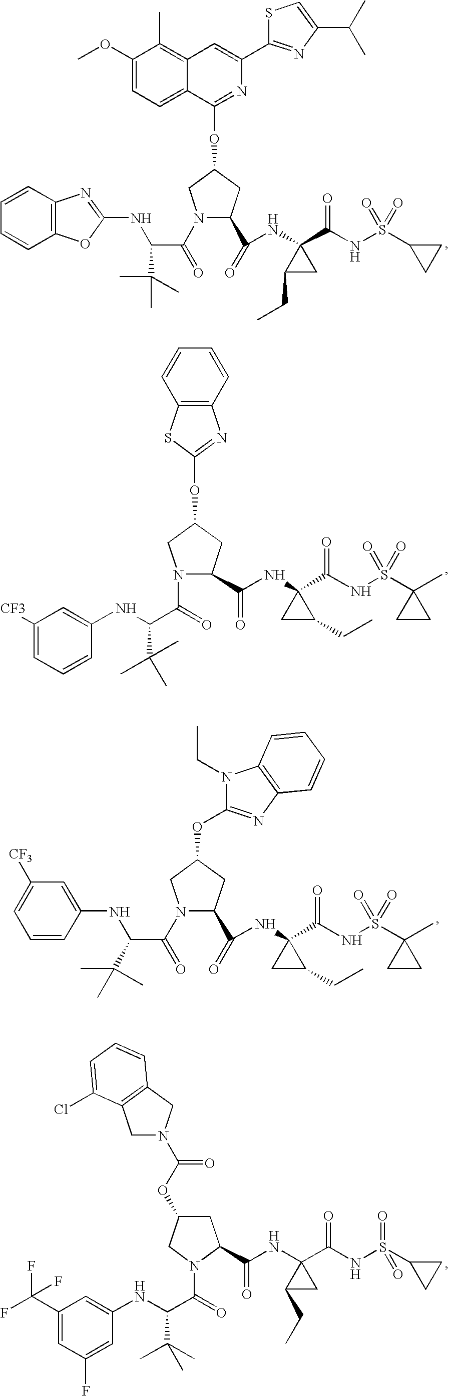

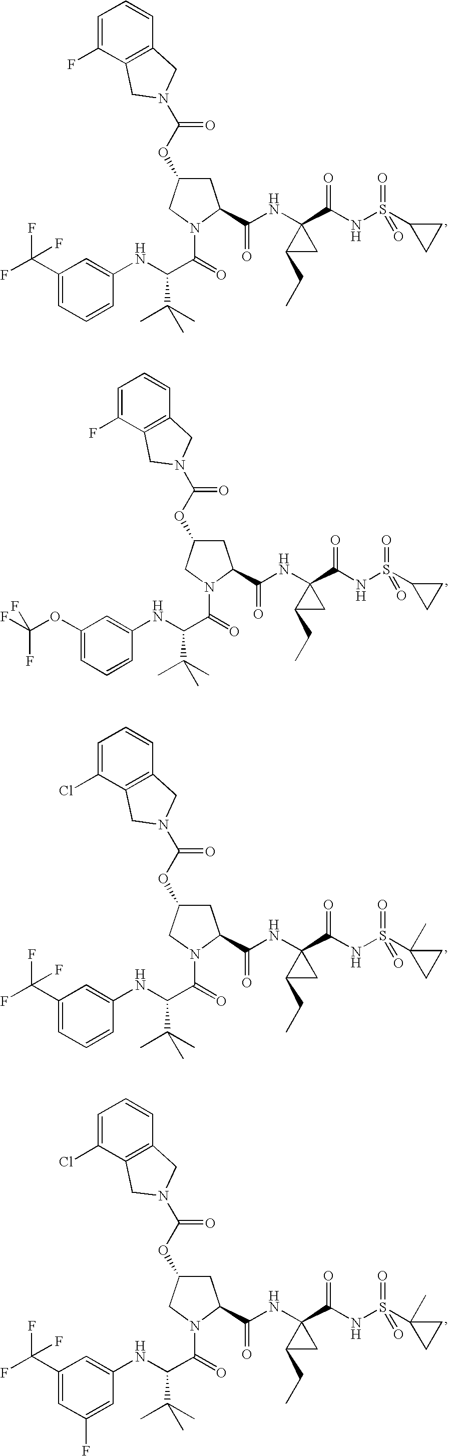

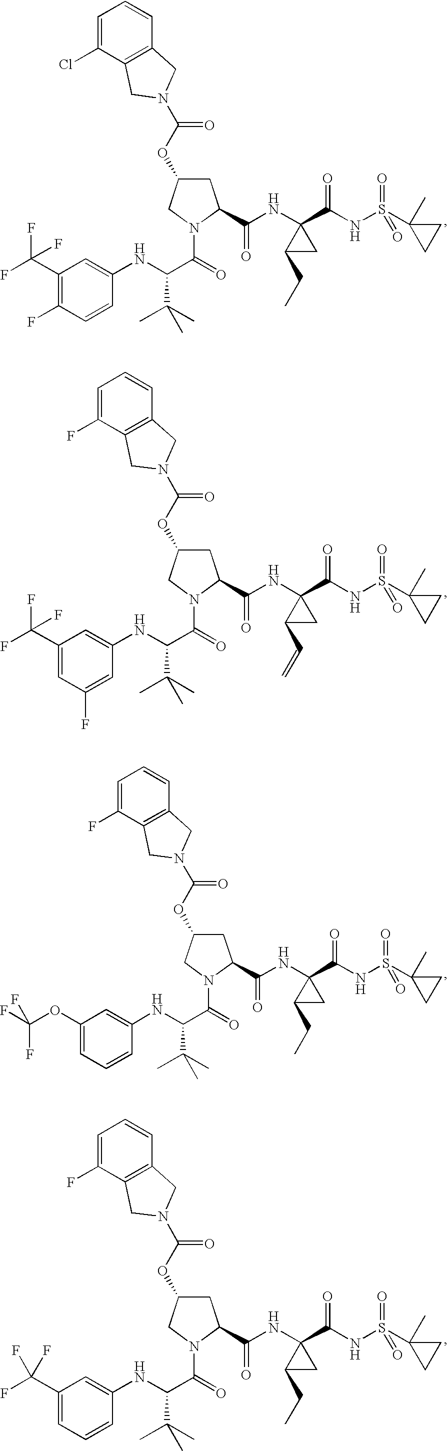

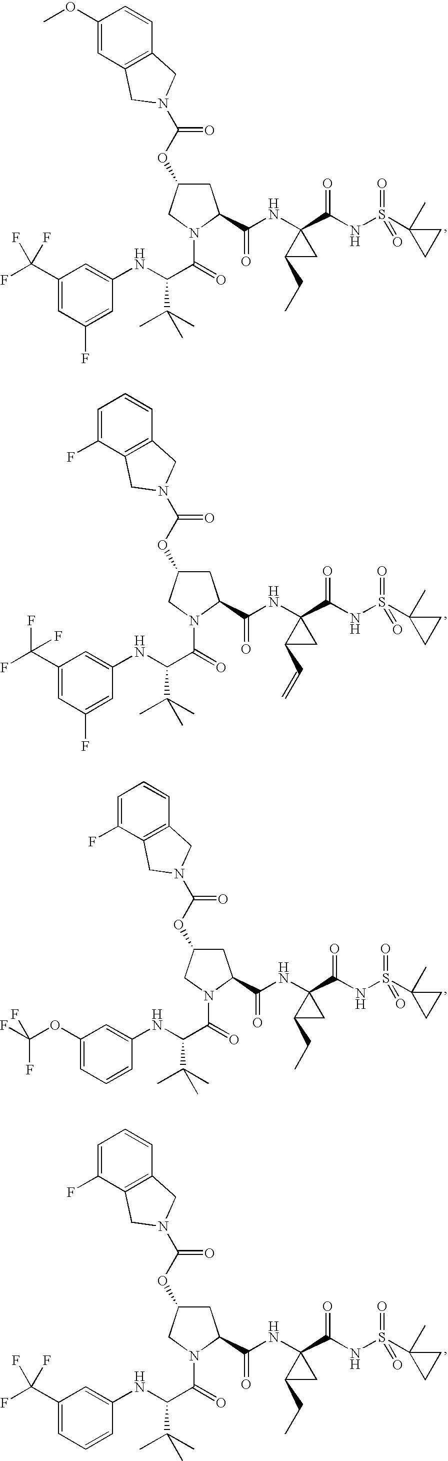

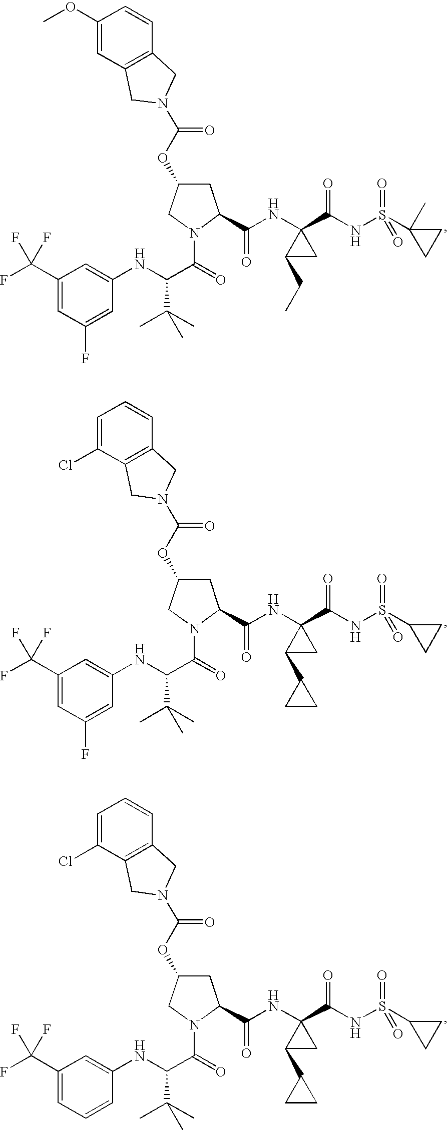

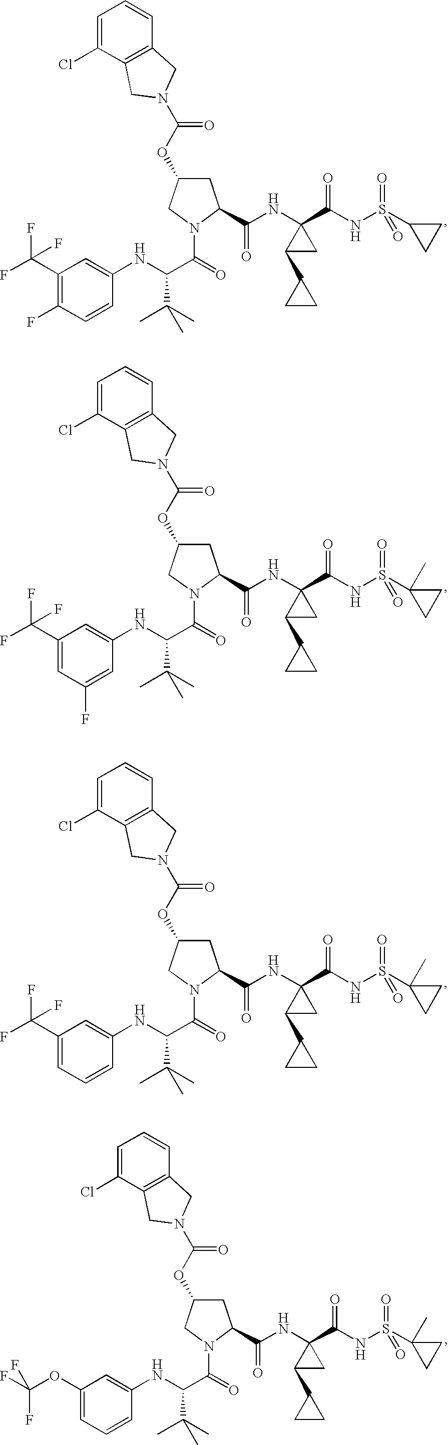

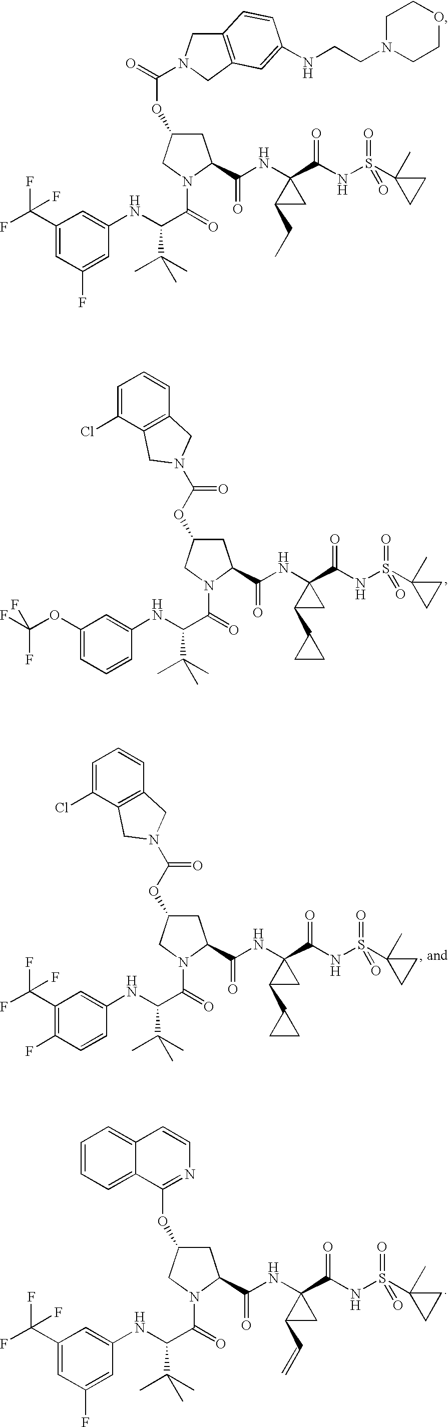

33. The compound of claim 1, further represented by a formula: 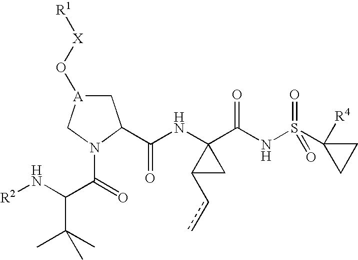
wherein a dashed line represents the presence or absence of a bond;
X is —CO— or a single bond;
R² is aryl or heteroaryl having from 0 to 3 substituents independently selected from: —CO₂H, —CO₂—C₁₋₄-alkyl, halo, —CF₃, —OCF₃, —CN, —CO(CH₂)₂NMe₂, 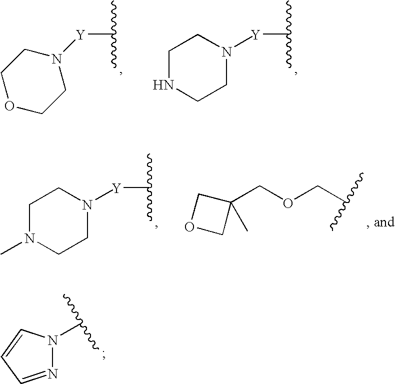
Y is —CO— or —SO₂—;
R⁴ is hydrogen or C₁₋₄ alkyl; and
1) A is 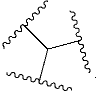
and
R¹ is isoquinolinyl having from 0 to 6 substituents; or isoindolinyl having from 1 to 3 substituents independently selected from —F and —NHCOR³; and
R³ is C₁₋₁₀ alkyl, C₁₋₁₀ alkyl ether, C₁₋₁₀ alkyl amine, or a combination thereof,
provided that if R¹ is 4-fluoroisoindolin-2-yl, R² is not 4-fluorophenyl, 3-trifluoromethylphenyl, or 5-trifluoromethylpyridin-3-yl; or
2) A is 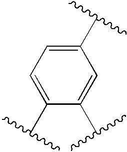
and
R¹ is 3-chlorophenyl,
provided that R⁴ is hydrogen, R² is not 4-fluorophenyl.

34. The compound of claim 33, wherein R² is phenyl having from 0 to 3 substituents independently selected from: —CO₂H, —CO₂CH₃, —CO₂CH₂CH₃, —OCF₃, —CN, —CO(CH₂)₂NMe₂, 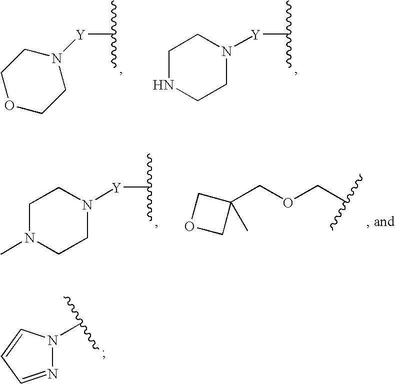
and Y is —CO— or —SO₂—.

35. The compound of claim 33, further represented by a formula:

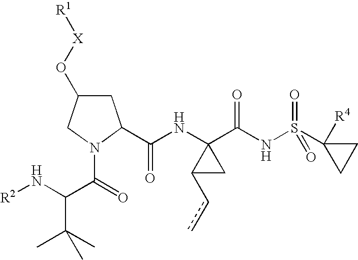

36. The compound of claim 35, further represented by a formula: 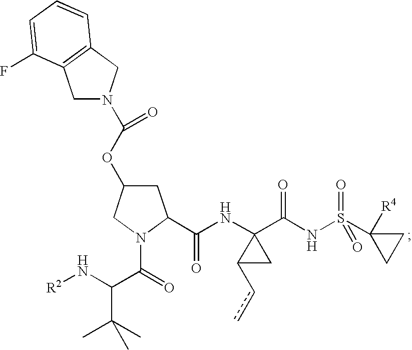
wherein R² is phenyl having from 0 to 3 substituents independently selected from: —CO₂H, —CO₂CH₃, —CO₂CH₂CH₃, —OCF₃, —CN, —CO(CH₂)₂NMe₂, 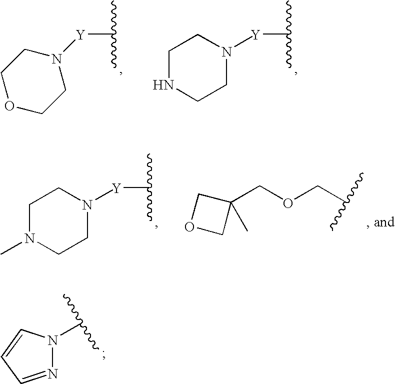
and
Y is —CO— or —SO₂—.

37. The compound of claim 35, further represented by a formula:

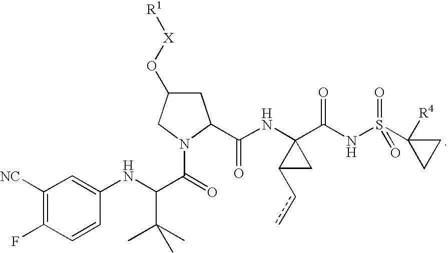

38. The compound of claim 35, further represented by a formula:

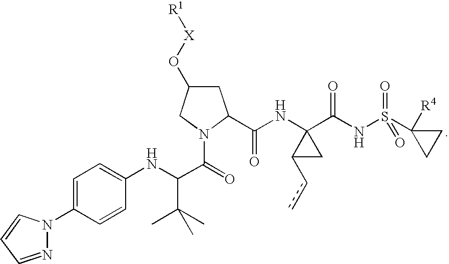

39. The compound of claim 35, further represented by a formula:

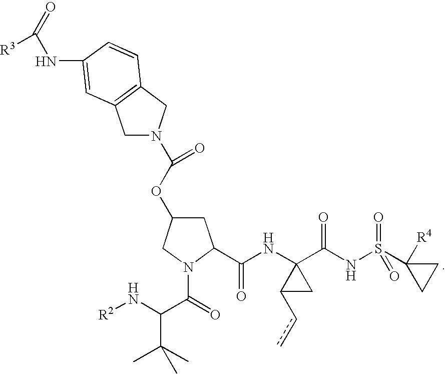

40. The compound of claim 35, further represented by a formula:

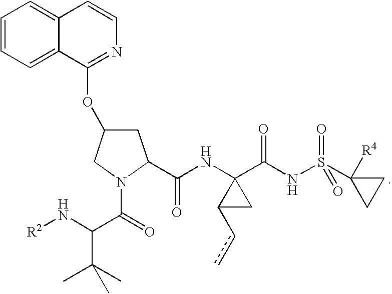

41. The compound of claim 33, wherein R⁴ is hydrogen.

42. The compound of claim 33, further represented by a formula:

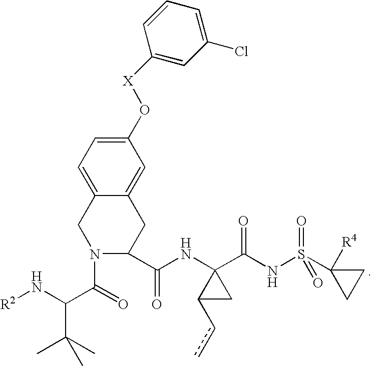

43. The compound of claim 33, wherein X is a single bond.

44. The compound of claim 33 selected from:

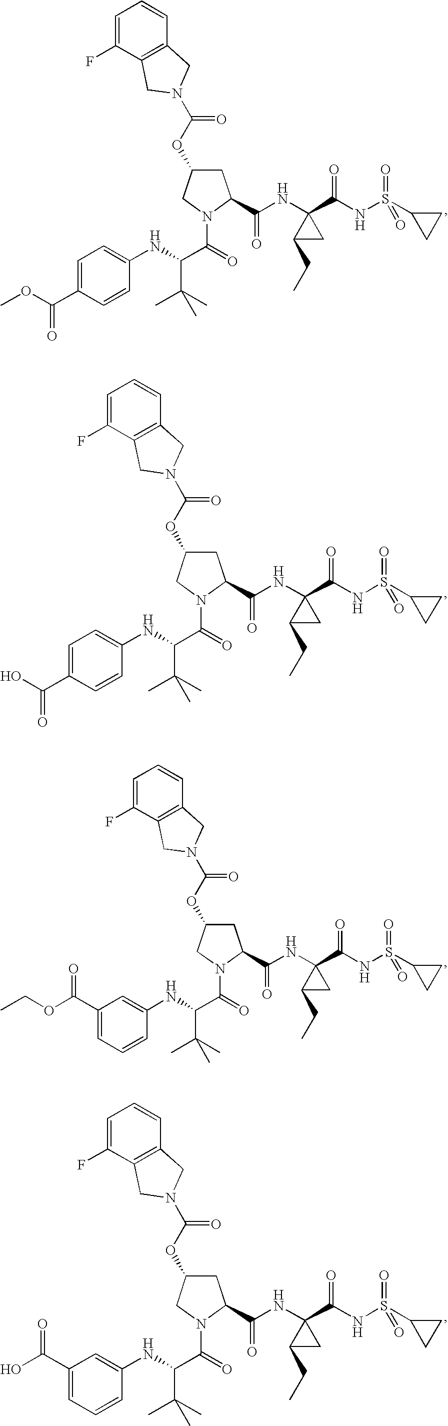

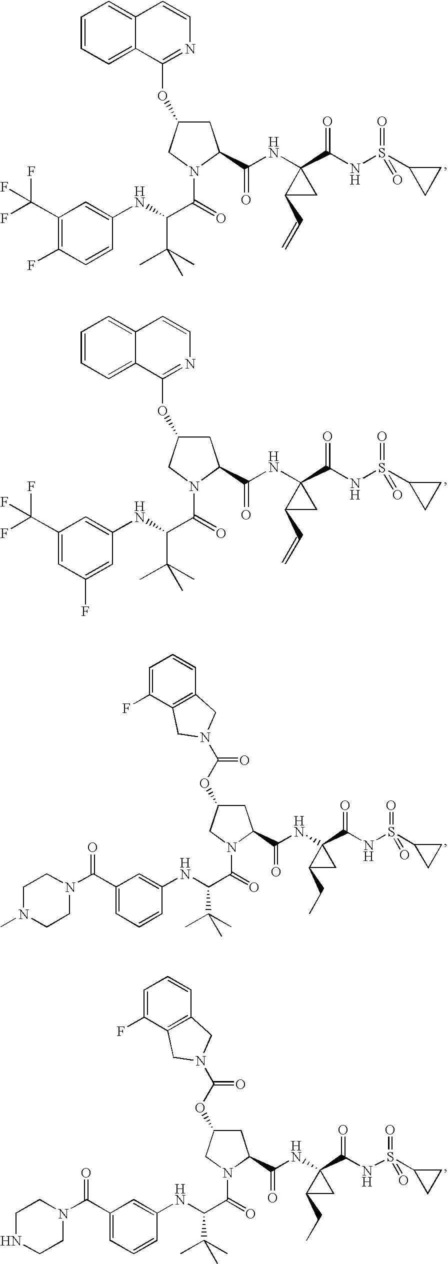

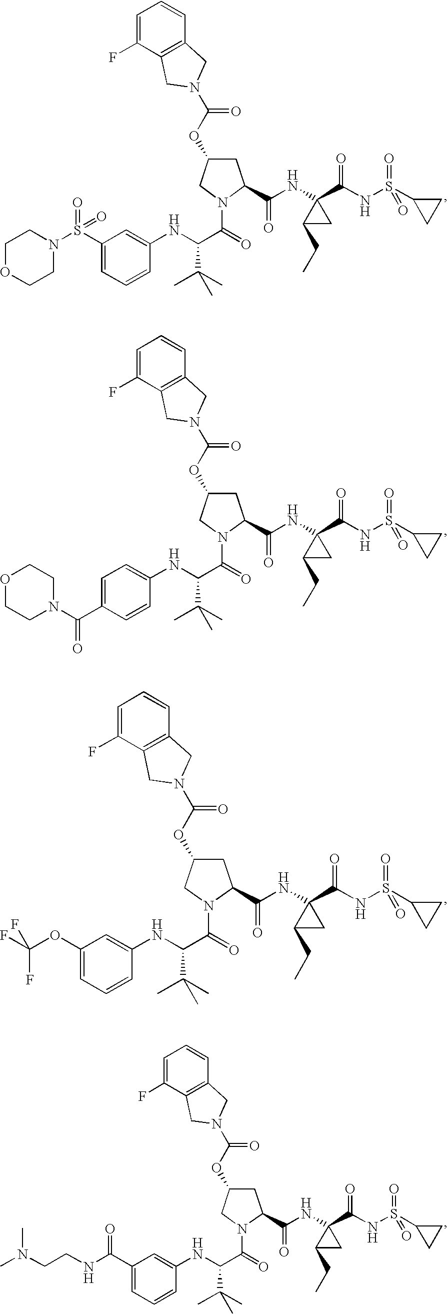

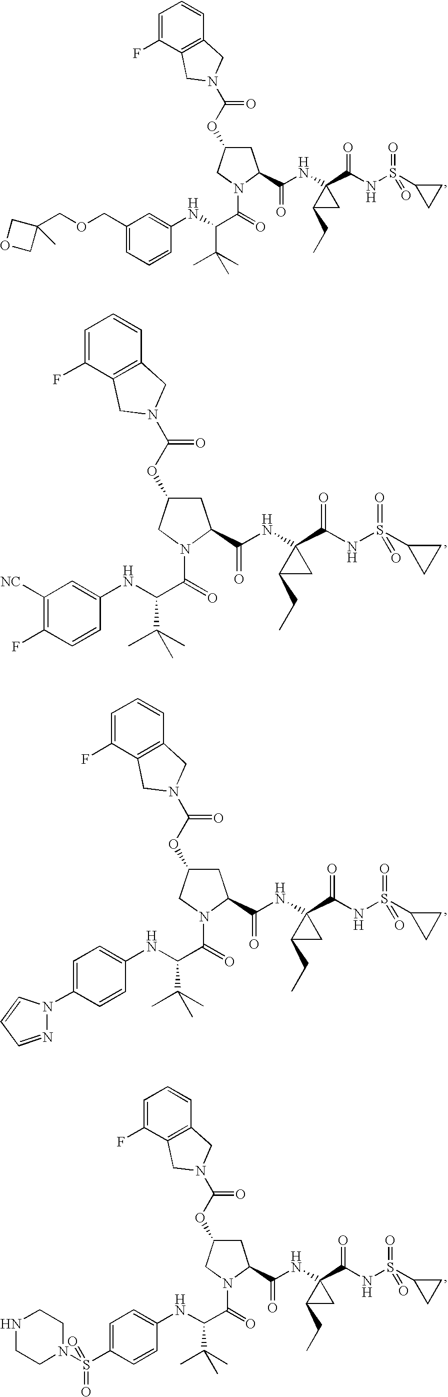

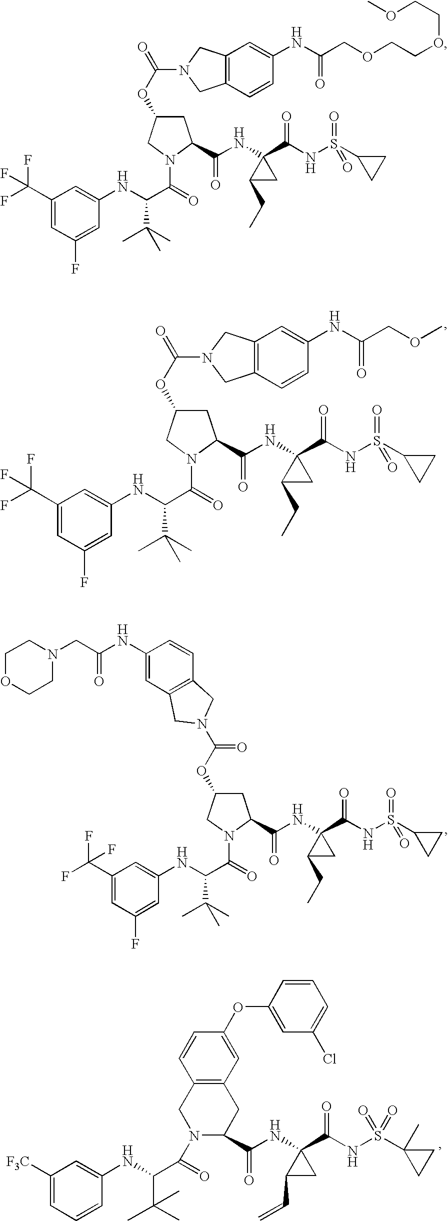

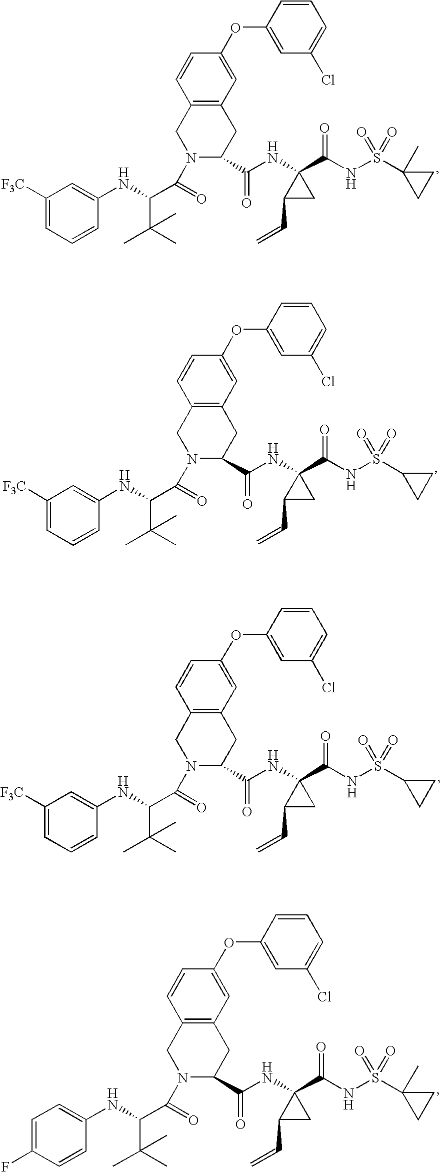

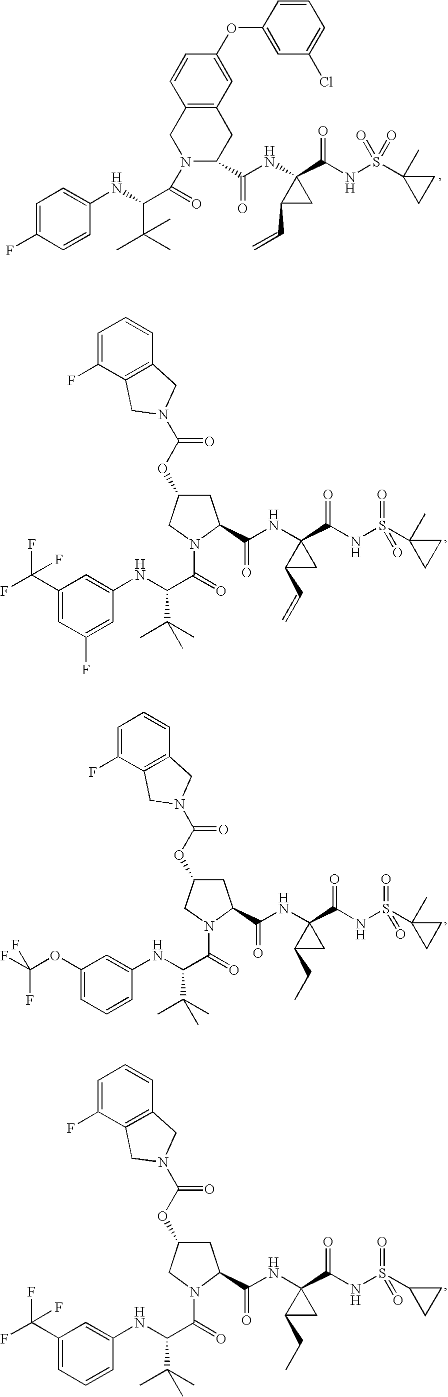

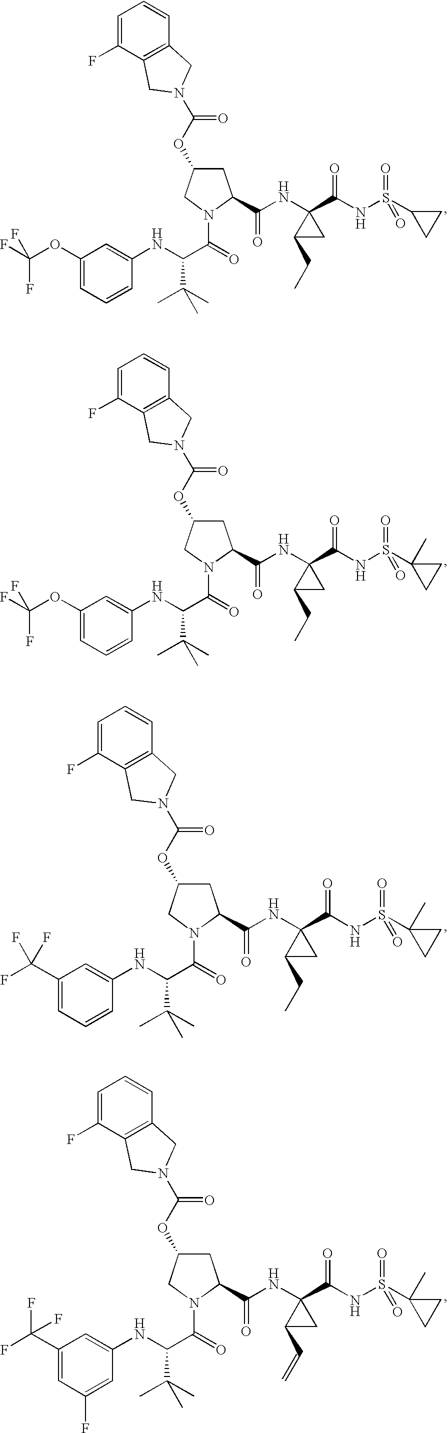

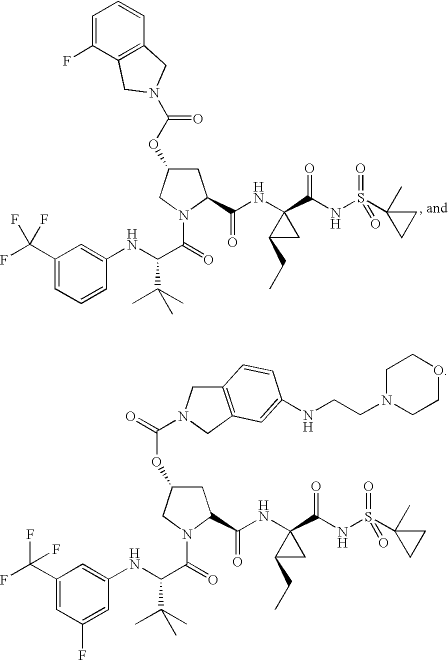

45. The compound of claim 33, further represented by a formula:

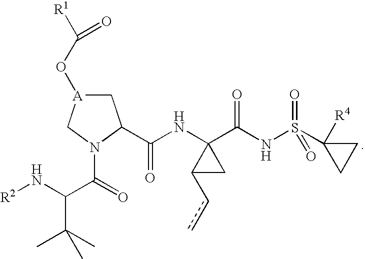

46. The compound of claim 33, further represented by a formula:

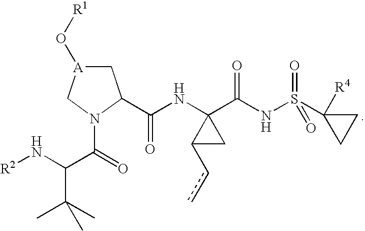

47. A pharmaceutical composition comprising a pharmaceutically acceptable excipient and a compound claim 1.

48. A method of inhibiting NS3/NS4 protease activity comprising contacting a NS3/NS4 protease with a compound of claim 1.

49. The method of claim 48, in which the contacting is conducted in vivo.

50. The method of claim 48, further comprising identifying a subject suffering from a hepatitis C infection and administering the compound to the subject in an amount effective to treat the infection.

51. The method of claim 49, wherein the method further comprises administering to the individual an effective amount of a nucleoside analog.

52. The method of claim 51, wherein the nucleoside analog is selected from ribavirin, levovirin, viramidine, an L-nucleoside, and isatoribine.

53. The method of claim 49, wherein the method further comprises administering to the individual an effective amount of a human immunodeficiency virus 1 protease inhibitor.

54. The method of claim 53, wherein the protease inhibitor is ritonavir.

55. The method of claim 49, wherein the method further comprises administering to the individual an effective amount of an NS5B RNA-dependent RNA polymerase inhibitor.

56. The method of claim 49, wherein the method further comprises administering to the individual an effective amount of interferon-gamma (IFN-γ).

57. The method of claim 56, wherein the IFN-γ is administered subcutaneously in an amount of from about 10 μg to about 300 μg.

58. The method of claim 48, wherein the method further comprises administering to the individual an effective amount of interferon-alpha (IFN-α).

59. The method of claim 58, wherein the IFN-α is monoPEG-ylated consensus IFN-α administered at a dosing interval of every 8 days to every 14 days.

60. The method of claim 58, wherein the IFN-α is monoPEG-ylated consensus IFN-α administered at a dosing interval of once every 7 days.

61. The method of claim 58, wherein the IFN-α is INFERGEN consensus IFN-α.

62. The method of claim 49, further comprising administering an effective amount of an agent selected from 3′-azidothymidine, 2′,3′-dideoxyinosine, 2′,3′-dideoxycytidine, 2-,3-didehydro-2′,3′-dideoxythymidine, combivir, abacavir, adefovir dipoxil, cidofovir, and an inosine monophosphate dehydrogenase inhibitor.

63. The method of claim 49, wherein a sustained viral response is achieved.

64. The method of claim 48, in which the contacting is conducted ex vivo.

65. A method of treating liver fibrosis in an individual, the method comprising administering to the individual an effective amount of a compound of claim 1.

66. The method of claim 65, wherein the method further comprises administering to the individual an effective amount of a nucleoside analog.

67. The method of claim 66, wherein the nucleoside analog is selected from ribavirin, levovirin, viramidine, an L-nucleoside, and isatoribine.

68. The method of claim 65, wherein the method further comprises administering to the individual an effective amount of a human immunodeficiency virus 1 protease inhibitor.

69. The method of claim 68, wherein the protease inhibitor is ritonavir.

70. The method of claim 65, wherein the method further comprises administering to the individual an effective amount of an NS5B RNA-dependent RNA polymerase inhibitor.

71. The method of claim 65, wherein the method further comprises administering to the individual an effective amount of interferon-gamma (IFN-γ).

72. The method of claim 71, wherein the IFN-γ is administered subcutaneously in an amount of from about 10 μg to about 300 μg.

73. The method of claim 65, wherein the method further comprises administering to the individual an effective amount of interferon-alpha (IFN-α).

74. The method of claim 73, wherein the IFN-α is monoPEG-ylated consensus IFN-α administered at a dosing interval of every 8 days to every 14 days.

75. The method of claim 73, wherein the IFN-α is monoPEG-ylated consensus IFN-α administered at a dosing interval of once every 7 days.

76. The method of claim 73, wherein the IFN-α is INFERGEN consensus IFN-α.

77. The method of claim 65, further comprising administering an effective amount of an agent selected from 3′-azidothymidine, 2′,3′-dideoxyinosine, 2′,3′-dideoxycytidine, 2-,3-didehydro-2′,3′-dideoxythymidine, combivir, abacavir, adefovir dipoxil, cidofovir, and an inosine monophosphate dehydrogenase inhibitor.

78. A method of increasing liver function in an individual having a hepatitis C virus infection, the method comprising administering to the individual an effective amount of a compound of claim 1.

79. The method of claim 78, wherein the method further comprises administering to the individual an effective amount of a nucleoside analog.

80. The method of claim 79, wherein the nucleoside analog is selected from ribavirin, levovirin, viramidine, an L-nucleoside, and isatoribine.

81. The method of claim 78, wherein the method further comprises administering to the individual an effective amount of a human immunodeficiency virus 1 protease inhibitor.

82. The method of claim 81, wherein the protease inhibitor is ritonavir.

83. The method of claim 78, wherein the method further comprises administering to the individual an effective amount of an NS5B RNA-dependent RNA polymerase inhibitor.

84. The method of claim 78, wherein the method further comprises administering to the individual an effective amount of interferon-gamma (IFN-γ).

85. The method of claim 84, wherein the IFN-γ is administered subcutaneously in an amount of from about 10 μg to about 300 μg.

86. The method of claim 78, wherein the method further comprises administering to the individual an effective amount of interferon-alpha (IFN-α).

87. The method of claim 86, wherein the IFN-α is monoPEG-ylated consensus IFN-α administered at a dosing interval of every 8 days to every 14 days.

88. The method of claim 86, wherein the IFN-α is monoPEG-ylated consensus IFN-α administered at a dosing interval of once every 7 days.

89. The method of claim 86, wherein the IFN-α is INFERGEN consensus IFN-α.

90. The method of claim 78, further comprising administering an effective amount of an agent selected from 3′-azidothymidine, 2′,3′-dideoxyinosine, 2′,3′-dideoxycytidine, 2-,3-didehydro-2′,3′-dideoxythymidine, combivir, abacavir, adefovir dipoxil, cidofovir, and an inosine monophosphate dehydrogenase inhibitor.
